(19) United States
(12) Patent Application Publication
(10) Pub. No.: US 2026/0199395 A1
(43) Pub. Date: Jul. 16, 2026

(54) COMPOSITIONS AND METHODS FOR MODULATING GENE EXPRESSION OR GENE SIGNALING FOR NON-IMMUNOGENICITY

(71) Applicant: REPLAY HOLDINGS, INC., San Diego, CA (US)

(72) Inventors: Anthony CONWAY, San Diego, CA (US); Renata MARTIN, San Diego, CA (US); Jaimeson VELDHUIZEN, San Diego, CA (US)

(21) Appl. No.: 19/138,065

(22) PCT Filed: Dec. 12, 2023
(86) PCT No.: PCT/US2023/083690

§ 371 Date: Jun. 11, 2025

(30) Foreign Application Priority Data
Dec. 13, 2022 (GB) 2218755.3

(51) Int. Cl. A61K 35/17; A61K 40/11; A61K 40/13; A61K 40/15; A61K 40/31; A61K 40/32; A61K 40/50; C07K 14/725; C12N 5/0781; C12N 5/0783; C12N 15/85

(52) U.S. Cl. CPC A61K 35/17; A61K 40/11; A61K 40/13; A61K 40/15; A61K 40/31; A61K 40/32; A61K 40/50; C07K 14/7051; C12N 5/0635; C12N 5/0636; C12N 5/0646; C12N 15/85; C12N 2506/02; C12N 2506/11; C12N 2506/1346; C12N 2506/45; C12N 2830/34

(57) ABSTRACT

An aspect of the present disclosure relates to methods and constructs for modulating gene expression in a cell. In some cases, the constructs are configured to drive expression of one or more genes (e.g., transgenes comprising a tolerogenic factor,) off inducible gene promoters. In some embodiments, the cell-specific promoter is an immune cell gene promoter. In some embodiments, the cell-specific promoter comprises a sequence having 95%, 97%, 99% or 100% sequence identity to a 60, 70, 80, 90, 100, 200, 500, 1,000, 1,500 or 2,000 base pair long fragment of any one of SEQ ID NO: 5 to 57.

### INCORPORATION BY REFERENCE

This application claims benefit of UK Patent Application 2218755.3, titled Compositions and Methods for Non-Immunogenicity, filed in Great Britain on Dec. 13, 2022, which is hereby incorporated by reference in its entirety.

### BACKGROUND OF THE INVENTION

Cellular therapeutics are expensive and complicated to manufacture and require a certain level of product characterization and consistency to establish efficacy. Stem cell derived cellular therapeutics (including iPSC) may pass through multiple cell types in their manufacturing and therapeutic journey; some of which are particularly efficacious and some which may not be. Since cells grow, divide, and change during their lifespan, even once administered the administered cells may be (or may become) a heterologous population; with some cells at one stage of development or therapeutic efficacy, and others at another. Further, upon administration, the subject's own immune system or co-administered cells may be attack, lyse, and “eat” the cellular therapeutic before it has the opportunity to localize to the appropriate compartment and treat the disease. Accordingly, there is a need for mechanisms to fully characterize the therapeutic product, increase the targeted cell populations and avoid off-target or non-specific immune activity both in vitro during manufacture and in vivo after administration to a subject.

### SUMMARY

The present disclosure relates to methods and constructs for modulating gene expression in a cell. In some cases, the constructs are configured to drive expression of one or more genes (e.g., transgenes comprising a tolerogenic factor,) off inducible gene promoters. Some embodiments include methods for gene expression in a cell, said methods comprising: differentiating a cell comprising: an inducible promoter configured to modulate an expression of one or more tolerogenic factor(s); an open reading frame (ORF) encoding said tolerogenic factor(s) in operable connection with said inducible promoter; and to conditions sufficient to differentiate said cell into a differentiated cell; wherein concurrent with or subsequent to such differentiation, said inducible promoter is activated in said differentiated cell to modulate said expression of or gene signaling via said tolerogenic factor(s). In one embodiment said inducible promoter is delivered exogenously to said cell. In one embodiment said inducible promoter is a cell-specific promoter.

In some embodiments of the methods above, the cell-specific promoter is an immune cell gene promoter. In one embodiment the cell-specific promoter is a white blood cell gene promoter. In some cases, the cell-specific promoter is a neutrophil gene promoter, an eosinophil gene promoter, a basophil gene promoter, a mast cell gene promoter, a monocyte gene promoter, a macrophage gene promoter, a dendritic cell gene promoter, a natural killer (NK) cell gene promoter, a memory-like NK cell gene promoter, a lymphocyte gene promoter, a B cell gene promoter, a T-cell gene promoter, a regulatory T-cell (Treg) promoter, a hepatocyte gene promoter gene promoter, a cardiomyocyte gene promoter, a renal cell gene promoter, a dopaminergic neuron gene promoter, a pancreatic islet cell gene promoter, a macrophage gene promoter, or a retinal pigment epithelium cell gene promoter. In one aspect the cell-specific promoter is an NK cell gene promoter or a T-cell gene reporter. In one embodiment the inducible promoter is an L selectin (CD62L) promoter, an interferon-gamma (IFNg) promoter, neural cell adhesion molecule (CD56) promoter, a CD56 promoter, a KIRs promoter, a CD16 promoter, a NKp44 promoter, a NKp46 promoter, a NKG2D promoter, a TRAIL promoter, a CD122 promoter, a CD27 promoter, a CD244 promoter, a NK1.1 promoter, a NKG2A/C promoter, a NCR1 promoter, a Ly49 promoter, a CD49b promoter, a CD11b promoter, a KLRG1 promoter, a CD43 promoter, a CD62L promoter, a CD226 promoter, a TRAC promoter, a TRBC promoter, a CD3 promoter, a CD4 promoter, a ThPOK promoter, a CD8 promoter, a FOXP3 promoter, a Helios promoter, a CD25 promoter, a GARP promoter, a GPA33 promoter, a CD14 promoter, a CD11b promoter, a CD68 promoter, a CD138 promoter, an IgH promoter, an IgK promoter, an IgL promoter, an IgG promoter, a CD19 promoter, a CD20 promoter, a TH promoter, a TUBB3 promoter, a FOXA2 promoter, a GIRK2 promoter, a Nurr1 promoter, a DAT promoter, an INS promoter, a chromogranin A promoter, a synaptophysin promoter, a neuron-specific enolase promoter, or a Leu7 promoter. In a particular embodiment, the cell-specific promoter is a memory-like NK cell gene promoter. In one embodiment, the cell-specific promoter is an L selectin (CD62L) promoter, an interferon-gamma (IFNg) promoter, or neural cell adhesion molecule (CD56) promoter. In one embodiment the cell-specific promoter is a CD16 promoter, a HLA-C promoter, a NKp46 promoter, or a DAP10 promoter.

In one embodiment the cell-specific promoter comprises a sequence having 95%, 97%, 99% or 100% sequence identity to a 60, 70, 80, 90, 100, 200, 500, 1,000, 1,500 or 2,000 base pair long fragment of any one of SEQ ID NO: 5 to 57. In one embodiment the cell-specific promoter comprises a sequence having at least 95% sequence identity to a sequence that comprises 500 bp of any one of SEQ ID NO: 5 to 57. In one embodiment the cell-specific promoter comprises a sequence having at least 95% sequence identity to a sequence that comprises 500 bp of any one of SEQ ID NO: 54 to 57. In one embodiment the cell-specific promoter comprises a sequence having at least 99% sequence identity to a sequence that comprises 500 bp of any one of SEQ ID NO: 54 to 57. In one embodiment the cell-specific promoter comprises a sequence having at least 99% sequence identity to a sequence that comprises 500 bp of any one of SEQ ID NO: 54 to 57.

In one embodiment the inducible promoter exists endogenously within said stem cell.

In one embodiment said tolerogenic factor comprises a SIRPα binding sequence. In one embodiment said SIRPα binding sequence is a CD47 sequence or functional variant thereof.

In one embodiment said SIRPα binding sequence comprises a sequence having at least about 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identity to any one of SEQ ID NOs: 1-653.

In one embodiment said tolerogenic factor comprises CD47, cluster of differentiation 24 (CD24), complement receptor 1 (CR1), complement decay-accelerating factor (CD55), cluster of differentiation 46 (CD46), cluster of differentiation 59 (CD59), HLA-A, HLA-B, HLA-C, HLA-E, HLA-F, HLA-G, cluster of differentiation 39 (CD39), cluster of differentiation 73 (CD73), programmed death-ligand 1 (PD-L1), group 2 cluster of differentiation 1 (CD1D), adenosine A2A receptor (A2AR), B7 Homolog 3 (B7-H3), B7 Homolog 4 (B7-H4), B- and T-lymphocyte attenuator (BTLA), cytotoxic T-lymphocyte associated protein 4 (CTLA-4), indoleamine-pyrrole 2,3-dixoygenase (IDO), killer-cell immunoglobulin-like receptor (KIR), lymphocyte-activation gene 3 (LAG3), NADPH oxidase 2 (NOX2), programmed death 1 receptor (PD-1), T-cell immunoglobulin and mucin-domain containing-3 (TIM-3), V-domain Ig suppressor of T cell activation (VISTA), Sialic acid-binding Ig-like lectin (SIGLEC7), or any variant, functional fragment, or combination thereof.

In one embodiment, said tolerogenic factor comprises CD47 or a variant thereof, and one or more tolerogenic factors selected from the group consisting of CD24, CR1, CD55, CD46, CD59, HLA-A, HLA-B, HLA-C, HLA-E, HLA-F, HLA-G, CD39, CD73, PD-L1, CD1D, A2AR, B7-H3, B7-H4, BTLA, CTLA-4, IDO, KIR, LAG3, NOX2, PD-1, TIM-3, VISTA, SIGLEC7, or any variant, functional fragment, or combination thereof. In one embodiment, at least a subset of said one or more tolerogenic factors is recombinant.

In one embodiment, said ORF is in operable connection with said inducible promoter.

In one embodiment, said stem cell further comprises an additional ORF encoding an additional tolerogenic factor.

In one embodiment, said additional ORF encoding said additional tolerogenic factor is in operable connection with said inducible promoter.

In one embodiment, said additional ORF comprising said additional tolerogenic factor is in operable connection with another inducible promoter.

In one embodiment, said additional ORF comprising said additional tolerogenic factor is in operable connection with a constitutive promoter optionally selected from a beta-2 microglobulin (B2M) promoter, a class II major histocompatibility complex transactivator (CIITA) promoter, and a CAG promoter.

In one embodiment, the method further comprises providing in said cell a cell-specific gene configured to modulate the expression of said tolerogenic factor.

In one embodiment, the method comprises tagging an activator or repressor molecule to said inducible promoter.

In one embodiment, said ORF encoding said tolerogenic factor is comprised in sequence having a transcription activator in operable connection with said inducible promoter.

In one embodiment, said differentiated cell is a natural killer (NK) cell, a T-cell, a B-cell, a hepatocyte, a cardiomyocyte, a renal cell, a dopaminergic neuron, a pancreatic islet cell, a macrophage, a nephrocyte, or a retinal pigment epithelium cell.

In one embodiment, said cell is an embryonic stem cell (ESC), a mesenchymal stem cell (MSC), an induced pluripotent stem cell (iPSC), or a hematopoietic stem cell (HSC).

In one embodiment, said stem cell further comprises a sequence encoding a translation control sequence.

In one embodiment, said sequence encoding said translation control sequence is positioned 5′ or 3′ of said open reading frame encoding said tolerogenic factor.

In one embodiment, said sequence encoding said translation control sequence is located between said ORF encoding said tolerogenic factor and another ORF encoding a native gene of said differentiated cell. In one embodiment, said native gene is B2M or CIITA.

In one embodiment, said cell further comprises another ORF encoding a chimeric antigen receptor (CAR) or T-cell receptor (TCR), or functional fragment thereof. In one embodiment, said another ORF encoding said CAR or TCR, or said functional fragment thereof, is in operable connection with said inducible promoter. In one embodiment, said cell further comprises another ORF encoding a costimulatory protein, optionally selected from a cluster of differentiation 28 (CD28) protein, an inducible T-cell costimulator (ICOS) protein, or any functional fragment or combination thereof. In one embodiment, said inducible promoter and said ORF encoding said tolerogenic factor are comprised in a nucleic acid molecule.

In one embodiment, said nucleic acid is comprised in a vector. In one embodiment, said vector is a plasmid, a minicircle, a CELiD, an adeno-associated virus (AAV) derived virion, a lentivirus, an adenovirus, or a herpes simplex virus (HSV).

In one embodiment, said nucleic acid is comprised in a genome of said cell.

Further, the present disclosure includes constructs. In one embodiment, said construct is a construct for gene expression in a cell, said construct comprising: an inducible promoter; and an open reading frame encoding a tolerogenic factor, wherein said inducible promoter is configured to modulate an expression of said tolerogenic factor, wherein said inducible promoter is configured to activate upon or subsequent to differentiation of said cell into a differentiated cell.

In one embodiment, said ORF encoding said tolerogenic factor comprises said inducible promoter. In one embodiment, said ORF encoding said tolerogenic factor does not comprise said cell-specific inducible promoter. In one embodiment, said tolerogenic factor comprises a SIRPα binding sequence. In one embodiment, said SIRPα binding sequence is a CD47 sequence or functional variant thereof.

In one embodiment, said SIRPα binding sequence comprises a sequence having at least about 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identity to any one of SEQ ID NOs: 1-653.

In one embodiment, said tolerogenic factor comprises CD47, cluster of differentiation 24 (CD24), complement receptor 1 (CR1), complement decay-accelerating factor (CD55), cluster of differentiation 46 (CD46), cluster of differentiation 59 (CD59), HLA-A, HLA-B, HLA-C, HLA-E, HLA-F, HLA-G, cluster of differentiation 39 (CD39), cluster of differentiation 73 (CD73), programmed death-ligand 1 (PD-L1), group 2 cluster of differentiation 1 (CD1D), adenosine A2A receptor (A2AR), B7 Homolog 3 (B7-H3), B7 Homolog 4 (B7-H4), B- and T-lymphocyte attenuator (BTLA), cytotoxic T-lymphocyte associated protein 4 (CTLA-4), indoleamine-pyrrole 2,3-dixoygenase (IDO), killer-cell immunoglobulin-like receptor (KIR), lymphocyte-activation gene 3 (LAG3), NADPH oxidase 2 (NOX2), programmed death 1 receptor (PD-1), T-cell immunoglobulin and mucin-domain containing-3 (TIM-3), V-domain Ig suppressor of T cell activation (VISTA), Sialic acid-binding Ig-like lectin (SIGLEC7), or any variant, functional fragment, or combination thereof.

In one embodiment, at least a subset of said one or more tolerogenic factors is recombinant. In one embodiment, said ORF is in operable connection with said inducible promoter. In one embodiment, construct further comprises an additional ORF encoding an additional tolerogenic factor. In one embodiment, said additional ORF encoding said additional tolerogenic factor is in operable connection with said inducible promoter.

In one embodiment, said additional ORF comprising said additional tolerogenic factor is in operable connection with another inducible promoter. In one embodiment, said additional ORF comprising said additional tolerogenic factor is in operable connection with a constitutive promoter. In one embodiment, said constitutive promoter is a beta-2 microglobulin (B2M) promoter, a class II major histocompatibility complex transactivator (CIITA) promoter, or a CAG promoter.

In one embodiment, the construct further comprises a cell-specific gene configured to modulate the expression of said tolerogenic factor.

In one embodiment, the construct further comprises an activator or repressor molecule tagged to said inducible promoter. In one embodiment, said open reading frame encoding said tolerogenic factor is comprised in a transcription activator in operable connection with said inducible promoter.

In one embodiment, said inducible promoter is a cell-specific promoter. In one embodiment, the cell-specific promoter is an immune cell gene promoter. In one embodiment, the cell-specific promoter is a white blood cell gene promoter. In some cases, the cell-specific promoter is a neutrophil gene promoter, an eosinophil gene promoter, a basophil gene promoter, a mast cell gene promoter, a monocyte gene promoter, a macrophage gene promoter, a dendritic cell gene promoter, a natural killer (NK) cell gene promoter, a memory-like NK cell gene promoter, a lymphocyte gene promoter, a B cell gene promoter, a T-cell gene promoter, a regulatory T-cell (Treg) promoter, a hepatocyte gene promoter gene promoter, a cardiomyocyte gene promoter, a renal cell gene promoter, a dopaminergic neuron gene promoter, a pancreatic islet cell gene promoter, a macrophage gene promoter, or a retinal pigment epithelium cell gene promoter.

In one embodiment, the cell-specific promoter is an NK cell gene promoter or a T-cell gene reporter.

In one embodiment, wherein the inducible promoter is an L selectin (CD62L) promoter, an interferon-gamma (IFNg) promoter, neural cell adhesion molecule (CD56) promoter, a CD56 promoter, a KIRs promoter, a CD16 promoter, a NKp44 promoter, a NKp46 promoter, a NKG2D promoter, a TRAIL promoter, a CD122 promoter, a CD27 promoter, a CD244 promoter, a NK1.1 promoter, a NKG2A/C promoter, a NCR1 promoter, a Ly49 promoter, a CD49b promoter, a CD11b promoter, a KLRG1 promoter, a CD43 promoter, a CD62L promoter, a CD226 promoter, a TRAC promoter, a TRBC promoter, a CD3 promoter, a CD4 promoter, a ThPOK promoter, a CD8 promoter, a FOXP3 promoter, a Helios promoter, a CD25 promoter, a GARP promoter, a GPA33 promoter, a CD14 promoter, a CD11b promoter, a CD68 promoter, a CD138 promoter, an IgH promoter, an IgK promoter, an IgL promoter, an IgG promoter, a CD19 promoter, a CD20 promoter, a TH promoter, a TUBB3 promoter, a FOXA2 promoter, a GIRK2 promoter, a Nurr1 promoter, a DAT promoter, an INS promoter, a chromogranin A promoter, a synaptophysin promoter, a neuron-specific enolase promoter, or a Leu7 promoter.

In one embodiment, the cell-specific promoter is a memory-like NK cell gene promoter.

In one embodiment, the cell-specific promoter is an L selectin (CD62L) promoter, an interferon-gamma (IFNg) promoter, or neural cell adhesion molecule (CD56) promoter.

In one embodiment, the cell-specific promoter is a CD16 promoter, a HLA-C promoter, a NKp46 promoter, or a DAP10 promoter.

In one embodiment, the cell-specific promoter comprises a sequence having 95%, 97%, 99% or 100% sequence identity to a 60, 70, 80, 90, 100, 200, 500, 1,000, 1,500 or 2,000 base pair long fragment of any one of SEQ ID 5 to 57. In one embodiment, the cell-specific promoter comprises a sequence having 95%, 97%, 99% or 100% sequence identity to a 60, 70, 80, 90, 100, 200, 500, 1,000, 1,500 or 2,000 base pair long fragment of any one of SEQ ID 54 to 57.

In one embodiment, the cell-specific promoter comprises a sequence having at least 95% sequence identity to a sequence that comprises 500 bp of any one of SEQ ID 5 to 57. In one embodiment, the cell-specific promoter comprises a sequence having at least 95% sequence identity to a sequence that comprises 500 bp of any one of SEQ ID 54 to 57.

In one embodiment, the cell-specific promoter comprises a sequence having at least 99% sequence identity to a sequence that comprises 500 bp of SEQ ID 5 to 57. In one embodiment, the cell-specific promoter comprises a sequence having at least 99% sequence identity to a sequence that comprises 500 bp of SEQ ID 54 to 57. In one embodiment, comprises a 500 bp sequence of SEQ ID 5 to 57. In one embodiment, comprises a 500 bp sequence of SEQ ID 54 to 57.

In one embodiment, said differentiated cell is a natural killer (NK) cell, a T-cell, a B-cell, a hepatocyte, a cardiomyocyte, a renal cell, a dopaminergic neuron, a pancreatic islet cell, a macrophage, a nephrocyte, or a retinal pigment epithelium cell.

In one embodiment, said cell is a stem cell, a pluripotent cell, or a multipotent cell.

In one embodiment, said stem cell is an embryonic stem cell (ESC), a mesenchymal stem cell (MSC), an induced pluripotent stem cell (iPSC), or a hematopoietic stem cell (HSC).

In one embodiment, the construct further comprises a sequence encoding a translation control sequence. In one embodiment, said translation control sequence is an internal ribosome entry site (IRES) or 2A peptide. In one embodiment, said sequence encoding said translation control sequence is positioned 5′ or 3′ of said open reading frame encoding said tolerogenic factor. In one embodiment, said sequence encoding said translation control sequence is located between said ORF encoding tolerogenic factor and another ORF encoding a native gene of said differentiated cell. In one embodiment, said native gene is B2M or CIITA.

In one embodiment, said construct further comprises another ORF encoding a chimeric antigen receptor (CAR) or T-cell receptor (TCR), or functional fragment thereof. In one embodiment, said CAR or TCR, or said functional fragment thereof, targets a tumor-specific antigen. In one embodiment, said CAR or TCR, or said functional fragment thereof, comprises a TCR-α chain, a TCR-β chain, a TCR-γ chain, a TCR-δ chain, a TCR-ζ chain, a CD3 molecule, or any functional fragment or combination thereof. In one embodiment, said another ORF encoding said CAR or TCR, or said functional fragment thereof, is in operable connection with said inducible promoter.

In one embodiment, the construct comprises another ORF encoding a costimulatory protein; wherein is costimulatory protein optionally comprises a cluster of differentiation 28 (CD28) protein, an inducible T-cell costimulator (ICOS) protein, or any functional fragment or combination thereof.

In one embodiment, said construct is a nucleic acid molecule. In one embodiment, said construct is a vector. In one embodiment, said vector is a plasmid, a minicircle, a CELiD, an adeno-associated virus (AAV) derived virion, a lentivirus, an adenovirus, or a herpes simplex virus (HSV). In one embodiment, said construct is integrated into a genome of said cell.

Further methods of the invention include: subjecting a cell comprising the construct of any one of as disclosed above to conditions sufficient to differentiate said cell into a differentiated cell.

Even further embodiments of the methods include: subjecting a cell that does not express a tolerogenic factor to conditions sufficient to differentiate said cell into an immune cell, wherein upon or subsequent to differentiation, said immune cell expresses said tolerogenic factor.

In one embodiment of said methods, said tolerogenic factor is a CD47 sequence or functional variant thereof. In one embodiment of said methods, said tolerogenic factor comprises CD47, cluster of differentiation 24 (CD24), complement receptor 1 (CR1), complement decay-accelerating factor (CD55), cluster of differentiation 46 (CD46), cluster of differentiation 59 (CD59), HLA-A, HLA-B, HLA-C, HLA-E, HLA-F, HLA-G, cluster of differentiation 39 (CD39), cluster of differentiation 73 (CD73), programmed death-ligand 1 (PD-L1), adenosine A2A receptor (A2AR), B7 Homolog 3 (B7-H3), B7 Homolog 4 (B7-H4), B- and T-lymphocyte attenuator (BTLA), cytotoxic T-lymphocyte associated protein 4 (CTLA-4), indoleamine-pyrrole 2,3-dixoygenase (IDO), killer-cell immunoglobulin-like receptor (KIR), lymphocyte-activation gene 3 (LAG3), NADPH oxidase 2 (NOX2), programmed death 1 receptor (PD-1), T-cell immunoglobulin and mucin-domain containing-3 (TIM-3), V-domain Ig suppressor of T cell activation (VISTA), Sialic acid-binding Ig-like lectin (SIGLEC7), or any variant, functional fragment, or combination thereof. In one embodiment of said further methods, at least a subset of said one or more tolerogenic factors is recombinant.

In one embodiment of said methods, said cell comprises an inducible promoter and an open reading frame (ORF) encoding said tolerogenic factor. In one embodiment of said further methods, said inducible promoter is delivered exogenously to said cell. In one embodiment of said methods, said inducible promoter exists endogenously within said cell.

In one embodiment of said methods, said ORF is in operable connection with said inducible promoter.

In one embodiment of said further methods, said ORF is provided in or on a vector.

In one aspect, said vector is a plasmid, a minicircle, a CELiD, an adeno-associated virus (AAV) derived virion, a lentivirus, an adenovirus, or a herpes simplex virus (HSV). In another aspect, said ORF is integrated into a genome of said stem cell.

In one embodiment of said methods, said cell further comprises an additional ORF encoding an additional tolerogenic factor. In one embodiment of said further methods, said additional ORF encoding said additional tolerogenic factor is in operable connection with said inducible promoter.

In one embodiment of said methods, said additional ORF comprising said additional tolerogenic factor is in operable connection with another inducible promoter. In one embodiment of said further methods, said additional ORF comprising said additional tolerogenic factor is in operable connection with a constitutive promoter.

In one embodiment of said methods, said constitutive promoter is a beta-2 microglobulin (B2M) promoter, a class II major histocompatibility complex transactivator (CIITA) promoter, or a CAG promoter.

One embodiment of said methods comprises tagging an activator or repressor molecule to said inducible promoter. In one aspect, said inducible promoter is a cell-specific promoter. In one embodiment, the cell-specific promoter is an immune cell gene promoter. In one embodiment of said methods, the cell-specific promoter is a white blood cell gene promoter. In some cases, the cell-specific promoter is a neutrophil gene promoter, an eosinophil gene promoter, a basophil gene promoter, a mast cell gene promoter, a monocyte gene promoter, a macrophage gene promoter, a dendritic cell gene promoter, a natural killer (NK) cell gene promoter, a memory-like NK cell gene promoter, a lymphocyte gene promoter, a B cell gene promoter, a T-cell gene promoter, a regulatory T-cell (Treg) promoter, a hepatocyte gene promoter gene promoter, a cardiomyocyte gene promoter, a renal cell gene promoter, a dopaminergic neuron gene promoter, a pancreatic islet cell gene promoter, a macrophage gene promoter, or a retinal pigment epithelium cell gene promoter.

In one embodiment of said methods, the inducible promoter is an L selectin (CD62L) promoter, an interferon-gamma (IFNg) promoter, neural cell adhesion molecule (CD56) promoter, a CD56 promoter, a KIRs promoter, a CD16 promoter, a NKp44 promoter, a NKp46 promoter, a NKG2D promoter, a TRAIL promoter, a CD122 promoter, a CD27 promoter, a CD244 promoter, a NK1.1 promoter, a NKG2A/C promoter, a NCR1 promoter, a Ly49 promoter, a CD49b promoter, a CD11b promoter, a KLRG1 promoter, a CD43 promoter, a CD62L promoter, a CD226 promoter, a TRAC promoter, a TRBC promoter, a CD3 promoter, a CD4 promoter, a ThPOK promoter, a CD8 promoter, a FOXP3 promoter, a Helios promoter, a CD25 promoter, a GARP promoter, a GPA33 promoter, a CD14 promoter, a CD11b promoter, a CD68 promoter, a CD138 promoter, an IgH promoter, an IgK promoter, an IgL promoter, an IgG promoter, a CD19 promoter, a CD20 promoter, a TH promoter, a TUBB3 promoter, a FOXA2 promoter, a GIRK2 promoter, a Nurr1 promoter, a DAT promoter, an INS promoter, a chromogranin A promoter, a synaptophysin promoter, a neuron-specific enolase promoter, or a Leu7 promoter.

In one embodiment of said methods, the cell-specific promoter is a memory-like NK cell gene promoter.

In one embodiment of said further methods, wherein the cell-specific promoter is an L selectin (CD62L) promoter, an interferon-gamma (IFNg) promoter, or neural cell adhesion molecule (CD56) promoter.

In one embodiment of said methods, the cell-specific promoter is a CD16 promoter, a HLA-C promoter, a NKp46 promoter, or a DAP10 promoter.

In one embodiment of said methods, the cell-specific promoter comprises a sequence having 95%, 97%, 99% or 100% sequence identity to a 60, 70, 80, 90, 100, 200, 500, 1,000, 1,500 or 2,000 base pair long fragment of any one of SEQ ID 5 to 57. In one embodiment of said further methods, the cell-specific promoter comprises a sequence having 95%, 97%, 99% or 100% sequence identity to a 60, 70, 80, 90, 100, 200, 500, 1,000, 1,500 or 2,000 base pair long fragment of any one of SEQ ID 54 to 57.

In one embodiment of said methods, the cell-specific promoter comprises a sequence having at least 95% sequence identity to a sequence that comprises 500 bp of any one of SEQ ID 5 to 57. In one embodiment of said methods, the cell-specific promoter comprises a sequence having at least 95% sequence identity to a sequence that comprises 500 bp of any one of SEQ ID 54 to 57. In one embodiment, the cell-specific promoter comprises a sequence having at least 95% sequence identity to a sequence that comprises 500 bp of any one of SEQ ID 54 to 57.

In one embodiment, the cell-specific promoter comprises a sequence having at least 99% sequence identity to a sequence that comprises 500 bp of SEQ ID 5 to 57. In one embodiment, the cell-specific promoter comprises a sequence having at least 99% sequence identity to a sequence that comprises 500 bp of SEQ ID 54 to 57.

In one embodiment, the method further comprises a sequence encoding a translation control sequence. In one embodiment, the translation control sequence is an internal ribosome entry site (IRES) or 2A peptide.

In one embodiment, said sequence encoding said translation control sequence is positioned 5′ or 3′ of said open reading frame encoding said tolerogenic factor.

In one embodiment, wherein said sequence encoding said translation control sequence is located between said ORF encoding said tolerogenic factor and another ORF encoding a native gene of said differentiated cell. In one embodiment, said native gene is B2M or CIITA. In one embodiment, said cell further comprises another ORF encoding a chimeric antigen receptor (CAR) or T-cell receptor (TCR), or functional fragment thereof.

In one embodiment, said cell further comprises a cell-specific gene configured to modulate the expression of said tolerogenic factor. In one embodiment, said cell is an immune cell. In one aspect, said cell is a natural killer (NK) cell, a T-cell or a B-Cell.

In one embodiment, said cell is an embryonic stem cell (ESC), a mesenchymal stem cell (MSC), an induced pluripotent stem cell (iPSC), or a hematopoietic stem cell (HSC).

A method for reducing immune response in vivo, administering a cell comprising an inducible promoter in operable connection with a tolerogenic factor to a subject, subjecting the cell to conditions that induce the inducible promoter to modulate signaling from the tolerogenic factor.

Further the present disclosure includes methods for enriching a cell population in vivo, comprising administering a homologous or heterologous cell population to a subject, wherein said cell population comprises at least one cell comprising an inducible promoter in operable connection with a tolerogenic factor, subjecting the cell populations to conditions that induce the inducible promoter to modulate signaling from the tolerogenic factor. In one embodiment, said heterologous or homologous cell population includes stem cells, in particular iPSC, ESC, HSC or other stem cells. In another embodiment said population of cells comprises pluripotent or multipotent cells. In yet another embodiment, the population of cells comprises immune cells.

In one embodiment, modulated signaling from the tolerogenic factor reduces the immunological rejection of the cells comprising the inducible promoter. Yet another embodiments include methods of treating a disease comprising administering cells comprising the constructs of the invention.

### DETAILED DESCRIPTION

### Incorporation by Reference

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates example methods and constructs for modulating expression of a tolerogenic factor (as exemplified here by using a SIRPα agonist or binder), in accordance with aspects of the disclosure.

FIG. 2 illustrates an advantage conferred by the methods and constructs of the present disclosure.

FIG. 3 illustrates two strategies to identify cell specific promoters or loci for insertion of tolerogenic factors to be expressed in a cell specific manner. In strategy 1, we cloned approximately 1 kb to 3 kb upstream of the transcription start site of a protein that is endogenously expressed in cell-specific manner (here NK cells were chosen). From those upstream sequences, four different promoter sequences were designed (e.g., CD16 TSS, NKp46 (NCR1) promoter, HLA-C TSS promoter, and DAP10 TSS promoter). The promoter sequences were inserted into a vector in operable connection with a GFP sequence which would normally be a tolerogenic sequence, and the vector was cloned into CD56+ NK cells. As shown in the plot below, 28.2% of the CD56+ NK cells were GFP positive after transfection with the cell specific promoter sequence tied to GFP. This shows that cell-specific promoter constructs can be used to induce the expression of genes. In strategy 2, we utilized endogenous gene tagging at the 5′ and 3′ ends of genes highly expressed in a cell specific manner (i.e., a target gene). We generated a construct encoding GFP and a translation regulatory sequence 2A, having no promoter. We introduced our GFP construct into primary NK cells at the 5′ or 3′ of the endogenous target gene (here CD56). We demonstrate that 1.3% of the NK cells were also GFP positive, demonstrating that we can drive expression off an endogenous cell-specific promoter.

FIG. 4 is a cartoon of four of the constructs generated comprising a cell-specific promoter, including transcription regulatory elements such as TS bind sites, TATA box, Pmed1/inPmed1, and miniTK. The cell specific promoters were for DAP10, HLA-C, NKp46 and CD16.

FIG. 5 shows a computer system that is programmed or otherwise configured to implement methods provided herein.

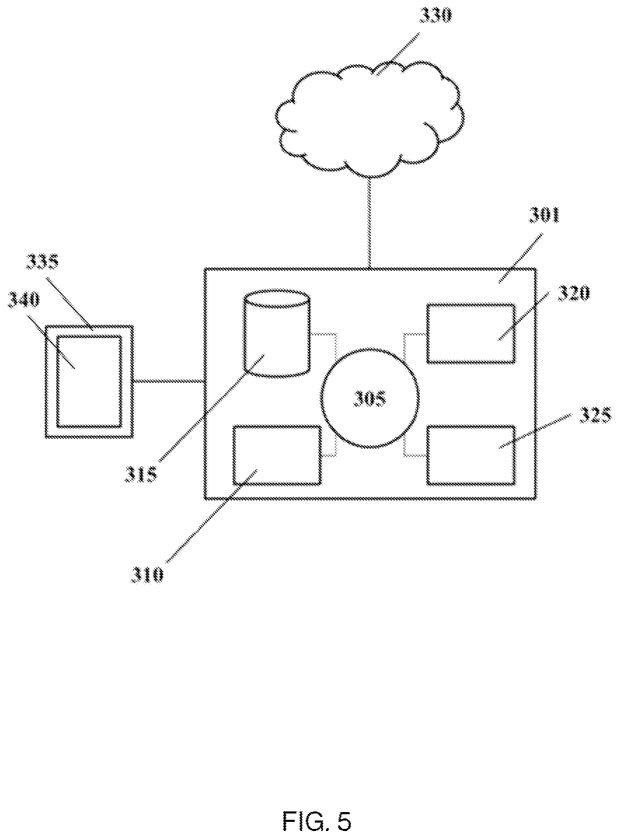

FIG. 6 shows a schematic overview for modulating expression of a tolerogenic factor in stem cells and differentiated cells.

FIG. 7A shows the derivation of the cell-specific promoter from a desired cell type, and its incorporation into a vector comprising the construct of the present invention including the cell-specific promoter and two tolerogenic factors. FIG. 7B shows a specific example of a vector comprising a construct of the invention disclosed herein.

FIG. 8A. demonstrates expression of our inserted gene (which would be a tolerogenic factor, but is GFP here) in the population of differentiated cells, FIG. 8B shows viability of cells post insertion of the construct, FIG. 8C shows % expression of the target gene in the population of cells and FIG. 8D shows the percent of cells that were both GFP+ and also expressed the target gene.

FIGS. 9A and 9B shows that a cell specific promoter (here DAP10) does not drive any GFP transgene expression in iPSCs. FIG. 9A shows control populations: negative control naïve iPSC without any construct, iPSC transfected with a constitutive promoter driven GFP construct, and a CAG ubiquitous promoter (known for driving high expression levels in mammalian cells), which appropriately shown no GFP+ iPSC, and high percentages of GFP+ iPSCs (CMV and CAG promoter constructs). FIG. 9B demonstrates that the four NK cell specific promoter constructs of the present disclosure show no (or very little) GFP in iPSCs, showing the specificity of the expression of the promoters hereunder.

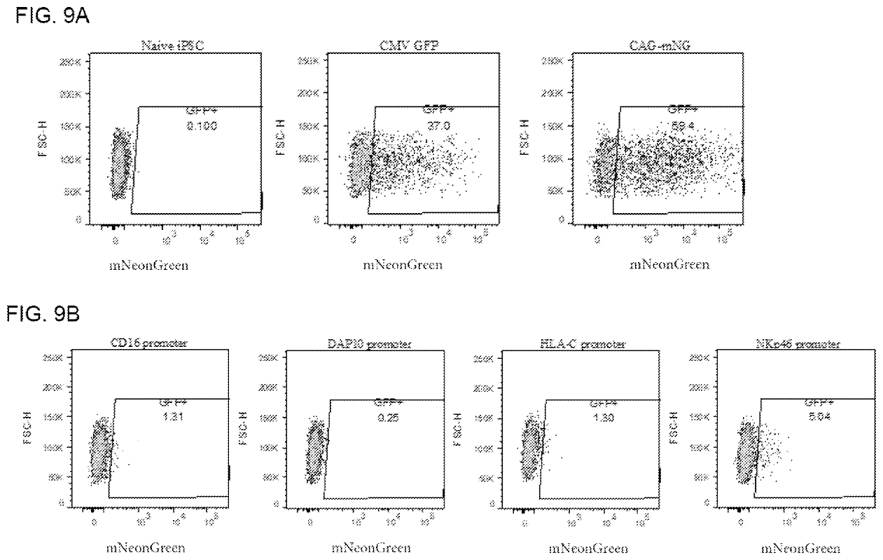

FIG. 10 demonstrates that the NK cells lacking MHC Class I and II, but expressing tolerogenic factors are preferentially protected from cell lysis by allogeneic NK cells.

### DEFINITIONS

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. Inhibition may refer to reduction of a disease or symptoms of disease. Inhibition may refer to a reduction in the activity of a particular protein or nucleic acid target. The protein may be deoxycytidine kinase. Thus, inhibition includes, at least in part, partially or totally blocking stimulation, decreasing, preventing, or delaying activation, or inactivating, desensitizing, or down-regulating signal transduction or enzymatic activity or the amount of a protein.

“Endogenous level” as used herein means the level of expression or signaling the cell type would express in the absence of the ORF of the present invention.

The term “exogenous” as used herein refers to a polynucleotide (such as one encoding a gene product or part of a gene product) that is not present endogenously in a mammalian cell, such as an immune cell, or is synthetically generated outside of a mammalian cell, such as by recombinant technology. In a specific case, a particular gene may be provided to a cell exogenously via a construct, and the cell may or may not also express the corresponding endogenous gene product in the cell.

The term “promoter” means a region of DNA, typically 100-3,000 bp in length, e.g., 300-2,500 bp, 500-2,000, or 1,000-1,500 in length, upstream of where relevant proteins (such as RNA polymerase and transcription factors) bind to initiate transcription of the gene to be modulated. Promoter sequences describe the direction of transcription and identify which DNA strand will be transcribed. Some promoters contain one or more upstream promoter elements (UP element) subsites upstream of the Transcription Start Site (TSS).

The term “modulator” refers to a composition that increases or decreases the level of a target molecule or the function of a target molecule or the physical state of the target of the molecule.

The term “modulate” is used in accordance with its plain ordinary meaning and refers to the act of changing or varying one or more properties. “Modulation” refers to the process of changing or varying one or more properties. For example, a modulator of a target protein changes by increasing or decreasing a property or function of the target molecule or the amount of the target molecule, including but not limited to protein expression, protein signaling, and pathway signaling. A modulator of a disease decreases a symptom, cause, or characteristic of the targeted disease.

As used herein, “operably connected” or linked refers to positioning of a regulatory region relative to a nucleic acid sequence in such a way as to permit or facilitate transcription of the target nucleic acid.

“Tagging” as used herein means that the sequences encoding or containing (for non-translated elements like translation regulatory elements) the listed elements are spatially arranged to be transcribed in a single transcript.

A “tolerogenic factor” as used herein generally refers to one or more molecules, the expression of which by or presence on or in a cell gives rise to a reduced immunological response in vitro or in vivo, as compared to a cell which expresses the factor at endogenous levels or as compared to a cell which does not express or present the tolerogenic factor at all. In some cases, the immunological response is an immunological rejection response when the cell is transferred into an allogenic host. In some cases, a tolerogenic factor is an immune checkpoint molecule, or fragment or variant thereof. In some cases, a tolerogenic factor is a stimulatory checkpoint molecule, or fragment or variant thereof. In some cases, a tolerogenic factor is an inhibitory checkpoint molecule, or fragment or variant thereof. In some cases, a tolerogenic factor is an agonist of an immune checkpoint molecule. In some cases, a tolerogenic factor displays its immunological modulatory power only in the presence or absence of other molecules, such as other tolerogenic factors or other biomolecules described herein. In some cases, a molecule may be described as belonging to a certain class of tolerogenic factor or being a certain tolerogenic factor if it displays or is predicted to display substantially similar immune modulatory capabilities to another tolerogenic factor through substantially the same biochemical mechanism. By way of non-limiting example, molecules disclosed herein which are observed or predicted to bind to and are observed or predicted agonize the signal regulatory protein alpha (SIRPα) receptor may be referred to here as “CD47” or “variants of CD47” due to displaying a substantially similar immunomodulatory behavior to CD47 through substantially the same biochemical mechanism, even though such variants of CD47 may display low sequence identity to wild-type CD47 sequences (e.g., less than a cutoff level of identity, such as less than about 80%, 70%, 60%, 50%, 40%, 30%, or 20%, or less, sequence identity).

Unless clearly indicated otherwise, the term “individual” as used herein refers to a mammal, including but not limited to, bovine, horse, feline, rabbit, canine, rodent, or primate (e.g., human). In some cases, an individual is a human. In some cases, an individual is a non-human primate such as chimpanzees and other apes and monkey species. In some cases, an individual is a farm animal such as cattle, horses, sheep, goats, and swine; pets such as rabbits, dogs, and cats; laboratory animals including rodents, such as rats, mice, and guinea pigs; and the like. In some cases, the invention find use in both human medicine and in the veterinary context.

As used herein, “mutation” refers to an alteration in the sequence of a nucleic acid molecule. Mutations include, but are not limited to, insertions, deletions, and substitutions.

As used herein, the abbreviations for amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Other amino acids include citrulline (Cit); homocysteine (Hey); hydroxyproline (Hyp); ornithine (Orn); and thyroxine (Thx). Examples of amino acids that are not charged at physiological pH include, but are not limited to, alanine, asparagine, cysteine, glutamine, glycine, isoleucine, leucine, methionine, phenylalanine, proline, serine, threonine, tryptophan, tyrosine, and valine.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The present disclosure relates to methods and compositions to modulate gene expression and/or gene signaling in the manufacture and use of a cellular therapeutic.

### Constructs

Disclosed herein, in one aspect, are constructs for modulating gene expression in a cell. In some cases, the constructs are configured to drive expression of one or more genes (e.g., transgenes comprising a tolerogenic factor,) off inducible gene promoters.

An aspect of the present disclosure provides a construct for gene expression in a cell, the construct comprising: either a an inducible promoter, an open reading frame (“ORF”) encoding a tolerogenic factor, or both an inducible promoter and an ORF encoding a tolerogenic factor, wherein an inducible promoter is configured to modulate the expression or signaling of the tolerogenic factor in comparison to cells not containing the construct, wherein the inducible promoter is configured to activate concurrent with or subsequent to differentiation of the cell into a differentiated cell. In a preferred aspect the construct further comprises an inducible promoter.

Tolerogenic Factor. In some embodiments, the tolerogenic factor comprises a SIRPα binding sequence, CD47, cluster of differentiation 24 (CD24), complement receptor 1 (CR1), complement decay-accelerating factor (CD55), cluster of differentiation 46 (CD46), cluster of differentiation 59 (CD59), HLA-A, HLA-B, HLA-C, HLA-E, HLA-F, HLA-G, cluster of differentiation 39 (CD39), cluster of differentiation 73 (CD73), programmed death-ligand 1 (PD-L1), group 2 cluster of differentiation 1 (CD1D), adenosine A_{2A} receptor (A2AR), B7 Homolog 3 (B7-H3), B7 Homolog 4 (B7-H4), B- and T-lymphocyte attenuator (BTLA), cytotoxic T-lymphocyte associated protein 4 (CTLA-4), indoleamine-pyrrole 2,3-dixoygenase (IDO), killer-cell immunoglobulin-like receptor (KIR), lymphocyte-activation gene 3 (LAG3), NADPH oxidase 2 (NOX2), programmed death 1 receptor (PD-1), T-cell immunoglobulin and mucin-domain containing-3 (TIM-3), V-domain Ig suppressor of T cell activation (VISTA), Sialic acid-binding Ig-like lectin (SIGLEC7), or any variant, functional fragment, or combination of the foregoing. In some embodiments, the tolerogenic factor comprises one or more tolerogenic factors selected from the group consisting of CD47, CD24, CR1, CD55, CD46, CD59, HLA-A, HLA-B, HLA-C, HLA-E, HLA-F, HLA-G, CD39, CD73, PD-L1, CD1D, A2AR, B7-H3, B7-H4, BTLA, CTLA-4, IDO, KIR, LAG3, NOX2, PD-1, TIM-3, VISTA, SIGLEC7, or any variant, functional fragment, or combination of the foregoing. In some embodiments, the tolerogenic factor comprises one or more tolerogenic factors selected from the group consisting of CD47, CD24, CR1, CD55, CD46, CD59, HLA-A, HLA-B, HLA-C, HLA-E, HLA-F, HLA-G, CD39, CD73, PD-L1, CD1D, A2AR, B7-H3, B7-H4, BTLA, CTLA-4, IDO, KIR, LAG3, NOX2, PD-1, TIM-3, VISTA, SIGLEC7, or any variant, functional fragment, or combination of the foregoing. In some embodiments, at least a subset of the one or more tolerogenic factors is recombinant. In some embodiments, the tolerogenic factor comprises a SIRPα binding sequence, cluster of differentiation 24 (CD24; e.g., UniProt Accession P25063), complement receptor 1 (CR1; e.g., UniProt Accession P17927), complement decay-accelerating factor (CD55; e.g., UniProt Accession P08174), cluster of differentiation 46 (CD46; e.g., UniProt Accession; P15529), cluster of differentiation 59 (CD59; e.g., UniProt Accession P13987), HLA-A (e.g., e.g., UniProt Accession P04439), HLA-B (e.g., UniProt Accession P01889), HLA-C (e.g., e.g., UniProt Accession P10321), HLA-E (e.g., e.g., UniProt Accession P13747), HLA-F (e.g., UniProt Accession P30511), HLA-G (e.g., UniProt Accession P17693), cluster of differentiation 39 (CD39; e.g., UniProt Accession P49961), cluster of differentiation 73 (CD73; e.g., UniProt Accession P21589), programmed death-ligand 1 (PD-L1; e.g., UniProt Accession Q9NZQ7), group 2 cluster of differentiation 1 (CD1D; e.g., UniProt Accession P15813), adenosine A_{2A} receptor (A2AR; e.g., UniProt Accession ADORA2A), B7 Homolog 3 (B7-H3; e.g., UniProt Accession Q5ZPR3), B7 Homolog 4 (B7-H4; e.g., UniProt Accession Q7Z7D3), B- and T-lymphocyte attenuator (BTLA; e.g., UniProt Accession Q7Z6A9), cytotoxic T-lymphocyte associated protein 4 (CTLA-4; e.g., UniProt Accession P16410), indoleamine-pyrrole 2,3-dixoygenase (IDO; e.g., UniProt Accession P14902), killer-cell immunoglobulin-like receptor (KIR; e.g., UniProt Accession P55040), lymphocyte-activation gene 3 (LAG3; e.g. UniProt Accession P18627), NADPH oxidase 2 (NOX2; e.g., UniProt Accession P04839), programmed death 1 receptor (PD-1; e.g., UniProt Accession Q15116), T-cell immunoglobulin and mucin-domain containing-3 (TIM-3; e.g., UniProt Accession P35625), V-domain Ig suppressor of T cell activation (VISTA; e.g., UniProt Accession Q9H7M9), Sialic acid-binding Ig-like lectin (SIGLEC7; e.g., UniProt Accession Q9Y286), or any variant, functional fragment, or combination thereof. The references provided above are for human wild-type tolerogenic factors, however, the present invention encompasses variants (human and non), functional fragments, modifications and combinations (including fusion proteins) having tolerogenic activities.

In a certain aspect, the tolerogenic factor is a variant, functional fragment, modification, or combination of a wild type tolerogenic factor which also has tolerogenic activities. One example of the variant, functional fragment, or modification is the modification of a SIRPα agonist. In one aspect of the present disclosure the tolerogenic factor comprises SIRPα agonist mutations based at least in part on any one of WT CD47 splice variants 1-4 that enhance ‘don't eat me’ signaling, thus preventing phagocytosis of cells which express the mutated SIRPα agonists (e.g., induced pluripotent stem cells (iPSCs)). In some embodiments, the mutations are from computational in silico calculations (e.g., all atom molecular dynamics, normal mode analysis, coarse grain simulations, frustration analysis, and protein stability).

Another aspect of the present disclosure comprises tolerogenic factors comprising engineered SIRPα agonist sequences based at least in part on any one of SEQ ID NOs: 1-4 that retain binding of human SIRPα but contain peptide insertions within the RVVSWF peptide linker (e.g., residues 114-119 of any one of SEQ ID NOs: 1-4) connecting the extracellular domain (ECD) and transmembrane domain (TMD) of any one of SEQ ID NOs: 1-4, display altered/detuned signaling function (e.g., do not bind or have reduced binding of endogenous protein partners (other than SIRPα), do not signal through (or have decreased signaling functions) via said SIRPα agonist), are more likely to dimerize or oligomerize, and/or have enhanced SIRPα agonist stability. In some embodiments, the peptide insertions are based at least in part on one or more orthologs or homologs of SEQ ID NOs: 1-4 as may be shown in PCT/US2023/071212, which is herein expressly incorporated by reference in its entirety.

In some cases, the ORF comprises sequences encoding 1, 2, 3, 4, 5, 6, 7, 8, 9, 10, 11, 12, 13, 14, 1,5 16, 17, 18, 19, 20, or more tolerogenic factors or variants or functional fragments thereof. In one embodiment, the ORF includes two or more tolerogenic factors or variants or functional fragments thereof. In one embodiment, said factors comprise CD47 or a fragment or variant thereof and HLA-E or a fragment or variant thereof separated by a 2A sequence. In a further embodiment, the ORF includes two or more tolerogenic factors and a cytokine. In one embodiment, the cytokine is selected from IL-15. In another embodiment the ORF comprises sequences encoding CD47 or a fragment or variant thereof, HLA-E or a fragment or variant thereof, and IL-15 or a fragment or variant thereof; each coding sequence separated by 2A sequences.

In some cases, the ORF comprises an immune receptor or fragment thereof. In some cases, the ORF comprises a chimeric antigen receptor (CAR) or functional fragment thereof. In some cases, the ORF comprises a T-cell receptor or functional fragment thereof. In some cases, the TCR, or functional fragment thereof, comprises a TCR-α chain, a TCR-β chain, a TCR-γ chain, a TCR-δ chain, a TCR-ζ chain, a CD3 molecule, or any functional fragment or combination thereof. In some cases, the ORF comprises at least 1, 2, 3, 4, 5, 6 7, 8, 9, 10, 11, 12, 13, 14, 15, 1,6 17, 18, 19, 20, or more immune receptors or fragments thereof.

In some cases, the ORF comprises a costimulatory molecule. In some cases, the costimulatory molecule comprises cluster of differentiation 28 (CD28). In some cases, the costimulatory molecule comprises cluster of differentiation 80 (CD80). In some cases, the costimulatory molecule comprises cluster of differentiation 86 (CD86). In some cases, the costimulatory molecule comprises inducible T-cell costimulator (ICOS).

In some embodiments, the ORF encoding the tolerogenic factor comprises an inducible promoter. In some embodiments, the ORF encoding the tolerogenic factor does not comprise the inducible promoter. In some embodiments, the ORF is in operable connection with the inducible promoter.

In some embodiments, the ORF encoding the target gene (e.g., a tolerogenic factor, such as a SIRPα binding sequence) is operably connected with an inducible promoter, and a corresponding activator or repressor molecule is operably connected to the cell-specific promoter.

### Inducible Promoters

In some cases, constructs for modulating expression of a tolerogenic factor as described herein are introduced to be in operable connection with or comprise an inducible promoter configured to modulate expression of the gene in vitro or in vivo. The inducible promoter may be modeled after a promoter driving transcription of a cell-type specific endogenous target gene (“cell-specific promoter”). In one embodiment the cell-specific promoter's activity changes when the cell undergoes a chemical change during differentiation or cell maturation into the cell type that expresses the cell-specific endogenous target gene. Such chemical change may include expression of certain other proteins in the differentiation or cell maturation pathway. In one embodiment, the inducible promoter is inactive until the cell differentiates into a differentiated cell of the specifically desired cell type. In another embodiment the inducible promoter may be inactive until the cell matures to a certain point, as demonstrated by cell type specific markers. In another embodiment, the inducible promoter may be active until the cell expresses certain other proteins in the differentiation or cell maturation pathway or is subjected to chemical or physical stimulus during the differentiation or maturation process, upon which the promoter may decrease or halt transcription inducing activity.

Upon subjecting a cell containing the construct of the invention to conditions sufficient to activate the inducible promoter (e.g., subjecting the cell to conditions sufficient to differentiate the starting cell into the corresponding cell type, or subjecting the cell to the chemical activator), the expression of the sequence encoding the tolerogenic factor may thereby be modulated. In some cases, the sequence encoding the tolerogenic factor is not expressed before differentiation of the cell into the corresponding differentiated cell. In some cases, the sequence encoding the tolerogenic factor is expressed or overexpressed following differentiation of the cell into the corresponding differentiated cell. In some cases, a plurality of variants of the sequence encoding the tolerogenic factor are expressed following differentiation of the cell into the corresponding differentiated cell.

Constructs as described herein may comprise any inducible promoter. In some embodiments the inducible promoter is a cell-specific promoter. In some cases, the cell-specific promoter is an immune cell gene promoter. In other embodiments, the inducible promoter may be synthetically designed to become induced upon or subsequent to differentiation.

In some cases, the cell-specific promoter is a white blood cell gene promoter. In some cases, the cell-specific promoter is a neutrophil gene promoter. In some cases, the cell-specific promoter is an eosinophil gene promoter. In some cases, the cell-specific promoter is a basophil gene promoter. In some cases, the cell-specific promoter is a mast cell gene promoter. In some cases, the cell-specific promoter is a monocyte gene promoter. In some cases, the cell-specific promoter is a macrophage gene promoter. In some cases, the cell-specific promoter is a dendritic cell gene promoter. In some cases, the cell-specific promoter is a natural killer (NK) cell gene promoter. In some cases, the cell-specific promoter is a memory-like NK cell gene promoter. In some cases, the cell-specific promoter is a lymphocyte gene promoter. In some cases, the cell-specific promoter is a B cell gene promoter. In some cases, the cell-specific promoter is a T-cell gene promoter. In some cases, the cell-specific promoter is a regulatory T-cell (Treg) promoter. In some cases, the cell-specific promoter is a hepatocyte gene promoter gene promoter. In some cases, the cell-specific promoter is a cardiomyocyte gene promoter. In some cases, the cell-specific promoter is a renal cell gene promoter. In some cases, the cell-specific promoter is a dopaminergic neuron gene promoter. In some cases, the cell-specific promoter is a pancreatic islet cell gene promoter. In some cases, the cell-specific promoter is a macrophage gene promoter. In some cases, the cell-specific promoter is a retinal pigment epithelium cell gene promoter. In some cases, the cell-specific inducible promoter is an L selectin (CD62L) promoter. In some cases, the cell-specific promoter is an interferon-gamma (IFNg) promoter. In some cases, the cell-specific promoter is neural cell adhesion molecule (CD56) promoter. In some cases, the cell-specific inducible promotor is a CD56 promoter. In some cases, the cell-specific promoter is a KIRs promoter. In some cases, the cell-specific promoter is a CD16 promoter. In some cases, the cell-specific promoter is a NKp44 promoter. In some cases, the cell-specific promoter is a NKp46 promoter. In some cases, the cell-specific promoter is a NKG2D promoter. In some cases, the cell-specific promoter is a TRAIL promoter. In some cases, the cell-specific promoter is a CD122 promoter. In some cases, the cell-specific promoter is a CD27 promoter. In some cases, the cell-specific promoter is a CD244 promoter. In some cases, the cell-specific promoter is a NK1.1 promoter. In some cases, the cell-specific promoter is a NKG2A/C promoter. In some cases, the cell-specific promoter is a NCR1 promoter. In some cases, the cell-specific promoter is a Ly49 promoter. In some cases, the cell-specific promoter is a CD49b promoter. In some cases, the cell-specific promoter is a CD11b promoter. In some cases, the cell-specific promoter is a KLRG1 promoter. In some cases, the cell-specific promoter is a CD43 promoter. In some cases, the cell-specific promoter is a CD62L promoter. In some cases, the cell-specific promoter is a CD226 promoter. In some cases, the cell-specific promoter is a TRAC promoter. In some cases, the cell-specific promoter is a TRBC promoter. In some cases, the cell-specific promoter is a CD3 promoter. In some cases, the cell-specific promoter is a CD4 promoter. In some cases, the cell-specific promoter is a ThPOK promoter. In some cases, the cell-specific promoter is a CD8 promoter. In some cases, the cell-specific promoter is a FOXP3 promoter. In some cases, the cell-specific promoter is a Helios promoter. In some cases, the cell-specific promoter is a CD25 promoter. In some cases, the cell-specific promoter is a GARP promoter. In some cases, the cell-specific promoter is a GPA33 promoter. In some cases, the cell-specific promoter is a CD14 promoter. In some cases, the cell-specific promoter is a CD11b promoter. In some cases, the cell-specific promoter is a CD68 promoter. In some cases, the cell-specific promoter is a CD138 promoter. In some cases, the cell-specific promoter is an IgH promoter. In some cases, the cell-specific promoter is an IgK promoter. In some cases, the cell-specific promoter is an IgL promoter. In some cases, the cell-specific promoter is an IgG promoter. In some cases, the cell-specific promoter is a CD19 promoter. In some cases, the cell-specific promoter is a CD20 promoter. In some cases, the cell-specific promoter is a TH promoter. In some cases, the cell-specific promoter is a TUBB3 promoter. In some cases, the cell-specific promoter is a FOXA2 promoter. In some cases, the cell-specific promoter is a GIRK2 promoter. In some cases, the cell-specific promoter is a Nurr1 promoter. In some cases, the cell-specific promoter is a DAT promoter. In some cases, the cell-specific promoter is an INS promoter. In some cases, the cell-specific promoter is a chromogranin A promoter. In some cases, the cell-specific promoter is a synaptophysin promoter. In some cases, the cell-specific promoter is a neuron-specific enolase promoter. In some cases, the cell-specific promoter is a Leu7 promoter.

In some embodiments the cell-specific promoter is specific to an NK cell. In some embodiments, the cell-specific promoter is a memory-like NK cell gene promoter. In some embodiments, the cell-specific promoter is an L-selectin (CD62L) promoter. In some embodiments, the cell-specific promoter is an interferon-gamma (IFNg) promoter. In some embodiments, the cell-specific promoter is neural cell adhesion molecule (CD56) promoter. In some embodiments, the inducible promoter is comprised in a plurality of promoters.

In one example, the differentiated cell is an NK cell, and the cell-specific promoter is a CD56 promoter. In another example, the differentiated cell is a memory-like NK cell, and the cell-specific promoter is a CD62L promoter. In yet another example, the differentiated cell is a memory-like NK cell, and the cell-specific promoter is an IFNg promoter. Still other combinations of differentiated cells and cell-specific promoters corresponding to those differentiated cells are possible. In some embodiments the cell-specific promoter may be selected from a CD16 promoter, a HLA-C promoter, a NKp46 promoter and a DAP10 promoter sequence.

In some embodiments, the plurality of promoters comprises a constitutive promoter. In some embodiments the constitutive promoter is a B2M promoter, a CIITA promoter, or a CAG promoter.

Examples of cell-specific promoters are found in the sequences provided in Table 1. In one embodiment the promoter comprises a sequence having 95%, 97%, 99% or 100% sequence identity to a 60, 70, 80, 90, 100, 200, 500, 1,000, 1,500 or 2,000 base pair long fragment of any one of SEQ ID 5 to 57. In some embodiments, the cell-specific promoter comprises a sequence having at least 95% sequence identity to a sequence that comprises 500 bp of any one of SEQ ID SEQ ID 5 to 57. In some embodiments, the cell-specific promoter comprises a sequence having at least 99% sequence identity to a sequence that comprises 500 bp of any one of SEQ ID SEQ ID 5 to 57. In some embodiments, such cell-specific promoter drives gene expression in a specific cell type manner.

In some embodiments, the inducible promoter may be a chemically inducible promoter such that the gene is not expressed until the cell is exposed to the corresponding inducing agent. In one aspect such inducing agent is present during in vitro differentiation, and such inducing agent may be a reagent used during the differentiation process. In another aspect the inducing agent may be administered to the individual in vivo (e.g., administration of rapamycin in a FKBP ligand system). An example of a chemically inducible promoter is the tetracycline (tet)-on promoter system, which can be used to regulate transcription of the nucleic acid. In this system, a mutated Tet repressor (TetR) is fused to the activation domain of herpes simplex virus VP 16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The present methods, constructs, and cells also contemplate an inducible system where gene expression or signaling is inhibited. For instance in the Tet-Off system, the presence of tetracycline or doxycycline prevents the interaction of the tetracycline transcriptional activator and the DNA operator sequence (tetO), and gene transcription is inhibited in the presence of tetracycline or doxycycline.

### Translation Control Sequences

In some embodiments, the construct further comprises a sequence encoding a translation control sequence. In some embodiments, the translation control sequence is an internal ribosome entry site (IRES) or 2A peptide. In some embodiments, the sequence encoding the translation control sequence is positioned 5′ or 3′ of the open reading frame encoding the tolerogenic factor. In some embodiments, the sequence encoding the translation control sequence is located between the ORF encoding the tolerogenic factor and another ORF encoding a native gene of the differentiated cell. In some embodiments, the native gene is B2M or CIITA.

### Transcription Regulators.

In some embodiments, the construct further comprises tagging an activator or repressor molecule to the inducible promoter. In some embodiments, the open reading frame encoding the one or more tolerogenic factor(s) is comprised in a transcription activator in operable connection with the inducible promoter. Further in one embodiment the ORF containing a tolerogenic factor may contain a stop sequence and/or a sequence to cleave the transcript after transcription of the final tolerogenic factor in the ORF. Such stop sequence may include polyadenylation sequences, either endogenous to the cell or inserted with an exogenous sequence encoding a tolerogenic factor. Poly A sequences may include the human beta globin poly A tail or other poly A sequences known in the art.

In some cases, the construct comprising a sequence encoding the tolerogenic factor (e.g., encoding the tolerogenic factor) or both is comprised in a nucleic acid molecule. In some cases, the inducible promoter is comprised in the nucleic acid molecule. In some cases, the nucleic acid molecule is comprised in a vector. In some cases, the vector is a plasmid, a minicircle, a CELiD, an adeno-associated virus (AAV) derived virion, a lentivirus, an adenovirus, or a herpes simplex virus (HSV). In some cases, the nucleic acid is comprised in the genome of the provided cell. In some cases, the construct is an expression vector.

The construct comprising the ORF encoding the tolerogenic factor, the inducible promoter, or both the ORF and the inducible promoter may be inserted into a cell at any appropriate location. For instance, the ORF may be integrated into a genome of the cell. In a particular aspect, the promoter is generally inserted within 3,000 base pairs of the transcription start site of the tolerogenic factor. For instance, the promoter and transgene can go anywhere in the genome (e.g., placed in a “safe harbor,” i.e., anywhere where the cell is still functional for growth and division and cell maintains normal karyotype).

In some cases, the construct comprises the inducible promoter. In such cases, the construct may be integrated at a safe harbor locus (e.g., AAVS1, Rosa26, CLYPL) or any other locus in the genome (e.g., B2M, CIITA). Alternatively, the inducible promoter may be introduced to the cell exogenously. For example, the cell-specific promoter may be comprised in a construct comprising a transgene (e.g., ORF encoding a tolerogenic factor, such as a SIRPα binding sequence). Alternatively, the construct may comprise a cell-specific promoter to be inserted in operable connection with an endogenous sequence in the cell, so as to change the expression of the endogenous gene in a cell-specific manner.

In some cases, the construct does not comprise the inducible promoter. In such instances, the cell-specific promoter may exist endogenously within the cell (e.g., stem cell, such as an induced pluripotent stem cell). Where the construct does not comprise the inducible promoter, the sequence encoding the tolerogenic factor may be located 3′ or 5′ of the endogenous gene whose expression is normally driven by the endogenous promoter. Alternatively, the construct comprising the ORF may be integrated at the site of the endogenous cell-specific promoter. In one instance, the sequence encoding the tolerogenic factor may be inserted in close proximity to the endogenous gene, where “close proximity” may mean within 100 base pairs, within 50 base pairs, within 30 base pairs, within 20 base pairs, within 10 base pairs, or immediately adjacent to the of the sequence encoding the endogenous gene.

Alternatively, the inducible promoter may be introduced to the cell exogenously. For example, the inducible promoter may be delivered to the cell as part of a construct comprising a transgene (e.g., ORF encoding a SIRPα binding sequence) and the cell-specific promoter.

### Additional Elements.

The constructs of the present invention may further include additional promoters, tolerogenic factors, cytokines, and other proteins. These additional promoters, tolerogenic factors, cytokines, and other proteins may confer increased specificity in expression level, decreased immunogenicity, or an expansion or survival advantage to the desired differentiated cell. The additional promoters may also be inducible promoters or constitutive promoters. Further the other proteins may include the CAR/TCRs described above or the costimulatory molecules described above.

In some cases, the construct may comprise a second ORF. The second ORF may comprise a second tolerogenic factor as described herein. Alternatively, the second ORF may comprise another sequence. In some cases, the second ORF is under the control of (e.g., in operable connection with) the same inducible promoter as the first ORF. Alternatively, in some cases, the second ORF is under the control of (e.g., in operable connection with) a second promoter. The second promoter may be an inducible promoter, such as a cell-type specific promoter or a chemically inducible promoter. Alternatively, the second promoter may be a constitutive promoter. By way of non-limiting example, the constitutive promoter may comprise a beta-actin promoter (such as a chicken beta-actin gene promoter), ubiquitin promoter, CAG promoter, beta-2 microglobulin (B2M) promoter, class II major histocompatibility complex transactivator (CIITA) promoter, glyceraldehyde-3-phosphate dehydrogenase (GAPDH) promoter, or 3-phosphoglycerate kinase (PGK) promoter.

In some cases, the second ORF comprises a cell-specific gene. For example, the second ORF may comprise the gene or a portion thereof whose expression is typically driven by the cell-specific promoter. In some cases, the second ORF comprises a second target gene. In some cases, the second target gene is a tolerogenic factor as discussed above. In some cases, the second target gene comprises a chimeric antigen receptor (CAR), a T-cell receptor (TCR), or a functional fragment thereof. In some cases, the CAR, TCR, or functional fragment thereof targets a tumor-specific antigen. In some cases, the TCR, or functional fragment thereof, comprises a TCR-α chain, a TCR-β chain, a TCR-γ chain, a TCR-δ chain, a TCR-ζ chain, a CD3 molecule, or any functional fragment or combination thereof. In some cases, the cell comprises a third ORF. For example, the second ORF may comprise a cell-specific gene or the portion of the cell-specific gene whose expression is normally driven by a cell-specific promoter and the third ORF comprises a CAR, TCR, or functional fragment thereof. In some cases, the cell comprises a further ORF. For example, the second ORF may comprise the gene or the portion thereof of the cell-specific gene of the inducible promoter; the third ORF comprises a CAR, TCR, or functional fragment thereof; and the fourth ORF comprises a human leukocyte (HLA) molecule, such as an HLA-A, HLA-B, HLA-C, HLA-E, HLA-F, or HLAG, or a functional fragment or variant thereof. Still other numbers and combinations of ORFs are contemplated herein.

In some cases, the second ORF is not operably connected with the cell-specific promoter and is instead operably connected with a second inducible promoter. In one such example, the inducible promoter is in operable connection with a sequence encoding a transcriptional activator. The inducible promoter is configured to activate upon expression of the transcriptional activator, thereby giving expression of the sequence encoded in the second ORF only after differentiation of the cell into the differentiated cell type.

The ORFs (e.g., the first and second ORFs or the first, second, and third ORFs) may be flanked or separated from one another by additional regulatory regions. By way of non-limiting example, the additional regulatory regions may comprise one or more polyadenylation sequences, translation control sequences (e.g., an internal ribosome entry segment, IRES or 2A self-cleaving peptide), enhancers, inducible elements, or introns. In some cases, the ORFs are separated by one or more IRESs or 2A peptide sequences. Such IRESs or 2A peptide sequences may allow for multi-cistronic expression.

In one particular aspect two ORFs are introduced into the cell; with the first ORF containing a cell-specific promoter and a sequence encoding a tolerogenic factor, a CAR or TCR, separated by 2A sequences and a second ORF may include a sequence encoding a costimulatory factor, which may or may not be driven by the same or a different cell specific promote as the first ORF. Alternatively, the second ORF may be omitted, and the CAR or TCR expressed by the first ORF may be stimulated by an exogenous costimulatory factor contacted to the cell.

### The Cell

The insertion of the constructs of the present invention into the cell may be done in any suitable manner. For instance, the genome of the cell may be modified in any manner. In some embodiments the construct is inserted in a site-specific manner, in others, the construct is inserted randomly into the cell. Site specific insertions may be performed by CRISPR-Cas9 (or the variants on such technology), TALE nucleases or zinc-finger nucleases. In specific embodiments the genome is modified by CRISPR gene editing. In some embodiments, any gene editing in the cells is carried out using one or more DNA-binding nucleic acids, such as alteration via an RNA-guided endonuclease (RGEN). Typically, “target sequence” generally refers to a sequence to which a guide sequence is designed to have complementarity, where hybridization between the target sequence and a guide sequence promotes the formation of a CRISPR complex. The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

The constructs of the invention may be inserted into any cell type. Preferably, the cell is a stem cell or multipotent cell (able to differentiate into all cell types within one lineage). In some cases, the cell is an induced pluripotent stem cell (iPSC). In some cases, the cell is an embryonic stem cell. In some cases, the cell is a mesenchymal stem cell (MSC). In some cases, the cell is a hematopoietic stem cell (HSC). The cells may be allogeneic, autologous, or xenogeneic with respect to an individual, including an individual in need of the cells, such as an individual with cancer.

The cell type expressing or signaling via the tolerogenic factor may be a unipotent, or differentiated cell-type that may or may not be fully mature. In one aspect, the cell type expressing or signaling via the tolerogenic factor is an immature unipotent or differentiated cell. In another aspect, the cell type expressing or signaling via the tolerogenic factor is a mature unipotent or differentiated cell. The present disclosure contemplates that the constructs may be active (either modulating expression of the tolerogenic factor or signaling via the tolerogenic factor) in differentiated cells of any kind, and in particular, in cells that correspond to the cell-specific promoter cell type. In some cases, the differentiated cell is an immune cell. In some cases, the differentiated cell is a white blood cell. In some cases, the differentiated cell is a neutrophil. In some cases, the differentiated cell is an eosinophil. In some cases, the differentiated cell is a basophil. In some cases, the differentiated cell is a mast cell. In some cases, the differentiated cell is a monocyte. In some cases, the differentiated cell is a macrophage. In some cases, the differentiated cell is a dendritic cell. In some cases, the differentiated cell is a natural killer (NK) cell. In some cases, the differentiated cell is a memory-like NK cell. In some cases, the differentiated cell is a lymphocyte. In some cases, the differentiated cell is a B cell. In some cases, the differentiated cell is a T cell. In some cases, the cell is a hepatocyte. In some cases, the differentiated cell is a cardiomyocyte. In some cases, the differentiated cell is a renal cell. In some cases, the differentiated cell is a dopaminergic neuron. In some cases, the differentiated cell is a pancreatic islet cell. In some cases, the differentiated cell is a macrophage. In some cases, the differentiated cell is a retinal pigment epithelium cell.

In some cases, constructs as disclosed herein may be used in methods for modulating expression of a gene in a cell. The methods may comprise providing to a cell any of the constructs disclosed herein. The methods may further comprise subjecting the cell to conditions sufficient to differentiate the cell into the differentiated cell type.

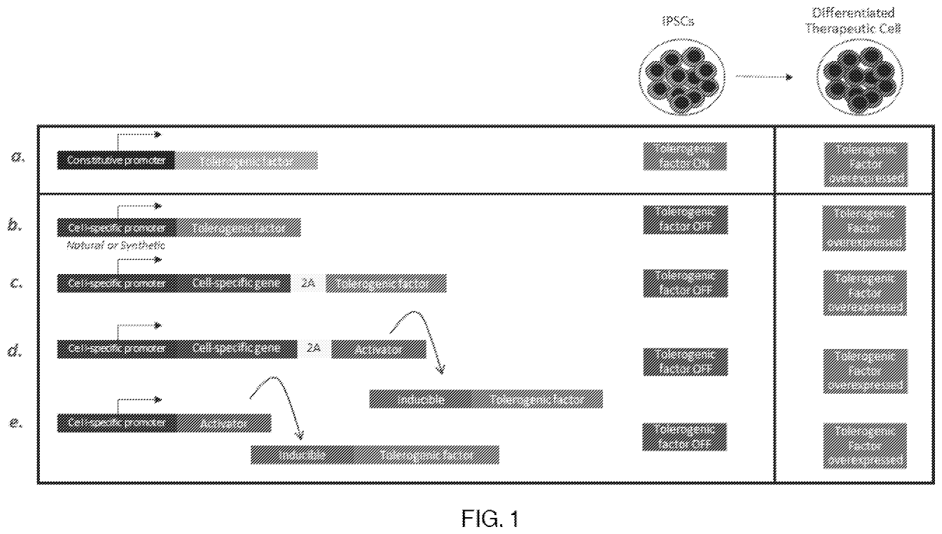

Several examples of constructs as disclosed herein are illustrated in FIG. 1. Row (a) of FIG. 1 illustrates a construct comprising a constitutive promoter in operable connection with an ORF encoding a SIRPα agonist. Because the promoter is constitutive, the SIRPα agonist is overexpressed in all cells comprising the construct (e.g., both a wild-type pluripotent stem cell as well as any differentiated therapeutic cells, as illustrated in FIG. 1). Rows (b)-(e) of FIG. 1 illustrate additional constructs for modulating expression of a gene. Row (b) of FIG. 1 illustrates an example of a construct comprising a cell-specific promoter in operable connection with a SIRPα agonist sequence as described herein. As illustrated by way of non-limiting example in FIG. 1, when the construct is provided to an iPSC, the iPSC displays only wild-type levels of the SIRPα agonist. In contrast, upon differentiation of the iPSC into the differentiated cell of the cell type corresponding to the cell-specific promoter, the transgenic SIRPα sequence is expressed, and the differentiated cell exhibits overexpression of the SIRPα agonist. Row (c) of FIG. 1 illustrates another example of a construct comprising a cell-specific promoter in operable connection with a SIRPα agonist sequence as described herein. The construct of row (c) comprises a cell-specific promoter in operable connection with a first ORF encoding a cell-specific gene as well as second ORF encoding a SIRPα agonist with a 2A peptide sequence between the first ORF and the second ORF. As illustrated by way of non-limiting example in FIG. 1, when the construct is provided to an iPSC, the iPSC displays only wild-type levels of the SIRPα agonist. In contrast, upon differentiation of the iPSC into the differentiated cell of the cell type corresponding to the cell-specific promoter, the transgenic SIRPα sequence is expressed, and the differentiated cell exhibits overexpression of the SIRPα agonist. The additional ORF comprising a cell specific gene relative to the construct of row (b) allows the differentiated cell to also express (e.g., overexpress) the cell-specific gene. Row (d) of FIG. 1 illustrates another example of a construct comprising a cell-specific promoter as described herein. The construct of row (d) comprises a cell-specific promoter in operable connection with a first ORF encoding a cell-specific gene as well as transcriptional activator. The complex further comprises a second ORF encoding a SIRPα agonist in operative connection with an inducible promoter which is activated by the transcriptional activator. As illustrated by way of non-limiting example in FIG. 1, when the construct is provided to an iPSC, the iPSC displays only wild-type levels of the SIRPα agonist. In contrast, upon differentiation of the iPSC into the differentiated cell of the cell type corresponding to the cell-specific promoter, the transcriptional activator is expressed, leading to expression of the transgenic SIRPα sequence, and the differentiated cell exhibits overexpression of the SIRPα agonist. The additional ORF comprising a cell specific gene relative to the construct of row (b) allows the differentiated cell to also express (e.g., overexpress) the cell-specific gene. Row (d) of FIG. 1 illustrates another example of a construct comprising a cell-specific promoter as described herein. The construct of row (d) comprises a cell-specific promoter in operable connection with a first ORF encoding a transcriptional activator. The complex further comprises a second ORF encoding a SIRPα agonist in operative connection with an inducible promoter which is activated by the transcriptional activator. As illustrated by way of non-limiting example in FIG. 1, when the construct is provided to an iPSC, the iPSC displays only wild-type levels of the SIRPα agonist. In contrast, upon differentiation of the iPSC into the differentiated cell of the cell type corresponding to the cell-specific promoter, the transcriptional activator is expressed, leading to expression of the transgenic SIRPα sequence, and the differentiated cell exhibits overexpression of the SIRPα agonist. Although FIG. 1 illustrates the cell as an iPSC, and the tolerogenic factor as a SIRPα agonist (e.g., binding sequence), other combinations of cell types and tolerogenic factors as contemplated herein

### Overview of the Methods

Disclosed herein, in one aspect, are methods for modulating gene expression or signaling via a gene in a cell. In some cases, the methods comprise driving expression of one or more genes (e.g., transgenes comprising a tolerogenic factor, such as a SIRPα agonist as described herein) off cell-specific gene promoters. Driving expression of tolerogenic factor transgenes from a cell-specific gene promoter (e.g., therapeutic cell-specific gene promoter) permits preferential survival of cells expressing the intended therapeutic cell phenotypic marker(s) (such as the tolerogenic factors) within a heterogenous population of cells which can be particularly beneficial when, e.g., administered in an allogeneic adoptive transfer setting. Accordingly, the methods described herein include methods for increasing survival of a cell population in vitro or in vivo.

Additionally, the present disclosure contemplates methods for enriching a cell type in vitro or in vivo. In one embodiment a specific phenotype can be enriched for within the final (e.g., therapeutic) cell population by driving expression of the tolerogenic factor off a gene promoter which is inducibly and/or preferentially expressed by the desired therapeutic cell subpopulation. In such a method the cell could be subjected to conditions under which cells not expressing the tolerogenic factor or signaling through the tolerogenic factor would be preferentially lysed or “eaten”, for instance, by other cell populations. In a particular embodiment, such enrichment occurs in vivo.

Further, the present disclosure includes methods for minimizing cell contamination in a heterologous population of cells. A distinct major advantage is to minimize contaminating cells, such as pluripotent and other multipotent stem cells which are starting and/or intermediate cell types during the therapeutic cell differentiation process during drug product manufacturing or in vivo, will not express the tolerogenic factor(s) and will preferentially be rejected (e.g., “eaten” or lysed by the subject's immune reaction, or by co-administered cells), for instance within an allogeneic adoptive transfer setting. In another embodiment, the enrichment may occur when the expression of the tolerogenic gene imparts a growth advantage or a survival advantage (e.g., IL-15 provides a growth advantage) to the cell population to be enriched. Thus, the present invention includes methods for enhancing the growth or survival of a cellular therapeutic in a time specific, or cell-type specific manner.

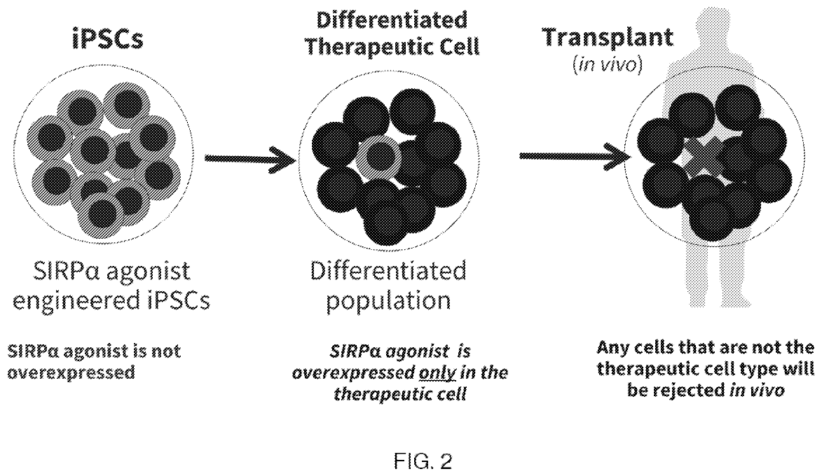

FIG. 2 illustrates one of the advantages of employing the expression strategies illustrated in rows (b)-(e) of FIG. 1 as opposed to that illustrate in row (a) of FIG. 1. The engineered iPSCs of the example methods illustrated in FIG. 1 do not overexpress the tolerogenic factor because the tolerogenic factor agonist is under the control of an inducible promoter which only becomes active once the iPSCs are differentiated into cells of the target therapeutic cell type. Any remaining undifferentiated cells which contaminate the differentiated therapeutic cell population are then rejected in an in vivo setting because they do not express high levels of the tolerogenic factor; i.e., do not protect the undifferentiated (i.e., stem, pluripotent, or multipotent) cell from the host's immune response.

One major advantage is that a specific phenotype can be enriched for within the final therapeutic cell population by driving expression of the tolerogenic factor off a gene promoter which is expressed preferentially by the desired therapeutic cell subpopulation.

For example, memory-like NK cells which have been shown to promote more durable anti-tumor responses by engrafting longer in the host and maintaining a stem-like phenotype (doi: 10.1126/scitranslmed.aaf2341) preferentially express CD62L, IFNg, NKp44, TRAIL, etc. as compared to more terminally differentiated NK cell subpopulations. To assess the advantage of expressing tolerogenic factors from one or more of these memory-like NK cell gene promoters, CD47 will be driven off CD62L, IFNg, 2 separate constitutive promoters (B2M and CAGG), and one pan-NK cell promoter (CD56).

Further methods include a method of making a cell having modulated expression of a tolerogenic factor. Such methods might include introducing a construct comprising either a tolerogenic factor, a cell-specific promoter or both a tolerogenic factor and cell-promoter into a stem cell, a pluripotent cell, or a multipotent cell. In one specific embodiment, iPSCs are site-specifically targeted for integration of these transgene cassettes which may also express a CAR and/or TCR targeted to a tumor-specific antigen. The integrated site can be B2M, CIITA, or expressed in-frame at the intended memory-like NK cell genes (i.e. at CD62L, IFNg, etc.) either at the 5′ or 3′ of the coding sequence of the gene, separated from the native gene by an IRES or 2A self-cleavable peptide sequence to allow for multi-cistronic expression. The iPSCs are then be differentiated into NK cells in vitro. Differentiated NK cell populations are then be injected into humanized NSG mouse tumor models and anti-tumor response and engraftment durability is tested. As a proxy for in vivo tumor killing efficacy, differentiated cells may also be assayed in vitro for IFNg expression and memory-like NK phenotype in culture alone or in co-culture with allogeneic effector immune cells which will preferentially kill non-CD47-expressing therapeutic cells. The fraction of memory-like NK cells after co-culture will be indicative of preferential survival of these cells when CD47 is driven off memory-like NK cell promoters (CD62L and/or IFNg). A factor of 25%, 50%, 100%, 200% more enrichment of these cells may be therapeutically beneficial.

Another distinct major advantage is contaminating pluripotent and other multipotent stem cells which are starting and/or intermediate cell types during the therapeutic cell differentiation process during drug product manufacturing will not express the tolerogenic factor(s) and will preferentially be rejected within an allogeneic adoptive transfer setting. To evaluate this, the same transgene design as in the previous paragraph will be constructed and cells will be differentiated into NK cells or maintained at the iPSC stage. A subset of the iPSC-gene edited product will contain Akaluc reporter that will be constitutively expressed and the other subset will contain FireFly luciferase reporter that is also constitutively expressed. The Firefly luciferase reporter iPSC population will be differentiated into NK cells and the Akaluc reporter iPSC population will remain pluripotent. iPSCs will then be spiked into NK cell at increasing fractions and then total mixture of cells will then be injected into humanized NSG mice immediately. Groups with constitutive expression of CD47 will not reject the iPSCs and thus form teratomas at increasing amounts with increasing iPSC fractions in the injected cell mixture. Groups with NK cell-specific CD47 expression will immediately reject the iPSCs and thus not form teratomas at any fraction. As a proxy for this in vivo experiment, the same iPSC/NK mixture experiment will be conducted in vitro in co-culture with allogeneic effector immune cells for extended periods of time. The percentage of surviving non-NK cells within culture will be indicative of the surviving iPSC-derived non-NK cell progeny. A factor of 1%, 2%, 5%, 10%, 25%, 50%, 100%, 200% fewer surviving iPSCs and iPSC-derived non-NK cell progeny either in vitro or in vivo will be therapeutically beneficial from a safety and regulatory perspective.

Also disclosed herein are the strategies of using inducibly expressed tolerogenic factors to overcome immune rejection in cell-based transplantation therapies using universal donor stem cell lines.

An aspect of the present disclosure comprises expression of or signaling through rationally designed tolerogenic factor sequences that retain binding to the wild-type protein's corresponding native binding partner and display ‘don't eat me’ signaling response comparable or higher than wild-type protein, with the advantage of having” skewed” signaling function towards the “don't eat me” signal and optionally detuned binding of one or more other endogenous binding partners.

### EXAMPLES

### Example 1a: Screening Promoter Sequences and Tagging Endogenous NK-Expressing Genes to Identify NK-Specific Promoters

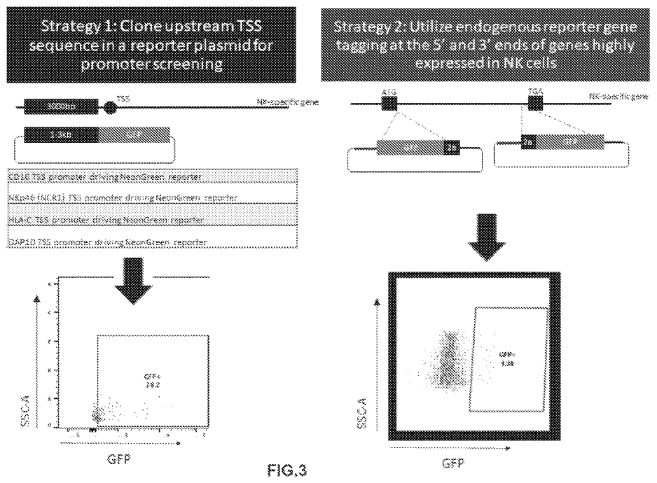

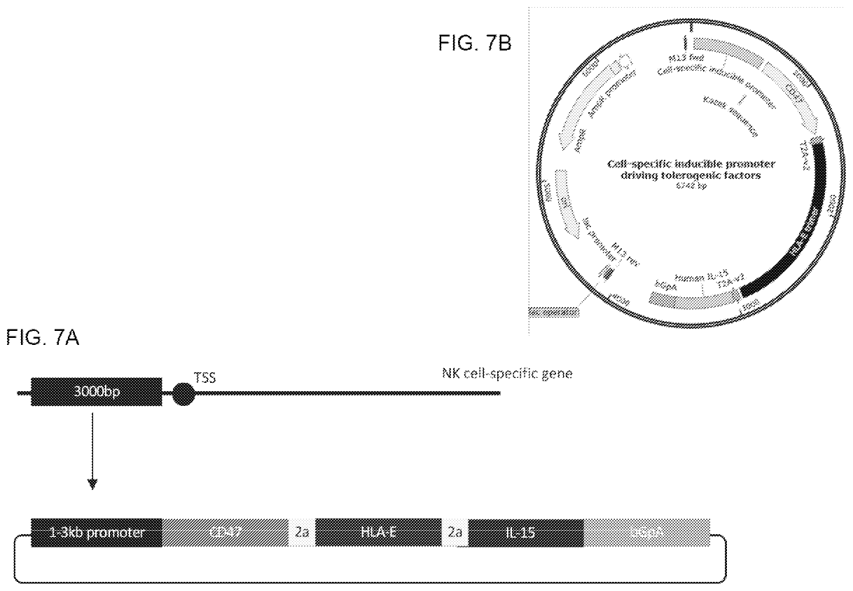

In this example one of the differentiated cell types disclosed herein were used to identify cell-specific promoters and inserted into a reporter plasmid to screen for NK-selective expression of a reporter gene. In particular the sequence upstream of the transcription start site of genes selectively expressed in NK cells was cloned. The sequence up to 3,000 base pairs upstream of the transcription start site was identified, and promoters within that identified sequence were cloned (see FIG. 3, strategy 1). Customized gBlocks containing the cell-specific promoter sequence and homology arms were Gibson cloned into a backbone plasmid. (FIG. 7). The plasmid containing the cell-specific promoter was transformed into E. Coli, grown up to obtain multiple colonies and the sequence was verified. Once the sequence was verified, iPSCs, primary NK, or iPSC derived NK cells were transfected with the plasmid and checked for expression of the fluorescent reporter after 24-48 hours.

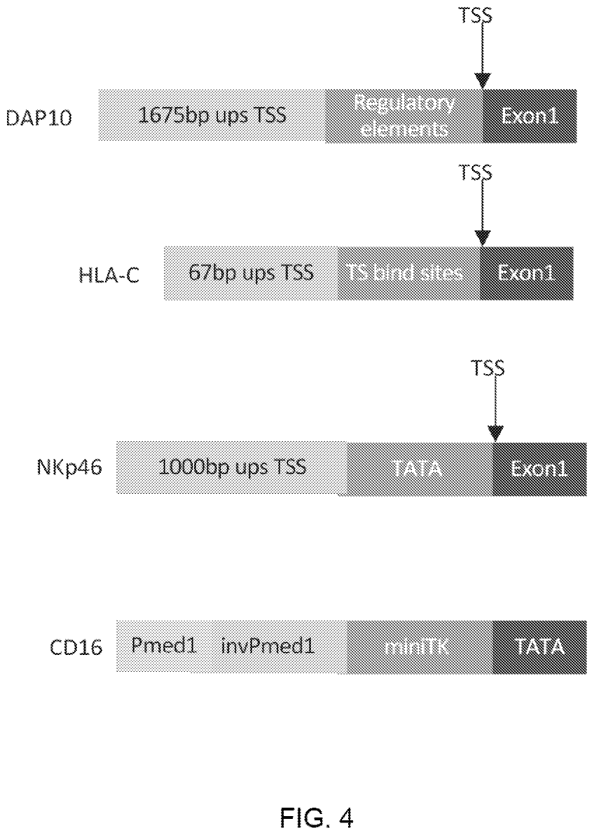

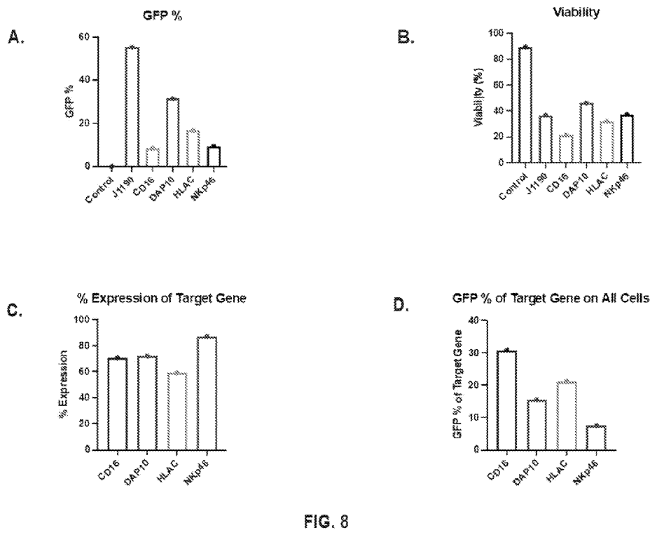

In particular the cell-specific promoters driving expression of CD16, NKp46 (NCR1), HLA-C, and DAP10 were identified. Constructs were designed comprising the identified cell-specific promoter sequence, optionally transcription regulatory elements (e.g., TATA box, TS bind sites, and miniTK) and included in a construct comprising a reporter sequence (GFP) (see e.g., FIG. 4). For instance with DAP10, 1675 bp upstream of the TSS were included; likewise for HLA-C, 675 bp; and for NKp46 1,000 bp. For CD16, multiple promoter elements were included, such as Pmed1 and an invert Pmed1. In one particular experiment, the construct was cloned into differentiated NK cells and iPSCs at the location of the target gene (i.e., the gene from which the promoter was identified) (10 microgram pulse coded DN100). GFP expression, viability, expression of the target gene, and GFP % of the target gene were assessed (see FIG. 8).

### Example 1b: Utilize Endogenous Reporter Gene Tagging at the 5′ and 3′ Ends of Genes Highly and Selectively Expressed in NK Cells

In another strategy, we generated constructs to be inserted near a target gene (i.e. the gene selectively expressed in the desired differentiated cell type, e.g., CD56 is shown in FIG. 3, Strategy 2) in the cell genome. These constructs did not comprise a promoter, and were inserted into primary NK cells' genomes either near or at the 5′ end (“5′ gene tagging”) or the 3′ end (“3′ gene tagging”) of the sequence encoding the target gene. Fluorescence was read. Fluorescence was particularly demonstrated with the 5′ tagging strategy in primary NK cells.

### Example 1c: Example of a Tolerogenic Construct Under a Cell Specific Promoter

As described herein, we generated constructs containing multiple tolerogenic factors separated by translation regulatory sequences. As shown in FIG. 7 we designed a backbone construct to incorporate the cell-specific promoter and a tolerogenic factor, with CD47 or a variant or fragment thereof as the cell specific tolerogenic factor, separated by a transcription regulatory sequence (T2A-V2); a second cell-specific tolerogenic factor, HLA-E; again separated by a transcription regulatory sequence; and an additional protein IL-15.

### Example 2A. Cell Type-Specific Expression of SIRPα Agonists

In this example, the advantages of expressing tolerogenic factors (e.g., SIRPα agonists, such as CD47 and variants thereof) in NK cells are assessed.

Memory-like NK cells are documented to preferentially express certain molecules such as L-selectin (CD62L), interferon-gamma (IFNg), natural cytotoxicity triggering receptor 2 (NKp44), and TNF-related apoptosis-inducing ligand (TRAIL). Expression of a tolerogenic factor (e.g., CD47) in induced pluripotent stem cells (iPSCs) may be driven off any of the CD62L and IFNg promoters, as well as one or more constitutive promoters (e.g., a beta-2 microglobulin (B2M) promoter and a CAGG promoter) and a pan-NK cell promoter (e.g., a neural cell adhesion (CD56) promoter). The iPSCs are targeted for site-specific integration of transgene cassettes comprising the tolerogenic factor as well as a chimeric antigen receptor (CAR) or T-cell receptor (TCR) targeted to a tumor specific antigen. The cassettes are integrated 3′ or 5′ of the coding sequence of the gene for each of the promoters above. The tolerogenic factor and therapeutic molecule (e.g., TCR, CAR) are separated from the native gene by an IRES or s2 self-cleavable peptide sequence to allow multi-cistronic expression. The iPSCs are then differentiated into NK cells in vitro. The differentiated NK cell populations are then injected into humanized NSG mouse tumor models and anti-tumor response and engraftment durability are tested. Differentiated NK cells are also assayed for IFNg expression and memory-like NK phenotype in culture alone or in co-culture with allogenic effector immune cells which will preferentially kill non-tolerogenic-expressing (e.g., non-CD47-expressing) immune cells. The fraction of memory-like NK cells after co-culture is indicative of preferential survival of these cells when the tolerogenic factor is driven off memory-like NK cell promoters (e.g., CD62L or INFg).

### Example 2B. Rejection of Non-Tolerogenic Factor-Expressing Immune Cells

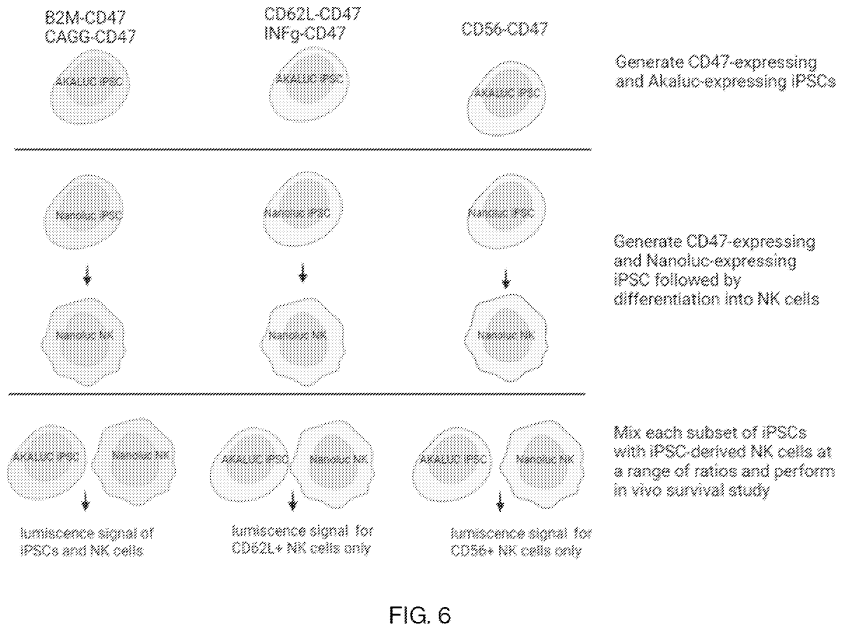

The same transgene design as described in Example 2A is constructed and iPSC cells are targeted as in Example 2A. Subsets of cells are differentiated into NK cells or maintained at the iPSC stage. A subset of the iPSC-gene edited product contains an Akaluc reporter which is constitutively expressed, and the other subset contains a FireFly luciferase reporter which is also constitutively expressed. The FireFly luciferase reporter iPSC population is differentiated into NK cells (middle row of FIG. 6) and the Alakluc reporter iPSC population remains pluripotent (top row of FIG. 6). iPSCs are then spiked into NK cells at increasing fractions the total mixture of cells injected into humanized NSG mice. Groups with constitutive expression of the tolerogenic factor (e.g., CD47) will not reject the iPSCs and thus form teratomas at increasing amounts with increasing iPSC fractions in the injected cell mixture. Groups with NK cell-specific tolerogenic factor expression will immediately reject the iPSCs and thus not form teratomas at any fraction. The same iPSC/NK mixture co-cultured in vitro with allogeneic effector immune cells for extended periods of time. The percentage of surviving non-NK cells within culture will be indicative of the surviving iPSC-derived non-NK cell progeny. A factor of fewer surviving iPSCs and iPSC-derived non-NK cell progeny either in vitro or in vivo is observed.

### Example 3 Induced Expression of Tolerogenic Factors Leads to Enhanced Protection in a Cytotoxicity Assay

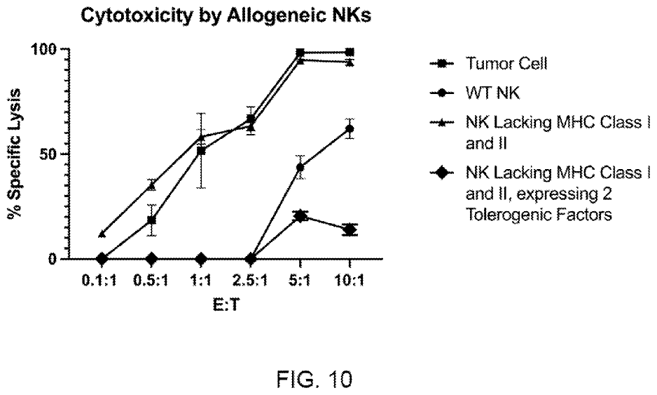

In NK cells lacking expression of both MHC Class I and MHC Class II, transgenic expression of tolerogenic factors enhances their protection against cytolytic allogeneic NKs. Normal healthy cells express MHC class I molecules on their surface which act as ligands for inhibitory receptors and contribute to the self-tolerance of NK cells. In a mixture of NK cells and cells not expressing MHC class I molecules, the NK cells will lyse the cells not expressing MHC class I. Accordingly NK cells not expressing MHC class I or MHC class II. In a competition assay between allogeneic NKs which would normally kill non-self cells (e.g., tumor cells or cells not displaying self antigens via MHC Class I or MHC Class II), cells expressing the tolerogenic factors were not lysed. Tumor cells, WT NK cells (expressing MHC Class I and MHC Class II), NK cells lacking MHC Class I and MHC Class II, and NK cells lacking MHC Class I and Class II, but that were transfected with a construct of the invention containing two tolerogenic factors, each a target cell, were incubated with cytolytic allogeneic NK cells (effector cells) at a ratio of 0.1:1; 0.5:1; 2.5:1; 5:1, and 10:1 effector cells to target cells. As shown by FIG. 10, tumor cells and NK cells lacking MHC Class I and MHC Class II were specifically lysed between 80% of the time, whereas WT NK cells were lysed around 60% of the time, and NK cells lacking MHC Class I and Class II but expressing the two tolerogenic factors had at most 20-30% specific lysis even when contacted with a high ratio of effector cells.

### Example 4 Engineered Cells

Pluripotent stem cells (e.g. iPSCs) are engineered to express one or more tolerogenic factors selected from SEQ ID NOs 1-4 or any one of the tolerogenic factors described in PCT/US2023/071212.

TABLE 1
Tolerogenic Factors

| SEQ | |
|---|---|
| ID | |
| NO | Sequence |
| 1 | MWPLVAALLLGSACCGSAQLLFNKTKSVEFTFCNDTVVIPCFVTN |
| | MEAQNTTEVYVKWKFKGRDIYTFDGALNKSTVPTDFSSAKIEVSQ |
| | LLKGDASLKMDKSDAVSHTGNYTCEVTELTREGETIIELKYRVVS |
| | WFSPNENILIVIFPIFAILLFWGQFGIKTLKYRSGGMDEKTIALL |
| | VAGLVITVIVIVGAILFVPGEYSLKNATGLGLIVTSTGILILLHY |
| | YVFSTAIGLTSFVIAILVIQVIAYILAVVGLSLCIAACIPMHGPL |
| | LISGLSILALAQLLGLVYMKFVASNQKTIQPPRKAVEEPLNAFKE |
| | SKGMMNDE |
| 2 | MWPLVAALLLGSACCGSAQLLFNKTKSVEFTFCNDTVVIPCFVTN |
| | MEAQNTTEVYVKWKFKGRDIYTFDGALNKSTVPTDFSSAKIEVSQ |
| | LLKGDASLKMDKSDAVSHTGNYTCEVTELTREGETIIELKYRVVS |
| | WFSPNENILIVIFPIFAILLFWGQFGIKTLKYRSGGMDEKTIALL |
| | VAGLVITVIVIVGAILFVPGEYSLKNATGLGLIVTSTGILILLHY |
| | YVFSTAIGLTSFVIAILVIQVIAYILAVVGLSLCIAACIPMHGPL |
| | LISGLSILALAQLLGLVYMKFV |
| 3 | MWPLVAALLLGSACCGSAQLLFNKTKSVEFTFCNDTVVIPCFVTN |
| | MEAQNTTEVYVKWKFKGRDIYTFDGALNKSTVPTDFSSAKIEVSQ |
| | LLKGDASLKMDKSDAVSHTGNYTCEVTELTREGETIIELKYRVVS |
| | WFSPNENILIVIFPIFAILLFWGQFGIKTLKYRSGGMDEKTIALL |
| | VAGLVITVIVIVGAILFVPGEYSLKNATGLGLIVTSTGILILLHY |
| | YVFSTAIGLTSFVIAILVIQVIAYILAVVGLSLCIAACIPMHGPL |
| | LISGLSILALAQLLGLVYMKFVASNQKTIQPPRNN |
| 4 | MWPLVAALLLGSACCGSAQLLFNKTKSVEFTFCNDTVVIPCFVTN |
| | MEAQNTTEVYVKWKFKGRDIYTFDGALNKSTVPTDFSSAKIEVSQ |
| | LLKGDASLKMDKSDAVSHTGNYTCEVTELTREGETIIELKYRVVS |
| | WFSPNENILIVIFPIFAILLFWGQFGIKTLKYRSGGMDEKTIALL |
| | VAGLVITVIVIVGAILFVPGEYSLKNATGLGLIVTSTGILILLHY |
| | YVFSTAIGLTSFVIAILVIQVIAYILAVVGLSLCIAACIPMHGPL |
| | LISGLSILALAQLLGLVYMKFVASNQKTIQPPRKAVEEPLN |

The pluripotent stem cells are further engineered to express a distinct promoter driving expression of the one or more tolerogenic factors. In one group of pluripotent stem cells, the cells are configured such that expression of the distinct promoter driving expression of the one or more tolerogenic factors occurs in conjunction with the cell's wildtype genome expression. In another group of pluripotent stem cells, the cells are configured such that expression of the distinct promoter driving expression of the one or more tolerogenic factors replaces, in part, the cell's wildtype genome expression. The cells are engineered to express any one or more of the distinct promoters, or functional portions thereof, described in SEQ ID NOs 5-57.

TABLE 2
Distinct Promotors

| SEQ | | |
|---|---|---|
| ID | | |
| NO | Sequence | Description |
| 5. | CCTATTTCAACAAGTGGAGACAGAATAACTGGATTTCCATACAGG | TP53 |
| | AAAGATACCAGAGACTGACTCCTACACCTCACACCATAAACAATT | regulatory T- |
| | AATTTTAAGAATTAATTAATGGCTCAAGGACCTTACTGTAAAACT | cell (Treg) |
| | TACAACCATAAAGGTCCTAAAAGAAAATGTAAGATAATATCTTCA | |
| | TGACCCTGGGGTTAAAAAAAAAAAAAAAGATGTCCTAAACAGGAC | |
| | AAGGCAAATACTGAACATAAAAAAGATAAATCCACTCCTCTTAAG | |
| | ATACTGTAAACTCTGTAAAGCAAACAAATAGGCAAGCAACAGATC | |
| | AGAAGAAAACATTCACGACACATGGATCTGATAAAGGACTTGTAT | |
| | CCAGAATGTATAAAGCAGTCCCACAACTGAACAATAAAAACAAAC | |
| | AAAAAACCAAAATAACAGGTAAAAGACTCGAAGAGCTACTTTACA | |
| | AACAAAATACGAATGGCCAATAGGCACATGAAAAAATGCTGAACA | |
| | TCCTTAGTCAATAGAGAACTGTAAATTACAACCACAAGGATATAC | |
| | CACATTAGAAAGACTGACAATACCTAATGTCCGGAAGGCTGTGGC | |
| | ACAACCATAATAACTCCCATACCTTGCTAGTTGGAGTGTAAAATG | |
| | GTACAACCGCTCTGGAAAACTCAGAGCTTCTGAAAAAGTTCAAAA | |
| | TACAGCTACTTTTTACTTCCAAACTCGCAATTCCCCTCCTAAGTA | |
| | TTTCTCCAAGAAACACGAAAACATATGATCACAAAAAGAATTGTA | |
| | CAAGAATGTTTATAGCAGCTTTATTTCATAACCGCAAATGGGAAA | |
| | CAACTCAAAAGGCCATCAAAAGGACGGATATACAATCGATGGACT | |
| | ATACTAAATGAAAAGGAGCAAAATACTGATATATACAACATGAAC | |
| | GAATGTCAGACAGTACATTGAAGGACAGAAGCCCGACAAAAATGA | |
| | GCACATAATGTATGATTCCCCCCTTTTTTTTGAGACGGAGTTTCG | |
| | TTCTTGTCGCCCAGGCTGGAGTGTAGTGGCACGATCTTGGCTCAC | |
| | TGCAACCTCTGCCTCCCGGGTTCAAGCGATTCTCCTGCCTCACCC | |
| | TCCCGAATAGCTGGGATTACAGGCACCCACCACGCCCAGCTAATT | |
| | TTTGTATTTTTTAGTAGAGACGGGGATTCACCACGTTGGCCACGC | |
| | TGGTCTGGAACTCCTATCCTCAAGTAATCCGCCCGCCTCGGCCTC | |
| | CCAAAGTGCAGGCGTGAGCCACAGCGCCCAGCCTGATTCCATTCT | |
| | ATATGAAGTTCTCCAACAGGCAAAATGGTTATGGAGATCAAAATA | |
| | AAGGTGGGGTCGGGAATCGACTGGGAAGAGACGTGATGAAACGTT | |
| | TCTGGGACGATGAAAAGGGTCTGTGACTTGGTAGGCATCACGGAG | |
| | CGGTTAGGGGCCAAAACTCATCTTCCTGTGCACTTGCTGTGTGCA | |
| | CTGGCGCTGTGTGTAAATGCCACCTCGATTTAGGAAAAAGATGAC | |
| | GTAAGTACGGCACAAAGTGGCCGGTACGCGGCAGGTGCATGGGAA | |
| | GAAACTGCGGAATGAAACAACCGCGAGCTAAGAGATGGGGCAGCG | |
| | GGAGAAATGAATTCGAGTTCCGCCTCCTACCAGGAAGAACCGGCT | |
| | CGGGCCGGAGGGCTGCACGGAGGACCACACGGACGCCTGCGGGCC | |
| | CGCCCCTTCCGCTTCACGACGTTCAGCCTGCGTCTGGAACTGGAA | |
| | TGGCCTAGCCCAAAGCTAGATAACAGGTAGATTGTTTTTCCGACA | |
| | AATTATCAAACGACCCATCATTGCACTCTTTCAAAATTTGATTCT | |
| | CAGACGTACCCATTCTTTTTTTTTTTCCTCCGGGAAGATGAGATA | |
| | TACTCATTCTTGAAAATACCTCCGGGCTTGCCTTCTGCACACTTC | |
| | TTTCCCTCCCTGTCTCACGCCATGGTAGCGTCCGCCTAGGTTGCA | |
| | GGCGACCCGCGGGGTGGGGCACACCATTCAAAGAAGGGGAGGGAT | |
| | TGAGGTTTGCATCAAAACAAATACCCCTGCCTTTGCAAAGGCCAT | |
| | AACTAAGTAATCCAGAAAAAGAAATGCAGGCGGAGAATAGCAGCC | |
| | TCCCTCTGCCAAGTAAGAGGAACCGGCCTAAAGGACATTTTCTCT | |
| | CTCTCTCCTCCCCTCTCATCGGGTGAATAGTGAGCTGCTCCGGCA | |
| | AAAAGAAACCGGAAATGCTGCTGCAAGAGGCAGAAATGTAAATGT | |
| | GGAGCCAAACAATAACAGGGCTGCCGGGCCTCTCAGATTGCGACG | |
| | GTCCTCCTCGGCCTGGCGGGCAAACCCCTGGTTTAGCACTTCTCA | |
| | CTTCCACGACTGACAGCCTTCAATTGGATTTTCTCCATCTAGCGG | |
| | AGCCGGGGGCTGCCTGGAAAGATCGCTCCAGGAAGGACAAAGGTC | |
| | CGGAAGTTGTGGGACCTTAGCAGCTTGGGCTCCCCGGATCACCCC | |
| | CAAATGATCATTTCGGAATGGAGCCCCAGTTTTCACTAGGATGCC | |
| | ATGGGCTCTAAAATATACAGCTATGAGTTCTCAATGTTTCGAGAT | |
| | CCAAAAGTCTCAGACCTCAATGCTTTGTGCATCTTTTATTTCAGG | |
| | GATTCCCTACGCCCAGCACCGGGTGGATGTGCAAAGAAGTACGCT | |
| | TTAGGCCGGCTCAAGGTTCCCCAAAGCTCCACTCCTCTGCCTAGG | |
| | CGTTCAACTTTGAGTTCGGATGGTCCTAACATCCCCATCATCTAC | |
| | ACCCAGGTCTCCCAACAATGCAACTCCTATGATGATCCCTCTAGC | |
| | CAAGCTTCCATCCCACTCACCCCCAAACTCGCTAAGTCCCCACTG | |
| | CCCCACCCCCAGCCCCAGCGATTTTCCCGAGCTGAAAATACACGG | |
| | AGCCGAGAGCCCGTGACTCAGAGAGGACTCATCAAGTTCAGTCAG | |
| | GAGCTTACCCAATCCAGGGAAGCGTGTCACCGTCGTGGAAAGCAC | |
| | GCTCCCAGCCCGAACGCAAAGTGTCCCCGGAGCCCAGCAGCTACC | |
| | TGCTCCCTGGACGGTGGCTCTAGACTTTTGA | |
| 6. | TCTGTCTCCCTCTGCTCACCTTGGGGTTTCTCTGACTGCATCTTG | TNF IFNg |
| | TCCCCTTCTCTGTCGATCTCTCTCTCGGGGGTCGGGGGGTGCTCT | |
| | CTCCCAGGGCGGGAGGTCTGTCTTCCGCCGCGTGCCCCGCCCCGC | |
| | TCACTGTCTCTCTCTCTCTCTCTCTTTCTCTGCAGGTTCTCCCCA | |
| | TGACACCACCTGAACGTCTCTTCCTCCCAAGGGTGTGTGGCACCA | |
| | CCCTACACCTCCTCCTTCTGGGGCTGCTGCTGGTTCTGCTGCCTG | |
| | GGGCCCAGGTGAGGCAGCAGGAGAATGGGGGCTGCTGGGGTGGCT | |
| | CAGCCAAACCTTGAGCCCTAGAGCCCCCCTCAACTCTGTTCTCCC | |
| | CTAGGGGCTCCCTGGTGTTGGCCTCACACCTTCAGCTGCCCAGAC | |
| | TGCCCGTCAGCACCCCAAGATGCATCTTGCCCACAGCACCCTCAA | |
| | ACCTGCTGCTCACCTCATTGGTAAACATCCACCTGACCTCCCAGA | |
| | CATGTCCCCACCAGCTCTCCTCCTACCCCTGCCTCAGGAACCCAA | |
| | GCATCCACCCCTCTCCCCCAACTTCCCCCACGCTAAAAAAAACAG | |
| | AGGGAGCCCACTCCTATGCCTCCCCCTGCCATCCCCCAGGAACTC | |
| | AGTTGTTCAGTGCCCACTTCCTCAGGGATTGAGACCTCTGATCCA | |
| | GACCCCTGATCTCCCACCCCCATCCCCTATGGCTCTTCCTAGGAG | |
| | ACCCCAGCAAGCAGAACTCACTGCTCTGGAGAGCAAACACGGACC | |
| | GTGCCTTCCTCCAGGATGGTTTCTCCTTGAGCAACAATTCTCTCC | |
| | TGGTCCCCACCAGTGGCATCTACTTCGTCTACTCCCAGGTGGTCT | |
| | TCTCTGGGAAAGCCTACTCTCCCAAGGCCACCTCCTCCCCACTCT | |
| | ACCTGGCCCATGAGGTCCAGCTCTTCTCCTCCCAGTACCCCTTCC | |
| | ATGTGCCTCTCCTCAGCTCCCAGAAGATGGTGTATCCAGGGCTGC | |
| | AGGAACCCTGGCTGCACTCGATGTACCACGGGGCTGCGTTCCAGC | |
| | TCACCCAGGGAGACCAGCTATCCACCCACACAGATGGCATCCCCC | |
| | ACCTAGTCCTCAGCCCTAGTACTGTCTTCTTTGGAGCCTTCGCTC | |
| | TGTAGAACTTGGAAAAATCCAGAAAGAAAAAATAATTGATTTCAA | |
| | GACCTTCTCCCCATTCTGCCTCCATTCTGACCATTTCAGGGGTCG | |
| | TCACCACCTCTCCTTTGGCCATTCCAACAGCTCAAGTCTTCCCTG | |
| | ATCAAGTCACCGGAGCTTTCAAAGAAGGAATTCTAGGCATCCCAG | |
| | GGGACCACACCTCCCTGAACCATCCCTGATGTCTGTCTGGCTGAG | |
| | GATTTCAAGCCTGCCTAGGAATTCCCAGCCCAAAGCTGTTGGTCT | |
| | GTCCCACCAGCTAGGTGGGGCCTAGATCCACACACAGAGGAAGAG | |
| | CAGGCACATGGAGGAGCTTGGGGGATGACTAGAGGCAGGGAGGGG | |
| | ACTATTTATGAAGGCAAAAAAATTAAATTATTTATTTATGGAGGA | |
| | TGGAGAGAGGGGAATAATAGAAGAACATCCAAGGAGAAACAGAGA | |
| | CAGGCCCAAGAGATGAAGAGTGAGAGGGCATGCGCACAAGGCTGA | |
| | CCAAGAGAGAAAGAAGTAGGCATGAGGGATCACAGGGCCCCAGAA | |
| | GGCAGGGAAAGGCTCTGAAAGCCAGCTGCCGACCAGAGCCCCACA | |
| | CGGAGGCATCTGCACCCTCGATGAAGCCCAATAAACCTCTTTTCT | |
| | CTGAAATGCTGTCTGCTTGTGTGTGTGTGTCTGGGAGTGAGAACT | |
| | TCCCAGTCTATCTAAGGAATGGAGGGAGGGACAGAGGGCTCAAAG | |
| | GGAGCAAGAGCTGTGGGGAGAACAAAAGGATAAGGGCTCAGAGAG | |
| | CTTCAGGGATATGTGATGGACTCACCAGGTGAGGCCGCCAGACTG | |
| | CTGCAGGGGAAGCAAAGGAGAAGCTGAGAAGATGAAGGAAAAGTC | |
| | AGGGTCTGGAGGGGCGGGGGTCAGGGAGCTCCTGGGAGATATGGC | |
| | CACATGTAGCGGCTCTGAGGAATGGGTTACAGGAGACCTCTGGGG | |
| | AGATGTGACCACAGCAATGGGTAGGAGAATGTCCAGGGCTATGGA | |
| | AGTCGAGTATGGGGACCCCCCCTTAACGAAGACAGGGCCATGTAG | |
| | AGGGCCCCAGGGAGTGAAAGAGCCTCCAGGACCTCCAGGTATGGA | |
| | ATACAGGGGACGTTTAAGAAGATATGGCCACACACTGGGGCCCTG | |
| | AGAAGTGAGAGCTTCATGAAAAAAATCAGGGACCCCAGAGTTCCT | |
| | TGGAAGCCAAGACTGAAACCAGCATTATGAGTCTCCGGGTCAGAA | |
| | TGAAAGAAGAAGGCCTGCCCCAGTGGGGTCTGTGAATTCCCGGGG | |
| | GTGATTTCACTCCCCGGGGCTGTCCCAGGCTTGTCCCTGCTACCC | |
| | CCACCCAGCCTTTCCTGAGGCCTCAAGCCTGCCACCAAGCCCCCA | |
| | GCTCCTTCTCCCCGCAGGGACCCAAACACAGGCCTCAGGACTCAA | |
| | CACAGCTTTTCCCTCCAACCCCGTTTTCTCTCCCTCAAGGACTCA | |
| | GCTTTCTGAAGCCCCTCCCAGTTCTAGTTCTATCTTTTTCCTGCA | |
| | TCCTGTCTGGAAGTTAGAAGGAAACAGACCACAGACCTGGTCCCC | |
| | AAAAGAAATGGAGGCAATAGGTTTTGAGGGGCATGGGGACGGGGT | |
| | TCAGCCTCCAGGGTCCTACACACAAATCAGTCAGTGGCCCAGAAG | |
| | ACCCCCCTCGGAATCGGAGCAGGGAGGATGGGGAGTGTGAGGGGT | |
| | ATCCTTGATGCTTGTGTGTCCCCAACTTTCCAAATCCCCGCCCCC | |
| | GCGATGGAGAAGAAACCGAGACAGAAGGTGCAGGGCCCACTACCG | |
| | CTTCCTCCAGATGAGCTCATGGGTTTCTCCACCAAGGAAGTTTTC | |
| | CGCTGGTTGAATGATTCTTTCCCCGCCCTCCTCTCGCCCCAGGGA | |
| | CATATAAAGGCAGTTGTTGGCACACCCAGCC | |
| 7. | AAAATACAAAAAGTAGCTGGGCGTGGTCGCATGCATCTGTAGTCC | IL6 CD56 |
| | CAGCTACTCAGGAGGCTGAGGCAAGAGAATTGCTTGAACCTGGGA | |
| | GGCGGCGGTTGAAGTGAGCCAAGATCATGCCATTGCCCTCCAGCC | |
| | TGGGCAACAGAGCAAGACTCCTTCTCAAGAGAAAAAACAAAACAA | |
| | AACAAGAAAAAACAAAGAATGAGCTCTCCACGCGAAAAATCCATT | |
| | GAGATGCAAAGGAAGGAAGCTATCATTGTGGAATTGCACATGTCA | |
| | GTTACATTAACGTTTTTGGAGCAAGGTAGAGCTCATCTCTCCCAC | |
| | AAGCAAATTCCAGCCCAAAGCATTGATACTAATAAAGTGCCATGC | |
| | TGCGATGTGCAGGGGGCAGACAGTGTCTCCAAGCTCCCTACACAC | |
| | ATGCCTTCCCACAGTTTGCCCTTTCTTGACCCCAGAAGCATCAGG | |
| | CCCCTTCACCCTCGAGGGCCACTATCAGGAGTTTGAATTAATGGC | |
| | AATCACCATGCACAGGGAAGGCTGTGGAATTCTGACATAAAAACA | |
| | CTTAGTGGAGGGCTTGGAAAAAGTCTAGTAGGAGCAAGACGCAAG | |
| | CTGGACTAATTATCTAAAACAAGAGACCTGGTTTGGGGATCTTAA | |
| | TGTTCTCAAAAAAGAAAATTATTATTATTTTTCATTTTGCACTTT | |
| | GTGCCATAAAACATTTTCAACAAAACATAGAATCTCATTTCTTTT | |
| | GAGGGAAAATGATTGGGAGACCAGCTCATTGCTGGCACAGAGGCC | |
| | TGGTTCATTCATAATTCCTTCATAGGCAAGACACCAGGTGAACCG | |
| | ATATAGCCGAGCTGGAAGAGCTCTCCAAGGCAGAGACTCTGAGCC | |
| | AAGGAATGTTCAAAGAGCTAGCATGTATTGTGGGATTACTATGCG | |
| | CCAGGAATTTTTTACACTGCATCACGTTCCATCTTCACAACAGCC | |
| | CTAGAAAGGAAGAACTATTATTACCCCCGTTTTATAGGTGAATAA | |
| | ACAAGGGCACAGGTCCTTGATGTAACAGCCAGGATCAAACAGCTG | |
| | GGAAGACGAGAAAACCTTTCCCAGGCTAGGATAACAGAGGATTTG | |
| | GTTGAAAATACAGGCAATTAGGTGCTACCTCTGGGAAAAGGGGCC | |
| | AGGAGAGGAAGGAGACACTTTTCCCTGCATGCCCTGATGTCCTAT | |
| | TTGAACATTTTATCATGAACACGAACTTCCTATTTAAAAAACACT | |
| | TTTTATTGAAAAGATAAATCTGTGTGTTGTATTGTGTCACTCAGT | |
| | TCAAGTACTTGAAATTTATTGAATTGTATTTTCTAAAAAATAGAT | |
| | AGTTGAGTAAAAGCAAGCTCACATTACATAGACGGATCACAGTGC | |
| | ACGGCTGCGGAGCTGGGAGCAGTGGCTTCGTTTCATGCAGGAAAG | |
| | AGAACTTGGTTCAGGAGTGTCTACGTTGCTTAAGACAGGAGAGCA | |
| | CTAAAAATGAAACCATCCAGCCATCCTCCCCCATTTTCATTTTCA | |
| | CACCAAAGAATCCCACCGCGGCAGAGGACCACCGTCTCTGTTTAG | |
| | ACAATCGGTGAAGAATGGATGACCTCACTTTCCCCAACAGGCGGG | |
| | TCCTGAAATGTTATGCACGAAACAAAACTTGAGTAAATGCCCAAC | |
| | AGAGGTCACTGTTTTATCGATCTTGAAGAGATCTCTTCTTAGCAA | |
| | AGCAAAGAAACCGATTGTGAAGGTAACACCATGTTTGGTAAATAA | |
| | GTGTTTTGGTGTTGTGCAAGGGTCTGGTTTCAGCCTGAAGCCATC | |
| | TCAGAGCTGTCTGGGTCTCTGGAGACTGGAGGGACAACCTAGTCT | |
| | AGAGCCCATTTGCATGAGACCAAGGATCCTCCTGCAAGAGACACC | |
| | ATCCTGAGGGAAGAGGGCTTCTGAACCAGCTTGACCCAATAAGAA | |
| | ATTCTTGGGTGCCGACGCGGAAGCAGATTCAGAGCCTAGAGCCGT | |
| | GCCTGCGTCCGTAGTTTCCTTCTAGCTTCTTTTGATTTCAAATCA | |
| | AGACTTACAGGGAGAGGGAGCGATAAACACAAACTCTGCAAGATG | |
| | CCACAAGGTCCTCCTTTGACATCCCCAACAAAGAGGTGAGTAGTA | |
| | TTCTCCCCCTTTCTGCCCTGAACCAAGTGGGCTTCAGTAATTTCA | |
| | GGGCTCCAGGAGACCTGGGGCCCATGCAGGTGCCCCAGTGAAACA | |
| | GTGGTGAAGAGACTCAGTGGCAATGGGGAGAGCACTGGCAGCACA | |
| | AGGCAAACCTCTGGCACAGAGAGCAAAGTCCTCACTGGGAGGATT | |
| | CCCAAGGGGTCACTTGGGAGAGGGCAGGGCAGCAGCCAACCTCCT | |
| | CTAAGTGGGCTGAAGCAGGTGAAGAAAGTGGCAGAAGCCACGCGG | |
| | TGGCAAAAAGGAGTCACACACTCCACCTGGAGACGCCTTGAAGTA | |
| | ACTGCACGAAATTTGAGGATGGCCAGGCAGTTCTACAACAGCCGC | |
| | TCACAGGGAGAGCCAGAACACAGAAGAACTCAGATGACTGGTAGT | |
| | ATTACCTTCTTCATAATCCCAGGCTTGGGGGGCTGCGATGGAGTC | |
| | AGAGGAAACTCAGTTCAGAACATCTTTGGTTTTTACAAATACAAA | |
| | TTAACTGGAACGCTAAATTCTAGCCTGTTAATCTGGTCACTGAAA | |
| | AAAAATTTTTTTTTTTTCAAAAAACATAGCTTTAGCTTATTTTTT | |
| | TTCTCTTTGTAAAACTTCGTGCATGACTTCAGCTTTACTCTTTGT | |
| | CAAGACATGCCAAAGTGCTGAGTCACTAATAAAAGAAAAAAAGAA | |
| | AGTAAAGGAAGAGTGGTTCTGCTTCTTAGCGCTAGCCTCAATGAC | |
| | GACCTAAGCTGCACTTTTCCCCCTAGTTGTGTCTTGCCATGCTAA | |
| | AGGACGTCACATTGCACAATCTTAATAAGGTTTCCAATCAGCCCC | |
| | ACCCGCTCTGGCCCCACCCTCACCCTCCAACAAAGATTTATCAAA | |
| | TGTGGGATTTTCCCATGAGTCTCAATATTAGAGTCTCAACCCCCA | |
| | ATAAATATAGGACTGGAGATGTCTGAGGCTC | |
| 8. | TACCCAGGTCCACCTGTTCTTATCTTATCTTGGAATTGCAAATCT | NCAM1 |
| | GGGCTCCTTTTTGCCTCCTTTCCCACTGTCGCTTTCTTAATTAGT | CD56 |
| | CCCTGTCACCCTATCCAGAGCTATCATGGCCTCCTAGCTTGTCTC | |
| | CTTGCCATGGCTGTCTTCTTGGACCATTCTCTGCTTCCTCTCTAA | |
| | AGAACTTTCTCTCTAAAGCACAGTCTGGAGTCTCCTTCCTCTGCC | |
| | TCAATCCCATGGATGTTCACTTTGTAGATGCTCATTAATGTGTGC | |
| | CTGACAGCCTGGGCCTCTCTCTGGGGAGAGAGGTCCAGTGAGGGC | |
| | CACAGTTTTGTCCATTTTAAAATGAGGATCAGGAAGGGATTGAAA | |
| | CGCAACAGAGCTCACAGATGTAGGCACCGACTAGCGGAGATATAC | |
| | AGAATTGGAGACAAGTGACATATCCTGTTAAAAGAATTTGGGAGT | |
| | GATACTGGGAGTGTGGACTGGAATGACTGGGGCAGCATTCTAGAA | |
| | TGAATCATTACAACAGAGATCAACAACCACACGGGGTAGGGAGGG | |
| | GCTGGAGAGAGAGAGGGAAAAAAAAGAAATTCAGTCATACTCAAG | |
| | TGAGATTGGATTTCCACACTTGCCTACATTGAAAAGCTAAAAATA | |
| | GGGACATTTGGGGAGAAAAATAGTGAGGAGTCTTTTAATTCTACT | |
| | GACAGGACTATAAGGAGGGAGGTTTGCAGGGAAGAATGTGTCTCT | |
| | CACTCTTAGGGGCAAAATTGGAAAAATTTTTATTTTGGGGGCCCC | |
| | AACCTTCACTCTCCCCACTCTACCCCCTGCCTGTTCTCTCTATCT | |
| | TTCCTCACCATCAAAACCTCTTTTTTAAGGAAAGAGTCTGATAAC | |
| | ATTCATTTACATAAATCTGTAATATTTAATGTGGTGATTTTAGAT | |
| | GTTAGAAGCAGAAGTCTGAAATCAAAGACACTTAAATACAGCAAC | |
| | CATTAAAAAAAGACGTATATAATTCACTGCATCTAGCACAGGGCT | |
| | AAACCCAGTGGTTGCTCCGCAAATATTTGTTGAAAGATGTTCAAA | |
| | AAAGCCTTATTTATATTATCAAACTCCACAAACAATATAAATATG | |
| | CAAAAATGGGGAAACCATTGCAAAATCATCCATATGAAGAAATAT | |
| | TACATAGCTATAAAAATATCTAGAAAAGCTTTTTAATGATCCAGA | |
| | GGAATAAATGCTTATACTGATGTTAGGCAAAATCAGCAGGATTCA | |
| | AATTGCTACTATAATATGTCAATTATAAAAATCATATGCACATAC | |
| | AGAAGACAAAATCTATGAGTATCTCTGGAATCTGTGGGATTTGGG | |
| | GTTATTTTTAGTTCTTTCTACTTTTCTGTACTTTTGAATTGTTTA | |
| | TACTTGGAATGAGAGAAATAGCCCAAAGTAAACGTTGTGGAAGAT | |
| | TTACTTGGGGGAAGAAATACTTTTCAGGGGTCCAAGCTAATTACA | |
| | CTGTACTGGTGTGGCCCACTTGGTCTATTATAGAGGAGGAAAGTG | |
| | GGGAAGAGATGGGGCCTGTCCAATAAGGATGGATGTCCTGACAGT | |
| | AGAGGATGGACTTTTAAAAACTGCTACAGCGGCTGGGTGCGGTAG | |
| | CTCACGGCTGTAATCCTAGCACTTTGGGAGGCTGAGGTGGGCAGA | |
| | TCACGAGGTCAGGAGTTCGAGACCGGCCTGACCAACATGGTGAAA | |
| | CACCGTCTCTACTAAAAATACAAAAATTAGCTGGGCGTGGTGGCG | |
| | CGCGCCTGTAATCCCAGCTACTCAGGAGGCTGAGGCAGCAGAATC | |
| | GCTTGAACCGGGAGCAGAGGTTGCAGTGAGCAGAGATGGCGCCAC | |
| | TGCACTGCAGCCTGGGCGACAGAGCGAGGCTCCATCTCAAAAAAA | |
| | CAAAACAGAAACAAAACAAAACAAAACAAAACAAAAAACCTGCTA | |
| | CAGGAGTGGGGAGGCCGACCTTTGAAGAAAAACGGAGTACCCGGT | |
| | AACATTAGTGCTTTAATGCCTTTGAACTTATGCAGACTTCCTCTG | |
| | TTAGAGGGTTTCAGTGTTCTAGGCTAATGGGTTAACCTGACATCT | |
| | AGAACACCTTTCTCACATTAGTTCCTTACATACCCAAGCCTTCAG | |
| | GTGCTGAGACATGATTCTTTTCACCCCGCTTTCTCCACCCCCTAC | |
| | TTTTGAAAACACGGGTGGAATTTTAATTAAAGCCTATTGTGTTGG | |
| | TACCTCAGTAATATTATACATTAATATCTTTAAGAATTAAGGTCA | |
| | CGTCCCCATGTAAGAAAATATTATTTAATGACGCTTCTATATCAT | |
| | AATACCTATATAAAAGCCTGGCTATTTTAATAAAGAGACCACAGA | |
| | TTTCAGAATTTATAAACAGGAAAACATTTTCTTCGGGTTATTTCT | |
| | GGAAATCTCTTCCAAACATCGGAGTTTTCTTCTAACTTAAGTCTC | |
| | TTCCCACCTCCTTCCCAGGGGATCTGCTGAAGGGTGTTGTATGTC | |
| | TCTCTGTGGGAGAGCAAACTTCACAGTTAGGATAAAACAAACAAA | |
| | CAAACAATATCCAAACAACACCCAGCAACGGCAACCCCCATCCCT | |
| | CTCCAAAGTTCTAATTTCCCGCACTTAAAGTCCTGGGCTATCCTT | |
| | GTGTTGCAAGGATCTTAGAATCGAAATGGAGGGATTTGACAACTT | |
| | TACCTAACCAAATCTAAAATTTTGCTTTTATTATTTACTAGTTAT | |
| | CAAAATATGCAAACTGCTGATTAAGGAAGGCTGGGTAGCAGGAGC | |
| | GCCTGCGAAGGCGTAGGGTAGAAGTGTGAAAAGAAATCCCAGCTC | |
| | TCCCAGGGAGACTGCGTGTGAAAGAGCCCGGCTCCCCCAAAAGCT | |
| | CCAGGCCGCGTTTTGCAGGCTTCCGCATCTGCCTCCCCTGTCTCT | |
| | CTTACCTCCTTGATGTTCGGCACTATTTGTGGCCGGCGTGGTGGA | |
| | AGGACACAGTGAGGTTCTCACCCCCGCCCCCCGCTCCTCGCTCCC | |
| | ATCCCAGTTCCATCAAAACGAACCCGGGCCAGCGCAAGGATCTCC | |
| | GAGTTGCGAGTGTGCTGAGGCTGGGACTGTC | |
| 9 | GGAGATTGAGGTCCTAACACCAAATTCATTAATTTATTCAATAAA | FCGR3A |
| | TATTATTGGCCAGGTGTGGTGGCTCACGCCTGTAGTCCCAGCACT | CD16 |
| | TTGGGAAGCCAAGGCTGGTGAATCACCTGAGGTCAGGAGTTTGAG | |
| | ACCAGACTGACCAACATGGTGAAACCCTGTCTCTACTAAAAATAC | |
| | AAAAATTAGCTGGGCGTGGTGGCGGGTGCCTGTAATCTCAGCTAC | |
| | TCGGGAGGCTGAGGCAGGAGAATCACTACAACCCAAGGGGGCAGA | |
| | AGTTGCAGTGAGCTGAGATTGCCGCATTGCACTCCAGCCTGGGTG | |
| | ACAGAGTGATACTCTGTCTCAAAAAATAAAATTAAAAAAAATGAA | |
| | AATAAATATATAAATAACTATTATTGAGCATCTATGCCATGGCCT | |
| | AACCTTCACATGACACATTCACATTGTATGCACTCCATATGGGGA | |
| | TTCTTGGAATTGTGCAATGCAGCAGTCCTATATCTACTGTCTGAC | |
| | AGGCTTTGACATTTTGCCCAAGACCTACTTAGAGCTAGGACTACA | |
| | ACTCTGATACCATTCAGTGGGACCACACATCATCTCATCTTAGCT | |
| | TTTATCTCTAAGAATATCATCTCTGGCTCTTACCAAGTATTTCAG | |
| | GACCCTTTGTTTCACCCTTTATTGGTGATTTTCCTCTTCCCCTTC | |
| | ATCAACTCACCGATATGGACTTCTAGCTGCACCGGGTCACTGAGG | |
| | GTGGAGAGGTTTGTCTGGCACCTGTACTCTCCACTGTCGTCGACT | |
| | GTGGCAGCGTCAATGAAGTAGCTCGAGGCCTGGCTTGAGATGAGG | |
| | CTCTCATTGTGAAACCACTGTGTGGAATTGTCCTCAGGGGAGTAG | |
| | GCTCCCTGGCACTTCAGAGTCACACTGTCCTTCTCGAGCACCCTG | |
| | TACCATTGAGGCTCCAGGAACACCACAGCCTTTGGGAGATCTTCT | |
| | GAGGAGCCAAGATAATGTGGGGTGAGGACAGGGAGAGGAGCAGGC | |
| | TCTACACTGCCATTCCCAGGGAGCCTCAAAGCCAGAATGAGCTCA | |
| | TTGCAAACCCATGCTTGGTGGCTCAGTCTTAGAGCATCTTGGCCC | |
| | CATTTTTGGCCTGTTCAGTATCTTAAGGAAAGCTGGCCAGAGAAG | |
| | CACAGGGCCAAGTTCTGCTGTGTTGGAGGAACTATCCCTGCTAAC | |
| | CCCACATCAGCATTTTCCCATTCAACAAGCATTTCCCAATATCTT | |
| | ATGGCCTTTGTCCCCATATGTGCCCCACTGGGTCAATCCAAGACC | |
| | ATGAAGCTGACTCACCAGTCCGCATGCCAGCTGAAACTGCAAGAA | |
| | AAAAGATAAATCAAATATTGAGTAGGGGCAGAGATCAGAGTGATT | |
| | AGAACATAGAGTGAGTTTAAAACTCCCCTGCCCTCCTCTGCCCCA | |
| | GGAGCCCAATTTTCCCAAGAATCAGGATGTTTCTGGTGGAAACCT | |
| | TGCTACCTGCTCTCTGGTCTCCACTGTTCATGCCTCTTGCGCCAC | |
| | TGTCAACACAAATCCCTATTTTCAACACTGCTCCCTTACCCCTTG | |
| | CTCCCTGTGCAAACTCACAAATTAAGGGTACAGGTTGAATTTCCC | |
| | TGAACCAAGCTACAGAAAGAGCCTCAACCCATATCCCCACAAGAA | |
| | AGGGTAGAAATTGAAAATCATAGAGGAGAACCCTGGAATGTCAAA | |
| | CTGAAAGAGACAGACCCTAGGGACCATCTAGTCGAAGCTCTTTGG | |
| | TTCCACAGAGTGATTCTGGGACCCAGAGGGGTGAAGTGACTGGCC | |
| | TCACTCATGAGTATGCCCCAATTGGAACCAGCATTCTCCTCATTT | |
| | CTAGCCCCATCTTGGCTTGTCCTAGGAGCTCAATCCACAGCTATA | |
| | GATGTGGTGAGGGGTCCCATCCCTTCGTGGGAGTCTCATTCGTAG | |
| | CCTGAAAAGGGGTCTCTGCTGAACCCAAGGCATCTCAAACTTCTC | |
| | CCTCAACCAGGGAGACCCTGACTTACCTAGAAGTAGCAGAGCAGT | |
| | TGGGAGGAGCAGCTGCCACATGATGCCACACTGGAGTGGACAAGT | |
| | CACCAAAGATATCCGGAGCCCTAAAGGGACCAAACCGACTAGACA | |
| | GGAGGAAGTAAACAGCCTTTCCCCAGCCCCTCCACCCATCTCTGT | |
| | CACCCACCAATTTCCTTTTCTTGAAACTTCATCTGACTTCTCAGT | |
| | CTGAAGTCTGGCAAGGGAGCCCCACCATAGAACAGGACCAGGAAG | |
| | GAAAGAGCCTGGAGGCAAGGTGGGTGGGTCTGCCCCCTTTACTCC | |
| | CTCAAAGGTCTGTGGCTGAGCATCTGAGGACACACACAGAATCTG | |
| | CCAGAGTGTGCCCTCAGCCATCCCAGGATGCTTGCCCCATCTCCT | |
| | GGATTTGGATCCACCCAGCACCAAGAATGGGACAGTGAGACCCTG | |
| | GGGATGAGATTCAAGGTGGGAGGAGCATTCTCTGAGGGCTTCCTT | |
| | CATTTCACCTCAGAACTTGTCACTCTCCTGCCTCGCCCAGACCCA | |
| | TCTATCTCCAGCTGAGGCCCTGCCTGCACACAGAAAGTGGTCCTT | |
| | TCAAATCTTCAGACCACTAGCAGTGTCTCTGCCTCAATATTATCT | |
| | CCACGCAGAATTTCTTTCAAAATTCAAAATTCAAAATCTATATGC | |
| | TCCTGGGATATGTAATCCACAGGAGCTACTGACTTTTTAGTGTGA | |
| | TTTGATCATTAGATTTCCAGGTTAGAAGAAACCCAGGTAGGGTAG | |
| | AGAAATGGGCCCTGGAAGAACAAGTCACCAGCGAAAGGCTGAAAA | |
| | GATCACAGAAAAGGTGGGGGTGGGGAGGGGCGAGGACAGGAACTC | |
| | TTTACCTTCCTCGTGTTACCCAGGTCCTGCGGATTTAGCTCAGGC | |
| | CCCTCCGGGCCACTGGATCTGGGCTGGTCTGTCAGCCTAGGAGCC | |
| | GGGGCTCCAGGGCTCCTTACCAGAAAAGGTGGAGGGGGGTGGCCA | |
| | GGAAGTGGGAGGGTTGTTAACCTCTTCTCTTCTCTCCAGATTCTC | |
| | CCCCGTAACCCCACGTTGGTTACTCTCACAA | |
| 10. | CCTGAGCCCAGGCCTCCCGCAGGGTGCTTAGGGTGGAAGCCTGGT | NKp44 |
| | TCTGTGCCTGACCCACCCTGAACTTCCTGTGAGGGAGCTGTGCGC | |
| | CCAGACTGCAGCCTTGTGTGGGCAGTAGCTCAGGTCCCTCTGACC | |
| | TCTCTGGCAGGGAAAAAGCTGCTCTGAGACCAGATGCTTTGAACC | |
| | TGGCATCCTGGGACCAGGAAGGGCCATTGTTATGGGACCATTGTC | |
| | ATGGGAGCAGGAATAACCCTGTCAGGGCAATTGATGACTGAAGGA | |
| | TCCTGCTGGAGCTGGGGCAGAGAGCCATTGGGGTCAGAGGTATAA | |
| | GCAGACTTGTTGGGGTTCAGGGAGAAGACTCCTTGGATCTGCAAA | |
| | AGGCAAGCCTGGATGAAGAGTAAGGAAGTGCCCATGGAGAATCTC | |
| | TGAGCCCAGGAGGAGCACCAGGTCAGGAGAGAGGAGGCCTGGGTG | |
| | CTAGTCCTGGTTTGGCCCTGAAATAGCTGAGTGACTGTGGAAAGG | |
| | CTACTTGCCTTCTCTGGACCTCATCTCCCTCAGTTGTGAAATCAG | |
| | CACTGAGAGAAATTCCCTTTTGTCCCTGCTACTTCTGGTTCTATC | |
| | ATGGGGGAAATTTACAGCTACCCCTTCACCCCTGCCCAGCCTAGC | |
| | TTAGCCCTGTGCTGCGTTTTGGGGATCACCTCCAGACCTCTACGG | |
| | CTCCCTGCTTTCCCCATTTCTGCATCAGAGGCCAGCCCTCCCTTC | |
| | CCATGGACCTACCCAGGCTCGGAGCCACTGTTGCAGGCTAGACTG | |
| | GGCCTTGCTAGAAAAGCAAGTACATGAAGTCTCCAGCTCTCCCAG | |
| | CTCCTTCTCTGGGTTCCTGTGAGTGTGTGTGACAATGAGTTTGGG | |
| | AGAGGAGGTTGCTGAGGTTCCCAAGTTCTCTGAGGGACCTTCTCA | |
| | GAGGGTGGGCAGGGAACCCAGAATCCCCCAGAACCCCAGGCTCTT | |
| | TGACAGGACTTTGCTGTGCCCTGCTCTGATCGTGCCAAGGGGGAG | |
| | GCACTATCCCTAGCTCTTCAAGAGACTTCTGAAAGGGCCCCAGAT | |
| | CCCACCACAGCTACCTGCCCCAGTCCCTCCTGCAGAAATTCAACT | |
| | TGTGACAAGGGCTGTTGTGGGTCCCCAGGGAGCAGGGCCAGTCCC | |
| | AGCCTGGATGAGGGGCTGCCTAAGCCCAGGCTTCCCTGCCCCCAG | |
| | GTCCCAGATGCCAGAGGAAGCTGAGCGTGCAGATGGTTCCAGCAG | |
| | GGGCCTCCCCCTAGGGTGCTCAAGGCCCCAAGTCTATGTGGAGGG | |
| | TGCCCACCCCACTCACACAGGTGGGGAAGCCACAACACAGAGAGA | |
| | GGGGCAGATTCCCCGCTTTGAGAGCTCCTGTCCCCTGGGGAGAGA | |
| | CGACACACTCTGACACAGCAGAGAACGATACAGAGCTGGCTGCAG | |
| | GCTGAAGAGACACACAGCTAGTGGCACATAGCAAGGGGTGCTCTT | |
| | GTAAGCTACAAGAAGGAGCTGTCACTTAGAGTCAGCAGGGGTCAG | |
| | AGAAGGGGAAAGTCAGCATGGGAACACAGTTGCTGACACACACAC | |
| | ACACACACACACACACACACACACACACACGGTGACGTGCCTCGG | |
| | TGCCATCTGAGGACAGGGCCGCCTTCCTCCCCCTCGGGAGGATGC | |
| | CCCCACCCTGATTTGGTATGTAATTATGTTCAGGGAGAAGCTGGG | |
| | CCCCACTGTCGGCGAGGAAACCCCCTAAAAGCTCACCTCCGCCTG | |
| | TGTGTGCAATTCAGTGGGAGCCTCTTTCTCACCCCTGGTTTCCTG | |
| | TCATGTCTGTGGGTCGCATTTCAACCAGACCAGCCGGAAGGCTCC | |
| | AGACCGAAGTCAGGAGGCCTGTTCTGGTCCTGTGTGACTCTGAGC | |
| | AGGTGGCTGACCATATCTGGGCCTCAGTTCTCACATCTGTGAAAA | |
| | AGAGAAGTAATGTCTCCCTCGCGAACCTGCTGAGGGTCTTGAGAA | |
| | AGTGTGCATGAGAATGCTTTGTAAATAATGCTTGTGTGAGTGAGT | |
| | GGCGGGCATCGCTCCCTGAGCTGTCTTCTAGCTCTGGAGCTCCAG | |
| | CCTGCTTTCCCACCTTGGGCCCTTGGGCGCCAACATCTGATAGCC | |
| | CTACTGTGTCCTCCTTTTGCATACCCCTCTGCCTTTCCCATTCTC | |
| | AGTCTCTGCATACTCCTACTGTAAGGAGAAGGGCAGTGTACTGAG | |
| | ATGGAGTGGAGAGCAGGAGTCCAGGGGCAGGCAAAGCAGTGGACT | |
| | TGGAATCGAGTCCACTTGACAAGTTCCAATTCCCGTCTCAGTTAT | |
| | TACCTCTGTTCACAGAGCTCCCGGCAGCAGATCGCCAGTCCAGCA | |
| | GAGCCCCCAGTGGTGCAACTGCAATTACACACAGTGTCTCTACCA | |
| | GTCCTAAGATATATTAGCTCGTTCTCTTTGTAAATTGCATCGTAT | |
| | TTTAAAACTGAATCTTCAAAGTGCTGACATTAACTAAAGAAAAAT | |
| | ATGCTAGAGTCTTTCATCTGTTTATGTTTTAATTAAACAATATTT | |
| | CTCCAGGGTCCTTAAGTGGCAGAGGTCCAGGATGCAGAGATGAGG | |
| | CCAGGTCCTGGTTCCTGGGCTCCCTCTACCCCACCCCACTGATGC | |
| | ATCCCATGCTCCGTGTGCTCTTCCCACCCTCCCCTCCCCGACCTT | |
| | TGCCAGGGACCCTCCCTAGCCTCCCTTGCTGGTTTTGAATCTCCA | |
| | CCTCCTCTCCCCTCTCCTTTCCCGCACCCACGAAACTCCCAGAGT | |
| | GGAAGCACATCACTGACCCACCATGGAGGCCTGGGCATGACGTCC | |
| | CTGGCCATCCTGTGACCAGTGAGGCTTGGGGCTTGTGATCAGGGT | |
| | TCCAAGTGCCCCTGAGTTTGTCATCCACCCTCAGCCTGCCTTTCC | |
| | CTGGAAAGGGCGGCAAAGCTAGGGCCCTGCAAAGAAGAGAGGCAG | |
| | CACACAGAGGGAGCAAGGGGGAAGCAGTCCATAACTCGCCCACAG | |
| | TTTCCTCCTGGCCTTTTCAGCTCCTCCCTCAGGGGTAGGTCATGG | |
| | TGGGGAGGGGCAGCTGGGCACAGGGGAGAGC | |
| 11. | GTGTAAACAAGAGCAGGGCATGTGTGAGTAGTTGAGAATGGTGAA | NKp46 |
| | TAGGAGTATGACTAGACAGAAGATAGTAGGGATGACAAGTTTTTG | |
| | GGGGCACATTCCAAGTTGGTCTGGTGTCTGGAATGAGACTGGGGC | |
| | TTAATAAAAAGGAGCGTCTATACAGGAGCTCAAATGGGCTGTACC | |
| | CTTTAGCATTCTGAGGACAGGCCTGAATTCTGAGAAAAGAAAGTG | |
| | GTAAAAGTATTGTCCAGTCTTTTTTAAGTTGGTGGCTGAGCTTGG | |
| | TGAGGTGTGTTTTTAAAAGACTATTAGTCTGTTCTACTTTTCCTG | |
| | AAGACTGAGGACTGTAAGGGATATAAAGGTTTCACTGAATACCAA | |
| | GAGCCTGAAAAACTGCTTGGCTGATTTGACTAATAAAGGCCGGTC | |
| | TGCTATCAGACTGTATAGAGGTGGGAAGGCCAAACTGTGGAATTA | |
| | TGTCTGACAGAAGGGAAGAAATGACCTCGGTGGCCTTCTCAGACC | |
| | CTGTGGGAAAGGCCTCTACCCATCCAGTGAAAGTGTCTACCCAGA | |
| | CCAAGAGGTATTTTAGTTTCCTGACTCAGGGCATGTGAGTAAAGT | |
| | CAATTTGCCAGTCCTAGGCGGGGGCAAATCCCCGAGCCTGATGTG | |
| | TAGGGAAGGGAGGGGACCTGAGCAATCCCTGAGGGGTAGTAGAAT | |
| | AGCAGATGGAACACTGAGAAGTGGTTTCCTTGAGGATAGATTTCC | |
| | AGGATGGAAAGGAAATGAGAGGTTCTAAGAGATGGGCTAGCAGCT | |
| | TGTAACCTACATGGAAGAGGCTATGAAATATCGACCGAATAGAAT | |
| | GGGCCTGTGAGGCTGGAAGGAGGTATTTTCCTTGGTCTAAGAACC | |
| | ATTIGCCTTGTGTGGGAAGAGATTGATGGGTGGAAGTTTCAGTGG | |
| | GGGAGTAGGTGGGAGTGACTGATGAGAAGGAGAAAAACTGGCTGT | |
| | GGGGGACAGAAATTGGCATGCTAGCTGCTTGTCTAGCTACCTTAT | |
| | CAGCATAAGCATAGATGTGAGAGACAGAAGTTGGAAAGCTAGCTG | |
| | CTTGTCTAGCCACCTTGTCAGCATAGGCATTGTCTAGAGCAATGG | |
| | GATCTGATGACTTTTGATGGCCTTTGCAGTGAATGACTCCAGCTT | |
| | CCTCTGGGAGTAAAGCGGCCTTGAGCAGAGTTTTTATTAAGGAGG | |
| | CATTAAAGATGGAGGACCCTTGTGTAGTGAGGAAACCTCTTTCAG | |
| | CCCATATGACCGCATGGTGGGGCAGAATATGAAAGGCATATTTAG | |
| | AGTCAGTATAAATATTGATGCATAGTCCTTTTGCATCAGTGAGGG | |
| | CTTGAGTTAAGGCAACTAATTCGGCTTGCTGAGAGGTAGTGGAGG | |
| | GGGCAGAGCGGTAGCCTCAATGATAGATGTGGAAGATACTATAGC | |
| | ATAGCCTGCCTTTGCTGGTGAGTGGCGATTAGGCCTGGTGGAACT | |
| | GCCATCAATAAACTAAATGTGATTAGGGTGAGGAATAGGAAAGAA | |
| | GGAAATGTGGGGAAATGGGGTGAATGTCAGGTGGATCAGAGAGAT | |
| | ACAGTCATGGGGGTCAGGTGTGGTATCCGGAATAATGTGGGAGGC | |
| | CGGATTGAAGTATGGGCCAGTAACAATGGTAATTGTGGGAGACTC | |
| | AACAAAGAGTGAGTACAGCTGAAGGAGCCGGGGAGCAGAAAGTAT | |
| | ATGCGTCAGGTGTGAGGAAGAAAATAGATTTTTGGAAGTTATGAG | |
| | AACTGTAGAGAGTGAGTTGAGCATAGTTTGTGATTTTGAGGGCCT | |
| | CTAAAACTATTAAAGCAGCGGCAGCCGCTGCTCACAGACGTGAGG | |
| | GCTAGGCTAAAACAGTAAGATCAAGTTGTTTGGACAGAAAGGCTA | |
| | CAGGGTGCGGTCCTGGCTCTTGTGTAAGAGTTCTGACCACGCTAA | |
| | CCATGCCTAGGAAGGAAAGGAGTTGTTGTTTTGTAGAAGGTGCTG | |
| | GGGTTTGAGAGATCACTAGGACACGATTGGCAGGGAGAGCACGTG | |
| | TGTTTTTATGAGAATTATGCCGAGATAGGTAACAGAGGAGGAAGA | |
| | AATTTGGGCTTGACTGAAGTAATGGGGGCTGTCTGTGAAGCCTTG | |
| | CAGCAGTACAGCCTAGGTAATTTGCTGAGCTTGATCGGTGTCAGG | |
| | GTCAGTCCAAGTGAAAGCGAAGAGAGGCTGGGATGAAGGGTGCAA | |
| | AGGAACAGTAAAGAAAGCATGTTTGAGATCCAGAACAGAATAATG | |
| | GGTTGTAGAGGCAGGTATTGAGGATAGGAGAGTATATGGGTTTGG | |
| | CACTACGGGGTGGATAGGCAAAACAATTTGGTTGATAAGGTGCAG | |
| | ATCCTGAACTAATGTGTAAGCCTTGTCTGGTTTTAGGACAGGTAA | |
| | AATGGGAGAATTGTAAGGGGAGTTTATAGGCTTTAAAAGGCCATG | |
| | CTGTAGCAGGCTTTAATCCTTTTAAAGCATGCTGTGGGATGGGAT | |
| | ATTGGCATTGAGCGGGGTAAGGTTGATTAGGTTTTAATGAGATGG | |
| | TAAGGGGTGCATGATTGGTCACCAAGGAGGGAGTAGAGGTATCCT | |
| | ATACTTGTGGGTTAAGGTGGGGGGATACAAGAGGAGGACACAAAG | |
| | GAGGCTTTGGATTGGGAAGAAGGGCAGCAATGAGATATAGCTGTA | |
| | GTCCAGGAATAGTCAGGGAAGCAGATAATTTAGTTAAAGTGTCTC | |
| | AGCCTAATAAGGGAACTGGGCAGGTGGGGATAACTAAAAAGGAGT | |
| | GCTTGAAAGAGTATTGTCTAAGTTGGCACCAGAGTTGGGGAGTTT | |
| | TAAGAGGTTTAGAAGCCTAGCTGTCAATACCTACAACAGTTATGG | |
| | AGGCAAGAGAAACAGGCCCTTGAAAAGAAGGTAATGTGGAGTGGG | |
| | TAGCCTCCATATTGATTAAGAAGGGGACGGGCTTACCTTCCACTG | |
| | TGAGAGTTACCTAGACTGTCTGTGATGGTCCTGTAGGCTTCCGAG | |
| | GCGATCGGGATCGGGCAGTGTCAGTCTTCAGCTGCTAAGCCGAGA | |
| | AGATCTGGGAAGGAGTCAGAGAGCCTTGGGC | |
| 12. | GTCATGGCCTTTCTGCCATTTGGGGCATAAATTTTAAAAATTCAG | NKG2D |
| | CTTGCCATTTCCATCTATGTACTTCCCCTGACTATCCTTTTTAAA | |
| | GTACAACATGAACTTACTTTTGTTTTATAGTACTACATTATTATG | |
| | TTTACACTTTACTATATAAATAAAACTATATTTATGCCATGGAAA | |
| | GTACCCTTAGCAAAATCCTATATACTATTAAAATTAGTTAATTCG | |
| | TAATGTATAATGAAACCTAACATTACCAGAAAGGGACTTGGTTAG | |
| | TGAAAAGGCAGATGTAGGGAGAAAGACATGTATTTAAATCACAGA | |
| | TCCACATATACAAACTTGATAACTTCACAAATAACCTAGCTTTTG | |
| | CATCCCAGTTTTCCTAATTTGCAAAAGACGAAAGAAATAAAAAAG | |
| | AAAAGGATACTGTCACAATCCAGTATGGGTATTTTAAAAATAAAA | |
| | TAAAAATATAATGAACTTAGCATCAATAACATCAATCATTTAGTA | |
| | AGCTTTTAGTAAATAGCAGTGTGTCTAATTTTCTTCTTTTCTTTT | |
| | TTTTTTTTTGAGATGGAGTGCAATGGCGTGATATTGGCTCACTGC | |
| | AACCGCCGCCTCCTGGGTTCAAGCGGTTCTCCTGCCTCAGCCTCT | |
| | TGAGTAGCTGGGATTACAGGCTTGTGCCACCATACCTGGCTAATT | |
| | TTTGTATTTTTAGTACAGACGGGGTTTCACCATATTGGCCAGGCT | |
| | GGTCTCGAACTCCTGACCACAGGTGATCTACCCACCTCAGCCTCC | |
| | CAAATTTCTGGGATTATAGGCATGAGCCACTGTGCCCAGCCATCT | |
| | AATTTTCTTTATAATGAAGGACATACATAGGCTAAAATAGTGGGG | |
| | TCTAATGATTTAGAACAAAGACAGATAATCTTGCCTCTGAACAAA | |
| | ATACCTAAAGAATCATTTTGCTCTCTTGAAATATTAATTATTTAC | |
| | TGATGATTAATATTTACTTAAAAGCCTTTTTTAAACTTTTATTTT | |
| | TGGTTCAGGGGTGCATGCGAAGGTTTGTTACATATGTAAATTCAT | |
| | GTCACGGGGGTTTGTTGTACAAAAGCCTTTTATAAATTAAATGAT | |
| | AATATGAATTATATTTTATAGGTAAAATTAGACATTGATTGCGCA | |
| | TGTAATACCTAACTCAGTTATTTTACATATGGAGTAAAGAGAGTA | |
| | GGTATCACTTTATGGATGAAGAAATTGAATGCTAGAGATGGCCAA | |
| | AATCATAAAGCTTATTTAATATAATGTTACCAACTGTGAATAAGT | |
| | TATAAATTTATAAACACCTTTAGGATAAAAAGTGCTTTAACATAC | |
| | ATGTCAGTTGATTTGAATATTACACAAATCTAAGATAAATATGGA | |
| | ATGTTTCATGCCTGTATTTGATAGAAAAAGAAACTAAACTAAACT | |
| | ACACAGAGGTTGCAAATTGACCCTTCAAATCTAGAGAGCGGTAAA | |
| | AGTTTGAGTAAAATTCAAGTCTGACTCCACATCTATTTCACTATT | |
| | TCAAGCTGTCCCAAGTIGTGAGCGACTTTACCATAATATAGTTAA | |
| | TAAAATAAAGGGAAAAAAGTTTTTAAGTTTTGGAAGACTTGATTT | |
| | AAAATACAATATGAACATAACATGAAATCAACATAAATAGAACAT | |
| | GAAAAGAATCAGAGTAAATTGCTTGCCTATGCCTTATATGCTTGT | |
| | ATGAAATTCTTGGTATCTTAAAATTGTATAAAAACAAAACTACAC | |
| | ACTTATGTGGTAAAAACATACCCCAGCTGTGTCGAGACCTCCGAC | |
| | CACGAATCCACCCCATCAAATACTTATAAGTGCACGTCTACCGCA | |
| | GAGAGGAATCTAAAGTCTTCAATGCACAAAGGATTCCTGAATAAA | |
| | ATAAAACTGGGCATTTGTTTTTTGTTCTCTTTCCCGTGGTGAGAT | |
| | AAAATATTTTGAATAGAAAAGTAAAATGCAGAAACTTTTCCCCTG | |
| | TGCATGCCTTTAAGAAAAGAATAGAAATGACAATTCACATAAAGC | |
| | CAGGCAAAATTAATTTTATACATTTCTCTTGGTTGTAAAATAATC | |
| | GCCAATTACTTCAAAATGTCATAACAATATAATGAACCTATTAAT | |
| | TCACCTACATTGAGATATTTAATACCAATATTTTACTTTACTGAT | |
| | ATTAGATAACAACACACCCAGTTTTCTGTTTATAGTCGTTTATTG | |
| | CTTCTTGGCCTTTTAGCTAAGATCAAGTGTTTCCAGTTGTTTATT | |
| | TTATCTGGCAATAATTTAGTTTTCTTTCTGCAAGCTAAACTCAGG | |
| | TAAGTTATATGCACTTTTGTTGAATGTCCTTTTCTAGTGCATTTT | |
| | AATCCAACGTGCCCATATCATCATCATCAACAAATTATCATCACC | |
| | ATCATCATTAACAAAACTAATCTCACATGTTTATATAGTAACATC | |
| | TTAGCAATGCACAGCAGAGCTTCCAGAAAAAACCATTTAATAAAT | |
| | AAGCATAGTGCTTAGATTTCAAAAATAAAAGCTATTTCATATTTG | |
| | ATTATGATAGTTGGAAAGCACAGATATAACTTGTTTCACCTTGCA | |
| | TGTAAAGATGTGTATCAAAGGTAAGTGCCCCCTACAGCATCAGAA | |
| | AACTCAAAAATTTGACAATTTAAATTGAATCTCAATTCTTCTCAT | |
| | CCTAAATGTAAAGATTCATTTTTCTAGTTTTGCTTAGAGACAAGG | |
| | CTTCTTCTTATAGTAATATTACAACTTCTGATTCTTCAATAAGTA | |
| | TATCCTAATTACCTATCATCAGATACAGTGATAGAATATACATCA | |
| | GAAATGATAAGCATTCCGTATCCTCAGGAGTTCTATTCAAGCATC | |
| | TAAGAAACAATTAGAATTACCTTATAGTGTAAATATTTTTATTTT | |
| | TTTGAGGTTGAAATATAATTTCTCATATCTTGAATAATAAACACA | |
| | CACACATACACACATACACACACCTGCTCAGAGAGGGAAGATCTT | |
| | GATTCTTGTGGATAAAAGCCTTCTATAAAACTAGAAAATTTAGAT | |
| | ATGTCCTCAGTTCCTGATCTAGAATTGAAAG | |
| 13. | ACTTATTGTCAACACAGGAATGCCACATAACTAAAAGCAAAACAT | TRAIL |
| | TTCAAAGATGTGAAAGTTCACCAGGACTAGCTACATCTGGAATAC | |
| | AAAACGGGAGTGTTCAGTGACTTAGGGCCTGGCTTTGGGTTGAGC | |
| | CTGACTCTACCCTTGTCGCCTGCATATTCGTGGTGCCTACCACAT | |
| | CTGTGAAGGCTGTTCATTCACTCAATAAGTATGTAGTGAACCACA | |
| | AGTATTAGTATTAGTGGCTACTTGGGAACCACATGCTAGACCCAG | |
| | AGGTATACTAATGAGCAGAAGCAGACACAGCTCTGATACAGCTAC | |
| | CCTCACCCTCAAGAATTCATGAGTCAAGAACTGTAACTCCAACCT | |
| | GCAACAACAAAGCAGGGAAGATGGCTACAGTTACCAAAACAGCAT | |
| | GTTACTGGTATGGAAACAGATACATAGACCAATGGAACAGAACAG | |
| | AGGCCTCAGAAATAACACTACACATCTACAACCACCTGATCTTTG | |
| | ACAAACCTGACAAAAACAAGCAATGGGGAAAGGATTCCCTATTTA | |
| | ATAAATGGTGCTGGGAAAACTGGCTAGCCATATGCAGAAAACAGA | |
| | AACTGGGCCCCTTCCTTACAGCTTCTACAAAAATTAACTCAAGAT | |
| | GGATTAAAGACTTAAACGTAAAACCTAAAAGCATAAAAACCCCAG | |
| | AAGAAAACCTAGGCATACCATTCAAGATGGCATGGGCAAAGACTT | |
| | CATGACTAAAACACCAAAAGCAATTGCAACAAAAGTCAAAATTGA | |
| | CAAATGGGATCTAATTAAACTAAAGAGCTTCTGCACAGCAGAAGA | |
| | AACTATCATCAGAGTGAACAGGGAACCTACAGAATGGGAGAAAAT | |
| | TTTTGCAATCTACCCATCTGACAAAGGTCTAATATGCAGAATCTA | |
| | CAAGGAATTTGTTTCTTGTAAAACAAATTGACAAGAAAAAAACCA | |
| | CATCAAAAAGTGGGCAAAAGATATGAACAGATACTTCTCAAAAGA | |
| | AGACATTTATGTGGCCAACAAACATGAAAAAAAGCTCATCATCAC | |
| | TGGTCATTAGAGAAATGCAAATCAAAACCATAATGAGATACCATC | |
| | TCACTCCAGTTAGAATGGTGGTCATTAAAAAGTCAGGAAACAACA | |
| | GATGCTGGAAGGATGTGGAGAAATAGGAATGCTTTTACACTGTTG | |
| | GTGGGAGTATAAATTAGTTCAACCAGTGTGGAAGACAGTGTGGCA | |
| | ATTCCTCAAGGAACCAGAAATACCATTTGACTCAGCAATCCCATT | |
| | ACTGGGTATATACCCAAAGGATTATAAATCATTCTACTATAAAGA | |
| | CACATGCACACATATGTTTATTCCAGCACCATTTACAATAGCAAA | |
| | GACTTGGAACCAACCCAAATGCTCATCAGTGATAGACTGGATAAA | |
| | GAAAATGTGGCACATATACACCATGGAATACTATGCAGCCATTAA | |
| | AAAGAATGAATTCATGTCCTTTGCAGAGACATGGATGAAGCTGGA | |
| | AGCCATCATTCTCAGCAAACTAACACAGGAACAGAAAACCAAACA | |
| | CTGCATGTTCTCACTCATAAGTGGGAGTTGAACAGTGAGAGCACA | |
| | TGGACACAGGGAGGGCAACACACACACTGGGGCCTGTCGGGGGTG | |
| | GGAGGCTAGGGGAGGGAGAGCATTAGAACAAATCCTAATGCCTGT | |
| | GGGGCTTACAACCTAGATGATGGGTTGATGGGTGCAGCAAACCAC | |
| | CATGGCACATGTATACCTATGTAACAAACCTGCACGTTCTGTACA | |
| | CGTATCCCAGAACTTAAAGTAAAATAAAAAAATTTAAAAATAAAG | |
| | AAATCTGTTTGCCAGAATTCACAAAGAGCAATAGACCTCATGTGA | |
| | GTTATGAATCTGAACTTCATCCTGCTGTGTGCTATTGGATAAAGA | |
| | CTCTAAGAGCTACATTACAGATTTTTTCGACAGGAAGTATCACAG | |
| | TAACAAGGCCCTATTTAAAGGCTGTTGCTACTGCTGTGGAACAGA | |
| | TTTACCCATTTGTGTTTGAAAGCAGGAAAGAAATTTTATCATTCA | |
| | CCACTTAATTGGTTAGAATCTCTTACTGTGCACCTTTTAAAACCT | |
| | GCTGCACATTGGACTCAAAAGGAAAACTGAACCAACGGTAATTGA | |
| | GGAAATAGACTCTTTTATTTATTCATGGCTACAGTGTAAGCTCCA | |
| | GTCCCTTTGGATTTTATTCCAAACCTTGTTATAATATAAAAAAGG | |
| | AAGTTTACAAGACATGTCATTGCTGCTTTTACAAAAGGACATTCT | |
| | ATTTATTTTCGCAGGAATTCTCATGTCCCCATAAGCAGAGCTGTC | |
| | ACAGTGTGCACTACCTTAGGTTGTTTTATTGTTGTCATTGTTATT | |
| | TTTTTCCATTTTGAGCTAATGTGTTTTATCTGTGAATAGTCTTTT | |
| | ACATTTTTGTATGCTGAAAATGGGCACCAAAGAACCTGTAAAAAT | |
| | TATCTTTTTCAATTGAATGTGCACAAATAAAAGTTTGGAAAAGGA | |
| | AAACAAAAGAACTACAAAGCATTTAGCAGAAAGCTTTCATGAAGA | |
| | GTTGCAATGGCTGATATTATAAAATACACATACTTTTAAAAACAA | |
| | AAAATCCCTCAGGAGTTTTGTTGCCCACATCTGCTAGTAACTAGT | |
| | GAAGCCAGTTGTGTAACTGCACTTAGTTTACACAATTTCTTGAAC | |
| | TTACAGTTTTGCAAAGACCTCAGTTGTTATGTGATTTAGCAGTTA | |
| | GAGTGTACCCACAGAGAAAGGAAGCAGGAAAGTCTTCAGAGAGGG | |
| | GCAAGCTGACATGCAAAGAAGCAGGTAACCAGACATTTGCTAAGC | |
| | GCCTCGAAGACTGAGTGCACTGCACTGCACTGACCTGCTTCAGCT | |
| | CGTTGGTAAAGTACACGTAAGTTACAGCCACACAGAGAGACTGCA | |
| | GGAGCACTGTGAAGATCACGATCAGCACGCAGGTCTGTCCCAGGC | |
| | TGGGTCCCCCCTGGACCTCCATCATAGCCATGATCCTGTCAGAGT | |
| | CTGACTGCTGTAAGTCAGCCAGGCAGCCGGT | |
| 14. | CTCTCAAGTAGGTGGTTTGCATTTCTGGGACCTTTGGCACCTCAG | CD122 |
| | TTGAGGCTCTCTGGTTGTGAGCAAGAAAACTCAACTATTAACTAA | |
| | AGGTAAAGTAAAAAAAAAAAAAAAAAAAGTGATGTTGGGGATGGA | |
| | GGCAAGGGGCTAGAGCCCCAACCAAACCTGCAGGAAGGAAAAGCC | |
| | AGGGTAGCCCCAGCTCCTTGGCGCCCGCTGTGCTTGGGATCCCTG | |
| | GCTGCACCTCCTAATGCACTCGGCTCCATCAGCTCCTGGCTGCGG | |
| | TCCTGCTTCCCAAATCTGCAGAGCAGGACTCAAGCTGGCCCTGCT | |
| | AGGGTTGGGGCCTCCCTTGGAGCCGTCAACCATCCAATAGGGCCC | |
| | AGAATGAGGTAGAGAGATGAGCCCAAGGGTGGGGTAAGGGGCTGA | |
| | GGTTTTGGGGGTGGGCAGTGGCGAGGGGTCCCTGGAGAAGAGGGG | |
| | AACTTGTGAGCCTGGGGCCACACCCAAAGTGCCTCCTTCACTGGG | |
| | CCCAGCCCCCACCCAAGCCGCATTTCTAATGAAGGCTTTGTTTTG | |
| | TAAGAATTTTTAAATGCTTTAACTAGAAATTGGCAAACTGCAACA | |
| | TCTGCCAATTTCTAGCAGTATATATCCTGCCACCATGACCCATTT | |
| | CAAGCTAGCATCGTGATGTCTCTGAACCTGCGATCGGGAAGACAT | |
| | GCATGCAGTCGGCTCTGGTGCAAGCTGGCTCCAGCCACCATTGGC | |
| | TGTGGGGCTGAGGGCACAAGCCTCCTCATAGACTCCTGTAAGGAG | |
| | CCCTGCCCTGTCTTGAGAACCACCGAAAGAGCTAGAGAGTGTTTC | |
| | CCATCCTCCAGCCATCCTCTGAGAAGTCATAGCTCAGGTCCTGGA | |
| | TGAGCCTCCCCTCTTTCCCAGGAACCATCCCTTCCTTCTGGTGTG | |
| | GCCCACTGGACAAGATCGAATATCCCAGGTCCTGCAAAACCTGGC | |
| | TCCTTCCGGCCTCTGGGTCTTTCTTTCAGTTCCTGGGAGGGGCTG | |
| | AGCAGCCTCCTACCTCCTGGCCTTGACCCATATTGGCCCCTCCAC | |
| | CAGCTGTGCTATGTCTCTCATCTTTGGGTCTCAGCTGAACCACCT | |
| | CTTCCTCAGAGAGGCCTTCCCTGACCACTTCTCCCTCCCAAATTT | |
| | GCTTCTCCCTTGCCAGCACTTACCCCACGCTGAAGCCCTGCTGTG | |
| | ACTGTGTAATGTCTCTCTCCCCTATTAGACTGGAAGCCGCATGAG | |
| | GGCAGGGACCATGTGGGGTTTTGGCTCACCACACTGTCCCCTGTG | |
| | CTTAGCACAGGGCCAAGCCTATAGAATAGGAATCAATCCATAATT | |
| | ATTGATTGGGTAAACAAATGACACATGAAGGAAGGAAATTAATCA | |
| | GTTTTTCTGTACTTCACAGTTTCTTCTTCTGTAAAATAAATATAA | |
| | GAAAGTGTCTACCTTAAAATAATTTTTAACATTTATAGAGCACCT | |
| | CCTACGTACCAGTATTTTACATATACTTACTCCTTTAATCCTCAC | |
| | AACAGCCCAGATAGTGTTAATATCATCCCCATTTTACAGGTGACA | |
| | AAACTGAGGCACAGAGAGTTTCAGTGACCTCCCCTCATTTACTCA | |
| | GATTTGAACCCAGGCAGTGCCTCCAGCAGCCTCAGCATTAACCAC | |
| | ACACTAAACTGCCAGGGGGGGTTGGGGAGGGGTCCTGGAGGTGGT | |
| | AGCTTTGGCGCTGCTGTAGCCCTGCTGCCCCAAGAGCTTGAAGCC | |
| | TCGTGAGCAGAGCCAACTATTGCTAAACACTATAACCAGGGGACG | |
| | CTCAGGGATCCAGCTGCCTCTCACATCTGGGAAGATTTCCTGGAA | |
| | CCAAGACACCCTGGCTGAGTCAGGCACCCGCATCTGACAATATGG | |
| | CCAGAGAGTTTTTACGAAATGTGAACCAACTGGACTCTCAACAGC | |
| | TTCTCCCCACCCCACCCCAACCTGTGAGTGGGACTCTGATCTTGC | |
| | CTGGGGCTCCAGCCCAGCCCCTCAGGGTTCAGGGGTAAATCTCAC | |
| | TTCCCCGAGGTTGGGTTAGGTATGGTTATGTGACCAGCTGTGGCC | |
| | AATGAGAAGCGAGAAGAGGCTTTTGGACAGAAGAATCTCTGATTC | |
| | TCTTTCACCCACCCTTGCCACATCCATGAAACGGAAGGGTTCGTC | |
| | GCTGGGTTTTGAGACTTTTTTCCATGAAGCTGAAGGCCAAGGGAG | |
| | CTGAAAGCCTTGCCACTGAATGCTGAAACAACCTTCACTGGCTAC | |
| | TTTAGAGATAACATTCATAGGTCACCATGGTAATGGTGGCTTCGG | |
| | TTGTTTTTCAGGAATTGGGCCAGCTGAAACCAGTTGAGACCCTTC | |
| | AGCTGGGGAACCGTATATGTGCCCAAGAGATGGCCATTTGATATT | |
| | GGAGGACCAAAAGCACCACGCTCAGATCAGGCCAATGCATCTGTT | |
| | TTCTGTATGTGTCCTATGAAATGCCATGAACCCCAACTATGCTTG | |
| | TACAGAACAAACCGGTTACGTCATTCTTCCCCACTGCCAATCACC | |
| | TGTCCCCACACCTTAGATCACCCCACTTCTCTAACCCATTCATAT | |
| | CCCTAAACCTTGTCTTTGGGGAGGCAGATTTGAACGCTGTTCTCC | |
| | CACCTCCTTGCTTGGCAGGCTTGTGAATCAATCTTTTCTCTTTTG | |
| | CAAATCCCAAGTCACAGCGATTGGCTGCACACTGGCAGAACAGAT | |
| | TGGGACCTGGCGGGTGATACCCAGATATGACACCTGGACCCAGAA | |
| | CAGAAATCTCATGGCCACCATCTTGAGACTGCCATCCTGTGGCCC | |
| | TGAGGGAAGCTGGCTGTATTAGTTCGTTCTCACACTGCTATGAAG | |
| | AACTGCCTGAGGCTGGATAATTTAGAAAGGAAAGAGGTTTAATTG | |
| | ACTCACGGTTCCACATGGCTGGGGAGGCCTCAGGAAACTTACAAT | |
| | CGTGGTGGAAGGCGAAGGGGAAGCAAGGCACCTTCACATGCTGGC | |
| | AGGAGAGCGAAGTGCAGAGTGAAGGGGGAAGGGTCCCTTATACAA | |
| | CCATCAAAACTCGTGAGAACTCACTCACTAT | |
| 15. | CCACAGGCACGTGCCACCACACCCAGTTAATTTATTTTTATTATG | CD27 |
| | TGTAGAGACGGGGTGTCACTCTGTTGCCCAGGCTGGTCTTAAACT | |
| | CCTAGGCTCAAGCAATCCTTCCGCCTCGGCCTCCCAACGTGACTG | |
| | GGATTCCAGGTGTGAGCCGCCTCACCTGGCCAATTTTACATTTGT | |
| | CTTTGAGATTATATGATCGATGTCTGACTCTCACACTGGTGTGTC | |
| | AACTCCGTCAGGGCAGAAACCACATATTTGTCTGTTTGTTTTTCA | |
| | TCTTTGTGCCCCATTACTTGGCTGGTTCCTAACACAAAATGAATG | |
| | TGCAAAAAACGCTTGTCAAATGAATGAGGAGGTCTAAGGGGCCTT | |
| | GAGCCCTCTGACGTTTCCATTTTATAATTGTGGACAGAGCCGCTA | |
| | CAGCACAAAGCTGAGCTGGACCATCCAATGTTTAGCCAGTGCCTT | |
| | ACGTGCTAGGAGACACTGGGAGCTGTGCTGGGCTTCTGAGGAGTG | |
| | GGCAGAGGCTGGAAATACCCATAGTTGGTCAGTCTCCTGTGTCAC | |
| | CCAGACTGGGGAGGTGCAGAAATGAAAATTACAGAGCTGCGAGGA | |
| | CCCAGAACCTCATTAATGAGCTTAACTCACTGTCCTGACCAGGTG | |
| | GCCTCTCAAAGCCTTGCTGGGAAGGAGGCTTTAAGGTGGTGGGGG | |
| | CAGCAGTGCTGGTGGTGGTCATCGAAGAAAGTGGGAGTCGGGGAG | |
| | GAAGCAATGGGGAGCAGGGGACAAAGAGGGCCAGACAAACTCAGA | |
| | GCTTCAGGTGATGAGACTCAGGAAGCCTAAGTCAGGAACAGTGAT | |
| | TGCGGACATCTTTGTCCCATAACAGAACTTCCTCCTAAGGAAAGG | |
| | TCTTGCAGAGACTGGTCCTGCTGAGCGCTTTAACAGGTTATTGAT | |
| | TCATAAAAATTGGAGAAAAAAGACAAAGTCTGCCACACCTAACAG | |
| | CCTTCTGTTTAGAGCTGGTTTAGAGGAAACTTTAAGCAAGTCTTG | |
| | TGTCCAACAATTTGGGCCTGAGGCAGGGTACAGGTAAGGGAACAG | |
| | ACAGGAGACACGGAGGCAAGAACAAGACTTTAGCTGAAAGAGAAA | |
| | TCAGAGCCAGAGGCAGCCACAAGAACACCCAACCTACTGGAACTC | |
| | ATGCAGGTGGACAGCCTGCCCAGGACTTGCCCTGAGTGGGAAGCA | |
| | AAGAGCATCTTATCTAGATTTTAAGGAGCAGGGTGCCCCTGGCTG | |
| | ACATCTAGGCTACAGTCACTGGGCCAGGTGGAGGGTTGGTCAGGC | |
| | CCTCTCAAAAGTCATGAGAGGCCAGGCACAGTGGCTCACGCTTGT | |
| | AATCCCAACACTTTGGGAGGCCGAGGAGGGCAGATCACTTGAGGC | |
| | CAGGAGTTTGAGACCAGCCTGGCCAACATAGTGAAACCCCGTCTC | |
| | TACTAAAAATACAAAAAATTAGCCGGGTATGGTGGCCGGTGCCTA | |
| | TAATCCCAGCTACTTGGGAGGCTGATGTATAAGAATCGCTTGAAC | |
| | CTGGGAGGCAAAGGTTGCAGTGAGCTGAGATCATGCCACTGCACT | |
| | CCAGCCTGGGCAACAGTGAGAGACCCTTTCTCAAAAAAAAAAAAA | |
| | AAAAAAAGAACCAAAAAAAGTCATGAGAGTCCTCAGAGTTCTCAA | |
| | GAAGGGCTGCTGTGGGCCTGTTTCTGGAAAGGAAAGAAAAGCAGG | |
| | GAGCTGTTGAGAAAATACCATATGTTTTTATGCCCCAGCCAGGCA | |
| | TGGGGGAGAGGCTGACCCCAAAAGAGAAAGTGGTTGTGTGAGATT | |
| | TCAAGGAAGATGTCAGCGGGCTGCATCCCAGGGCCTCTAGACAAA | |
| | AGGAAGGGCCCAGCCAGGCATGGTGGCTCGTGCCTGTAATCCCAG | |
| | CACTTTGGGAGGCCGAGGCAGGCAGATCACCTGAGGCTAGGTGTT | |
| | CTGGTCCAGCCTGGCCAACATGACAAAACCCCATCTCTACTAAAA | |
| | ATACAAAAAATAAGCTGGGCGCGGTGGCTCTCCCCTGTAATCCCA | |
| | GCCAAGGTGGGCGGATCAACTTGAGGCCAGCCTGACCAACGTGGC | |
| | AAAACTCCATCTCTACTAAAAATACAAAAATTAGCTGAGCGTGGT | |
| | GGCACGCACCTGTCATCCCAGTTACTCAGGAGGCTGAGGCGTGAG | |
| | AATGGCTTGAGCCTGGGAGACAGAGGTTGCAGTGAGCTGAGATTG | |
| | CATCATTGCACTCTAGCCTGGGTGACAGAGTGAAATGGAGGGAGG | |
| | AAAAAAAAAAAAAAGGAAGGTCCCGTCCAGGGAGCCAGCCTAGGA | |
| | TGGGGAAGGCTCACCAGAAGTGGATGCAAAGAGTGCTGGAGATGA | |
| | GCTCATTCTATTTGCCTAGGAAAGAAAAACGTCCAGAAACCTGGC | |
| | CTTCCTCCTCTTGGCCGAGGCCCTCCAGGAAAGCCAGGCAGACCC | |
| | TGCTCCTGCTCTGACCCCCTGGAGAAGTGATATGTAGAAAAGAGG | |
| | AGATGGGCTTTGGCCCAGAGGACAGCAGCGTTAGGCTAGTGAGCA | |
| | GTTCCCAGTCGGTTCAAGGTTGCTAGGCTCAGCGAACTGCAAGTC | |
| | CCTGTTCGACAAGTTTCTTCCTAAAGGTCCCCAGTTCCTCATGAT | |
| | TCTTCTGAGGGTCTCATCCACCCTCCCAGGGCCTGCAGTCAGCTA | |
| | GCCACCCCACTGCCCCATGCCTGCAGTGAACACATTTACCACAAC | |
| | CTGGAAATGGCTGGTGACAGAAAAGTCCTCACGGCCACAACACTC | |
| | TCTGCGGTGGGAATTCAGGTGCCTTGCCTGATGGTGACAAAGCTG | |
| | GTTTTGCATAAACAGCTCTCTGAATCACACCCAAATGTGGCTTCT | |
| | GACTTGAGTGGACCCCCATGAAGAGCTGCAGAGGGGGCTGGGAAA | |
| | AGACAAGAGAATGGAGAAAATGAGGAGAAAGGAAACTACAGAGTG | |
| | AGATAGAGAACAGATCTGACAGGTGTGACCACAGCAGTGTGACAT | |
| | TTATGAACAGTGTGGAAACAACGGCAAAGCCCCTAAATTAAAAAC | |
| | TTCCTGGGGAAATAAGCCACTCCACATATCG | |
| 16. | GGTGAAAGAACCAGATCCTCTCTTGAAAAATAATAATAGGCCGGG | CD244 |
| | CACAGTGGCTCACACCTGGTATCCCAGCACTTTTGAGAGGCGGAG | |
| | GTGGGTGGATCACTTGAGGTCAGGAGTTTAAGACCAGCCTGGCCA | |
| | ACATGGTGAAACCCTGTCTCCACCAAAAATACAAAAATTAGCCAG | |
| | GCTTGGTGGCATGCATCTGTAGTCCCAGCTACTCGGGAGGCTGAG | |
| | GCAGGACAATCACTTGAACCCGGGGGAGCAGAAGTTGCAGTGAGC | |
| | CGAGATTGGACCACTGTACTCCAGCCCAGGCCACAGAGCGAGACT | |
| | CCATCTCTAAAAAATAATAATAATGATGATATAAAATAAATAAAA | |
| | ATGCATATGATAACCTGAATCATATGAGGGAACATCAGACAAACC | |
| | CAGATTAAGGAACCCTCTACAAAACAACTGGCTTGTCTTCTTCAA | |
| | GAATGCAATATCATGAAAGATGAAGAAAGGTTAAAGTATGGTCAT | |
| | AGACTGAACATGAGAAACATAACAACTGAATGTAATACATGATCT | |
| | TGATTGCATCTTGGGTCAGCAAAAAAAAAAAATAAAGCATATTCT | |
| | TGGGACAATTGGTAATATGTGAATATGGAAAGCATATTTGATGGT | |
| | ATTAATGTTAAATTTACTGAATTTGACAATTGTGCTGTAGTAACA | |
| | TGAGAATGTCTTTGTTCATAGGATATAAGTGCTAAAATATTTAGG | |
| | AGTAAGGGGACATAATGTCACAACTTGTTCTCAAGTGGTTCAGCA | |
| | AAAATTATTGTACATTTATACACACATATACATACAGCTAGAGAA | |
| | AGAGAAATAAAGTAAATGTGATAAAATGTTAATTGGTGAATCTAG | |
| | ATGAAAGAAATTCAAGAATCGTTTGAACTATTCTTGCAGCTTCCC | |
| | TGGAACTTCACAATTATTTCAAACTAAAAAGCTTAAAAATTAAAG | |
| | AAATAGAGCAGCTTCAGTCAACTTATGCCAGGCCTGCCCTATAGA | |
| | CACAGGATGGAGGAGGAAGCAGCTCAGGAAGACCCAAGTCCATCC | |
| | CTCCCTGTGCCTAACAAGTCACTCACCAGCTTCACCGTGCAAGGG | |
| | CCGGCATAGCCAAGCCCAGCTATGGTGCTGAAGCCCTGGGAATGG | |
| | GGGTGGAGGGAGCATTGCCCTCTGTCACACAGGCTCATGACCTCA | |
| | GGGTCATAACCTCAGCTTCTCTGTTACTCTCATCCCCACAGCCAG | |
| | GCACCAATCCCATCAGTCCTCCTTCGGCTGTCTGAGCTGCATTCA | |
| | AGGATCCTTAGACCAGGTTCCAACCTAGCTCTCCTGCCTAGAATC | |
| | CTGGGCCACTAGGCCCCCCAACCCACCCACAAGAGCTGCAGGCTC | |
| | CACTCCTCCCTCCCTGCTCCCTGTTCCTCATCATGCTCTGCCCTA | |
| | GGCTGCCTGCATCCTTACAAATGTTGAGTCCACCTGAGCCTTGAC | |
| | TGTGTCTTCCTGAAGGACACAAGAATGCTCAGGGACTCGGTAACT | |
| | GAATTGAGTACCTGGGCATGGGACTTTATTGCATATGGGGCTTCT | |
| | ACCTGGCCCTGAACCCACACAACACTATGCCTCAGTTTCCCCTCT | |
| | TTGTGCCCAGAGTGTACCCTCCTCTAATTCCACGCTCTCCACCTC | |
| | CGTGAAGCCCCTGCAGAGTCACTGATCTAAAATGCAGAACCCTGG | |
| | CTGGGCATGGTGGCTCACACCTGTAATCCCAGCACTTTGGATGGG | |
| | GGAGACCAAGGTGGGCAGATCACCTGAGGTCAGGAGTTCAAGACC | |
| | AGCCTGGCCAACATGGTGAAACCTGGTCTCTACTAAAAATACAAA | |
| | AATTAGCCAAGCATGGTGGCGCACGTCTGTAATTTCACCTACTCG | |
| | GGAGGCTGAGGCATGAGAATCACTTGAACCCGGGAGGCAGAGTTT | |
| | GTAGTGAGCCAAGATCACGCCACTGCACTCCAGCCTGGGTGACAG | |
| | AAGGAGACTCTGTCTCAAAAAATAAATAAATAAATAAAAATTAAA | |
| | AATGAAATAAATAAATTAATTCAATTAAATTAAATGCGGGATCCA | |
| | GGTTTCAGAGCATCCTGGACCCCAGAACTGGAGAAAGAGGGGAGC | |
| | AGCCCTCAGCTGGGATCCCCAGGAGTAAATGTTAGATGGCTCCAT | |
| | GCCTGGCTGACAGCTTTAGGTTTGCTGTGGAGCTTCCTCATATTG | |
| | TCTCCTTCTTCTGCCCCAACCCTGCATGTCCCTAATAATTTCCAG | |
| | GCTCCTGTGGCATCTGGATGGCTGAAGAAGCCGGGGAGATGAGGG | |
| | ACCATGCCTTCCAGCTTCTTCCTGGCTGCTGGCCTCGCCCTCCCC | |
| | TCCCTGGGCTCAGCTTTCCTGCCTCAGGGGTCCATCCATCTGTCT | |
| | CTGAGCTCGAGAACCACCCACTTCCTTGGGACTGCTTCTCTTCTC | |
| | TCATGCCACAATTGGAGGATGCAATTTTCATCATCTTTACAGGCG | |
| | GCTGGAAGGAGGCTTCTCTCTCCACTACACGGAAGGGTAAATTGA | |
| | AGCCAGTGGGAAGGAATCTCCTCACCCAAGACTTCCTGGATTGCT | |
| | GGTGGCAGGGCCAGATACCCCTCTCACAGACCTCCCAGTGCACTG | |
| | CCAGCCTGTCCTGTCTGATTTAGACTGTCAGAGCTGTCAGGAGCT | |
| | GCTCGGAGTCAAGTTCCAGATGACTGTGTATGTGGCACAAGCAGA | |
| | GGCTCTGGCTCTGATCTCCCTGAGGGGCACAGCTCCTAGTCCCTC | |
| | CCTCGCCCCACGCCAGGTAGGACCTCCTTACCTTTGCCCTGATAC | |
| | ACCTTGAGGAGCAGGAGGAGTATGAGGGTGACCACTTGCCCCAGC | |
| | ATTTCCACAGGACAGAGGGGCCAGGCCAGCCCCTCCACCCCACCA | |
| | GACTCTCTGCCGTGCACGGGCTCAGCAGTCCCCAGTCAGCAAGAG | |
| | GACGATGGGGAGCAGAACTGCCTTGCAACCTGTCCAGCCACAGTT | |
| | TCCTCAATTAGAGGCTGTTAACGCCTGCTGTGAGCTGACAAGGCC | |
| | ACTGAGAAAGCCCCAGCGCCTGGAGCTGGTC | |
| 17. | GTGTAAACAAGAGCAGGGCATGTGTGAGTAGTTGAGAATGGTGAA | NCR1 |
| | TAGGAGTATGACTAGACAGAAGATAGTAGGGATGACAAGTTTTTG | |
| | GGGGCACATTCCAAGTTGGTCTGGTGTCTGGAATGAGACTGGGGC | |
| | TTAATAAAAAGGAGCGTCTATACAGGAGCTCAAATGGGCTGTACC | |
| | CTTTAGCATTCTGAGGACAGGCCTGAATTCTGAGAAAAGAAAGTG | |
| | GTAAAAGTATTGTCCAGTCTTTTTTAAGTTGGTGGCTGAGCTTGG | |
| | TGAGGTGTGTTTTTAAAAGACTATTAGTCTGTTCTACTTTTCCTG | |
| | AAGACTGAGGACTGTAAGGGATATAAAGGTTTCACTGAATACCAA | |
| | GAGCCTGAAAAACTGCTTGGCTGATTTGACTAATAAAGGCCGGTC | |
| | TGCTATCAGACTGTATAGAGGTGGGAAGGCCAAACTGTGGAATTA | |
| | TGTCTGACAGAAGGGAAGAAATGACCTCGGTGGCCTTCTCAGACC | |
| | CTGTGGGAAAGGCCTCTACCCATCCAGTGAAAGTGTCTACCCAGA | |
| | CCAAGAGGTATTTTAGTTTCCTGACTCAGGGCATGTGAGTAAAGT | |
| | CAATTTGCCAGTCCTAGGCGGGGGCAAATCCCCGAGCCTGATGTG | |
| | TAGGGAAGGGAGGGGACCTGAGCAATCCCTGAGGGGTAGTAGAAT | |
| | AGCAGATGGAACACTGAGAAGTGGTTTCCTTGAGGATAGATTTCC | |
| | AGGATGGAAAGGAAATGAGAGGTTCTAAGAGATGGGCTAGCAGCT | |
| | TGTAACCTACATGGAAGAGGCTATGAAATATCGACCGAATAGAAT | |
| | GGGCCTGTGAGGCTGGAAGGAGGTATTTTCCTTGGTCTAAGAACC | |
| | ATTIGCCTTGTGTGGGAAGAGATTGATGGGTGGAAGTTTCAGTGG | |
| | GGGAGTAGGTGGGAGTGACTGATGAGAAGGAGAAAAACTGGCTGT | |
| | GGGGGACAGAAATTGGCATGCTAGCTGCTTGTCTAGCTACCTTAT | |
| | CAGCATAAGCATAGATGTGAGAGACAGAAGTTGGAAAGCTAGCTG | |
| | CTTGTCTAGCCACCTTGTCAGCATAGGCATTGTCTAGAGCAATGG | |
| | GATCTGATGACTTTTGATGGCCTTTGCAGTGAATGACTCCAGCTT | |
| | CCTCTGGGAGTAAAGCGGCCTTGAGCAGAGTTTTTATTAAGGAGG | |
| | CATTAAAGATGGAGGACCCTTGTGTAGTGAGGAAACCTCTTTCAG | |
| | CCCATATGACCGCATGGTGGGGCAGAATATGAAAGGCATATTTAG | |
| | AGTCAGTATAAATATTGATGCATAGTCCTTTTGCATCAGTGAGGG | |
| | CTTGAGTTAAGGCAACTAATTCGGCTTGCTGAGAGGTAGTGGAGG | |
| | GGGCAGAGCGGTAGCCTCAATGATAGATGTGGAAGATACTATAGC | |
| | ATAGCCTGCCTTTGCTGGTGAGTGGCGATTAGGCCTGGTGGAACT | |
| | GCCATCAATAAACTAAATGTGATTAGGGTGAGGAATAGGAAAGAA | |
| | GGAAATGTGGGGAAATGGGGTGAATGTCAGGTGGATCAGAGAGAT | |
| | ACAGTCATGGGGGTCAGGTGTGGTATCCGGAATAATGTGGGAGGC | |
| | CGGATTGAAGTATGGGCCAGTAACAATGGTAATTGTGGGAGACTC | |
| | AACAAAGAGTGAGTACAGCTGAAGGAGCCGGGGAGCAGAAAGTAT | |
| | ATGCGTCAGGTGTGAGGAAGAAAATAGATTTTTGGAAGTTATGAG | |
| | AACTGTAGAGAGTGAGTTGAGCATAGTTTGTGATTTTGAGGGCCT | |
| | CTAAAACTATTAAAGCAGCGGCAGCCGCTGCTCACAGACGTGAGG | |
| | GCTAGGCTAAAACAGTAAGATCAAGTTGTTTGGACAGAAAGGCTA | |
| | CAGGGTGCGGTCCTGGCTCTTGTGTAAGAGTTCTGACCACGCTAA | |
| | CCATGCCTAGGAAGGAAAGGAGTTGTTGTTTTGTAGAAGGTGCTG | |
| | GGGTTTGAGAGATCACTAGGACACGATTGGCAGGGAGAGCACGTG | |
| | TGTTTTTATGAGAATTATGCCGAGATAGGTAACAGAGGAGGAAGA | |
| | AATTTGGGCTTGACTGAAGTAATGGGGGCTGTCTGTGAAGCCTTG | |
| | CAGCAGTACAGCCTAGGTAATTTGCTGAGCTTGATCGGTGTCAGG | |
| | GTCAGTCCAAGTGAAAGCGAAGAGAGGCTGGGATGAAGGGTGCAA | |
| | AGGAACAGTAAAGAAAGCATGTTTGAGATCCAGAACAGAATAATG | |
| | GGTTGTAGAGGCAGGTATTGAGGATAGGAGAGTATATGGGTTTGG | |
| | CACTACGGGGTGGATAGGCAAAACAATTTGGTTGATAAGGTGCAG | |
| | ATCCTGAACTAATGTGTAAGCCTTGTCTGGTTTTAGGACAGGTAA | |
| | AATGGGAGAATTGTAAGGGGAGTTTATAGGCTTTAAAAGGCCATG | |
| | CTGTAGCAGGCTTTAATCCTTTTAAAGCATGCTGTGGGATGGGAT | |
| | ATTGGCATTGAGCGGGGTAAGGTTGATTAGGTTTTAATGAGATGG | |
| | TAAGGGGTGCATGATTGGTCACCAAGGAGGGAGTAGAGGTATCCT | |
| | ATACTTGTGGGTTAAGGTGGGGGGATACAAGAGGAGGACACAAAG | |
| | GAGGCTTTGGATTGGGAAGAAGGGCAGCAATGAGATATAGCTGTA | |
| | GTCCAGGAATAGTCAGGGAAGCAGATAATTTAGTTAAAGTGTCTC | |
| | AGCCTAATAAGGGAACTGGGCAGGTGGGGATAACTAAAAAGGAGT | |
| | GCTTGAAAGAGTATTGTCTAAGTTGGCACCAGAGTTGGGGAGTTT | |
| | TAAGAGGTTTAGAAGCCTAGCTGTCAATACCTACAACAGTTATGG | |
| | AGGCAAGAGAAACAGGCCCTTGAAAAGAAGGTAATGTGGAGTGGG | |
| | TAGCCTCCATATTGATTAAGAAGGGGACGGGCTTACCTTCCACTG | |
| | TGAGAGTTACCTAGACTGTCTGTGATGGTCCTGTAGGCTTCCGAG | |
| | GCGATCGGGATCGGGCAGTGTCAGTCTTCAGCTGCTAAGCCGAGA | |
| | AGATCTGGGAAGGAGTCAGAGAGCCTTGGGC | |
| 18. | TGCACAATCAGTGGTTACTGCTGCTACTCGGACGAGAAAGCTGTA | Ly49 |
| | TTTTATTACTCACTATTTGTCACCAACACTGTGACTATCATCAGA | |
| | AGAAGTAAACAGAGGATCCCAAGAGTCACTGCAATGAGGTGCCAG | |
| | GGCACTGAAAATTCTTTAAATACAAAAATAAGGAAAATAATCACC | |
| | AGGGGAAGAGCTTGGCTCACTACTAATCTTGACCTGAGAAACCAT | |
| | TTGAAACAAAGAGACAATATCCTCTAAAACTCTGACAAAAATATA | |
| | TCACACAAGTATATTTAAATTTGTAATCTGGAAATTACATGCATG | |
| | CATTGTAGAAATCCCGCACACAGGAATAGTTCTATAGGATTTAAA | |
| | AAGTCCATCCCACATAAAAGCACCAATAGCAGTAGACACAAAACA | |
| | TGTAGCAAAATCTAAGCTAACGTGTGTGTTCCTTCCTGTGATCAC | |
| | AACTAATCATGAAGAGAAAGGAAAAAGCACTCAATAATGCTAAGA | |
| | AATTAGTAGTCAATAGTGGATATTCTGAAGCTGCAAATGTGGATG | |
| | GGTCCATCTTTGAGGGAGTTTCTAGAAATCAACTTTTCTTTCTTT | |
| | TTCATCAATTCTATTTTTCTCTAAACAATGCTATATTTATCCTTT | |
| | AGCTTTTGAGATCCTCAGTGTCTGAAAGTTGTATCTCTGATTATC | |
| | TCTATATTAAAGCAGCAAAAGTTATTCTCAATGCATAGTTTAACA | |
| | ATGACCACATTTGAAGGGAGAGAGAGAGAGCATAGAAAAATTAGA | |
| | ATGCCCCTTTTGATTCAAACATTGTTACTGCATCATTGCCATTAA | |
| | GATTTGGTAAATACTTAAGGCACATAAAATATCTGCTATCTGCAT | |
| | AAAAATGTTGTTTTTAACATTGATTTTTCATTTCTTACACTTCCT | |
| | CTCATACTTTTATTTCTCTAGGAAATGTCATGTTTTCTTCAGATC | |
| | TAGTCTGTCCTTTGTTATCAAGGCCTCACAAATTCTTCTTGACCA | |
| | CATTTAAATAAAAACATTCTACTAGTTTCTCAAATTTAAAAGAAT | |
| | TCACTCATTTCTCCATTCTCACAACTTAAAACAAAAATACTTCTA | |
| | CTACTCTGCCCCATTATAGTTTCTTATCACCAGCCAAGAACAAGA | |
| | AGTACAGATAGGAATCTTTTTTCAAATGTCAAAAGTTTGACAATC | |
| | CATAAGAAACAGGTGAATGTAAATGCTTAAAACGCATACCTTTGT | |
| | CATCAGTTTTCCCAGGCCTTTGAGTATCATCAGGCCTTAATCTAT | |
| | TCTGTGACTCTGAAGGAGACTGCAAAAATCTCAGGGTTGAATAAA | |
| | TCTCTCCCTGATCATTCATCTTTAGAGTAAGAAGGTAAGTTTTGC | |
| | ATTCAAATCCTGAAATAGGATGGTTTCTGTTGAAAGAAAAATTAA | |
| | AATGTCAGCCAATAAGAACACACCAGGATTTGATGCTGAGAGATG | |
| | TATGTGTGCCTGTCTGTGTGTCTATTGGCGTGTGTTTATTAACTA | |
| | AGTGCCTAAAGCATTTAGAGTGAGCCTCATTTATAGAAGAAATGA | |
| | AAAATCTTTCAATACTTTTGTTAAAGTCATTTATACTTCTAGTGA | |
| | TCACAGCCATGATAATGAGAAAATACAGAAATGATGAGAAAAAAA | |
| | TCTACCAATAGATTATTGTTCTATTCACCCAGCCAATTTTGCTCA | |
| | AAATATTCAACATACATATACAAATGTTAGGTAAAATTGATACCA | |
| | CTGCAAATCACTGAAACACTTTATGAGTTCAACAAACAAAAAATC | |
| | TAATAAATTCTGGAAAAATAAAAATATCTTGGCTTTTAAAAACTT | |
| | ACATGTATTCTAATATACATGTAAATATAACAAAGTTCTTGCATT | |
| | CTTTCCAACTATACTTGACTCTATCAGAGTGCTTTCCAAAATGTA | |
| | TCGCATATACCTTCTACAAAAGAATTATTTGAGGGTTAATATTGC | |
| | AAATTGGTTAACATGCCAGACTCAGTGCCTTTTGCAAGATGTACT | |
| | AAATAAAAATCACTTCGGTTGGAGCCCAGATAAATGTATTTTAAA | |
| | TCTCAAATGATTCCATGTACGTTAAAATTTTAGAAATAATCTTCC | |
| | GGTATATTGCCAGAGAAGCCTATGTGAAAATAACAGTTGTCAACA | |
| | GGCAGTCATTAATGTCCATAAAAGATTCTATTGATTAATGAATGT | |
| | CAATATAGAAAAGAATAATTAAATAGTAAATACCTAAAATTTACT | |
| | TCTTATGTTAGTCAGTATAAAAAGATGATATCCAAGAAATGAGAA | |
| | ATATGAAAAAACAAATTGCACATGTAACCATATTCTTTTCTAATT | |
| | TTTTTAATTTACAAAATTTTCTAACCTAGTCACATTTTCCCTAAT | |
| | TTCCTAAGAAAAAGAAGTATTTTCTATTATAGAGTCATTCAAGAT | |
| | GGATAAAATATGTAAATCATTAGACTTAAGTAAAAAGTAATTAAC | |
| | CCCTCAAATCTCTATTCTTTATTTTGATAACACAAAGCAAAAATC | |
| | ACAGCATATTGAGAAAGAGAAAATAACTTTTTGTTAATACTGGAC | |
| | TACAATTTCCTTCAAAGTAGTGCAGCATTAATACAATCAGTAGCG | |
| | AATGCTTTCCTATCATTTCTGTTATAATGCTAAGTCATTTGTAGT | |
| | TAAATGCCGTATTTCTAGAATTAGTAAGACTAAGGATAATCTGGT | |
| | AGAAAGATCTGAGAAAATAAAAGTGGATATTACCTGTATCTGTGC | |
| | TATGCAGTGGTGCAGGTGGAGAAAACTGATTTCAAAATGAGGGAG | |
| | AAGCTGGCCTTTCGGGTCCTGGATCCCTTAGGGGCTATAACATGA | |
| | AAGGCAGAAATAAATTCTCTTCGAATGATACTGTAACTCAAAATT | |
| | TTCTGCCCCACCTCTTGACCAAAGTGATAAGAGGAAATTGTATCA | |
| | TGAAAGCTGGTACTGACGGGAAAGGAAAGATAGTCAAAAGCTGCA | |
| | TATTCCTCTATCTCAACTAACCAACCACTTAGTACTCTTTGCAAA | |
| | ATAATGTTCAAAATCAATATTTGAGGGCTGA | |
| 19. | TTTGGATAACTGTGACAACAGGTATAAAAGATGAACTGGAACAGG | CD49b |
| | AGAGAAGGAAGCACAGAGAGATTATATGGGAGTTTGTCACCATGG | |
| | TGAAAGATCAAAAGGGTTTGAACCAAAGAGAGATTAGCAGTGGAA | |
| | CAGAAAATAGGAGATGGCACAGAAAACCCTAGTAGGGTGTAAAGA | |
| | CAGAATTGGAATGGGATGTTAGCAGGGGGCTGGTGGTGCACCTGG | |
| | GGAAGAAAGCAGAGGTTTCTGGTTTGGGTGATTGTCTATATGAAA | |
| | ACGGGATAAACAAAGGAGGGCAGGGCATGACGATACAACAGATCA | |
| | GAAGGAAACACTGGGGAAACTTATCTGAGCCGTGTTCCCCAACAA | |
| | AACTCTGTCTTGAGCTAAGGAAAAGAAAAAGAGGGGAAGGCCTCA | |
| | CTTCACCCCTGAGTGCTTTTTGGAGTAGGGTCTCTGGGTCTCTCA | |
| | AGATCCTTTCAAGTGGCCAGTGAAGTCAAAACTATTTTCATAACA | |
| | ATACTGAGACGTTACTGCGTTTTTCACTGTGTTGACATTTGCCCT | |
| | CATGGTGCAAGAACAAGGATGGGGACTGGTGGTCATTGTATTCTT | |
| | CATCACTGGGCACTCACAGGAAGAGAAAGGGAGAGGGACACGGAG | |
| | AGGCCAGCAGATCAATGAATTTTAATGCCACGGAATAGGAAAAAC | |
| | CATTGATATGGTTTCAGATTCCACATTGCAACTTTAAGAAACTAC | |
| | CACTTGTTGAGTTTTGGTGTAGTATCAGGGGATTATCCAGAATTA | |
| | TCTCCTGTTTTTCTAAGTCCATATCTGTGTAACACCAGATTAATC | |
| | AATATAATTTCACCAAAACTACATGCCGTAACAGATTGGATGCAG | |
| | AAGCAGATCTGAGAATCCAAATGTCTGTTTTTAAGCCAGATATTT | |
| | AAAATATTAGCAAAAATGTAAAGCAATGCCACTCTTCTCCCGGAT | |
| | TCTTTCATTTGGAGAAATATAGTTATTTTTCATAAAATGTACATT | |
| | ATTTATGTTCACACATAATAGGATTTTTGTTAAAGGACTTACTGA | |
| | GTAAAGGGCTCTTGAGACCAAAGAGTTTGAGAAGCCCCAGCTTAA | |
| | CCTTCCATATTTCCTGCTCTGAATGTGGTGTCCCTCACAACTGCA | |
| | ACTAGGCAGGAGAGCACCCTGGAAGGGGTTCTCAAACTACTTTGT | |
| | CTCAGGAACACTTTATCCTCTTAAAATTAATGTGAGGCAGGAGTT | |
| | TGTTCTTTGTTTCTAAAAGTTTTGCCTGGCTCTGGAAAATCGTAG | |
| | CTCCTGACTTATTCTGCACTTCATGACAGTGTAATGTTAATTTGT | |
| | CCAGTTCTGAGAACAAAAACCAAGAAGGATGCATGTTATTTCCCC | |
| | CCACCCCCAGAGATAAAACACAAATTCCTGTCTCACATTAAAGGA | |
| | AGTGTCCTGCCTCCCAAACACAGGTCTGTTCCAAACACTTACTCA | |
| | CATTTCAGTGAGTCAGAAGGAAACAGACAGATATTCATCTCCCTG | |
| | AAACCTGGCTGACTTCCTTCAAAAAGTCTGTAAGGAGCGTCAAAA | |
| | TCGTGTCACCGGTTAGTGAGCAGCCACCTAAGAGCTCTCAGGTGA | |
| | TAGCCGGAGTTTTAGTAAGCAGGTGAGTACCCCAGACAATTCAGA | |
| | AAAAAATGCCTTCAGGTGTTTGTCAAGGTGAGTAAGTGCAGCCAG | |
| | TAAGTGACTGATAAGAAAACAGGAAAGAGCCTGGGACTATATAAT | |
| | TTGGGTTCAAAGGGTATTAGTCATAAGCCATCAGCAGCAACATCT | |
| | GATGCATACCAATTTAATACTTTAACAATGTGACACATCAGCCCT | |
| | ATCTATAGTAAAGAGAAGAGCTTGATTTTTATGAATTATTAAGTC | |
| | CTTCCCACCTTGAGCCACTTTGCTTCCTAGAGGCAGTATCTCAGG | |
| | CATCCCATTCAGCTCATAGTAGCATTCCATGAAGATCTGGAATGA | |
| | TGACAATAATGATGATTATTAGACTCATAACTCCTTAAGCTTGAT | |
| | TTATGAAAACAGTTCGTTCCTTCACAAAAGTAGTCACTGGAAGAA | |
| | TTCTGGTTTCAGCAATGTGCTGGAAATTTGTGGCCAAACTTTTGG | |
| | ATAAAACATAAGATTCAGACAAGAAAAATTAATATCCTATTTCTT | |
| | AGGTTTTTAACTAAAAATCTCGGATATAGAATGTTCTCTTTCCAG | |
| | ATGTGAGACTGAAATAATGGAAGAAAATGGTCACAGTCTATGTCA | |
| | TGGTGTAATGTAGACAGGGAGAGTCACAAGTGCATGTATGGGCTC | |
| | TCAGCCTTTTCTCTGGCCTTGAAGTTAATCTCCTAAACTCAGTCA | |
| | TTAAATCAGCTCTTAATTATCTCGGAGGAGAGGAAGTAATGATGA | |
| | TTTCTCTCTCTCTGTCTCTCTGTCACACACACACACACACACACA | |
| | GCTCTTGCAGCAGGTATTGCTTAAATATCACCTTGGATAATCATA | |
| | ACTTGTGAGCAGATCTTCTTTCCTGATGAGTTAGTTTCTAACCTG | |
| | GTCATTCTGCGCTTATTTTTGTCCCTTTCTCCACCCACTTAGGAA | |
| | AAACAGAGAAAGGGACGCACCGCGCAGCCCCTAGGCACTGTGGTT | |
| | TAGGGCTAGTGCCCTCGGCACCCGCTGCCAGGAGCCGGGCGCTGC | |
| | CAAGGGCTGCGGAGGGGCCACGTTCTCCCGGGGACTGGGGCATCT | |
| | CCTGCGTGCTGGCGACAGGCTGCGGGGGCGGAGTGGTGCCAGGGC | |
| | GGGCGCTCGCCCGTCGGATATGCCCACCCGTCCCGTCCAGGGCAG | |
| | GAAAGCCTGCCAGGGCGCCATCCCCATCCCCACCGCCTCCAGGCT | |
| | GCCGGGGCTGGGCCGCTGTACGGGAGCCAAGGTGCGGTGCCCCGC | |
| | GTGTGGACGAGCCGAGGTGCAGCCCGCGGGGCCGCAGGGCCGGGG | |
| | TGGGGCGGGGCGCGGCCGGAGCAGATCCGGTGTTTGCGGAATCAG | |
| | GAGGGGCGGGCTGGGGGGGGCCCTCGGCGCTGCAGGAGCTGCCCA | |
| | GAAACTTTTCCCTGCTCTCACCGGGCGGGGG | |
| 20. | TGGTTTTCCTGCATATATATATATATATATATATATATATATATA | CD11b |
| | TATATATATATATGATTTATTTTTGTATGTATGTACTTATTTATT | |
| | TAATTTTAATTTTTTATTTTTTGAAACTGAGTCAAGCTCTATTGC | |
| | CCAGGCTGGAGTGCAATGGTGCAATCTCAGCTCACTGCAACCTCC | |
| | ACCTCCCAGGTTCAAGTGATTCTCCTGCCTCAGCCTCCCAAGTAA | |
| | CTAGGATTACAGGTGCCTGCCACCACGCCTGGCTAATTGTTGTAT | |
| | TTTTAGTAGAGATGGGGTTTCACCATGTTGGCCAGGCTGGTCTTG | |
| | AACTCCTAATCTCAGGTGATCCTCCCACCTCAGCCTCCCGAAGTG | |
| | CTGGGATTACAGGCGTGCCTGTATATATTACATGCCCTGCCTATA | |
| | TATATTTTAGAGACAGGGTCTGGCTCTGTTTTCCAGGCTGGAGTG | |
| | CAGTGGTGCAATCATATCTCACTGCAGCCTCAACCTCCTTGGCTC | |
| | AAGCGATCCTCCCACCTCAGCCTGTTGAGTAGCTGGGACCACAGG | |
| | TGTGCATCACTATGCCCAGCTAATTTTTAAACATTTTTTTGTAGA | |
| | GTCAGGGTCTTGCTGTGTTGCCCAGGCTGGTCTTGAGTTCCTGAG | |
| | CTCAAATGATCCCTCTGCCTTGGCCTCCCAATGTGCTGAGATTAC | |
| | AAGCAGGAGTCACCACACCTGGCTATATATAAAAATTATAAACAG | |
| | ATAATTTTTAACCAAATGTACATAATTTTAATTGCTTTTTTTTTT | |
| | TTCAGCCAGCAAGCATTTTTTTCCATCTAGTTATCGGGAAAGTAG | |
| | AACTAGAGGCAAAAGAAAATGTGTTGAATGGTGAAACCCTGTCTC | |
| | TACTGAAAATACAAAAAATTAGCCAGGCGTGGTGGCAAGCACCTG | |
| | TATTCTCAGCTACTCGGGAGGCTGAGGCAGGAGAATCACTTGAAC | |
| | TCGGGAGGCAGAGGTTGCAGTGAGCTGAGATCACGCCACTGCACT | |
| | CCAGCCTGGGCGACAGAGGGAGACTCCGTTTCACAAAAAAAAAAA | |
| | AAAAAAAAAAAAGAAAATGTGTTGAGAAAGAGTGAAAAAAATCAT | |
| | TGGCTTGAGAATCACATTGGCGGTCACATAAAGGTGAGGTTTGTG | |
| | TTCCATGTATGTGTGGAAGGATACCTATTTTCCTGTGCCTTTGAA | |
| | AACAGAATTAAAAGTGACAGACACAGGGGAGGGTAAGGATATATC | |
| | ACAGAGTGAAGTATCATTGAACCATTCCTCTGTTGAATATTCAGG | |
| | TTTTTTTAGCTTTTCATATTAACAATAATGCTGCAATGAACAAGC | |
| | TTGCAAATATATATATATATATATATTTTTTTTTTTTTAGAGATA | |
| | AGAGTCTTGCTCTGTCGCCTAGGCTGGAGTGCAGTGGCACAATCT | |
| | CTGCTCACTGCAACCTCCGCCTCCAGGGTTCAAGTGATTCTGCTG | |
| | CCTCAGCCTCCCAGGTGGGATTACAGGTGCCTGCCACCACGCCTG | |
| | GCTAATTTTTTTGTCTTTTTAGTAAAGATGAGGTTTCACCATGTT | |
| | GGGCAGGCTGGTTTCAATTGCTGACCTCAAGTGAGCCACCCCGCC | |
| | TCAGCCTCCCAAAATGCTAGGATTACAGGCATGAGCCACCGCACC | |
| | CAGCCAAGTTTGTACATATATTTTTGACTACACTTCTTAACTATT | |
| | CTTAGGATAAATTACTAGAAGTGAAAATTCTTGGGTGAAGAGCTT | |
| | GAGGCCTTTACACACACACACACACACACACACAAAAATAGGCTG | |
| | GATGCAGTGGCTCACACCTGTAATCTCAGCAGTTTGGGAGGCTGA | |
| | GGAAGGAGGATCACTTGAGTCCAGGAGGTTGAGAATAGCCTGAAC | |
| | AACATAGCAAGATCTTGTCTCTACAAAAAATTTAAAAAAAATTAG | |
| | CTGGCCATGGCAGCATGTGCCTGTAGTACCAGCTACTCGGAAGGC | |
| | TGAGGTAGGAGGATCGCTTGAGCCCAGGAGGTTGATTGAAGCTGC | |
| | AGTGAGCTGTGATTACACCACTGCACTCCAGCCTGGGCAACAGAG | |
| | CTAGACTCTGTCTCTAAAAAAAGCACAAAATAATATTTAAAAAGC | |
| | ACCAGGTATGCCTGTACTTGAGTTGTCTTTGTTGATGGCTACAAA | |
| | TGAGGACAGCTCTGGCTGAAGGGCGCTTCCATTTCCATGGGCTGA | |
| | AGGAGGGACATTTTGCAAAGTGTGTTTTCAGGAAGACACAGAGTT | |
| | TTACCTCCTACACTTGTTTGATCTGTATTAATGTTTGCTTATTTA | |
| | TTTATTTAATTTTTTTTTTGAGACAGAGTCTCACTCTGTCACCTG | |
| | GGCTGGAGTGCAGTGGCATTATTGAGGCTCATTGCAGTCTCAGAC | |
| | TCCTGAGCTCAAACAATCCTCCTGCCTCAGCCTCTGGAGTAGCTA | |
| | GGACTACAGGCATGTGCCACCATGCCTGGCTAATTTTTTAAATGT | |
| | ATTTTTTTGTAGAGTCGGGGTCTCCCTATGTTGCCCAGGCTGGAG | |
| | TGCAGTGGTGTGATCCTAGCTCACTGCAGCCTGGACCTCGGGCTC | |
| | AAGTAATTCTCACACCTCAGCCTGTCCAGTAGCAGGGGCTACAGG | |
| | CGCGCACCACCATGCCCAGCTAATTAAAAATATTTTTTTGTAGAG | |
| | ACAGGGTCTCTCTATGTTGCCCAGGCTGGTTTCAAACTCCCAGGC | |
| | TCAAGCAATCCTCCTGCCTTGGCCTCCCAAAGTGCTGGCATTACA | |
| | GGCGTGAGCCACTGCGCCTGGCCCGTATTAATGTTTAGAACACGA | |
| | ATTCCAGGAGGCAGGCTAAGTCTGTTCAGCTTGTTCATATGCTTG | |
| | GGCCAACCCAAGAAACAAGTGGGTGACAAATGGCACCTTTTGGAT | |
| | AGTGGTATTGACTTTGAAAGTTTGGGTCAGGAAGCTGGGGAGGAA | |
| | GGGTGGGCAGGCTGTGGGCAGTCCTGGGCGGAAGACCAGGCAGGG | |
| | CTATGTGCTCACTGAGCCTCCGCCCTCTTCCTTTGAATCTCTGAT | |
| | AGACTTCTGCCTCCTACTTCTCCTTTTCTGC | |
| 21. | AGACTGACTTACCCTTGCAAAACACTTTACTCTGGTATCTGTTAA | KLRG1 |
| | TGATCCCTCAGGCCATTGCTACTTGTTTATTCCAAGAGTATCACG | |
| | GACAATTTCCTGATTTAACTCTTCTAAACTTTTCTCACTTACAAA | |
| | AAACGCTGCAAGTCTGGACTTTGGTTTGAATATTCTTTAAAGTTC | |
| | TCTAGTTCTTTTTCATGCGGGTCCACTTACTCCACCTCATTTTTA | |
| | AAATATAAAACTTGAAACAGGCAGAATTTTCATGGGTACTAATGA | |
| | GTTTGAAGATGGGAAAATGGAAGCTCCAACCTTGGCCGTGAATGC | |
| | ACTTTCCCCATTCCCCTAAACAACCTAACATCTGACCTTCCACTT | |
| | CCCACACCCCACTAAAGCAAAAAATCCCGGGAAATTGTCTACCTG | |
| | ACTTCCTGTACCCCAAACATCAATTCTCTACTGTAATATTATGGT | |
| | AGTTTCCATAGAATAATCATAGCTAACCAAGAGGTCCAAGGCCCT | |
| | CATAACACATTTAATGCACTGAAACCACACAACAGCAGGAAACAA | |
| | CAGACTACTGCCTTGGGCCAGGTGCTGTTCAAAGCACTTAACATA | |
| | TATTAATTAACTAATTAATCCTCACAACCTTATGAGTAAATACAA | |
| | TTATTGTTGCTATTTTACCAGTAAGAAAACTGAGACACAGCTAGT | |
| | GGAGTGAGCCAGAGAGTCTAGCTCCAGAGCTCATGGTCTGTGTTG | |
| | AACAACTACATGTATATTTCCACTATGTGCATAACAATTTTTTGG | |
| | AGATGAGAGTAGACAGCCAAAACTGTGGGTTTTTTTTTTTTTCTA | |
| | CTAAGAAGTGAAGGGGAAGTGATTCCTCACACAGTTGACTGAATA | |
| | GGAGGTGAAATAAAGAACTCAGACCTCCAACTCCCACCTTCCCTA | |
| | AGTTAACCCACTGCTCAACACACAACTTGACCAGCTGACTCACTA | |
| | AAAAGAAATAGGAGACTCCTTCTTCTAGACTGACTTAGCCTGCCA | |
| | TTTTCCTTTCCAGTCTCTCAGGACCTAATTAAAAACTGACAGTTG | |
| | ATTTCTTTTCCTAAATTAGAACAAGGAATCTCACAAAATTAGGAA | |
| | GGGATGAAAGGTTAAACACCTCAGCCATTAGCTGAGTTGAACTAT | |
| | CTTTCATTTGCTTATTATTCAAAAAGTTAGGATAATAAATTTATA | |
| | CTGTATTCTTCTTTCCTGCTTAAGGCTTGTCTTTAAATTTAAGCT | |
| | GCTTCTCTTTAATTCAGTGGCATATGCGCTAAATGTTTTCCCTAG | |
| | ATTATGTTCATTCATCTTCACAACCCAGAAAATAGGTATAATTTT | |
| | TCATACTTCACAGATGAGAAAACTGATGCTTTACAGAATTACTTG | |
| | TTAAATTACTTGCTGTACATTACACAGATATTAAATGTGTGTGAA | |
| | GTTACTGGGGCAATTTAGACGATTCGTGGCATAAAGATAACTGCT | |
| | GGAAAACTTATAAACATAGGGGTGCAACAATCAGGAAGAGAATGA | |
| | AATGGAAGGGCCCAGTAGCCCCCTTTTAATATGCTACAATTAGAT | |
| | TCTCAAGGTACAAGAGTTTGTCTATATACTTTGGAAATAAGGAGC | |
| | AGGATTTTATGATCTTGAAAAATCTTTCAGTTTTCTTATTCTCTA | |
| | ACGACAGATCTATTTTATTTGGCAATGCTAAAAATTATCCTTTAT | |
| | TAGGGTGGCCAATGACTAAGCCAGCAACTGGGAAGCAGCTTACAC | |
| | CACTGGGCCTCTGTAGCACTCACTTCCCCATCTGTTAAGACAGCC | |
| | ATGGCCATTTACCAGTGGAATTTCTACTGTCTTCCCGCTGGCTCC | |
| | TTTCTGATGTAGCAGTCCTTATCCCTTTTTCTCCAGCAGCCTACA | |
| | TGTTTAGAAAAGCAGCCTCAATTGGCTAAAAGGCCTAATGCTGAA | |
| | AGAACTCAAGAGAGAAGGAGCCTCAGACAAGTAGCGGCTATGTCC | |
| | CAAGAGCGGAAGCTCCCCACAACGAGGAACACAAGATAATCCGTC | |
| | CATCAGCCATTCTTATCCACACCACAGTCGCATCCCCCTCAAAGC | |
| | TCTCCCAGGGTGCCAGAAATTCTCGCTCTGTCTAGTGTCCCGACT | |
| | TTCCAAGCGCTTGTAGCCATACCCTCTCCAAATGATGCCACTTGA | |
| | CCTCCCACCGTGATGCAGGCCAAAATTCCTCCTACAAATAAATCC | |
| | ACCAACACCTCCAACGGACTCCATACTTGGGGTATTTTCCTTTCC | |
| | CTGCTTTCTAAATATCGTAAATCCCCTCTCTGGTCTTGGAAGTCC | |
| | TTTCTCCCTCACACCTTTCGGAACTCCGTGACTCTCCGCTCTCAT | |
| | GCCTCCGCCTCCATTCTACCCTTAGGGCTCCTCAGCCAGATCTTT | |
| | GGCTAGAACCATGGTCAAGGGAGAATGCGATTCCGGCTCCTGTCA | |
| | CAGACGCTTAAGTGTGAGCGCAACCCAGCCCGCCTCCTCCTCCAC | |
| | GCCCCTCACACTCACGTGTCGTGGGAAACTGGAGTAGCCAAAGCT | |
| | CAGAATCCCCAGTGAATACCCTTCTGGGGATGGGAGACGGGGCCA | |
| | GCTAGGGGCCGGCCGCAGACCCCGGTCCCAGAGCTAGTTTCAGAG | |
| | GCCAGCGTTCCCCCTAGCGCCTCGCTGTGCCCCACTCTGGGAACC | |
| | GGAAACAGGAAGCGCCAAGAGGCCGCTGGGCAACCGCTCCTCCCA | |
| | CCAGACCCCGCGGCATGTGACCATAGAACCATTTCGCCCCTTTCC | |
| | CCTTCCCGGGCTCCCTGATAATCTTAAACTCTGATTGGCCAGTAT | |
| | TCTGGGACTTTAGCTCCGCCCTCGTTCCATCAGCTCCAATCACTA | |
| | TCGAGGTTGTCGTTTCGCCTTTTCTGGTTGCCTTTCTTTCAATGG | |
| | GGTCCCAGAGTCCCGCTTCCAGGGCCTCTCGGCTCGCGCACCCCG | |
| | GTCACGCGGGGTCACGCGCCTGACCGCTCCCGGGTAGGGTTCGAG | |
| | TGAAGAGGGTGAGAGGGGCAGGTGGCGGAAGCGGAAGTTACGCAG | |
| | GCAGCTCGCCCTCGGGCTCCGCGGGAGTTGT | |
| 22. | CGGGCGCGGTGGCTCACGCCTGTAATCCCAGCACTTTGGGAGGCC | CD43 |
| | GAGGCAGGTGGATCATGAGGTCAGGAGATCGAGACCACGGTGAAA | |
| | CCCTGTCTCTACTAAAAATACAAAAAAGTTAGCCGGGCTTCGTGG | |
| | CGGGCGCCTGTAGTCCCAGCTACTCGGGAGGCTGAGGCAGGAGAA | |
| | TGGCTTGAACCCGGGAGGCGGAGCTTGCAGTGAGCAGAAATCGCG | |
| | CCACTGCACTCCAGCCTGGGCGACACTGTGAGACTCCATCTCAAA | |
| | AAAATAAATTAATTAAAAATAAATAAATAAATAAAATATAGTGTA | |
| | TTTCTGGCCGGGCGCGGTGGCTCACTCATGTAATCCCGGCACTTT | |
| | GGGATGCTGAGGCTGGCAGATCACAAGATCAGGAGATCAAGACCA | |
| | TCCTGGCTAACGCGGTGAAACCCTGTCTCTATTAAAAATACAGAA | |
| | ATTAGCTGGGCATAGTGGCATGTGCCTGTAGTCCCAGCTACTTGT | |
| | GAGGCTGAGACAGGAGAATCGCTTGAACCTGGGAGGTGGAGGTTG | |
| | CAGTGAGCTGAGATCGAGACACTACGCTCCAGCCTGGGTGACAGA | |
| | GCAAGACTCCGTCTCAAAAAAAAAAAAAATTATAGTGTATGCCAT | |
| | GGAATATTATGCAACTGTTATATATGTGTGTGTGTTCCCAAAGCA | |
| | TTGTTCAGTAACAAAGGAAAAGTGCAAAACACTAAAATGGATGAA | |
| | TACATGCTTGTGTAAACACAGACACTGTCAGGAATGTTTGTCTGG | |
| | AGGTGATGGGTATGGAGCCTTTTTGTCTGATATATTGGGTGTATC | |
| | AAGAATGCCAGGTCCTGAGCAGTTTTTTTTTTTTTTTTTTGAGTT | |
| | GGAGTCTCACTCTGTTGCCCAGGCTGGAGTGCAGTGGCACAATCT | |
| | CTGCTCACTGCAACCTCCACCTCCTGGGTTGAAGCGATTCTCGTG | |
| | CCTCAGCCTCCCGAGTAGCTGGGATTGTAGGCGCCCGCCACCATG | |
| | CCCAGCTAATTTTTTGTGTTTTTAGTAGAGACGGGGTTTCACCAT | |
| | GTTGGCCAAGCTGGTCTCGAACTCCTGACCTCAGGTGATCTGCCT | |
| | GCCTCGGCCTTCCAAAGTGCTGGGCTTACAGGCATGGGCCACCGT | |
| | GCCCAGCCGTTTATATGTATTAACTCTTAATCATCAAAACAAACT | |
| | ATGTGGTATTACCATAATTATCGCCATTTATACAAAGCAACAGAG | |
| | GGATAAGGAGATTAAGGCACTTGCCCAAGTGCAAGAGGTAGGACA | |
| | GGAACCCAGAGTCCCGTGCTGGTAGCTGCTTTTCTGTGCACTTAA | |
| | CATTATCTCATTTAATTCTTGCAGTCACTTCATGAAGAAGGTGGC | |
| | GGTCCTAGGGCATTGGAGAAGTTATGTGCTTGTTCAAGGTCATGC | |
| | AGCTGGAAGATGGTGGTGGTAGGACAGGAAGTCTGTAGGGCCTGA | |
| | TTCTGCAGCCCTCTAGTCTATACTCCTTTGCTCCTGTGTCCCTCA | |
| | TCCCCCCTGCAGAATGGGCACCCCGTTACCTTTCTGAGCCACTGT | |
| | GCGCAGAAAAGAGAGCATGTTGGCCAGGCTGGTCTCGAACTCCTG | |
| | ACCTCAAGTGATCAGCCTGCCTTAGCCTCCCAAAGTCCTGGGATT | |
| | ACAGGCGTGAACCACCACGCTCAGCCTCTGAATACTTTGTACTCA | |
| | AGCCATTTTTCAGTGCTGTGTTTGCAGTGAGCACACCCGAGGGAT | |
| | GAAGACACGTCTCCCTGTGGGAACCTGGGCTTACCAGGGCCCCTA | |
| | GAGGAGGGGAATCTCTCAAGCTCAGAGCTCTATGGCTGCGGTGCA | |
| | GGCCCACTGTGTGCATGGTGTCAGTCTGGGCCCTTCCATGTTGCC | |
| | CCCGTGGGACTTGGGGTAAGGGGAACTGATGCAAACATCACGCTG | |
| | CTGTTGCTTGGTGTGAGCAATTAATTCCTGTGGCTCTCACCCAGG | |
| | AGTCTCATGTCTTTGGGTCAGACAAACTCATCAGCTTGTAGAAAT | |
| | GGCACAGTCCCACGGGCCTGTTAGAATCTTCTATTGTGCACATGT | |
| | TGCTCTTAAAATATACAAATCAGTTTTGATTTTAAAAAATTATTT | |
| | ATTTTTTTAGTGATAGGAGTTTTGCTACGTTGCCCAGGCTGGTTT | |
| | CAAACTCTTGGGCTCAGGAGGTCCTCCCACTTTGGCCTGGACTGC | |
| | CAGCATAATGTATCACCACACCCGGGACTGATTTTCGTTTTTCAA | |
| | GAACAAAAACCAAAAACATACACAAACCGAGAGTCAAAGCTTGCT | |
| | AATTAGAGGAAAGTCAGGAAATGGGAACCATTCAAAGAAGAAAAT | |
| | ACCCCCACCTCCTACTCTCACCTATCCAAAGACAATTAGGTGAAT | |
| | CCCTTAGTAGATATCTTTCCAGACGGTTTTCCATATAGATTCCCA | |
| | TATCTGGCCAGGCGCGGTGGCTCACACCTGTAATCCTAGCGCTTG | |
| | GGGAGGCTGAGGCGGATGGACCACCTGAGGTCAGGAGTTCGAGAC | |
| | CAGCCTGACCAACATGGAGAAACCTCGTCTCTACGAAAAATACAA | |
| | AATTAGCCGGGCACAGTGGTGCAAGCCTGTAATCCCAGCTACTCA | |
| | GGAGGCCGAGGCAGGAGAATTGCTTGAACCTAGGAGGCAGACATT | |
| | GTGCTGAGCCGAGCCAAGATCATGCCATTGCACTAAACTCCGCCT | |
| | TAAAAAAAAAAAAAAAGATTCCCACATCTTTACTAGTTTGCAGAA | |
| | ATAAGATCCTAGCATATGCAGTGTGTAGGAACCACCTTGGTTTAG | |
| | CCACGTCTCTGTGACTGGGGGCCACTGTGGTGACCCCCAGCTCCC | |
| | CGGACAGAGTCAAGAGCTCACCAGCCTGCAAAGGTTTTCACGGCC | |
| | CCCAGCCAGACTCGGGGGCTTCCTCTTGCCCTGCTACTTCCTGGG | |
| | AGCTCTGAGGGCAGGAAATGGCGCCACTCAGCTCCTGGCCTAACA | |
| | GCTTGGGGACCACAAATGCAAAGGAAACCACCCTCCCCTCCCACC | |
| | TCCTCCTCTGCACCCTTGAGTTCTCAGGCTC | |
| 23. | TCCTATCCAGTAGTAAGAACGACTGAAAGGCAGAGTCTTCTCCAG | CD62L |
| | ATACTCAATTTCCGCCTTGTTTTGTATGGCAACTAAATCTGTGTA | |
| | ATTGTCTCGGCAGAATCTTCTAGCCCTTTGCCAGTTCATGGGTTT | |
| | TTCAGAATAATGGTAAGTCCAGCAGTCGGTTCCATGATGTGCCAG | |
| | GAAATCTGCAAGACATCAGTGTGACCTATGCAGACTTACATAATG | |
| | TTACAGCTAAAAAGAACCTAGCACTACTCCAGGCTGAGCTAGACA | |
| | CTTAGAGATGAGGAAACAGAGCCTAAGAGTGTATGTGACCATCTC | |
| | AGGATCACAGAATAGTIGTTTGCAGATTTGAAGTAGAACCTAGAC | |
| | CTTCTGGCTTGAATATAAGATGCTTTTATCTAAGGTTCTATTTGA | |
| | AACAAATTTAGTGGTTTTCTAGGTTTATTTTCTTATTAATTTTTT | |
| | TCTCAAAATTATTTCAGGTGAAATTTAACCAACATATTTTAGACA | |
| | TTCATATTTCTTTTTCTTTGTAGCTGTTAATGATTTACAACTAAT | |
| | TACCGTGTAATATCATATAACTATACAATTTACGTATACTTTTTA | |
| | ATCCTGGAATCATTTCTTGAAGGCCAACACATATGTACCTATGGG | |
| | AGAAGCATAATAAGGACAGGAAGAACAGTGACATACTTTTAAGTA | |
| | ACCTCTTTTACATAAAAAACATTTTATTTTACCATAGGAAGAACT | |
| | GCTTCTGGAAAAGCCCAATATACCACTCAACTCTTATATATCTAA | |
| | CTGTATAATTTTTAAAAAGAACAATTTACAAAGCCAAATGGTATA | |
| | GGATTATGAAATTCATTAGATCATGTTCTATACACAAAGAGACTC | |
| | AACTGATGATGTTTAATAAACATATGGACCCATCAAATATGAGGG | |
| | CTTTGAAGATATCTAATTAAACACATAATTACACAATGACTTCAT | |
| | AATAATATATGGCATTCTAAGCATGGTATGATCTACATGAATCAC | |
| | TATTTAATACAGTAAAGAAACAGATATAATTGATGGTAAAGAGCA | |
| | TCATAAAATAAACATTTTGAACAGAGTTTTGAATGAGCATTCCAC | |
| | TAGAATGCAAGTTCTAAGAGGGAAAAAACTGTTGTGTCCACTGCT | |
| | GTATCCTTAGTGCCTAGCATAAATTTCACACATTGTAGGGACTCA | |
| | GAAAATACCTGTTGTATGAAAAGAGCACTAAGTTTCTATGTGACA | |
| | CAGTGCAGACATGGCATAAGGAATGTGTGAACGGGAGAGTTAGCA | |
| | TGTTTGCTTGGCTAGAGCTGAAAATCCAGGCTAGGGAGAAAGAAG | |
| | ACATTAGTTTACTTAGGAAATGAAAAACCAAGTTCAAAGCTATTG | |
| | CTGGAGAGTCTTCAAGAATCAGATATAAAATTTGTCACAACAATG | |
| | GGAGAAGGACCAAAAAATGATAAACCCCCGTCCCTTAATAAGCTC | |
| | GTATTGTAATTGTAGAAATGACATTAATGTACACTGAACTATGAA | |
| | TAAAAAATAGAAAATGAGGTGCTAAATATTTGGTACAGATTGTAA | |
| | GTACCTTAACAGAGATTTCTTAATTAACATTATTCCTTTATAATT | |
| | GAGGGATTTTGTGGGGTTATTGGGATTTGAACTCTACAGCATGGG | |
| | CTATTATAGGTTAAAAATAGTGTTCAGGAGTTTCTGGGGAAGAAC | |
| | TAAAGGTAAGAAGAAAAGAGATGTTTACAGAAGGGATAGAATTAA | |
| | CAGCTCTGTGAAATAATTTTCCCTTAGACTATGTATAACTAGTGG | |
| | ATATTTAAGAAAAATGAATATAAGTAAAATAGACTTAGCGATATA | |
| | TAAATATCATAACATACCACAACAGAGCATTGTCCACCCCCACAA | |
| | CTTGAAGATGTTCCATAAGTCCCTCTGGGTGCTCTGACATTTCCA | |
| | TGGAAATATCTGCAAATGAAATACAAAATTATATTTAGATGTATA | |
| | CTCTTAAACCACACATTTATAGCCTTTGAGGTGGTGCTTACAACT | |
| | TTCTTAATAATCAGAATAAAACACATATGTCTACTAACCCTGTCT | |
| | GAGGTAACAGGTTTCTCAGACATAGATGAAAAATTACTTCAAATT | |
| | TACATCAGAACTGATGCACAGTTTTGTTTTGTTCTATTTTATTTT | |
| | TACGCTTTAGTCTCAAGTTGCTAATCGGTACTGCCCTGAATTTTT | |
| | TCTATGGTTTGGTAATTTTTATACCTGCTTTTCTGCTGAGCTATT | |
| | AGATAAAACTATTTAATATTTACTATGTATATTTTTTAAAGTATT | |
| | GTTGCTGCTTAATTAACTATTGATGCTTATATTTAATGTTATAGC | |
| | CTCACTCTTGATCATAATGGGTCAATGCCTCAAATACCTAAAAAA | |
| | AAAAAAAATTAGATAGCCAGACACCAGGAAAGAAAAGTATTTCTT | |
| | TTTTTAATAAAAAGAAATACCTTTTTGAGCAACTGAAATGACAAA | |
| | GTCACAAATTTCCTGCACACCTTAAAATATACTTAATGTAAATGA | |
| | CGAGTTAATGGGTGCAGCACACCAACATGGCACATGTATACATGT | |
| | GTGACAAACCTGTATGTTGTGCACATGTACCCTAGAACTTAAAGT | |
| | ATAATTTTAAAAAAATTCTATCTTCCAAAGCATATCACTTCTCAG | |
| | GTAGACACAGTGTTTATTGCAAAAGATCTGATTTCAATAGTATTT | |
| | CTTCAAGAGTCTCCCCAGAGACAAAGTCAAGAAGAGGAAATCAGC | |
| | ATATCTGAGAAGAAAGATTTCAGGATCACTTTTTTTGAGGGTCTG | |
| | AGAAAATGTTTAGTTTCTATATTATTTAAAACCAGAATTGAAATG | |
| | GGGTGATTCCTATCCTTGCCACCTGCCTCTACAACCCCAAGAGTT | |
| | TCTATCTGAGCATCTAAACGTCTTTTAGGCTGAAAGGCTCACCAT | |
| | GGCTTTGCTTGGTCCTTCTCTAGTTCTTCTGCAGCCCATTGAGCC | |
| | TCTTGACTTAGCACAAGGGTCTCAGGTCCTTGCCCAAAGGGAGTG | |
| | TGCTGTGCTGCAGGTAGACTGCACTGAATGT | |
| 24. | TCAGTGTTCACAGGCAAACACACACACACACACACACACACACAC | CD226 |
| | ACACACACACACATCCTTCTCCTTATCACCCATAATAAAGCTATT | |
| | GCTATTTTGTTTTTTATTTTTATTATTTAGCTATTGCTATTTTTA | |
| | AGGGAGGCACAATGGAGATACCTTTTGTTGTTTCTCTGTAGAGGT | |
| | TTATCCTCTCTGGTTTATTTAGCTTCCTCTTGGAGGCTCTCGAAA | |
| | TATGGTAATAAATACAAAGAACCCAAGACTTAGCTTTGAATCATT | |
| | TCCAAAGAAACACAGGCATCCCAGTATCTTGGCCCCAAGGATGCA | |
| | ACATTGTTCCCCCAACATGGTAACTACAGAATTTTACTCAGCAAA | |
| | CAGGGGAAATGTTGAAATATCACATGATCGATAATAAATTACAAA | |
| | ACAAACAATAAAAATAACCTGAGACAAATTAAATTGCCCTATCCC | |
| | TCCAGAGAACAGATGTCCTAAATGTTCTAACCAGCAGAACAAATG | |
| | GACGTAAAGAGGGAAAGCAGTGAGATAGAATGAAGATTACCACTC | |
| | AGAGATCAACAAGAGCCCTCTCTCCAACATTTTTTCATGACCCTT | |
| | GCAAAAGGCTTTTTGAACAACTTAAAGCAAAGACACTCTTCAACC | |
| | TTACCAGTTGCTGACAGAAGCAGGAAACAGCAAAAGTCATCACAT | |
| | CCCTAGTTGCAGCATCGCATCAGCTACCTCCTTCCTCTTTTTTTC | |
| | TAAGGTCACCAAGGCACTAACTTGCTGTTCTAACAAAGGGTAAGG | |
| | AAAGAAAGAATCAAGCATTTGCTCTGCCTTTCCTGTACAAATTAT | |
| | AATTCAAGGTGACCCGAGTTTCTCTTTATAGGAAAATTCAGCTAC | |
| | TAAGTGGAAAAAACAATCATAGTTATTTTTTAAGTCACCATTTTG | |
| | CAACCCCTATTAAAATAATAGATGGAGACAATTGTTAATAGATGC | |
| | TAAAACCATGAGGTGGAAGGTTAATGGGGAACCCTATGATGAAAC | |
| | GTGCTGCTGACTGACGATACCTGAGCCCTCAGATCAGCCGTAATC | |
| | TTGCTAAATATGGCACAGCCAGGCATGAGAGGGACTCCCAACACG | |
| | ATGCAGCAGGAAGAAAGCAGCGCCACCTAGGAAGTCCTCTTCCCC | |
| | GAGTAGCTGAGCCTGAATGCAGCCAAGCCTCTAGAAGTAACCAGC | |
| | AGTGCATAGAAAATGCAGGCAATGGCTGGGCACGGTGGTTCACAC | |
| | CTGTAATCCCAGCACTTTGGGAGGCCAAGGCAGGCGGATCACATG | |
| | AAGTTCGAGACCAGCCTAGCCAATATGGTGAAACCCCGTCTCTAC | |
| | TAAAAATACAAAAATTAGCCAGTTGTGGTGGTGCACGTCTGTAAT | |
| | CCCAGCTACTTGGGAGGCTGAGGCACGAGAACCACTTGAACCTGG | |
| | GAGGCAGTGGTTGTAGTGAGCCAAGATTGTGCCACTGCACTCCAA | |
| | CCTGGGTGACAGAGCGAGACTCCATCTTAAAAAAAAAAAAAAGAA | |
| | AAGAAAAGAAAAAGAAAATAAAAAGAATACAGGCAATAAAAGACT | |
| | GAGTTAAAGGACACTACGAGACCACAAGGAAGCAACGAACTGAAC | |
| | ATAGAAAGCAGGAGCTACTGCAGAACAAACATCTCAGTTTCCTCC | |
| | AGCAACAAAAGGCAATTATGATGAACACAACCAGTAAATGCCAAA | |
| | AAGATACTTGACAATTTTCGTTGTTGTTGTTGAGATGGAGGCTTG | |
| | CTCTATTGCCCAGGCTGGAGCACAGTGACACGATCTTGGCTCACT | |
| | GCAACCTCCACTACCTGGGTTCAAGTGATTCTCCTGCCTCAGCCT | |
| | CCCAAGTAGCTGGGATTACAGGCATGCACCAACACGCATGGCTAA | |
| | TTTTTGTAATTTTAGTAGAAACAGGGTTTTGCCTTCTTGGCCAGG | |
| | CTCGTCTTGAATTCCTGACCTCAAGTGATCCGCCTGCCTCGGCCT | |
| | CCCAAAGTGCTGGAATTACAGGCATGAGCCACTGTGCCTGGCCAG | |
| | ATACTTGATAATTTTAAATATCCTTTCCTGGGAAAGACTTACAGC | |
| | AATTTTACAATTTCATCAGCATGATAAAGAAAATTTATGAGAAAC | |
| | TAACAGCAACCTTGATAGTGAGACACTAGAAAAATCTCAATTCAA | |
| | TTTTGAAATAAGACAAGGATGGCCACAATCACTGTCAATATTTAA | |
| | CATATTCTGGAGGTTTTAACTAAATGTAATGGATTTTAAAAAGGA | |
| | ATAGGAGCTGTAAACCTTGAAAATTATTTAAAATGTAGGATTTGT | |
| | AGGTGATCCACCTAGAGAACATGGTCAAATAAAACAGAAAAGAAT | |
| | TTAATAAGCTGCCTAGCAAAATAATTTTATAAGACTCAGTAGCTT | |
| | TATTGTATATCCTATCCAAAAGAACAGCAAAATTTACACGTGTAT | |
| | GTATATGTAGGTATAATTGTGTGCCTGTGTGAATATATATGAATG | |
| | TACCTCAAGAATTTGTAGGATTTAAACAAAGAAATTTGCTAATTT | |
| | ATCAAAGGAAATAAAAGAAGTCTTGAATAAATGGAGGTATGTCCT | |
| | ATGCTCCTCTATGGCAAGACCCAATTTTGAGAATGCATCAAAAAT | |
| | ATTTTTTTAGTTACCTGGAATTTTATACTCTTTTACATTATTAAG | |
| | CAGGTCAGGCTATGAATGGCTATTCATGAGGCACTTATTGACGGC | |
| | ACTCTGCAAAAAGGTCTTCACCTAGTATATTGTGGAAATGTTATA | |
| | TGTTTGCACAATCAATCATTGAAGACCTTTTACAACACACATATC | |
| | CCCATGAAAGTAAGCAATGGATGAACTGCTGTGAAAGGTGCTCCC | |
| | TGGAGCAGAAATCCACACCCTGGTTCTTACCTGGTTTTAGCATTG | |
| | CCAAGGGCCCAGCAAACTTCACCCACGACTCCCTTGGCCACTGTT | |
| | CTTTTCTTCCTCCACTGTTTTTAGTCCCCCTCTCTTGAGGGTCTC | |
| | AAGGTTTCAAGGAAATAAGTACTTACACACACACCTTAAGGACTC | |
| | CAGACCTCAAGCATCAGGCCCGGGTAAGTGC | |
| 25. | GCCTGTCTCCTCTGCCTTAAAATCTCCCTTCCCACAGACAGGACC | TRAC |
| | CCAGGAGAACCCTCCGGCTGGGTTCTGCGTAGGTACCACCCCCGC | |
| | CTCCCATCTCCGAGTGAAGCACCAACTTCTCCACCCACACTCTCA | |
| | GATGGGGGTTGGGAACACAGAACTATGAATGCGCCCCCTCTCAGC | |
| | CAGGGGGAAACGGGGCCAGGCAGCCTCCACATCCAGCATGAATGG | |
| | CAGACGCTGGGGGCCAGGACTCCTGGGGCTAGCTTCCAACTCCCT | |
| | CGGTGGGTGTCTGCGCTGCCAGCGCCCCCTGCCCCCCACCCCCAG | |
| | GCCCCGAGAACAATCAAGCCGTCCCCAGCCCAGGGCTTCCATGCA | |
| | GTAAATTCGTGTTTGCAGAGCTCTGGGAAATCTTTAGGAGGTGGC | |
| | ATGAGTATTATTAATTGGGGAAAAAGAACAGATTATGCCAAATAA | |
| | ATCCTTCCCACATAACCAAACCCTCTCCGAGAGGGGGAGGGGGAG | |
| | AGCGCAGCCCCAGGAGGGGTCTCCATCCCTCACCAGGGCCTGACA | |
| | GCCACATTACCATTTACATGGATTTATCAATAGTATTGATCCCCA | |
| | GCAGCGTAAATAGCTCAGTGGGAAGAGGCCAGGGCTGGGGCCTGC | |
| | TGCGCTCCCGCCCCCATGTCTGGGCACCGGCACCACAGACTCCCC | |
| | CGGCACCCGCTGGCGGTGGCAGCAGTGAATTTCCAAGCAGGGACA | |
| | CGGCAGGCGGCTCAGGGAAGCCAGACAGAGCCCGGGATGCCCTGG | |
| | CAGCCGCGCTGCCGACTCGCCAGTCACCGCTGGCTCCACCCCCCC | |
| | TTCATTGCTCTGCTGGGCCTGACTCAGTCTTTGCATCTGTTCAAT | |
| | GGGCGGGGGCACCTGCCCTTCTCCATCCTCTCCGGACAAGCAGAG | |
| | GGGAGTGGGAAAAGGCCCCCTCCCTCCCCCAGATCCCCCCACTGT | |
| | CCACCCCCAGCCAAAGGTAAGGTGGCCCCTTTTCACAGGGCTTCC | |
| | AGACCACGTTGGATTAAAGGCCCAATCTTAGAACCCAGGAATAAT | |
| | AATACCGACACAGTAGTCATAGTAACACATACAAGTACCAAGGGA | |
| | TTCTTATGGCCGGACGGTCAGCTCTCACAACCACTCTGTGAACTC | |
| | AGTACCATTTTGACCCTCATTTTGCAGATGGGAAACCTGACAGGC | |
| | ACAGAGAAGTTAAGCATCTGTCCAGGGTCACACTGCCAGGAAAGT | |
| | GGCAAAGGTCAGAGTCAAACCCCAGGAGCTGAGGTGCAGAGTGGG | |
| | CCATCCCACCTCACTGCTGCTTGCCATTTCTCGGCCCCTCCTCAG | |
| | CACCCCCAAAGAGGCCACCCACGAGGTACACAGCCTGCCCCGGGT | |
| | CGCCCTCCCGGCGCAGTCTGGCCCGGAGAGCCCCTTATATCAAAG | |
| | CTGCCGCAAGGACCCAGATCTGGCTGAAAAGGGGCAAAGTCAAGG | |
| | CCACCGGTAACACATCCCAACGCGCCCAGAACTCCCCCACCCCGC | |
| | CCACAGGGTCCTCCCCTTATCCCCTCCTCCCCTCTTCCCTCCCTC | |
| | ACACATCCTTCCTCCAAATCTGCAAAAAGGGAAAAATAATAATAA | |
| | TCACAATAAAACCAGTTCTCACCAACGGGGGAGAGGAGGAGGGGG | |
| | AGGAAGGGAGGAGGCTGAGCCGACAGAACAGATCCGCCCCCGCTG | |
| | CCTGCACCAGACCCTCGGAGAGCCGGAGCGCGCGACAGGCGGCGG | |
| | GAGGACACTCGCTTCCAAAAAAATAAACCTACAATGTAAAAATAA | |
| | CGCCGGGCGCCCCACGCTAATTGGGCCCGGGCGACAGCAGCTCCC | |
| | AGGTACAGCCTGTCTCTCCCTCCCGCCCGCCGTGCCCTCCCGCCC | |
| | TCGGCTTGCTCCTCCTGCAAGTGTCTGGGGCGGGGAGGGCGTACC | |
| | CCACGGGTGCCCTCACTCCCCGCAGGCCTCTGAGGAAGCTGCCCT | |
| | CTAGACAAGGGTCTGGGTCTTCCCAGTCGTGCCCAAGTGGGATCA | |
| | GCTCTGAATTCCAACTTCGCAAAGTAGTGGCATCCCAAGTATCCG | |
| | CACAGCCCGGGGGAGGGACTAGGAGGCGGCCCAATGAGGCGTCAC | |
| | CAGCCACGGGAGGCAGGCCCAGACACCAGGAACACCGGCCCGCGG | |
| | GGGAGGGGGACCAGGGGCGACTCTCCCGAGGGACCCCGCCCAGCG | |
| | CCCCGTGGCCCCACGCCTAGGAGGGACAAGGCTGGCTCTCCCCCT | |
| | CGGCTGGTGAGAGACCCTCATGCGCCCCTCCACAACAGCCAGGCC | |
| | AGGGAGCTAGGGGTACCATCTCCAAGGGCTTTCAAACCCGCGCTG | |
| | CCCGATGGAAAATAAGGCGCAGTGTCGCCCCGCCAGGTCCGGGGC | |
| | TACACCGGGTACAAGGGACGCCTGGCGGCCAAGTCCCCAGAGCCG | |
| | GCGCACACTGTGGGGGGCCGCTCTGTGCATTTCCAGCGCGCCCCC | |
| | CGCCCACCCCAACGCCACTTCCCTCCTTCCCGCCCCCTCGCTAGG | |
| | GAGCTGGAGTTCCCCTAAGTTCCCCGGCGCCCCCGAGCACGCGGG | |
| | CCGCAGTGTCGGCGGGGCGCCTTGCTCCCCAGCAGGCCACCCCCC | |
| | GAGAGCGCCAACTCGGGAGTGCAGGGGCCCCGCCAACTCCGCCGC | |
| | GTGCGCCGCCCGATCCTCCCCGGATCCCCCGGAAGCGCGCTCCGC | |
| | GCCTCCCCCCGCCCTTCGCGGGGACCCTCGGCCCCAACTCACCTC | |
| | GCGGAGGTGGCAGCGGTGGCGCCCGGGCTTGGGGCGCGCTCGGGC | |
| | CGCCGGGGCGCTCAGGCAAGTGGCGGTGCCGATCGGGCTGGGGGC | |
| | GCGCAGCAGGCCCGGCCTCGGGTCGGGACGCGATCCTCCCGCCGC | |
| | CGCCTCCTAGGGCGCCGGCCCTGGGTCCCCGGCCCGCAAGTTGGC | |
| | GCGCATCGCGGAGCGCAGGGCGCGGGCGCAGGGCTCGGGCGGGGC | |
| | GCCGGCGGACGCGGGGCTCCGGTGCGGCCCGGCCTGCCCGCGGCT | |
| | CCGCGCTCCCCGGCGGCCGCGGCGGCGGTGG | |
| 26. | GACTCTGGGGAGGAAGTCCTGTGCAGGAGAATGGTAGAGCAACAA | CD4 |
| | AATATAAACAGCCCCAATCTTGGCCCTGGAGGACGGCGTGCTGCT | |
| | CCCTCTCTTCTGCACCACCCACCTCCACACTTCCTACATGTGAAA | |
| | AATAACTTCAATCTTGTTGAAGCCTCTATTATTTTGGGTTTTCTT | |
| | TTGCTTGTAGCAGAACCCAGTGCTTACCGATAGACAATTCTCCCA | |
| | GTTGAAAGAAGGTTCTTTTGTGATAAGATCTCCCAGCACCTTGTG | |
| | CTTTTTCCTTTGTAGCGCTTGTCGAATTGTAATGAAGTTGTTCGT | |
| | TGTTTAGTGTCTATGTCATAAGCTGGAGTGGTGACCATATCTGTC | |
| | TTCTTCAACATCACATCCCCAGCTACCTAGCAGAGTGCCTTGGGC | |
| | TGGTACACGCTTAACTACGTACATGAATGAATGGAGCAGGGCTCA | |
| | AAACATCAATGCCTACCAGAGCCTGCCAGGTAATGTAAATGAGAA | |
| | AAGCAGACTGGCTGTGAACAATAGGGAGGGTGTTTGCTTTCTAGT | |
| | CTGAAGGAAGAAGGCCTGAGGAGGATACATTTTCCCCACTTTGAG | |
| | GAGCAACTCTGAGGTTGCACCTAATACTTTCACTTCTATCTATTA | |
| | ACCAAAATTGAGTCACTTGGACACATCCAGCTGTGAGGGAGGCTG | |
| | GGAAGTATATTTTAAATCTGGGTAGCTAAGAGCTCAGCTAAAAAT | |
| | TGAGGGTTCTATTACCAAGAAGGAGAAAATAGACTTTGGGGACAG | |
| | CTAAAGCGATAGCTGAGATCTGAAGAATGAATAAAAATTACCTAG | |
| | ATCAGTGGTTCTCAGAGTGAAGTGTGGCCTGCAGACCCTTGGGAG | |
| | TCCCTGAGACCCTTTCAGGGGATCTTCAAGGTCAAGCTATTTTCA | |
| | TAAAAAAATACAGGATGTTATTTTCCTTTGTCACTATGTTGACAT | |
| | TGCAATAATGTTGCAAAAACAATCTGGATAAAACTGCTGCCTTAG | |
| | CATAAATGAAGGCAGTGGTGCCAAACCATATGATAGTCATTGCAT | |
| | TCTTCACTGCCAGGTACTTACAGTAAAAAAAGAAAAAAAATTAAT | |
| | GCCACTTTTACTTAAGAATGCCCTTAATGAGGCCGGGCGCAGTGG | |
| | CTCGTGCCTGTAATCCCAGCACTTTGGGAGGCCGAAGCGGGCAGA | |
| | TCAGGATCACCTGAGGTCAGGAGTTCAAGACCAGCCTGGCCAACG | |
| | AGTGAAACCCTGTCTCTACAAAAATACAAAAATTAGCCAGGCATG | |
| | TTGGCGGGTGCCTGTAATCCCAGCTACTCGGGAGGCTGAGGCAGG | |
| | AGAATGGCTTGAATCCAGGAGGCGGAGGTTGTAGTAAGCCGAAAT | |
| | CATGCTGCTGCACTTCAGCCTGGATGACAAAGCAAGACTCTGTCT | |
| | CAAAAAAAAAAAAAAAAAAAAGAGAGATCAAGACCATCCTGGGCA | |
| | ACATGGTGAAACCCCATCTCTACTAAAGATACAAAAATTAGCTGG | |
| | GCATTGTGGCGCATGCCTGTAGCCCCAGCTACTTGGGAGGCTGAG | |
| | GCAGGAGAATTGCTTGAACCCGGGAGGCAGAGGTTGCAGTGAGCC | |
| | AAGATCACGCCACTGCACTCTAGCCTGGGCAACAGAGTGAGACTC | |
| | CCTCTCAAAAAAAAAAAAAAAAAAAAATATATATATATATATATA | |
| | TAGAATGCCTTTAATGAAGCAGTAAATACTAATTTTATTAAATCT | |
| | CAACCCTTGAGTACGGTGTGTCATGAAATGAGAAGTAGCACACAG | |
| | TACTATATGCTACAGATGAAGTACAATGCTGTCAAATAGGGGTAC | |
| | TTGTGTTAATTGTTGGAGTCGCAAGCTGAACTAGAGTTTTCTTTT | |
| | CTTTTCTTTTCTTTTCTTCTTTTCAAGACAGGTTCTCACTCTGTC | |
| | ACTCAGGCTAGAGTGCAGTGGTGCAATCACGGTTCACTGCAGCCT | |
| | CAACTTCCTGGGCTCAAGCGATCCCCCCACCTCGGCCTCCTAAAA | |
| | TGCTGGGATTATAGGCATGAGCCACCACTCCCAGCCCCACTTTTT | |
| | TCAGACTGGAAAACGCACACTCACATGTGCATCTTTAAATGATCA | |
| | CTTGGGCTGTGGTATGGAGAATGGCGACCAGTGAGGAGGCAGGAG | |
| | CTGTTGTCCGAGCAAGGGATGATATTGGCATCTTGGATTGGCATG | |
| | GTGGCAGTAGTGGTAGTGCAGAGTGACTTGGGTAGATTTTGGAGC | |
| | CATTTAGAAGGTAACATCCACAGGAACTGGTAAATAAATACGTGG | |
| | GAGAAGTTGGGTGAAGGGGGTGTCAAAGATTACACCCAATTTATT | |
| | TTGCTTGGGCAAGTTGGTGGATGGTGAGCCCCTCACTGAGTGAGA | |
| | AGCCTGGAGAAGCAGGTTTGGAGGGTGGTAGTATGCAGGTGGTAT | |
| | GCATAGTTGGGGATGTGTGTTGAGTTTGCTATGTCCGGTGAGCTT | |
| | CCCAGTGGAGATGTCCAATGGGCAGACGGATACTCACATAGAGAG | |
| | TTCATGGTAGATTCGGGCTAGAGGAAAGCACCTGAGGCCTGGCCA | |
| | GAGACGCCTAGAGGAACAGAGCCTGGTTAACAGTCACTCCTGGTG | |
| | TCTCAGATATTCTCTGCTCAGCCCACGCCCTCTCTTCCACACTGG | |
| | GCCACCTATAAAGCCTCCACAGATACCCCTGGGGCACCCACTGGA | |
| | CACATGCCCTCAGGGCCCCAGAGCAAGGAGCTGTTTGTGGGCTTA | |
| | CCACTGCTGTTCCCATATGCCCCCAACTGCCTCCCACTTCTTTCC | |
| | CCACAGCCTGGTCAGACATGGCGCTACCACTAATGGAATCTTTCT | |
| | TGCCATCTTTTTCTTGCCGCTTAACAGTGGCAGTGACAGTTTGAC | |
| | TCCTGATTTAAGCCTGATTCTGCTTAACTTTTTCCCTTGACTTTG | |
| | GCATTTTCACTTTGACATGTTCCCTGAGAGCCTGGGGGGTGGGGA | |
| | ACCCAGCTCCAGCTGGTGACGTTTGGGGCCGGCCCAGGCCTAGGG | |
| | TGTGGAGGAGCCTTGCCATCGGGCTTCCTGT | |
| 27. | TAAAGACGGGGTTTGGCTCTTGTTGCCCAGGCTGGAGTGCAATGG | ThPOK |
| | CGCGATCTCGGCTCACCGCAACCTCCGCCTCCCAGGTTCAGGCAA | |
| | TTCTGCTTCAGCCTCCTGAGTAGCTGGGACTACAGGCGTGTGCCA | |
| | CCACGACTGGCTAATTTTTGTATTTTTACTAGAGACAGGGTTTCA | |
| | CCATGTTGGTTAGGCTGGTCTCGAACTCCTGACCTCAGGTGTTCC | |
| | ACCCGCCTCTGCCTCCCAAAGTGTTGAGATTACAGGCGAGAGCCA | |
| | CCGCGCCCGGCATGGGCATAGGCTCTTATTTGCATCATATTTACA | |
| | TCTGTCTTGCATGTGAGTTATTTCCCTCCACCCAACTTTCTCAGT | |
| | TACTCTCAGACTCTAGAGTGTTCGCCAGCCTTGGAGCGTACCCGT | |
| | TCCTCCAGCCATTCTTCGGCCTCAGTTCCTTTCCATCATTCGCCC | |
| | TCAGGCCTTCGCCTACCCCAGCTTCAAAGAAGCGACCGTAACCTC | |
| | CTAGCTGGCTGGGTTCGAGAAGCCGGGGACCTGCGCCTCCTGGTG | |
| | GCTGATCGGAAGGAGGCTGAGTGCAGAGGGGTTGTCTTGCCTTCT | |
| | CTGGGACGTGGAGTTTTGGAAACTGTTCCCCTGAGCTTCCTGAAG | |
| | CTAAATTTGCCTCCCCCGGCCTTGGGGGGCGCAGAGATCCCGGCG | |
| | GCGATTAGCGCTGCGCGGCAGCCGGCTCCAACCCAGAGGCCCGGA | |
| | ATAGGCGCGGAGTTATAAATAGTGCCACCCGCAGGTGTTGGGGGG | |
| | AGTCGGCGGGAGGGGGGTACCCCTGGCGGCCACGGCCCCTTCAGG | |
| | TGGGTTGGGCGGTCGCGGTGGGAGCGCTGGGGGGCGGGGGGCGTG | |
| | GGGGGTCTGCGGTCTGAGCGCCCCCAGCGGTTTCCTGGGCGGCGG | |
| | GTTTTTCGAGGGAAGCGGAGGCAGCGGAGGATGGGGGGGAGCAGC | |
| | GGAGAAGAGGTGGGCGCCTGGCGCATGCTGACGCTCCGCTTGCAG | |
| | CTCAGCCCCCCACATTCCGAAACAGGCCCCCAGACCCACTCACGC | |
| | AGGACCCCGCCCAGGCAAAGCCTCGCCTTTCTTAACACCTGAAGG | |
| | CCTCATTCCCCCAGGTTCTGCAAGCATCTAACTTCTATGCTTCTA | |
| | CCCTTCGGCGCACGTGGCCCGGCCAGTCCGCGCACCTGGCCGTCA | |
| | TTCCGAGTCGCTGTCCCCAGCCCCAGGTATTCAGTCCTCCCACTC | |
| | GGGGCTTTCATTCCTCTCTTAGGGTAAACACTTCCTTCTCTGGGT | |
| | ATTTGTCCAACTCGTGCTACTCTCCCCAAATTTAAGGTCTAGCTG | |
| | GTCACCTAAAAATTTAAGTCTTAACATGCGGATCTGGGACGCTCT | |
| | CCTATCCCCAGGCGGCCCCTGCAGGCCCCTCACTTGCCCTCCAGC | |
| | TCCCAGTTCCAGTCTGGGCCTAGTGGCGGGGCGGGGGCGGCCGGC | |
| | CCCTGGGGCCCAGCAGGGGTTTCATGGGAGGGGGCGGGGTCCCCA | |
| | GTCAGCGTTTCTCTGCCGCCGCTGTTTCAGTCGTTTGGCGTGGTT | |
| | AGGGGTTGGGGTAAGAGGGAATTTCAAGTAGGGGTTCCCCGCTCC | |
| | GATTTCTGAGTAAGGAGGGGAGCTATTTGCACCAGGAGAAAAAGA | |
| | GAGACAAGGGAGGGTGGGCGTCACTGAGGAACTAACTGCGGGTTC | |
| | CAGGACCATTGGGAATGAGGTGAGGAGTGGCAATCTAGCCGCAGA | |
| | AAGATCAGTCATGTTCAGCCAGACGCAGAACGCGACCCTCAAACA | |
| | TGGGGGAAGTTGGGCGAGGCGAGCCTGAGGCACTGGGCACCTTTC | |
| | GAGTTTCTGTACCCAAAGGCTGGTGCCAGGGAGCCCGCCCTTCTA | |
| | TTGCCTCTGGAAGCCCTGGCTCTGCCCTCCCCGGGCCACGTTCCC | |
| | ACCCCCCCCGCCCCCCGCCCCCTGCCCCCCGCCCCCTTTCCTTGG | |
| | CTATCCTCCTTCCTCCCCAAAGCAGCCCCCTCTCCATTTCACTTG | |
| | TGGCACTGCCGCCTAGCCCCTAGCACAGCCCTTCACCAGGCCTGC | |
| | ACGACCCAGACGGCTGGAGTAGGAAGGGGAAAACCCCGGCCTGGA | |
| | CGGCTCCGGGCCCCTCCGGACCAGGATCTGTCCGGCGGGGCTGAG | |
| | AAGAAAACCCGGAGCAGTGATCAGCCTCGCGACCCAAGGGGGTCA | |
| | GGGACGATAGGAAAACCCGGAGCGGATTACTCGAGCCAGAGCGCC | |
| | CCATGCCTGCCGCTACCTCCTCCTTCCTATAGCCCCCCTAATTCT | |
| | CCAGTGAGATAATGTCTGGGGTGGGGGGCTGTTTGGTTTGGCCTG | |
| | AGCCCTCAGGGGCGCCTCACACTTTCCGCCCCTGGCTGGGGCGCA | |
| | GGGCAGCGGCCCCGAGGCTGAGTCACGGCCCGGCGCGGAGAGGGA | |
| | GAGAGGGAGAAAGAGGGAGGGAGGGACCTGGACTCCGCCCCTCGT | |
| | GCGCGGGGATCCCGGGTCAGCCCGGGTGAGGGCCAGGGCCCCCCT | |
| | GGCGCCGCCCATTGTGGTCCAACAGGTTGAGCTGGTGTCCCGAGA | |
| | AGCCACCTGCTGGAAAGGGAGGGAGGCCCCCTGCGGAGGGGGGTT | |
| | GGGAGGGGAAAGCGAGTCCGACCCGGTTCGGCCTGGCTTGTCGCA | |
| | GGGGAGCCAAACCTGGCCCTAGGTAGCCGGATTTGATTCCATTTG | |
| | ACACTAGTGGGAGGGGCCCTTTGGGGTACGCAGCTCCGCAGGCTC | |
| | AGTCAGAAACATCCTTCACCCGCTACCTTTCTCAGTTGGGGGACT | |
| | CCTAAGTATTTATCCCGGTCCTCCCCAAAACCTACACATACCACA | |
| | CATCCCAAAACATCCAGGCACCCCTCGCCACCTGGGTCTCTCAGC | |
| | GAATTCATGTCAAGCTATCCCTCCTCGGTGGAGACTCAGTTTCTC | |
| | CACTTAGGGAACCCGCGGGATGGAGGTGGGGAGCAAAAGCGTCGG | |
| | CGCGCCCCCTCCCCCTTCGCAGACTGGGCCCCTCCCCTCCGACAA | |
| | TGCCAAGGCCGCAGCGGGCACTGGCGACAGC | |
| 28. | GCTTTTGATACATTTTTCTCCGAAGTCATGGTTCCAGTCCACACT | CD8 |
| | CACACCAGCTGTGGAGGAGTTTTCATTTACCCACGAGCTTTGTTA | |
| | TTTCTGGTTTTGTCAGTCTTTTCAATTCTGCCAATCTCATAGGGT | |
| | GTGATGGTATCTCGTTGTTTAAACCTGACTTCTCTTGTTAAGGTT | |
| | GAGACCGAGCATCTGGCCCCATGTTTGCTGGCTATTCAGCTTTCC | |
| | TCTTCTGCAGACTGCCCGTTTGTCCACTGCTCTGTTTCAGTAACT | |
| | TCCATATACCAAGAACTCTCTGCCTTCCATAACTCACATCACTTA | |
| | AAATACGAATTTTTTTGATGCTTAGAGAACCTGATTTCCTTCCAA | |
| | CCAATTGTGCTCTCCTAATTCCAACAACCAAATGAAGCTTCAACT | |
| | CTTCTTTTTGTTCAGAGCCCTCCTGAGTCCTGTCCTACTCACAGT | |
| | AAAGGCTGTGGCAAAATAAATAAATAAGTAAATAAAGGGGAAAAA | |
| | TACACTGCATCCAAAAAAGGCTCGTTTCTTTTTCTTTCTTTCTCT | |
| | CTCTCTCTTTCTTTTTCTTTTTCTCTTGTTGTGACAATTGTCACA | |
| | ACAAAATGTGTATGTGACAATTTGTTATCTTAAAACAGTTTACCC | |
| | AAAATAGAAACCTTACAAAGCCAGTGGAAACTTTTCTTTTTGCAT | |
| | AAGTTGGTTAATTCTTTTGAGTGCTGGCCTGGGATCCATGAAATA | |
| | ACCTCAACTCGACCATAAAACTTTTCACTCCCACGTTATGCAAAC | |
| | GGTGATGACCTTGTTGGTGGCAAGCCCCCCGTGTGACTTTCCGAG | |
| | GGGATCTGAGTGGTGCAAGGTGGAGGACGCAGTGATGGAAACATT | |
| | GCGAGAGGGAAGGCGTCTGTTTCCACCCACTTACCCTCAGAGCAT | |
| | GAGGGCTGGGCGAAGGCTCTGACTCCTGTAGGGGGTGACCCATTT | |
| | CTAGGACTATAGAAGGAGAGGTGTGGACCTGGAAAAAGGAAGGAA | |
| | AGACCACGGCAAGGAAAGTCAAGAAGTGGGAGAAGTCAAGGGCTC | |
| | TCTCCTCCCTTCCTCCTTGTCCTTGGCCTGCAGCAACTCCTCTTT | |
| | CTCTTTTTGTGGGGACAGACTGAAGAGGATTTCTTAGTATGGTTT | |
| | GTACCTTCCCAGTAGAGCAGGAAAAAGAGGAACGAGCTGCTCCCT | |
| | CCACACCTCAGACGCTGAGGTCAACTGCCCTTTGGGCGGGGCACG | |
| | TTGGCTCACGCATCTAATCCCAGCACTTTGGGAGGCCGTGAGGGG | |
| | AGCATCACTTGAGCCCAGGAGTTGGAGACCGGCCTGGGCCACATA | |
| | GTGAGGCCCCATCCTCCGGCTTGCACCACTCAAATTCCCTCGTAA | |
| | AGTCACACGGGGAGCTTGCCCCCAGCAAGGTCGTCAGTTTGCAAA | |
| | ACATGGGAGTGAAAAGCTTTATGGTGGAGTGTAGGTTATTTCATG | |
| | GATCCCAGGCCAGCACCCAAAAGAATTAACCATCTTGTGCAAAAA | |
| | GAAAATTTTCCACTACTTTTTTAAAGTTTCTATTTTGGGTAAATC | |
| | GTTTCAAGATAACAAATTGGAGAGAGACCTAGAGAGAAATGAGCC | |
| | TTTTTAAGATGCAGTTTATTTTGCCCCATGATGTTTTTGTTTTTG | |
| | TTTTTGCCAGAGCCTTTACTGTGAGTAGGACAGGACTCAGGAGGC | |
| | CTCTGAACAAAAAGAAGCGTTGAAGCTTCATTTGGTTGTTGGAAT | |
| | TGGGAGAGCACAATTGGTTGGAAGGAAATCAGGTTCTCTCAAAAA | |
| | ATAAAATAAAATAAAATAAAAAAAAAAGCCAGCGTGCTGGCGCGG | |
| | GTCCATGGTCCAAGCTACTTGGGAGGCTGAGGTGGGGGGATCGCT | |
| | TGAGCCCGGAAGTCGAGGCTGTAGTGAGCCGTGATTGCACCACTG | |
| | CACTCCAGCCTGGGCGATAGAACAAGACCCTGTCTCTTAAAACAA | |
| | ACAAGAAACAAAACAAAACAAACAACAAAAAAATTCATTTGGGAA | |
| | TGTTTCTGCGCGTGCCCATAAGCAGAGCCATGCTATAGGATCTCC | |
| | CCTGTGCCCCAACAATCAGCTTCTTACTTGGAGGGTAGAGAACGG | |
| | TGCTTCCCCACGCTGCTGTGCAATGGATAGGAGCTATGTGAAGCT | |
| | GGCGTATGGGTGGGGTTTGCAGGCTTCGTCCGGCTTCATCGCCGG | |
| | CTGCTGACCCGGCACCAATTCCTGTTCTGCAGGTCTCACCGCAGA | |
| | GGGGCACGCCAGCCATGAGGACAGATGAGGGAACACGTGATGCCA | |
| | CGATGGGGGTGCCAGGATGAGGTGGGTGCGGTCGCGGACAGGCGC | |
| | ACGAGGAGCCCAGCGGAGCGCCACCCGGAGCAGGCGCGGAGGAGG | |
| | GCTGGGGAGGGCCACCAAGGCGACGAGAGCCGGTGTGCCTGAATC | |
| | AGCCTAAAGGAGACGGAGGAGGAGTGTGGTGGGCGCAGGGGCAGG | |
| | GAGCTGGGGGAAGGGCGGGGGGCTAGCCCAGGCTGAAGGCAGGCA | |
| | GGAGCAGGGCCGCGATGTCAGACAAGAAACGGCAGCAGTGTGTTT | |
| | GGGAACCTAGCACCAACCGCACCGGTGGAGACAGGGTCTGCTGTA | |
| | GAGGAGGTGGGGCCGGGTCCCAGCTAAGTAAGGCGGTGGATCTTG | |
| | CAGCCCTTCCATCCTCAGCCGCTCATTCTGCGCAAATCTCGGGGC | |
| | CAGCCTTGGTGGAGCCGTAAAGCGTCCACCAGAACCTGGATCCCT | |
| | CCGCCACCTTTCCCATGAATTCACCTTTCTGTACACAGCAAGCGC | |
| | CTGAGCGGAGACGGCCGACACGTTTCCCACTGTTACCCCAGGAAA | |
| | CCGCGGCTCCTGAGGGGGTCAGGGCCTGGGCAGGGGGCAGAGCTC | |
| | AGCGCGCCGTACTGAGGCAGAAACGGGGTCCAGAGAGGGTGGGGT | |
| | GGGGGTACAGGGAAGGGTCGCCCGAAGGTCCCTGGCGCAGGGAGG | |
| | ACAGAGGAGGGATCTAGAATTCGTAGGGGAGAAGAGAACTCAGAA | |
| | AAGATCCGGCCCAGCGCATTTATTTTACAGA | |
| 29. | TGAGAAGGCAGAAGGGGGAATGGTAGCCCAGGTTCCCCTTCCCCC | FOXP3 |
| | TTCTGGGTGCTGAGGGGTAAACTGAGGCCTGCAGTTGGGGAGAGA | |
| | GCCAGAACCAGGGTCCCACCTAGAGTCCTGAGATCTAGGCTTGGA | |
| | TTTCAACTCTGCCGCTGCATTTCGGTGAGGCCCTGAGATCTCTGG | |
| | TCTTCAATTTGCCCTTCTACACTGAGCACGGAGAGGCGTGGAGTA | |
| | GACAAGGGCCAGGGCCCTTCTACGCTGTCTGGTTAAGTCATTAGG | |
| | TGTCTGCAGGGCTTCAAGTTGACAATTGCCCCTCTATCCAGGGGA | |
| | CTGGCTGAGAGATAGGGATACATAGAGACAAAGAGACACACACAA | |
| | AGAGCGAGCAAGAGAGAACAAGAGATAGTGAGAGACATTGAGAGA | |
| | AATGGACACATGCATGGAGAGCCAGAGTGCATGTGTGCGAGAGGA | |
| | GGATTGCCTCAAATAAGAACATTTGCTGGTCTCTGGCTGGTTCAA | |
| | CTGATGCTGCCTGAAATAATCAAGAATAAAGAAGGGCAAGGTGCC | |
| | AGGGACACCCATGGCTGGGTATTGAATTGTATTGCAAAGCAACAA | |
| | TCAGCAAAACAGTGTGGCCCTGGTATAAGAACAGATACTGGGGAA | |
| | GAGAACAGAATAGAAAGCTTGGACATGGACCCACATATGGAGAGA | |
| | ACTGAATTTGTGATGAATGTGGCATTTCAAACTGGAGGACCATGG | |
| | AGTATGGTTTAACAAATGTGTCTGAGATAATTAGGGAGAAGATAA | |
| | AGTTATTGAGTGAATAGTCAGTCCATTATCCCAACAACCCCTCCC | |
| | TGCCCAGTTTGAAATGTCACCATCATCATATACCCTAAAATGCCC | |
| | AGATCCATTCAAGATATAAGTTTTAACACCTAATGCTGATCTTGG | |
| | GTTTATTGTGTGTCAGGCCTTGTGCTAAGTATTTACTGTGGTTAA | |
| | AAATTTTAATCTAAACAAAGACTCCTGAGGAAGGTACTATTATAA | |
| | CCATTGCAGTACATATGAGGAAATGGAGGTATGGAGAGGTTAAGT | |
| | GCCTGGCTAAAAATCACACATAGGGCTTGGGGTGACGCTGGGTTT | |
| | GTCCCAGACAGTCTGGCTCCAGTACCCACACTCTTAACCTCTATA | |
| | GTAAATGGAAAAAATGAAGCCATAAAAGAGACTAGAAGCCAACAT | |
| | AGGTGAACATTTATCTCATTTTCAAGTAGGAAAGGACTTTCTAAG | |
| | CACAAAACCAGAGACAGAAGCCACAGAAAAAAAGACTAACCTATT | |
| | TGACTGTATAAAACCATCATAAGCATCACAAAAAACACCATAAAC | |
| | AAATAGAAAAAAAGCAAATGATGAATTGGGGAAAATATTTGCTAT | |
| | ATATGTAATGGCTGATGAAAGGTTAATAACCATTCCAAATAAAGA | |
| | GCTGTGGCAAATCAATAAGGGAAAAATAAGATGAACACCCTATTA | |
| | GGAGTAAGGACATGACCAGACAACCAAAAAAAAAAAAAAAAAAGC | |
| | ATGAATGGCCAATGAATAGTAAAAGTAATCACAAGATGCAAATTT | |
| | CAACAATGTGATAATGTGTTTTTCTTACCTGTCATGTTGGGAAAT | |
| | AATTAAAACATAATAATACTCACCTAGGGTTAGCTTAAGTAGAGG | |
| | GAGCATAAAATAGGACAACCTTTTGGAAGGAGAATTAGCAGAGAG | |
| | GGTCATAAACTTTAAAAATGCACGCCCCCTTTGCCCCAGCAACTC | |
| | CCTTTTCAGGAATCCAAGGAAGCAGTCAGGGATGTTTATAGAAAA | |
| | AAACGAGAAACAACCGGAATGTCCAACAATCGGCACTTGGTCAAA | |
| | TCAATCAAAGTTCATGCTGATGTGAGGACAGTCTTGTCCATCATG | |
| | AAAGATCATGTGTTCAAACAATTTTTAGTAAGCTTCAAAAACACT | |
| | ACTGTTAATTGAATAAAGCAGGAACAAAACCAATATATAGCATGA | |
| | TTCTAATTTGGTTACAGAAATACTAATAGCTAACACTTCGTGAGC | |
| | ACTTACTTTGTGCCAAACGCTGTGCTAAGCCTGCAGAATCGAGCT | |
| | CACCCCAGCCCTGAACAACCTGTTTGCTTCCTGAATATGGACTCT | |
| | GGTCACACACATGCAGTCCTGGGGTAGGTCCACACAGCTAAACTA | |
| | CGGTTGACAATGGTGTGAAGTGCTCCCTGCCCCCCCGCCCCAAGG | |
| | GTCTCCTCTAAAGCGATACAAGCAAAGTTCAGTTAAGTGCTCAGC | |
| | TTGCCCCGGCACCTTGCAATCCTCCTGCTACTAGGGTGAACAGAA | |
| | CTGATGCTCACTCTCATAAAATGTAAAGGTCCTCGGCGACATTAC | |
| | TATTATTAAACGCCAGCTGTGTACAAAGCTCTAGGCTGGATGCTG | |
| | GCTGGGAAGGCAGGTGGGGGAAGGCAAGAAAAGAGAGCGGGAGAG | |
| | ATGGAGGAAAGGAGATCGATGGAGTGTGGTCAAGATGGAGGAGAC | |
| | AGAGATAGGGGAGATGGTCAGAGGCCAGGAGAGATGCGGGGAAAG | |
| | AGAGTCTGAGTGTAGCGACAGACAGATGGCGGGAGAAAGAGAGGC | |
| | AGAGAAACATGTAAAAGAGCAAGACAGGGTGAGCAGAGAGACAGA | |
| | GAAGGATGAGAGGCATCAAGAGCTAAGAGACAAAGAGATGAGAGA | |
| | GATGCAGTTGAAATTTTCAGTTGCACCTGGACAGCATTTCAAGTT | |
| | GTTCAAAGCTCTGAAATCCATAAAGACTGGCAGCTGACATATTTT | |
| | AAAAATCCTATCCATCTACGTATCAATTGATGAATTCATTTATTT | |
| | TTGCCCCTGCCCATGCATTAAGTACTTCACCTTTAAGTCTTCTGC | |
| | CATTTATTCTATTATTTTTTTAAAGACCTTACCTGGCTGGAATCA | |
| | CGGTAGCTGGGTACATCCCACTGTACCAGAGGGCCCCTGACCCCC | |
| | CCGCCGTGCCTACCTCCCTGCCATCTCCTCCAATGGGGCCCACAT | |
| | CTGGTAGGGGAGAGCAGGGACACTCACCTTGGTGAAGTGGACTGA | |
| | CAGAAAAGGATCAGCCTGGCTTGTGGGAAAC | |
| 30. | CTTTGATTCACATAATGAAAAGATCGATAATCTAACTGTATTCTG | Helios |
| | TGGGGAAAAAAGTTTAAGAATTGACAAGTCCTGAGAAGATTTTTA | |
| | CTTTTTTTTCCCCCTTTGGCCTTGGTCCTTTTTGCCTTTAGTTTC | |
| | AAAACTCTCACTGCACCAGCAGCTAAATTATTCACAATGAGCCAT | |
| | TTCTGTATTGATCGCCAAGTATATTTAGCCCTGGCTCCTGGAATT | |
| | TCTCCATTACTTACTGGAAAACAGGCAGCTTCACTTCTTGTCTCC | |
| | AAAACCACTTCCCTTCTCCCTCCCCTCCCCTTCTTCACCACCACA | |
| | CTCCCTTACCCCCCCCCCAAAAAAAAACTTTTATTTCTTTCTTTA | |
| | TGGAATAATCAGATCAAATTCCCAACTCAGTCACTACATAGCACT | |
| | TAAATTTCAACCTGCTGTACTGTTATCACATTCTTTTAAATTATG | |
| | ATTACATAAGGCTTTAAAGAAAGAGATCCGTAAAGTTTAAAAGAG | |
| | GAGCAGTTTCTTCGGATATTTTACAAATGAGCTTATCTCTTTAAA | |
| | AATGTACACATTTAACACACACATATTAAAAAGAAAGAAACGTCA | |
| | GGGAGAAGCGAAAGAAGGCTGCCCCATCAATGAAATGGTTATTAA | |
| | CCCTCAGAGAAGGAAAGAAGGAAAAAGAAAAAGGAGAAAGAGAAA | |
| | CGAAATGTACATACAAAAGAAATTGTCCTTTGATTAAAAAAGATT | |
| | CATCACCATTTCCAGCTCTGTCGGGAGATCTCAGCTTCTTCTAAC | |
| | CCCTCAAAGAGGAGGTGACAATGTCGGGCTGAAGATAAACGGAGG | |
| | GAGAAAGAAAGAAGTTTTTTGTGTTTCCCCCTTCTCTCTTTCCCT | |
| | CCCTTGCCCCCTCCAAGCCAAGCCCCCTGCAGAGTTCAAGGGGAG | |
| | GAGGAAGGAAAAGCACTTTACAGGTGGGTCATTCCAAGCCCAAAT | |
| | CCAGCAAACAGATCGGCTGATAAACAAAACCAAGAAATGAGAGAG | |
| | AAGAACACCCCCCTCCTCCTCCTCCTCCTCCTCGTCCTCCTCCTC | |
| | CTCCTCCTTCCACCCCTCCCCCTCGTCCCTTAGGGATGGTCTAGT | |
| | AGGAAAAGTCACTCAGGAATGGCACCACGTACTTTCAGGAAAGAG | |
| | GAAAAAAAAGAGAGAGAGAGAGGTCAGTCAGTGCTATAGCAATGC | |
| | GATTAACAAGAGAAAAAACTTGATCCACCGGTTCCTTAAGAATCG | |
| | ACCAAAAGCTAAGACAAGAAAACTGAAAGAAAAGGGGGGGGGGGC | |
| | GGGTAGAGGGGAGGGACAGTCAGGCAGAACCCAGCTAGGAACCCA | |
| | GGCCAACTTCACAGGACTGTTTTTGGCCCTTGGCAAAAAAGAAAA | |
| | AAAATAAATAAATCTTAATTCCATCTCTTTCGCCCATACTAGAAC | |
| | CTGTCAAAGTGATTTAACTGCTGCCTGTTTACATGTGTCATACCA | |
| | GGGTTTTTGGTTTTTTTTTTTTTTTAATTCCAACAGGATCTGGAT | |
| | ATTACCTTCTATCTGGACAGCTTCAATTTATTCCAGCCTACCCAA | |
| | TCTTTCTTCTTGGAATTCAAGTTAAAACAAAGCAAAACAAGTCCA | |
| | AATTCATGACAGCTATAGAGATGAGAACTGATTAATCGATTAACA | |
| | TCCCAGAAACAGATTACAAGGAGGGGACGATAAATTAAGGCAGAG | |
| | GACATCATCTTCAACCCGATTCCCTGAGCGTCCTTTACAAAAATC | |
| | ATTACCCTAATCAGAACCACAATCCATCCCTCCCAGAGAAAAATA | |
| | CCTATATTATTATTATTATTACTGGTTAACAGCAACAAGTCATTT | |
| | GATCACATCACAGTGCAAAACGAGATGCTATATAATTACACTTAA | |
| | CTTTGAAAACACTCCCCCCTTTGCAAATCATACACATATTTATAT | |
| | ATAATCTATGAATCAGAAGACAAAACAAGAGCTGTTCTTCACCAC | |
| | GCAAAAGCCAAGCGGAGATTTACCTCAGCAGTGCATGCAGTAAAA | |
| | TAATCCCATCACCCTTTTGTTTGTGTTTACTGTTAATGTGTCCTC | |
| | TGTCTTTCTTTCTTTCCTGTGCCTAACGTGTGTTTGTGCACTGCA | |
| | GTTGGTGGTGGAAACTAAGGCAGTGGATCTGTAGCTAAGGGTAAT | |
| | CCTGTTTTTACTTCCTCCATCTTCAGCGAGGAGCAGGGTTAGCCC | |
| | GGGACAGCTGGTCAAACCCCGAGAAACCGATCCGGTGGAGCCCGA | |
| | GCACATCTCCCCCGCCGGCCGGGCTCGCGCAGACGCCCGCGGGCG | |
| | GAGGGCGGGCTGGCGGCGGCTGCGGCGGCGGCGGCGGCGGGCGGC | |
| | TGGCGGCGGGCAGCGGAGCCCCGGGCGCGGGCGAGCGGACGTGCG | |
| | TGCGCGCGCGTGCGTGTGTGCGGGGCCCGAGCGCCTGTGCGTGTG | |
| | TGTATGAGAGCGCGTGTGCGCGCGCGCGCGGGCTGGCGGGCGCGT | |
| | GTGCGCGTGTCTGCGCGCTAAGCCGCTCGGCGCGCTCGGCAATCG | |
| | ATTACAGGACAAGTGCTGCCCCGGAGGCTCCGCGACGCGCGCACT | |
| | CCCTCGCGCCCACCCGCGCGCCCGGCCGCGTCGCCCCCAGCCCGT | |
| | TGGTCCCCGGACTGCGGACCCCGCCCCCGACGGGGGCCAGGTAAC | |
| | CCGCTTCCGAGTGTGCCACGGCGACCCCCATCTCCCCTCCCCGTG | |
| | TCATAATAAATCTCACGTTTTCTGCCGGCTGGAGGCTTGGAGCGG | |
| | TGAGGTGACGTTCATTTCGGCCGCGTGAAGTTTTCTTTCAAACTG | |
| | GGATTGGTGGGGAGGCCGGGGGAAGGGAGCGGGGCGGCGATCTTG | |
| | GCGCCCTGCGCGGAGTCCGGGAGCCGCGTCCCGGCCGAGTCGGGG | |
| | AGTGTGCGTGGAGCCTCCCACCTACTGGTCTCCGCCGGCAACTCT | |
| | GCGGCCCCATTTCGGAAAGCGCACCGCATTGACCGGGACCTGAAG | |
| | CGGGATAAGAGGGGTCCTGGGGAATAGACTGAGAAAGACATTCAT | |
| | GTCTGAGATGAAGCTCAACTATTACTATTTT | |
| 31. | GGTTCTTGCTTCATCTAGGGGTTCATGATATCCATACTGCAAAGT | CD25 |
| | GGCTATGAAGATTACAATAGGACACATATCTGGCACATAGAAGTT | |
| | ACTTTATAAATGGTAGTTTTAAATTTTCTTGTAGTTCTTTATTGC | |
| | CTACTAGGTGAAGTTAAGAAGTTATCACTAGGAGAATTCTCTTTC | |
| | TGACCACATCCCAGGGCCCAGCAATATACAGACTAAAAATGCCTA | |
| | ATGCCTTAGATGATTCATTTTACCTCATCCTAACTTTCCAGTTTG | |
| | AATTCCCCATATCCCCACCCTCCCTGTCCAGGGCAGGAGCACATT | |
| | GGACCACCTGCTGCTCCCTAAGGCACTCCTGTGTTTTGGCTCATT | |
| | GGGGTCTCTCTCCCTGGAATCTCATTGATGGAAATTTACTGGTCC | |
| | CTTGGGGCTCCCTTTACATGTCATCCTCATGAAGCCTTCTCTGAT | |
| | AGCCCAACGCTCCGTAGATCTTACCACACTCTGACTGGTATTACT | |
| | ATATTTATATCTATGTTTTTCTCCTCCTCACCAGGTTATAAGTTG | |
| | CTTAAGTGTCAGGACTATATCTCCCTCATTTTTGTATTTCCTATA | |
| | GTGTTGAGGGGCAGATGGGGTATGGATAGATGTTGAGAATGGCGT | |
| | GAAGGAGTCCTTATGGGACTCTAGTTCCCCTGCTCCCTCCAAGAC | |
| | CACTCAGACCCTTGGATAAGTCACCAATAGCTAGAAATAACACGA | |
| | GTTTACTTGAAATGTGTCTTCCAGAGGCAGAGCATGATTAATTGT | |
| | CAAGATTTTCCTGTTCTTGGCTTAGACTGAGTTAGCAAAGACCCT | |
| | GTGGATGTAGAAATCACATTGAAAAACCTTCCTAAAAGTATGCCC | |
| | TCTTTATAAGAACGGCACGATGGACTCTGAGGACTCAGGGGAAAG | |
| | GGTGCGAGGTGGGTGAGGGAAAAGAGACTACGCACTGGGTACAGC | |
| | ATACATGCTCAGGTGATGGGTGCACCAAAATCTTACAACGCATTG | |
| | GGTACAGCATACATGCTCAGGTGATGGGTGCACCAAAATCTTACA | |
| | AATCACCACTAAAGAACTTATTCATGTAACCAAACACCACCTGTC | |
| | CTCCAAAACCTATTAAAATAAAACACATAGGTCAGGCGCGATGGC | |
| | TCACACCTGTAATCCCAGCACTTTGGGAGGCCATGGCTGGTGGAC | |
| | CACAAGGTCAGGAGTTTGAGACCAGCCTGACCAATATGGTGAAAC | |
| | CCCATCTCTACTAAAAATACAAAAATTAGCTGGTCGTGGTGGTGC | |
| | ACGCACCTGTAAACCTAGCTACTCAGGAGTCTGAGGCACGAGAAT | |
| | TACTTGAGCCCATGAGGTGGAGGTTGCAGTGGACTGAGATCACGC | |
| | CACTGCACTCCAGCCTGGGCAACAGAGTGAGACTCTGTCTCAAAA | |
| | AGTAAAAAAAAAATAAATAAAAATGTAATAAGTAAATAAAATGCC | |
| | CTCTTTAAAGACACTCCTACATCTGGGCCAGTACACAGCCCAGCA | |
| | TGACTGGGCACTTCCCCTAAACAAGCAAGCAGACTCCTGAAATGC | |
| | ATCATTATGTTTCATTTCTTAATAAATTCTTTCTACAGCCAGTTA | |
| | GCATTCTACTTGTTGCCATTATAGACTTTCAAAAAACACTATAAA | |
| | CTTTCTTATCTGTCTTCCTGAGTAAGCAAAGGGAAGACATTATTA | |
| | GAAAATTCACACTCAATTAGGAAGAGTGGTTTGAGGTAGAAGTTG | |
| | GTGGTTTTTTATTTCTTTAAAAAAAAATATTGGCCAGGCACGGTG | |
| | GTTCATGCCTGTAGTACCAGCAGTATGGGAGGCCAGGGTGAGTGG | |
| | ATCACTTGAGCCCAGGAGTTCAAGACCAGCTTGGGCAACATGGCA | |
| | AAAACCCCATCTCTACAAAAAAATAAATTAACCAGGTGTGGTGGC | |
| | GTATGCCTGTAGTCCCAGCTACTCAGGAGGCTGAGGTGGGAGGAT | |
| | CGCTAAGCCTGGGAGGCGAAGGTTGCAGTAAGCCAATATCGCGCC | |
| | ACTGCACTCCAGTCTGGCTGACAGAGACAGTGTCAAAAAAAAAAA | |
| | ATCTTCGAGAAAATCCATTTGAAACAAGGAATGAAATTTAAAATT | |
| | TGCATATATAAACACGTATACATATACATATCATGTATATACATA | |
| | GGATTATGAGAAGTAGTAATGTTCTAAAAGTGAGTATGCATATTT | |
| | TTAAAAATTTCTGTAAAGTTGCACTTGTAAGGAAATTCCTAGGTG | |
| | TTATTTCCTAAGACGTAAAAGCAGTGTATACATCCTTGAAGCTAA | |
| | ACTTTTCTGAGATCTTAAATTCTCCCAGTGTTTGATATATGAGTA | |
| | CTCATGTCTCCAGCAAATTAACCTGAACTTAAAGAGCTTCCTGAT | |
| | AGCTTCTTAGTTGGGGGGTGGGAGTGCAGAGAATAAAAGGAAATT | |
| | CATTGAAGAATAAAAGATAATTTCTTGAGAAACCTGCTCATCGGG | |
| | ACCCTGTTAACATCCAAATATCCCAAATATCATGCCACACTATTC | |
| | TTCCCAGAATTAATTGTCAAGGACTGAGAAGGACAGGAGAACCTA | |
| | ATGCTATTTCAGGCTCTCTTGACAGACCAAGGAACAGTGAGGCAT | |
| | AAACTGGGGTCCCCGTGGGTCACAGGCAAGAGGTGGAACCCAAGA | |
| | TTCAACTCCCTTCTTGGAACCATCTACCTGGGGACTCCCTCTGGT | |
| | TCTGTGGCTGGGAAGAGGGATGCCCATCAGCCTTCCCGGAATTCC | |
| | GGGGGCACCCACAGGCCCTTACCTGCCTGGCAGCCAGGCACCATG | |
| | ATGAACGTGAGCAGTCCCCACATCAGCAGGTATGAATCCATCTTC | |
| | CTGACCCTTGGGACCAGCCGGGGCAGTGAAGCGGAGGTCTTTCTC | |
| | TGCAGAAGGCCCAGTTGCCGTCAGCCTCTTTTTGGCATCGCGCCG | |
| | GAGGATGTGGGATGGGAAGATCGGTCCGCCTGGGCTGTCACCCTT | |
| | GTGGGTCCATCCAGTCTCTATCGGAGTCAGGAGTTGCTCTCTTTA | |
| | AGTATTGGGCTGGCGTGTTCAGCCAGGAAAC | |
| 32. | TGCCCTGCCTGGCCAGCTCTGCTCTCCAGCCTGCTTCCCCGGTGT | GARP |
| | CCCCTCTCACATTCTGGGTTCTGGACACCTCATATTTCCTTCAGT | |
| | TCCCTGAGCAGATTTTCACTGCCCCAAACCTTCACTTTCTCCCCT | |
| | CTCCTCTTCTGCTAAAGACCTCCTCATCCTTGGGGTCCCAGCTTA | |
| | GATGGGCCAAGAGGCCTTCTTTGGTTCCCCAAGTCTGGACTATGT | |
| | CCTCCAGTCCTCCCAGAGCCCCTGAGCTTCCAGTGAGCACAGCAC | |
| | CAAGCCCTCCGTGGCTACTGGGCCCCACCCCCTCCCATGGGAAGT | |
| | GCCCCTAGAGAGGGGCTCACCATCCTTTAAGATGCATCTCACAGC | |
| | ACACAACAGGTCAGCAGCGGACAGAAAACCGACACTAGGAGTCTC | |
| | GGTGACATGGGGTCGTTACTGAGAGCAGTTCTCCTTGGCTCTCCC | |
| | TTCACCTCTCTGCAACGTTCCCTTCACCCATCCATGCCGTCAGTG | |
| | GGCTCTGACTGCTCTCCCTGAAAGAGACCCCGGATCTTCCTACTT | |
| | CCCTCTTATCACTGCCACAGACCGAGACGCCACCCCAATCCTGGC | |
| | CTTGACCACAGCACCCTGACCTCTTATAATCTGTCCTGTACCTAG | |
| | CAGCCAGAGTGATAATATAAAATCATCTGTCAGATCCTAAACCTC | |
| | TTCTGTTTGAAAATCTCCAAGAAAATCCAGGCTATCCACAGTCTC | |
| | CTCTCTGACTCTTGCCCATTTTCCCTTCCCTCACCACATTCCAGC | |
| | TTTCCTGCTGTTCCTTCAACACAGCAAATTCATTCCTGCCACAGG | |
| | GCCTTTGCTCCTGCAGTTTCCTCTCCCTGAATGCTCTTCCTCAGC | |
| | TCCTCACACATGTCATTGTTTTCCCACATTTATCATTTCCTCATC | |
| | ATTTATTTCTTATCATGTGGGAACAGGGGCCTACTAAGTAGGTAC | |
| | TCACTAAAGACTAGTGAACAAGTGGCAAAATCAGGGCAGAAGCTC | |
| | AGCTGCAGTGGACTGAGTGGTGAGTGCCAGGTGAGGACAAGGAAG | |
| | GGTCCTAGGCCCTTATTTCCCCTTCCTCTGCCAAACCCCACTCTA | |
| | TCCCCAAGGTGGCCCTGTGCACATCCAGAAGGGATCGTCCCTGCA | |
| | GCACATGGGCACTCTGTCCTGGCATCTGACGGGAAGGGCCCCTCA | |
| | GCTGTGGGCCCTCCTCAGTGGTGCCCAAGTACAGGGTGTAGCATT | |
| | TAAAGACTACTCTCTCTCAAAAAGGAGGACGGTGTCACTTAGACC | |
| | CAGGCTTGCATCCAAAGCCCACCACTCACCAGCTCTGCTCCCTAA | |
| | GGTAAGTCCCTTCCTTTCTTGGTGCCCCAGTGTCCTGTCTGCAGG | |
| | GTAGGCAGCTGGGTTCCAGTTCTGCCACTGGCCAACCCCAACTAT | |
| | TGTGATGATCAGGCAAAGAATGGTGGCGAAGGTGCCCTGCATCCT | |
| | CACTGTCCTTCCCAGCTGACACAGTGACCCTCGCAGGGCTGAGCA | |
| | TCCCAGGTGAGGTGTGCTCAGTGGGGAGGCCCCAAGCCTGCAGAC | |
| | AAGTGGCCTGGGCAAGAGGAGGCAGGAGAGTCGGACTCTCAGGAG | |
| | TCCAACTCAGAAAAGTGCCAAAGAATGTGTGTGTGTGTGTGTGTG | |
| | TGTGTGTGTGTGTGTGTGTGTGAGAGAGAGAGAGAGAGTGAGAGA | |
| | GAAAGAGCAGGGGTACGGGGGGCAATGACTACTACCACAATAAGC | |
| | CCACCCCTACATTTAAGGATGTTAGTCTTCTAAGCACTTGACCTA | |
| | TATCATCTCATTTAATTCTCACAATCACCTATTGAGGAAATTACT | |
| | GTAATTCCCATTTTACGGATGAGAAAACAGTTTCCGGAAAGTGAA | |
| | GTGACTTACCCCCAGAGTTACCCAGCTCATAAGGGCTTTGGAGCT | |
| | GGGATTTATCCAGTTCTACATGACCTGAATACTGTGTTTTCTCCA | |
| | GCACATGCTGAGCCGACTGCCCCCAATAGAACTGTCTTTTTTTTG | |
| | CCCATGAAGAAAAGGAGGCTCAGAGAAGTGACTTACCCAAAGTCA | |
| | CACAAGGAGAAAATAGCAGGGTCACGATCAGAATCCAGTTCATCT | |
| | GTCTCCAAAGCAAATGTTCCCACTATACCAAATCAAGGTCTCCTT | |
| | CCCTAAGCCCTGCCAACGACCCCTCACACGCAGGAGCAGGGCAGA | |
| | CTAGCTCACAGTCTGGCCTCTGAGGTCCTCTCTCCCTCCTCTCGG | |
| | CCTCAGCTGACTTACTGGCAGAGTGGGGTCCTGGGAGAGCTGCTG | |
| | CGGACGTTCCTGTCAAGCAGTGAATCACTAAGGGCAAGCCTCGTT | |
| | CTGGAGACATGTGCATCCCACGGGGGAATTAGACCTTAACCCAAA | |
| | GGAATGTGGCTGTGATGGCGGAATTTCAGGCAGCTCGGCGTGGGG | |
| | GTGGGGGCCCCTCACATCTTACCACCCATGAAGAAGGCTGGGTTC | |
| | AGAGCCCCTTGGAGAGCGGTGGGTGCCCCCTACCCCACTACGAGA | |
| | AATGGGCAGGCCATCTAGTGCTGTGGGATGGGCAGCCCCACGCCT | |
| | GGCTGGGATCGGAGCGTTGCAGGAAACCTGAATTCAAATTCCGGT | |
| | TTGCCCTTGCCTAGCTGTGTGTTCCTGGGCAAGTCACGGTCTTCA | |
| | GGCCATTGTTTTCCCAGCTGTCAAATGGGAAGGCCGGCCTCACCC | |
| | CTTTGCGGGAAAGATCCGCCAAGACAGGAGCGCTGGGGAACTCCC | |
| | AGGGACCGAGGGAGGGTCAGCCCCGGCTCCACGCGCGCAGTGGGG | |
| | ATGGCGGGAGGGGAGTGTTTGGGGGGCGTCCCTTGGGACCAGCCC | |
| | CCGCCTTGCGCTCGCTAGAGGCGCGCGACAGTGCGCCCCCCGAGG | |
| | GCCGGCCAGCCTCGCCTGCCCGGCGGCTCCTACCTGGCTCAGCGC | |
| | GGCCGCAGCTCCTCTCCCGGGCAGTCTCGGCACATTTTGCGCAAA | |
| | CCCGCCCCCGGGTCCCACCCCGCGCCAGACCCCGGGGTCACGGCC | |
| | CGAGGAGGAGCAGGCCGCTCAGCTGCCCCAC | |
| 33. | CCTATAATAGCCCTGTTGGGGCACAGTGTGAATTTTACACCCCTC | GPA33 |
| | CATTTCAGACTAGAAGACTGTGCAAGAAGACTTTAGCAAAGTGAA | |
| | GCCATTTGCCCACACCATAGTCAGTTAAGAGGCACTGGGGTCAGC | |
| | CCCCTTTGGTCCCCTGGGATGGCAGTGGAGTGTTAGGAGGGACGG | |
| | GTGGAACATTTCCCAGGCATGGAGGTCTGTTTTGTCACTGACTCT | |
| | GTTTAGAATTGTCAGCACATGATAATCATAAAAAGCAGACCAACG | |
| | GTGGCTCACACCTGTAATCCCAGCACTTTGGGAGGCTGAGGCGGG | |
| | CTTATTGCGAGGTCAAGAGTTCAAGACCAGTCTGGCCAGCATGGT | |
| | GAAACCCTGTCTCTACTAAAAATACAAAAAATTAGCTGGGCATGG | |
| | TGGTGCATGCCTGTAATCCCAGCTACTAGGGAGGCTGAGGCAGGA | |
| | GAATTTCTTGAACCTGGGAGGCGGAGGTTGCAGTGAGCTGAGATC | |
| | ACACCACTGCACTCCAGCCTGAGCAAGCCAGACTCTGTCTCACAC | |
| | ACACACACACACACACAAAGCAGACCAACTTTTAGTCGGTTTTAT | |
| | GATTGCTTTTAAACTCCCTACTTTAAAAGCACGGCCTTGCGTTTG | |
| | TGCCCCCTGCATGGAACCTTATGAATAGCTTCTGCTTCCTCCAAC | |
| | ACCACTTGAGTCTTGAGGCCCATTCATCAGTGCAGTGCTTCCCTG | |
| | CCCAGGTCTTAGCAGAATCAATTCCTCGGAAAGGTCAGATGGGAA | |
| | GAGAAAGAAGAGAAAAGCCCAGTAAGTCCTCTGGAACCCAAGTCA | |
| | AGCTCCACCTCCTCCAGGAAGCCTTTCCTAATGTCCCTGCTCCTA | |
| | ACTGGGTCTCCCTTACCTGCGCAAAGGAAGAGGTGGTATCCTCTC | |
| | ATGAGGCATGGTGGGGAGGGCCGCCTGGTTGCTTTTGGTCGACCT | |
| | TTGAAGGTCTTCCTTGCTCACCTGCAGTGGCAATGGCTATTTCAC | |
| | TATTTGGTAAACTCTGTTTATATACCTCAAGTCGACCACCAAGCC | |
| | TGGCTTCTCCTGCCCCACTGCTCTGGCCCCTTCTTCCTCTTAATG | |
| | TAAGATATGGAGGAAGAAAAGTTGGTGGGCACGTGATTCTGTGCT | |
| | GGGCAGGCTGTGGTGGGAGGGATGGGGCGGAGGTAGAAGACACTT | |
| | CATCAGTGTGTCTGAAAACATGCCCAAGACTCGTTCTGCTGGGTG | |
| | AGTTCCCCAGGGGTCACTTGCCTTCTCATAGGAAGAAGCAAAATG | |
| | TCCTGCTCCAGGCAATTCAGAGTTACGGCCACTTTTCCCTCCAAG | |
| | CTGGGTGAGCATCTTGTTTTAAGATCGTTTTTCTAAGCCAAAATT | |
| | TCTTATTTTTATTCATGCATGGGTCGCAGCCAGTCTACAAGTGTG | |
| | GGGATATGTGTTGGGGGTTGGGGTGGTGCAGGCATGGAGGAGCTT | |
| | CTGGCTTACCAGGAGAAGATGGGAGAATCACTGGCCCAGCCAGGG | |
| | AAGGATGGGTATTTCCTTCAGCCTCCCTCTCTCAGGCCCTGGAGC | |
| | ATCCTCCCTGCCTGAATCTGATGGCTGAGATAGGCCCACTTATGA | |
| | CTAATCATTCCAGACTCCTCACTTCTATGGCACCTGCACAAACTA | |
| | TGTGCTCAGTAAAGAGTTGTTTGGATAAAGGAATGAGCCAACCAG | |
| | TCCATCTTGGTGGATGATGAGGGGCTTTCTGCCTTCAGTCAGATG | |
| | AACTTGAGCCAGCCTGAGAGAATGATCTTACCCAGATCTTCCTCC | |
| | AAACCGTGTGATCCAGCCCTTCCCCAGCAGCAGGCTGCCCTTCCC | |
| | TCGAGAGCCCCAAGGCCAGGTGTTGAGAGAAAAGCATGTCTAGGG | |
| | CCTGGGAGAACCAGAATGAGCTGTAGGCCCCGTGGCCCGGGGCTT | |
| | TCCTGTGTATGCTAAAAAGTGAGCTCTAAACCAGTCTCCTGGTTT | |
| | CCACTTCCCCAGGCTTGGTGCTCCTGAGTTTCCTGGTATAGCTCT | |
| | TCTCCTCCCCGTTGCAGCTGGATCCTGGAAGCCCTCAACAGTGTC | |
| | TGAATTGCAAGGTGATGGCCAAATACACTAAACCAAAGATATTCC | |
| | TAAACTTTGCCAAACCCTGCGTCTCTCAACTTCCTTCTCTCCATT | |
| | CTATTTCCTTCCAATGCCCAGCAAAATGGTTGGTACCTTAATGCC | |
| | AAGACTTTCATTCTAACCCTTAGACACTCCATAGGTCCCCACAGA | |
| | ACACTGTGCTATTAAGCACTTGATGTGGATTCAACCATATACTCA | |
| | TGAATGTCTGATTTTGTTCTGGGAGGTTTCCCTCCCAGAGGGAAA | |
| | TGCTCAGAGTCCCAGAATTTTCAATGACCAGGGTCCCAGAAGTGG | |
| | CAAGGAGCTTTGGCCTTTGGAAAAGACCCATCTAGTTTGTTGTTT | |
| | GTTTTTAGGGTTTGAATCTTCTCTTGCTCTGTGAATGATACGGGA | |
| | TAAGTTGCATTGTCTCTTCTGCCTCAGTTTCCTGTCTCTTTCTCT | |
| | GGTTTTAACACAAACGTTAATATACTAGATAATGAACATATCTAG | |
| | ATCCACACATCCTGGATTTTCTAGGACAACTCCGATTTCAAATAC | |
| | TTTGTCCACATTGTCAAATCCTGTGTCTGATCCTATAATCTAATT | |
| | TGGGATAAGAAAATATGGTCTTGAGAGTACTTTGAATTCTTCATT | |
| | TCAGCAGTGGGCTAGAATGTTATTTACTAATATGTAAATAAAAAC | |
| | CATTCTGATTCTGGGCACTGGTCCGGAATGTGTAATTCTGGCCCA | |
| | GCAGGAACGCAGCTGTGTTGCTGGAGGCCCTTCCTGGGAAGGCTC | |
| | TGACAGAGCCTCTCCTTCAGCCCTAGGTCCCCATGTCTCACCCAC | |
| | CCCTGGGCACTGGAGAGAAAAGGGGCCTACTCACCTGCACAGAGT | |
| | GTCCACAACACAGGCCACATCTTCCCCACCATGGTCTTGCTTCTT | |
| | CTCTGCCCTAAACCTAACCTGTCACTGGCAGCCTCCAGACAGGTC | |
| | TGAGCTGTCTGGTTAAAGCTACAGCAGCTTC | |
| 34. | CCAGTGAGTGTCCAGCTAATTTAACTGTTTTCTGACATATTTTGA | CD14 |
| | GTCTGGTTCTGGTAATGTCTGTAACTCTCTTATCAATGTTAATTT | |
| | CCATTCCCTATTCCTATCTGTGTGGCTCTGGACATTGGCCAGAGG | |
| | CAGGTGCTGGGCCTATATGCCTCCTCCCACCTTTCTCCCATGCTC | |
| | CCTGTGAGGCAGGCATCTAGCTCTCTGATGGATGTGGCTGCCTCC | |
| | TCATTGAAATGAAGAAGGGGCTCAGAAGAAACAGCACCTCTTGAT | |
| | GGCTGATGCTGTAGGTCTCTGAGCTAATGTGGACTAGAGGACAAA | |
| | TCCTGTTTATTCTGAAGTTTTGAAGAGTCTGAGATGCTTTCCTAT | |
| | TTGCCAATCAGTCAGCCTGAAAAGGTTATCTGCATATGTGTTCTA | |
| | ACAGAACACAGGATCTCTAATTCAGAGACCGACTGTATCTTGCGT | |
| | CAGTTCCCTAGGCCCAAATTTCCCATTATGTCGGGGAGTGACACA | |
| | GTGAAAGCCTCCGGGTGAAGAGAGGTTTTACAAGAGAGGAACCCT | |
| | AATATTATGAAACTCAGAGCTTGTGCAGGATAGGAGGCTGTCCCT | |
| | CTTCCTTGAGTGGTAAGGGAGACAGACAGAGAGGGAGAGGGAGGG | |
| | TGTGTGAGTGTGTGTGTGTGTGAGAGAGAGAAAGAATAAGGAAAT | |
| | AAGCAAATGGCTGTGGGTTTTATTACCTTTGAAAGGGAGCAGCTG | |
| | GCTCTTGGGAAGTGCATAGGAGAGGAAATATTTTGTATCTTCCAA | |
| | ATATCATGCAAATACCTTTTAATCCAGATGCCAATAATCTGCTCA | |
| | GAAAGCCCTGACTGCACACTCATTGTTTCTCCCATGCAGCCCTAG | |
| | CCAGGAGTCCTTTCCTGGCTCCTTCTATCCCCGCAGTTCTTTTCT | |
| | TGAGGAGGACAGATAGGGTTTCTTAGGGAGTTAGGATGAAGAAAG | |
| | CCTAAGTATGATGAATTCCCCATCCAGCACTGTGCCCAGCACATA | |
| | GCAGACATCCAATAAAGGTCTGTTAAATGAATGACACGGACCCGT | |
| | TGTTTAAGATTTTAATAAAGGTGGGGCAAAGGGTTGAATTGGTCG | |
| | AAAAGTCCTCAACGTCCTGACGGGACTCCCCTGAAGCCAAGGCAG | |
| | TTTGAGTCCATTCATTATTCTGTCTTGGATCTTAGGCAAAGCCCC | |
| | GGGCCCCTTGGAGCAGCACCAGGGTTCCCGACACCCCCACCGACA | |
| | GGGTCGAACGTGCACAGGCTGGGACCACGCCGGAGTTCATTGAGC | |
| | CCTCGTGGGGGAGGGCAGTTCCAGGGACCAGGAAGGGATTCCCGT | |
| | CCAGTGTCAGGTTATCCACCTCGGGCAGCTCGTCAGGCTGCGGCG | |
| | CCCTGTTCAGTCTGTTGCAGCTGAGATCGAGCACTCTGAGCTTGG | |
| | CTGGCAGTCCTTTAGGCACCTGTTCCAGCCCAGCGAACGACAGAT | |
| | TGAGGGAGTTCAGGGCGCTGGACCACATGCATCTCGGAGCGCTAG | |
| | GGTTTACGGTGGCGCGCAGCGAGTTGTGGCTGAGGTCTAGGCTGT | |
| | GGGGCTGCACACCTGCCGCCGCCAGTGCGGCGCACACGCCTGTGG | |
| | GCGTCTCCATTCCTGTGTTGCGCAGCGCTAGATTCTGGATGGCCG | |
| | GGAACTTGTGGGGACAGAGAGCCGCCATCAGTCCGCGTTCGCCCA | |
| | GTCCAGGATTGTCAGACAGGTCTAGGCTGGTAAGGGCCGGGAAGG | |
| | CGCGAACCTGTTCGCAGGAAAAGGCAGGCGAGTGTGCTTGGGCAA | |
| | TGCTCAGTACCTTGAGGCCTGGCTTGAGCCACTGCTGCAGCTCGG | |
| | CGAGCCAAGAACGCCCTGTCGCCCACGACACGTTGCGTAGGCGCA | |
| | AGCTGGAAAGTGCAAGTCCTGTGGCTTCCAGAGGCAGCGGAGGCA | |
| | TGGTGCCGGTTATCTTTAGGTCCTCGAGCGTCAGTTCCTTGAGGC | |
| | GGGAGTACGCTAGCACACGCAGGGCGCCTACCAGTAGCTGAGCAG | |
| | GAACCTGTGCGGCTCCCACTGTGAGCCGCCGCACGCGGAGAGCCT | |
| | TGACCGTGTCAGCATACTGCCGCGGGTCGGCGTCCGCATCGACGC | |
| | GCTTTAGAAACGGCTCTAGGTTGAGACCGCCGGCATGGATCTCCA | |
| | CCTCTACTGCAGACACACACTGGAAGGCTTCGGACCAGTCGGGCT | |
| | GAGGTTCGGAGAAGTTGCAGACGCAGCGGAAATCTTCATCGTCCA | |
| | GCTCACAAGGTTCTGGCGTGGTCGCAGAGACGTGCACCAGCGGCA | |
| | GCAGCAGCAGCAACAAGCAGGACGCGCGCTCCTGGGGAGAGAGCA | |
| | GAGGTCTAGGAGGCCCCATCCAACCCCTGTGGCTCCCGAGTGGCA | |
| | CGCGTTCGACCCCAAGACCCTACACTCACCATGGTCGATAAGTCT | |
| | TCCGAACCTCTGAGCTCCGGACAGGCTCTGGAAGTGCTTTAGCTT | |
| | CTTTCCTACACAGCGGCACCCGCCGGCTTCCAGGCTTCACACTTG | |
| | TGAACTCTTCGGCTGCCTCTGACAGTTTATGTAATCCTGGGATGT | |
| | CATTCAGTTCCCTCCTCTGTGAACCCTGATCACCTCCCCACCTCT | |
| | CTTCCTCCGAGCCAGCCCCCTTCCTTTCCTGGAAATATTGCAATG | |
| | AAGGATGTTTCAGGGAGGGGGACCGTAACAGGAAGGATTCTGCAG | |
| | GGCATCTAGGGTTCTGTGTCTCCTGGCAGTGTCCTGATGACTCAG | |
| | GCGCCCCAGGCGGTGAATGCCCTGTTGACTCGGGAGCCTAAGCCT | |
| | TCTCTGGTGGGTGTGGGAAAAGGATGATCCTCAGTGCCTTAGGCC | |
| | AGTACCATACTCTGCACTATCCAACCCCCCAATCCCCCTACCTTA | |
| | TATCCCAGAGAATCTACTTGATTCATTTCTTTGACTTCTTCCTTG | |
| | TCTTGGTTTATGTTGATCTCCTGCCACCAAATCCAAGTCCCTGAA | |
| | TATCCTCAGATATTTAACTGCATGTTTTGTGGAAGAGATTGTGAA | |
| | CCTCATCTGTTGGCACCAAGGGGGGTAGAAT | |
| 35. | TGGTTTTCCTGCATATATATATATATATATATATATATATATATA | CD11b |
| | TATATATATATATGATTTATTTTTGTATGTATGTACTTATTTATT | |
| | TAATTTTAATTTTTTATTTTTTGAAACTGAGTCAAGCTCTATTGC | |
| | CCAGGCTGGAGTGCAATGGTGCAATCTCAGCTCACTGCAACCTCC | |
| | ACCTCCCAGGTTCAAGTGATTCTCCTGCCTCAGCCTCCCAAGTAA | |
| | CTAGGATTACAGGTGCCTGCCACCACGCCTGGCTAATTGTTGTAT | |
| | TTTTAGTAGAGATGGGGTTTCACCATGTTGGCCAGGCTGGTCTTG | |
| | AACTCCTAATCTCAGGTGATCCTCCCACCTCAGCCTCCCGAAGTG | |
| | CTGGGATTACAGGCGTGCCTGTATATATTACATGCCCTGCCTATA | |
| | TATATTTTAGAGACAGGGTCTGGCTCTGTTTTCCAGGCTGGAGTG | |
| | CAGTGGTGCAATCATATCTCACTGCAGCCTCAACCTCCTTGGCTC | |
| | AAGCGATCCTCCCACCTCAGCCTGTTGAGTAGCTGGGACCACAGG | |
| | TGTGCATCACTATGCCCAGCTAATTTTTAAACATTTTTTTGTAGA | |
| | GTCAGGGTCTTGCTGTGTTGCCCAGGCTGGTCTTGAGTTCCTGAG | |
| | CTCAAATGATCCCTCTGCCTTGGCCTCCCAATGTGCTGAGATTAC | |
| | AAGCAGGAGTCACCACACCTGGCTATATATAAAAATTATAAACAG | |
| | ATAATTTTTAACCAAATGTACATAATTTTAATTGCTTTTTTTTTT | |
| | TTCAGCCAGCAAGCATTTTTTTCCATCTAGTTATCGGGAAAGTAG | |
| | AACTAGAGGCAAAAGAAAATGTGTTGAATGGTGAAACCCTGTCTC | |
| | TACTGAAAATACAAAAAATTAGCCAGGCGTGGTGGCAAGCACCTG | |
| | TATTCTCAGCTACTCGGGAGGCTGAGGCAGGAGAATCACTTGAAC | |
| | TCGGGAGGCAGAGGTTGCAGTGAGCTGAGATCACGCCACTGCACT | |
| | CCAGCCTGGGCGACAGAGGGAGACTCCGTTTCACAAAAAAAAAAA | |
| | AAAAAAAAAAAAGAAAATGTGTTGAGAAAGAGTGAAAAAAATCAT | |
| | TGGCTTGAGAATCACATTGGCGGTCACATAAAGGTGAGGTTTGTG | |
| | TTCCATGTATGTGTGGAAGGATACCTATTTTCCTGTGCCTTTGAA | |
| | AACAGAATTAAAAGTGACAGACACAGGGGAGGGTAAGGATATATC | |
| | ACAGAGTGAAGTATCATTGAACCATTCCTCTGTTGAATATTCAGG | |
| | TTTTTTTAGCTTTTCATATTAACAATAATGCTGCAATGAACAAGC | |
| | TTGCAAATATATATATATATATATATTTTTTTTTTTTTAGAGATA | |
| | AGAGTCTTGCTCTGTCGCCTAGGCTGGAGTGCAGTGGCACAATCT | |
| | CTGCTCACTGCAACCTCCGCCTCCAGGGTTCAAGTGATTCTGCTG | |
| | CCTCAGCCTCCCAGGTGGGATTACAGGTGCCTGCCACCACGCCTG | |
| | GCTAATTTTTTTGTCTTTTTAGTAAAGATGAGGTTTCACCATGTT | |
| | GGGCAGGCTGGTTTCAATTGCTGACCTCAAGTGAGCCACCCCGCC | |
| | TCAGCCTCCCAAAATGCTAGGATTACAGGCATGAGCCACCGCACC | |
| | CAGCCAAGTTTGTACATATATTTTTGACTACACTTCTTAACTATT | |
| | CTTAGGATAAATTACTAGAAGTGAAAATTCTTGGGTGAAGAGCTT | |
| | GAGGCCTTTACACACACACACACACACACACACAAAAATAGGCTG | |
| | GATGCAGTGGCTCACACCTGTAATCTCAGCAGTTTGGGAGGCTGA | |
| | GGAAGGAGGATCACTTGAGTCCAGGAGGTTGAGAATAGCCTGAAC | |
| | AACATAGCAAGATCTTGTCTCTACAAAAAATTTAAAAAAAATTAG | |
| | CTGGCCATGGCAGCATGTGCCTGTAGTACCAGCTACTCGGAAGGC | |
| | TGAGGTAGGAGGATCGCTTGAGCCCAGGAGGTTGATTGAAGCTGC | |
| | AGTGAGCTGTGATTACACCACTGCACTCCAGCCTGGGCAACAGAG | |
| | CTAGACTCTGTCTCTAAAAAAAGCACAAAATAATATTTAAAAAGC | |
| | ACCAGGTATGCCTGTACTTGAGTTGTCTTTGTTGATGGCTACAAA | |
| | TGAGGACAGCTCTGGCTGAAGGGCGCTTCCATTTCCATGGGCTGA | |
| | AGGAGGGACATTTTGCAAAGTGTGTTTTCAGGAAGACACAGAGTT | |
| | TTACCTCCTACACTTGTTTGATCTGTATTAATGTTTGCTTATTTA | |
| | TTTATTTAATTTTTTTTTTGAGACAGAGTCTCACTCTGTCACCTG | |
| | GGCTGGAGTGCAGTGGCATTATTGAGGCTCATTGCAGTCTCAGAC | |
| | TCCTGAGCTCAAACAATCCTCCTGCCTCAGCCTCTGGAGTAGCTA | |
| | GGACTACAGGCATGTGCCACCATGCCTGGCTAATTTTTTAAATGT | |
| | ATTTTTTTGTAGAGTCGGGGTCTCCCTATGTTGCCCAGGCTGGAG | |
| | TGCAGTGGTGTGATCCTAGCTCACTGCAGCCTGGACCTCGGGCTC | |
| | AAGTAATTCTCACACCTCAGCCTGTCCAGTAGCAGGGGCTACAGG | |
| | CGCGCACCACCATGCCCAGCTAATTAAAAATATTTTTTTGTAGAG | |
| | ACAGGGTCTCTCTATGTTGCCCAGGCTGGTTTCAAACTCCCAGGC | |
| | TCAAGCAATCCTCCTGCCTTGGCCTCCCAAAGTGCTGGCATTACA | |
| | GGCGTGAGCCACTGCGCCTGGCCCGTATTAATGTTTAGAACACGA | |
| | ATTCCAGGAGGCAGGCTAAGTCTGTTCAGCTTGTTCATATGCTTG | |
| | GGCCAACCCAAGAAACAAGTGGGTGACAAATGGCACCTTTTGGAT | |
| | AGTGGTATTGACTTTGAAAGTTTGGGTCAGGAAGCTGGGGAGGAA | |
| | GGGTGGGCAGGCTGTGGGCAGTCCTGGGCGGAAGACCAGGCAGGG | |
| | CTATGTGCTCACTGAGCCTCCGCCCTCTTCCTTTGAATCTCTGAT | |
| | AGACTTCTGCCTCCTACTTCTCCTTTTCTGC | |
| 36. | CATCATCGTGGGTACCCCTGGCCGTGTGTTTGATATGCTTAACCG | CD68 |
| | GAGATACCTGTGTGAGTAATTCGGTTCTCCAATCCCCTGGGTCAC | |
| | TTTGCTCTTGTGCACGCTTTCCAGTCTTTCAGCGTAAGCCAGAGT | |
| | CATTCCCAAGGATGCTGGTTTCTCTCTGGGGGAAGAGCTGCTCTG | |
| | TGATGGAGCCCATGCGTGTCATCTGAGCCTCTGGCTTCCCTGCCA | |
| | GTGCAGCCCTGGCAGTGTCCTACTTCCCAGGGCTGTTGTCTGCCT | |
| | GGCGGGAAGGTCCTGGGCAAAGGATCAGTCTTTGTACTCTGAGAG | |
| | CAGACTACTTGGCTCCTCTCTGTTTTTTATCAGCGAAGTTGGATA | |
| | TATCTCTCCCACATTTCCCTAATCATATGCTATATATTGGCTTTT | |
| | TTTTTCTTCTCTAGCCCCCAAATACATCAAGATGTTTGTACTGGA | |
| | TGAAGCTGACGAAATGTTAAGCCGTGGATTCAAGGACCAGATCTA | |
| | TGACATATTCCAAAAGCTCAACAGCAACACCCAGGTGAGGGCAGT | |
| | CTTGCTTGAATAGCTAATGATTCTTGAAAAATAGTAAGTGCCAGG | |
| | GGAACCATATACTGGATTCTTGAGCCTTTTTATGCATCTGCTTCA | |
| | GTTTTAGGTGTGGCTAGGGAAGGGAGCAGGCCTCAGGAAGGAACC | |
| | AGCACTCTAAGACTGGCCTTTTTTTCCACTAGGTAGTTTTGCTGT | |
| | CAGCCACAATGCCTTCTGATGTGCTTGAGGTGACCAAGAAGTTCA | |
| | TGAGGGACCCCATTCGGATTCTTGTCAAGAAGGAAGAGTTGACCC | |
| | TGGAGGGTATCCGCCAGTTCTACATCAACGTGGAACGAGAGGTGG | |
| | GGCCCAGTGCAGGAGGCGGGCCTGGTAGTGAGTTGTTGGGTATAG | |
| | CCCCTGACTGATTTTTGTCCCCCAACCTCCAGGAGTGGAAGCTGG | |
| | ACACACTATGTGACTTGTATGAAACCCTGACCATCACCCAGGCAG | |
| | TCATCTTCATCAACACCCGGAGGAAGGTGGACTGGCTCACCGAGA | |
| | AGATGCATGCTCGAGATTTCACTGTATCCGCCATGGTGTGTTTGC | |
| | CCGCTGCCAGCCTGTTGTGGGTCTGCCCGTCAGAAGTGTCCTACT | |
| | TGAAGCCAGGGTTCCTGGAACCCAGGTGCCTACCTGGTCTGCTGC | |
| | ATATTTGTTTTCTCTTCCAGCATGGAGATATGGACCAAAAGGAAC | |
| | GAGACGTGATTATGAGGGAGTTTCGTTCTGGCTCTAGCAGAGTTT | |
| | TGATTACCACTGACCTGCTGGTGAGTAGAGGGAACTGATAGCAAA | |
| | GGCAGAAGGGAGGATCCAAGGTGATTCCCTCTCCAAGGGGACATC | |
| | AGTGCCTCTCAGGAAAGTAGCAGCTTGGAATAGAATCTGGCATGC | |
| | CTAAGGCCTTTGGGGAACTGGGATGCTTATTTCCTCTGCCTTCCT | |
| | TGGCTGCCCACATGGATGCCTAAGTGTCTTCCCTCCGGGATAGAG | |
| | TGTCCTCCGTGCACATGCTGAAGAGTTGTCTTTCTTGACGTAGGC | |
| | CAGAGGCATTGATGTGCAGCAGGTTTCTTTAGTCATCAACTATGA | |
| | CCTTCCCACCAACAGGGAAAACTATATCCACAGGTAAGCGTAGAT | |
| | CTGGAACACTCCCCTACCCCTTCACACCTGGCCCTCCCTGGGCTT | |
| | AAAGCTCCTGATATTCCTCATCCCCTTCCTTGTTTTCCAGAATCG | |
| | GTCGAGGTGGACGGTTTGGCCGTAAAGGTGTGGCTATTAACATGG | |
| | TGACAGAAGAAGACAAGAGGACTCTTCGAGACATTGAGACCTTCT | |
| | ACAACACCTCCATTGAGGAAATGCCCCTCAATGTTGCTGACCTCA | |
| | TCTGAGGGGCTGTCCTGCCACCCAGCCCCAGCCAGGGCTCAATCT | |
| | CTGGGGGCTGAGGAGCAGCAGGAGGGGGGAGGGAAGGGAGCCAAG | |
| | GGATGGACATCTTGTCATTTTTTTTCTTTGAATAAATGTCACTTT | |
| | TTGAGGCAAAAGAAGGAACCGTGAACATTTTAGACACCCTTTTCT | |
| | TTGGGGTAGGCTCTTGCCCCAGGCGCCGGCTCTTCTCCCAAAAAA | |
| | AAAAAAAAAACACTAATCCATTTCCCTAACCTAGTAACCTCCAGA | |
| | TCCCAGAGGCTCTCCTCACCTCAGCTGAGCTCCTTTGAAAGTGAT | |
| | TCAAGGGACTATGTCACTCAGCCTCATTTGCTGGACCAAATCTGG | |
| | AGGGAGAACCCCTAAAACCCCTAAGTGAGGTTGCCCAGGGGGTTG | |
| | TCCCCAGGTGGGGGGAAGCAGGGGAGAGAAAATGGTAGCCATTTT | |
| | TACATTGTTTTGTATAGTATTTATTGATTCAGGAAACAAACACAA | |
| | AATTCTGAATAAAATGACTTGGAAACTGCCTGTTTGGGCTTCTCA | |
| | TTTCTTACCTCCCCTTCCCTCTCCCACCTGCTACTGGGTGCATCT | |
| | CTGCTCCCCCCTTCCCCAGCAGATGGTTACCTTTGGGCTGTTGCT | |
| | TTCTTGTCACCATCTGAGTTCTCAGACGCTGGAAAGCCATGTTCT | |
| | CGGCTCTGTGAATGACAATGCTGACTGGAGTGCTGCCCCTCTGTA | |
| | AAGGGCTGGGTGTGGATGGTCACAAGCCCCTCACATGCCTCAGCC | |
| | AAGAGGAAGTAGTACAGGGGTCAGCCCAGAGGTCCAGGGGAAAGG | |
| | AGTGGAAACCGATTTCCCCACCAAGGGAGGGGCCTGTACCTCAGC | |
| | TGTTCCCATAGCTACTTGCCACAACTGCCAAGCAAGTTTCGCTGA | |
| | GTTTGACACATGGATCCCTGTGGATCAACTGCCCTAGGACTCCGT | |
| | TTGCACCCATGTGACACTGTTGACTTTGCCCTGATGAAGCAGGGC | |
| | CAACAGTCCCCTAACTTAATTACAAAAACTAATGACTAAGAGAGA | |
| | GGTGGCTAGAGCTGAGGCCCCTGAGTCAGGCTGTGGGTGGGATCA | |
| | TCTCCAGTACAGGAAGTGAGACTTTCATTTCCTCCTTTCCAAGAG | |
| | AGGGCTGAGGGAGCAGGGTTGAGCAACTGGT | |
| 37. | CTTTGTAAGATCAGAAAAAGCAAAAAGAACACCTAAGCTTCAAGC | CD138 |
| | CAGAGTCTGACCCCAAAGCTCAGGTGGGGCTTTCAGGCTCAGCTC | |
| | AAGCACCTCCTCCAGGAAGCCTTCCCTCCCTGCAGCTCCAGGGAG | |
| | TCTCCCTCCTTTCCCCTTTTGAGACTCTCCTGCCTCCTGGGCCCT | |
| | ATGTCCAAGTTTCTGGGCAAGAAATAATGCCTCACACTGCAGCAT | |
| | CTGCCCCAGCCCACAGAAAACTCCAGGAAAGGCAGTTCACGGGGA | |
| | AGCCCAAACTCCACCTGCCTCCCCAGCATTAACGGGTGCGGTGGG | |
| | ATCCCAGGGCTCTGGAATGTCTGCTTTACCCCAGGCAGGCAAGGG | |
| | AACCAGCAGCCCTCCTAAATCCAGCCGGAAATAGGTGGAGGACCC | |
| | AACTTCAGCCGCCCCTGCTGAGGCCCCATCATGTGCGAGAATGGG | |
| | GCAGTGAGGTGATGCCCAGCGGTCCAGGGAGGTAAGGCACCCAGC | |
| | TCCATTCTCTTCTCCAAACTCGGAGCGAAGACTCCAAGCACAGTC | |
| | TCAGTTCCACAGAAACCACCACCCTAGAGAGCAGCTTCTTGGACC | |
| | TGAAAGCCAGGAGTGCATGAGACCCAGCTCGGAAATCATGGCCCT | |
| | CCCCGTGCTCCCAGATACCCCTCACAGTCCAGCCCATTCACAGTT | |
| | CAATAGGCACAGGGACCTCTCCTGGACCGGAAAATGTCTGGAAGC | |
| | ACGAGTCACCTGGGAAGCAGGGAGGGCGCTTTACACATCGGTCTC | |
| | AGAAACGACTGTCCCTACCTCATCTCCCATGAGACAGACTCTGTA | |
| | AAGAAAAAAAAAACCAAAGCGCTCAATAACTGGCAGCTACTACAC | |
| | GAGGCAACGCTTACGGAGGGTCAGCGCTGCTGAGACTTGTCCAGA | |
| | TGCCACATTAGCAGTGGAGAACCCGAGTGCCCACCCCGCCCCCGC | |
| | CCCTTCCCTGCCCACGTGCCTCCCTTTGCCAGAGCTGGGGAAGTC | |
| | GAACTGTCCAGATGTAGTGGCGAGACACCGCCACTTCCGGAAGGG | |
| | GAAGCGCCCCTGGCTCCGCACCTCCCCTCTGCATCCCTGGGGATG | |
| | GCCGAGCCGGCCCGCAGGAGGGACCCCGGGACGCCAGCACCGGGC | |
| | GCGGGGGCTGGGGAGGCGAACTCGGGCGGCAGACTCGGGCACCGG | |
| | GCGGCCCGCGCTGACACCTGGCTGCGGCCCAGGTCCCAGCCCCCG | |
| | TCAAGGCCGGCGTTACGGGGCTGTTCCCAGGAAAGGGTTACAGGG | |
| | GCCGAGAACAAAGCGCCCCCATTGGGGAAGGCGGGCTAGCGGCCG | |
| | GTGGCCGGGGCGGGAGAGCCACTTTCTGGGGACCCTCAGGCTGCT | |
| | CTTTGTCGGCCACGTCCCTCTTTAGTGGAGCGTCAGCCCCGCTGA | |
| | AGGTGGATGCTGCATCCCACCCAAAGGCCTCTTACAGCCCCGAGC | |
| | TCCACATCTGCCCCTCTTTTCTCCCAGCTGCACTGGGCGGGCCAA | |
| | GGGACCCGCCTTCCCTCCGCGCGGCTTCAGCACAAAGCCGAGCCG | |
| | GGGAGCGGGAGGCCATTCCCGGGTCCCCTCGCGTGGAAGGCGCCT | |
| | GCGCCTCGGCCGTGCCCGGCACGGGAACGCGCCCTCCGGGGCCAG | |
| | CTCAACTTCAGCAGCCCAGAAGTTGGGCTCCTCCGGGTCTCCAGC | |
| | GTTCCGAGGCCAACTTCCCGGAACCTCCCGCTGCCGGGCCGGCTC | |
| | AGCTCACCTCGAGCCGCCCACGGCAAGCCCGAGGGCCCTGCAGAC | |
| | GCTCGCCCGCGCCCCCCACGCAGCCCTGGCCCGGCTCCCCGGGCC | |
| | AACGCGGCCGCCTCCCGCTCCCGCGCCGGCCGCGCTGCGCCCAAA | |
| | CTTGCCCTGGCGCAAGGGGCGGGGCGCCCGGGCTCGGCGGCGCTG | |
| | GGGCGCAAGCCCGCGGGTCTGGTTTGAATTAGGGTCTGCAGGGGG | |
| | GGCGTTGTGGCCGCGGTCACTGCATCCCCCCGGGTCACCGACGGG | |
| | GGCCCGGCCGCCGCGGTGGCCGGGGCGAGGAGGGTGGGAACGGGC | |
| | GACCCCGGGCGCCGCTGTGGGCTGGCGGGCTCCGACAGATGTGGA | |
| | GACGTTTTACATAATTGAGCGCGGGGCCCCGGCCCCCCACCCTCC | |
| | CCAGCGCGGGACCGGTGCGGCACCCACCGACAGCGGAGGAAATTG | |
| | GGGCTGGAGCCCTGGTCTCGGGGCTCACCGTCCCGGGACCCGCTG | |
| | GGCTAGCGCGGGAAGAAGGGAAGTCTTCGCTCCCCCTCCCCCTCC | |
| | ACGTGCACCCGCCGGCATCCGCGGGTGACCAGTCCCGGCTTCCCG | |
| | CCGCCTCCCCGCCTGGCCGCCGGCCGCACTCACCGGCAGGGCCGG | |
| | CTGCAGGCTCAGCGCCAGCGCGCACAGCCAGAGCCAGAGCGCCGC | |
| | GCGCCTCATGCTGCCCGGACCGGCGGCGGGAGAGCGGCAGGCTGC | |
| | GCGGGTCGCGGCTGCGGGCCGGCTTCGCGGGTTCCGCTGCTCGAT | |
| | GCTCTCTTGGGCGCCTGCCCAGCGCGCCGCTGTCCCAGGCGAGGG | |
| | CTGCAGGGTCCGCCGGCTGGAGTCCGCTCTCTACTGCCGGATTCC | |
| | TCTCCGCTCGGCTCGGATTCGGCCCGCACCTCTCCCGCCGAGCTC | |
| | CGCCTTATAATAAACCCACAGGCCCTTCCTTAGCCGTTGCAAAAA | |
| | CTGGCCCCCCACCCCCAGCTCCGCCAGGTCTCCCGGCCAGCCCCA | |
| | GTCCACACCCCCCAGGACCCCGCCCCCCAGCCACCCCTCCCGGCC | |
| | CCTTCGGAACGCCCCACCCCCGGCCCGCTCCTAAGGTTCTTGCCT | |
| | GCTTGGCTCCCGCTCCTCCGGTGGCCGAACGTCGCTTTCGGGAGC | |
| | TGTGGTCCCCGCAAGCCGCGGTTCCCGGCCTTTCAGCTCGGCTGC | |
| | TCCCTGGGTGGTGGGGCCGAGGCGCACCCCCTTCTGCCCGGGCCC | |
| | GGAGTGCGGAGGCGCAGCCCGGGAGAGGGGCTGAAGGGGGGCAGG | |
| | CAGTCCTCGGGCGCGAGTGTGCAGCGCCCTC | |
| 38. | TAGAAAGATAAACTCAAGTCGCAAAACATGTTTTTCCTTGAAAAG | IgH |
| | GAAGAAATGATGTAATACATGTTTCAATTGAATAACTGTCTTTGT | |
| | TTCTCACTTCTGTAGTATGCTTCACCCTGCACAGATGTCCCCTCT | |
| | CCCACCCCACAAAATGCTTAAAAGGTAACTGAACTCTGTTCTGGG | |
| | CTCAGTCCTTTGGATGTTAATTCGACTTGGCCGGTGCACCTAAAT | |
| | AATAAATATCCTCCTGAACCTAAATAATAAATATCCTCCTGAACA | |
| | GAGTCGGTCTCTCTGATTCCTTAAAAAATCCCAGAACAGTATGGC | |
| | ACTTAAATGTGCAATGGTATTTATTTTAAATATCAGTCTGTTGTT | |
| | AATAAATTGAATAACTAATAAGACAAACATCACTTTTAAAACTGT | |
| | ATTACCTTATTTGTTGAATTTAGATGGTAATTTTCAAACTAGGCA | |
| | AGATAAAATTTTTTACTTGAAAAGTTTTGAAAAAAACTTCTTTGG | |
| | CATGAATATTCAATACAAACTGTAGTCTTTATTTGCCAACATGCC | |
| | TTTATAATAGAGAACATCCAGCAATATGAAGCTAAACCCAGCCCA | |
| | TGCTTTTTAGGGCTCACTCACAATGGCAGCTTCCTAGAGGGTGGT | |
| | CTGAGAGAGTGTAAGCACATGGGGATTTGGGCTTCATCATCAAAG | |
| | TGAAGAAGTAATTTATGAATTTTACAATATGTAAAGCCAGAAAAT | |
| | TAACTTTCCCCTCAGGGCTTACTGTAGTGTGTAGCCCCTCCCCCG | |
| | TAGTCTAAGTTAGAGAATACTAACTGCCTGTTTTTCCTTCTGTGC | |
| | TCAGTGAGCCTTATCTGTTCTCATTGGTTTCACATTCCTTGAGGC | |
| | TCAGAGGGTTCTTGCTTACCTCCCCAGCACAGCTGCAAGGTCATA | |
| | AGATTGTTAAGTATATGTTACAGAACCATGTATTCCCAAGGATGT | |
| | AAGACATGAAGTAACAAATAACTGCCTTTGTTCTCTCTTCTGTAA | |
| | TTACGCTTCCTGCATCATGTAGCTCCCAGCCACTGACTGTGTAAA | |
| | AGGTGGCTGCTTTCTTTGTCCAGGGCTCAGACTTTCCTGGATGCT | |
| | AGTCCAACTGAGCCAGGTGATCACCTTTTAATAAAGGACTCTCCT | |
| | GAACTCTGTTTGGTCTCTCCCATCTTTGATCGTCCTGCAACAAAA | |
| | GCACTAGGGAGCCCCAGGGCTGAGCACACAGAAGGCAGCAGGAGC | |
| | TGCAGAGCCCACTCTGTCGTACTTAGGGAAGGGAGGGAATGGAAT | |
| | GAGGGTGATGTCTGCAGGACCTTAGAAAAGGGTGAGGAGGGCAGA | |
| | GAGTCTGCAGGTAGATGAGCATATTCTAAGGAGAACTGTTATCCT | |
| | CCTAAACTTGGTTGGCTTCAGTTATTATGAAGAGAAGGAAACTGT | |
| | TCACCAGACTTGGAGGACAGAAAGTAAAGGGAATTTTTATTTCCT | |
| | GCATGGTGACTGAGGAAGATAAAAGACTTTTAAAGTAAAAGGGAA | |
| | GATGGAGAAATAGTCTGAAAAACAGGACACCAGGAGCCAACCAAA | |
| | GTGGAGAACAAGAGCCTTTAAAGTGGTGTTTAATTTCCATTCCAC | |
| | TTTCAGCTGATGAAAGAAAAAGAAACAAAACACCATGCATATGGT | |
| | GAGTTAATGTTAGAAAACATATATAATTGTTTGAGTATCACAGCA | |
| | CAAAAACACTAAACTCTATTCATGGAAACAATGTGTATCGAGGAT | |
| | ACACATTTTTTAAGACAGGGTCTCACTCTGTCAACCAGGCTGGAG | |
| | TGCAGTGGTACAATCACATCTCACTACAGCCTCAACCTCCCAGGA | |
| | TTAAGGGATCCCCCCACCTCAGCCTTCCAAGTATCTGGGACCACA | |
| | GGCAGCATCACACCCAGCTATTTTTTTTGTATAGAAAGGATTTCA | |
| | TTGTGTTGCCAGGCTGTTCTAAAACTCCTGAGCTCAAGTGATCTG | |
| | CCTGCCTCAACCTCCCAAAGTGCTGAGATTGCAGGTGCAATTTTT | |
| | ACAATTTTACTCTTTTAATAATAGTATTTTAATAAAATATATAAA | |
| | TAACCTATTTTAAAAATTGTTCTGACAGATCATTGCTAGCGTTAC | |
| | TCAGAAAACACGCAGCATTAAAACTTCAAATAGGCTGGGCATGGT | |
| | GGCTCACGCCTGTAATCCCAGCACTTTGGGAAGCCTAGCGGGGCA | |
| | GATCACCTGAGGTCATGAATTCGAGACTAGCCTGGTCAACATGGT | |
| | GAAACCCCGTCTCTACTAAAAGTACAAACATTAGCCAGGCATGGT | |
| | GGCAGGCACGTGGTGGCAGCTACTCAGAAGTATGAGGCAGGAGAA | |
| | TTGCTTGAACCCGGGAGTTGGAGGTTGCCATGAGCCAAGATCATG | |
| | CCACTGCACTCCAGCCTGTGCGACAAGAGGGAGACTTTGTCAAAA | |
| | AAAAAAAAATTCCCGGTTCTAAGTTAATGTTTTGGAAGTAACATT | |
| | TGTAAAGAAATCATGGATTTACAAGGAAGTAATGGTTCTGAGCGT | |
| | CCCCTGGTGTCCTGAGTGCCCCCTGGTGGCTCGGAGCACCCCCTG | |
| | GTGTCCTGAACACCCCCTGGTTGTCCTGAGCGTCCCCTGGTGTCC | |
| | TGAGCGCCCCCTAGTGGTTCTGAGTACCTCCTGGTAGTTCTGAGC | |
| | GACCCATGGTAGTTCCAATCAGCATCTACCATGCGATTCCCTCCT | |
| | GTCTCCCTGCAGAGGATTTTGTGTCTGGGCTCACGCAGATGTTCC | |
| | TGCTCCTATGTCACTCACAGTAATACAAGGTCTTGTCCTTGGCTT | |
| | TCACATTGGTCATTTTAAGGTAGACTGCACTTGAAAGGGTGTTGC | |
| | TTGGGACTGTTAATTTACTTGTACTCATGGAGAGTAACCCTGATA | |
| | ACTCACTGTTGCCACCCACACAAATCCCTGTCATGAAGCCTGCTG | |
| | GACCAAGCTCATGCTGTAGCCAGTACAAGTGAAATCAGAGGCTTT | |
| | GCAGGAGAGTCTGTGAGAACCGCTGGGCTGTACAATGTTTCCCCC | |
| | TCTGACTCCATCAGTAAGCTTCAACAGGACT | |
| 39. | CCTGCACCCAGTTTCTCCTGTTGGTTACTTCTTACGTAATTACAG | IgK |
| | TACGATATTAAAACCAAGAATTTGACATTGATAAAATAAGTGTGT | |
| | ACAGTTCTATGTCATTTTATCACATGTCTAGATGCCTGCAACTAC | |
| | CACTGCAATCAAGATACAGATTTATCCCATGATCACAAAGATCTC | |
| | CCTTTATAGTTTCGCCTACTCTTCTTTCCCCACCACCCTAACCCC | |
| | TGGCAACCAGAATCTGTTCTCTATCCCTATAATTTTGTCACTTCA | |
| | ACAATGTTACATAAAAGGAATCACATGGTATGTGACCCTTTCAGA | |
| | TCTTTTTTCACTCAGTAGAAAGTCCTTCAGAATCACTCCAGTTCT | |
| | CTGTATCAACACTTCGTTCCTTTTTATTGCCTCATAGCATTCTAT | |
| | GGCATGGATATATTACAATTTGTTAGGCTAGTTGCCTATTGAGGG | |
| | ATGGGTTGGCTCTTTCTAGCTTTGGCTACAACAAATAAAGCTGCT | |
| | GTGAACAATCATGTATGGGTTCTGTGTGGACATAAGTTTTTATTT | |
| | CCTGGGGGTAAATTCCCAAGGAGTACAATTCCTGGGTCATATGGT | |
| | AGGTGTCTTTTGTTTAGTTTTTCAAGAAACTGACAACTATTTTCT | |
| | AGAGTAGCTATACCCTTTTATATTCCCACCAGCAATATATGAATG | |
| | ATCCATTTTCACCGCATCCTCACAAACATTTCAGTATGACCTAAT | |
| | TTTGACTTGATTACCTCTGCAAAGACCCTATTTCCAAATAAAGTC | |
| | ACATTTATGTGACCCATTTATGAGTACCAAGAGCAAGGAGTTGAG | |
| | CATACCTTTTTGAAGGACACAATTCCACCTGCAGCACCTGTCTTC | |
| | ACTTGCTTAGGTTATGGGTGGGTTTCCCTTCAGAGTGTTATCTTC | |
| | TGAGAAGAAGGTTTGAATTCATAGCTGGATTATGCCTGCCTTTGA | |
| | TTGTTCAATTTGGCCCCCAGGGATGAGTGAGCAAGTTCATGTCAT | |
| | TGGAAGGGACATTACTGAGTCTCACCCCTACTCAAACCTCCAAAG | |
| | TTTGAAAGATTTGATACTCATCAACATTCTCAAGAGGCAGAAGGG | |
| | CAAGGTGCTTCCTTATGTAGTTTCTCCATGACTGTCCTTCCCCAC | |
| | TTTCCCAGTACTGACTGGAAAGAAAAACAAAGCAGGACAAAGGGA | |
| | TTGTCAGTCATTTGAGGGAAGGGATGCTGTGTTAGTTACTCCTGA | |
| | ACCCCTTCAGAGTGTAGGCAGAGAGAGGTACTTAGAAAGGTGTGT | |
| | TGGATGGATGAGTAGCTGAGAGGGGCAGGGCCAGAGTGCCCCATA | |
| | GAATGAGGAAGAGTCCTCATTTACATGTGCCCTTCCCCCTTCCCA | |
| | AACATGTGAGTGTTCATACATGGGTTTCCAGTGTCTGGGCAAGGT | |
| | TAATTCTGTCCTCTGTCCCCTTGCTGAAGGTCTGTTTCATTTAAT | |
| | TTCTATGTATGTGTGCTCCTAGCTAGCCAAACAGGGCACAGATGC | |
| | CAGGAAAATAGCAGGCCTGTTGTTTTGTTGAGCACCTTGAGCCCT | |
| | GGAAGGCAGACTCATTCAGGGCTCAAGAGGGCAGGATTAGGCAGG | |
| | TAGCTATTTGATCCCAGGCCCCCCCACTTACCAGCTGCGTGGTCT | |
| | TGACCTATTACTTATTCCTCTTCTATCTATTAACTGGGTATAAGA | |
| | TGTTCTTTATGTGAAGTTATTTTGAAGCATAATGTGTATACAGAA | |
| | CAGTGCACAGATCATAACTGCACAACCCCACACGAGGAACAGAAC | |
| | ATTACCAAGACCCCAGAAGCCCCTTCTTGTCTTCTCCCAGGCACT | |
| | AACTCGCCCACCCCCAGCTAAGAGTAATTTCCATCCAAATTTCTA | |
| | ACTTTATTTTTACCTGGTTTTGAACTTTATATGGATGAAAACATA | |
| | CAGAACATATCTTTTGCATTGAGTTTCTCTTATGTAACATTATTT | |
| | TTATGAGATTTGGCATCTGTGTGAGTAGGTATAGTTGTAGCTTGT | |
| | TAATTCTCCTCATTGGGTATTCTAATGTATAAACATACCACAGTT | |
| | TAATAACTCATTCTATTGTTGGACAATCGGGCTATTTCCAATTTT | |
| | TGGCTTTTGTAAATAAAGCTGCTTTGAACATTCTTGCATAGTTTT | |
| | TTGGTGGGTGGGGACACTCAGTCATCTAGAAGTGGGATTGCTGAG | |
| | TTATAGAATATATTTGTCTCTCAGCTTTATAGAACCTTTGCAACA | |
| | TTTTAATGAATGTTTTTAATCTCACAGTATAGTGTGGATTAAATT | |
| | AGGTTGTGGAAAGTACTCAATACGGCCCTTGGCAGACAGGAGTCC | |
| | CTCAGTACCTGTTTAAAAATGTAAATAAGGCATGACATTCCATTA | |
| | AGTCTAAGTACCATGATGAACTTAAACATTTCCCTATTGTTTGTT | |
| | TATTTAATTGCTTTTATTGTGTTGTTATTTTATATCATGTTGCAA | |
| | TGAATGATTCTCTTTATAGCAGGTTTCTTCTTTTGAAAAATTTGC | |
| | TTTGGTTGATTCTCAGGGAATTGAATCAAAGGATATATGACTGTA | |
| | AGACCTGTCACCCTTAAAAAGGACTATGAGGGCTTGCTGAGGAGG | |
| | GGAAAACAAGGAAGCAAGTCTCTCCTACCATGGCCCAGGGGACTG | |
| | TGAGGACAGAAGGCTTGTGGGTTTGAGGGAGGACTGTCTTGCAGA | |
| | GGATGATAGGGTAAAATAGAATGAAGGATGATTTTTATAAATGGT | |
| | TATGCGCCTTAGGATGACTACATATTTAGTCCCTTATAAGAGAAA | |
| | TTGAGTAGTTGGTAAAACAACAGATAATAATTATTAAATGAGGAA | |
| | AGAGAGAAACCACAGGTGCAAAGATTCACTTTATTTATTCATTCT | |
| | CCTCCAACATTAGCATAATTAAAGCCAAGGAGGAGGAGGGGGGTG | |
| | AGGTGAAAGATGAGCTGGAGGACCGCAATAGGGGTAGGTCCCCTG | |
| | TGGAAAAAGGGTCAGAGGCCAAAGGATGGGAGGGGGTCAGGCTGG | |
| | AACTGAGGAGCAGGTGGGGGCACTTCTCCCT | |
| 40. | GAACACAAGATCCATATGGGGAAGGATTTCTATGAGGGTGTGGCA | IgL |
| | GTGAGAGCGGGTGGCAAGTTGCCAAAGAGGCCTAAGCCACAGAAG | |
| | TGGGTTTCATGTGGGACTTTCGGAATGAGCAGGAGGGCGATGGGT | |
| | GGAGGGTGAGGGTCAGAAGATTCTAATCAGAGGTGAGAACCCAAA | |
| | GATGAGTTATTGGTCATTCAAGTCAAACAGTGAAATTACCTTGGC | |
| | CTGTGCCTCCAGTCAGTGGAGCAGGGAAGGGGCCAGGATGTGCTT | |
| | CCTGCAGGTGTTACTGGAAAGGGGTCCTGATTCAGATCCCAAGAG | |
| | AGGGTTCTTGGATCTCATGCAAGAAATAATTTGGGAGGAGTCTAT | |
| | AAAGTGGAAACAAGTTTATTAAGACAGTAAATGAATAAAGGAATG | |
| | GCTACTCCATAGACAGGGCAGCACCAAACACTGCTGGTCGGCTAT | |
| | TTGTATAGATAGTCCTTGATCATATGCTAAATAAGGAGTGGATTA | |
| | TTTATTAGCTTTTCAGGAATTCTTTTTAAAAATATAGAGACCTTG | |
| | GTCATCTTCCTCAGGTCCAAGTATCCATAAGAGAAGCAGACTTTT | |
| | CTCTCTTTCATAATGATGGTCTCTTCTTGAATAATTTCCCTGTGC | |
| | ATTTTCATTAAGGGTCTCACCCCATCAAGGGTGTGATGAGGACAG | |
| | TGATGAGGGTCACGGAGCCCGGCCTCCCACGGGGGGATGTGGGGT | |
| | ATGATGAGAGAGAGAAGCAGGTGCCCTCAGGCCCATATGGAGGCT | |
| | GGGGAGGGTTGTTAACCCTCCTGCCCTCCTGGCCACTGGGTGCAG | |
| | GACATGTTCTCTCTGAGGCTCACTCACCTATGGCCCCAACCACAC | |
| | AAAAATCTAAATGGGGAAGAAGCAGAGGATGACTTCATGCTGAGG | |
| | AGGCCCATCTTGCAGCGAGAAATGTGCTGTGGTGAGCACCTGCAG | |
| | CACAAAGAGCCTGGGGTGGGGGTGGAGGGGCGTGAGCGCTGCCTC | |
| | TGTGTGGGGATGGTGTGCGCAAGGCAGTGCTGAGGACCTTCAAGC | |
| | CAGTCAGGAATGAACCTGGGCTCTGAGACAGTGGAGGGAAGTGTG | |
| | GCCTGTCCCTGAGGGTCTGTGGGGGTTTCAACTGGGAAACCAGGC | |
| | CCTGGACCCCTACCCTGGGCCCTCTGCTCTGTGCTTTCTGGGGCC | |
| | CAGCAGCTGGGACCTCCCAGGAGCAGAAGAGGCTCCTCTAGCCCC | |
| | ACCCCACACTCAACTTTCCCCAAGTTCAGGCCCAGGGATAGCAGG | |
| | GGGAGGGGTGTGATTTGCATGAAGGGTGCTCTGTGCCCTCTTAGG | |
| | AGGAGAGGATAAGAGAGACTCTGTGCAGTTCTGCTCAGCTGTGGG | |
| | CTTAGGAAGCAGAGCCTGGGGCATCTCCACC | |
| 41. | CTCAGTCTCTCAAAGTGCTGGGATTACATGAGCTACCTTGCCTGG | CD19 |
| | CCCCTAACTTCTTTTCTTTCCTTTATCCAGCAAATACCTATTGAG | |
| | TGTCTACTAGATGCCTGTGCACATCTAGGGATGGTAAAGATTTCT | |
| | GTTCTCAAAGAACATTCTGGTGCAGGAGGCAGATGAGAAGACTAG | |
| | CAAATACACATGCATAATTTGAGATTGGGGCAAGTGCTGGGAATG | |
| | AAATAGGGGCATGCGATCAAAAAAGCATGTGAGTAAAGGGAATTT | |
| | GAGAAGGTTTTCTTTTTCTTTTTTCTTTTCTTTTCTTTCTTTTTT | |
| | TTTTTTTTTTTTTTTTGAGACAGAGACTCTGTCATCCAGGCTGGA | |
| | GTGCAAAGGTGCAATCTCGGCTCACTGCAACCTCTGCCTCCCAGG | |
| | TTCAAGCAATTCTCCTGCCTCAGCCTCCCGAGTAGCTGGGATTAT | |
| | AGGCATGAGTTAATTTTTTTGTATTTTTAGTAGATACTGGGTTTC | |
| | ACCATGTTGGCCAGGCTGGTCTCATTCTCCTGACCTCAAGTAATC | |
| | CACCCGCCTCAGCCTCCCAAAGTGCTGAGATTACAGGCATGAACC | |
| | ACTGTGCCCAGTCTTTTTTTCATATTTTTTGTAGAGATGGTGTTT | |
| | CACCATGTTGCCCAGGCTAGACTTGAACTCCTGGGCTCAAGTGAT | |
| | CCTCCCACCTCAGCCTCCTGAGTAGCTGAGACTACAGGTATGCAC | |
| | CATCATGCCCAGATACATTTTTTTTGGTATTTTTAGTAAAGACGG | |
| | ATTTCCCACATAAGGTAACTTGTGGATGTTGCCATAGCATTTCTA | |
| | ACTGTGAGGACAACCAGAGGTCACTTTCCTCACCGTCTTGGTTTC | |
| | AGTAGGTTTTGGCCGGCTTCTTTAGCGTATCCTGTCTTATCAGCA | |
| | GGGTCTTTATGACCTGTATCTTGTGATCCCAGTCTGGTTGACTTC | |
| | CTAGCTCATCCTGTGACTAAGAATGCCTAACCTCCTGGAAATGCA | |
| | GGCCAGCATGCCTAAGCCTCATTTAATCAAGCCCCTATTCAACAT | |
| | GGAGTCACTCTGGTTCTAACACCTCTGACTTAGGGTGGCTGCAGA | |
| | GCTGGAGGTGCAGATTGGGGCAGATCAGCCTCCTGAGAAAAGCAC | |
| | ACAGAGCCACTGCGGGGCTGTGAGGTGGGAGAGATGAACTCACAT | |
| | TGGCATTTGATCAGTGACTCTCCAGCTGTAATGAGAATAGAATGG | |
| | AGAGGCCTGGGACAGGAAGACAGGATGTGGTTGCTCATGCCATCC | |
| | TGGTGGGAAGATCTAGGTGAGTTACTGTGGGGTAAAGAGGAGAGA | |
| | GCAGATTCCTTGTCACTTTATGCTGTAGAATACATTGGACATGGA | |
| | GAGGGCTTGGAGCTTGTGTGTGAGAAAAGCAGATCTATCTCGGCT | |
| | GACTCCCAACAAACTGGCTGAGAATTTAGATGGAGGATTATTATT | |
| | AAATAAAAGAGGAAGACTGAAATGAATATTGTCATCGTATTTTTT | |
| | TACCCAGGGTTGGGATAGGTGGAAATGTGTGTGTGTGTGTGTGTG | |
| | TGTGTGTGTGTGTTTAAATATTCACTAATTTTTCAATTCAAAAAT | |
| | ATTTACTGCGTGACTACTAAGTAATAATATTATACAGGTTTTAGC | |
| | GTTACCATGGAGAATCAAATTGTCAAGGTCACAGGCATCTTTTTG | |
| | TTTTGTGTGGATCACTGTGAAAGATACTATGCTAAATTTTTTCTG | |
| | AATCATGCAGATGAAAGCATTAAACACATACACAAACATTTACGT | |
| | TTGTGTGTGTGTTGTGTGTGTGTGTGTGTGTATGTGTAATCTATC | |
| | TCAGCATACAATGCCACTGAAACTACACTTGCGGATTAGGTCCTC | |
| | GGAAAAATAATCGTGTTAACATTCACGATGCAAATAGAATGGTAC | |
| | CTGACACAAAGTAAGTGCTATAAAAGAATACTTGAGGCTGGGCGC | |
| | GGTGGCTTATGCCTGTAATCCCAGCACTTTGGGAGGCCGAGGCCG | |
| | GTGGATCACCTGAGGTCAGGAGTTTGAGACCAGCTTGGGCAACAC | |
| | GGTGAAACCCCGTCTCTACTAAAAATACAAAAATTAGTCAGGTGT | |
| | GGTGATGGGTGCCTGTAATGCCAGCTACTCGGGAGGCTTAGGCAG | |
| | GAGAATCGCTTGAACCTGGGAGGCGGAGGTTCAACAAGAGCAAAG | |
| | CCCATTCTCAAAAACAAAAAATTTGAATGGTTAAATCACATTGGA | |
| | CTGTGAGTAAAGGGCTTAGACAAGGTGTCTCGGAACTTTTGGGTC | |
| | ATCTTCTTGATCTTTCCATAAAACTCTAACCCTTCTATTCTTAAC | |
| | CTCCCCCATTCCCAATTTCACCCTGATGTGCACATCCTACCTGGT | |
| | GCCTAGTCATCCCGGTGGCCCCTCTCTGGGCTGTATCTAGGGAGA | |
| | GTGGTTCCATTTTGCCCAGGCTGGTTTCGAACTCCTGGGCTCAAA | |
| | TGATCTGCCCACCTGGGCTTCCCAAAGTGCTGGGATTACAGGCGT | |
| | GAGCCACCGCGCCCAGCCGAGATATATATATATATATATATATAT | |
| | ATATATATATATATATATATATATAGAGAGAGAGAGAGAGAGAGA | |
| | GAGAGAGAGAGAGAGAGAGAAAGAAAGAGAGCAGGCTCTTGTTGC | |
| | CCAGGCTGGAGTGCAGTGGTGCCATCTTGGCTCACTGCAACCTCC | |
| | ATCCCCCTGATCCTCCCGCCTCAGCCTCCCAAGTAGCTGGGACCA | |
| | CAGGCATGTGCCACCACACCCAGCTAATTTTTTGTATTTTTGGTA | |
| | GAGATGGGTTTTGCCATATTGCCCAGGCTGGTCTCCAACTCCTGA | |
| | GCACAAGCAATCTGCCTGCCTTGGCCCCACAAAGTGCTGGGAGGT | |
| | GTGAGTCACCTCGCCTGGCCTGAGATATATTTTTTTAAATAATAA | |
| | TATATAGATTTTTGAAGCACTGGGGGGCACAGGGAAGGCCTCTTT | |
| | GAAGAAGTACCATTTGAACTGAGACCTAAGTGATGAAAAGAACCA | |
| | GTCCAGCAAAGAACCAGAGCCCCCAGCAGAGATCCAGGAAAAAGG | |
| | GCTGCAAGTGCAAGGGCCCTGAGGCAGGGAAGCACTTGGCAAGGA | |
| | GAGTGGTGGAGGCACGAGGTGGAAAATGTAGGTAGGTCAGCAACA | |
| | CTCGGCCTACTAGGCCTTGGGTTGGAGTTTTTATTTTAGCTAGAT | |
| | GAAAAGCAACTGACATTTTTTGTTTTTTAAAAAATTTCTATAGAG | |
| | ATGGGTTCTCGCTGTGTTGCACAGGCTGGTCTCAAATTCCTGTCC | |
| | TCAAAGGATCCTCTCGCCTCGGCCTCCTAAAGTATTGGGATTACA | |
| | GGCATGAGCCTCTGTGCCTGGCTGTAACTGACATGTTTTAAGCAG | |
| | GGGAATGACATGCTCTAGTGAAAGCCAGTCTGGGCAGCTGGGTAG | |
| | CTAATGAGGGGATTAGAGAGATTTTGTTGAATGAAAGGCAGATTG | |
| | AGTCCTGCTACTCGCCCCCTTCATTCCCCTTCATTCATGCCTCAT | |
| | TCTTCCGCCTCCCAGCCGCCTCAACTGGCCAAAGGGAAGTGGAGG | |
| | CCCTGCCACCTGTAGGGAGGGTCCCCTGGGGCTTGCCCACAGCAA | |
| | ACAGGAAGTCACAGCCTGGTGAGATGGGCCTGGGAATCAGCCACT | |
| | GAGAAAGTGGGTCTCTTGGGTCCCTGAATTCTTTTTCTGAGTCCC | |
| | TGCAGCAGTGAAAAAGACACAGAGGCACATAGAGAGTGACAGAGA | |
| | AAGAGAGAGACAGAGAGGAGAGGCATGGGGCAGAATAAGAACAGA | |
| | TTTAGGAGTTAGAACTCCTGGGTTCTTTTAAAACAATTTTTCTTT | |
| | TAGAGACAGGGTCTTGTTGTGTTGCCCGGACTGGAGCACAGTGGC | |
| | TATTCCCAGGCATAATCATGGTGCACTGCAGCCTTGAACTCCTGG | |
| | GCTCAAGCGATCCTTCTACCTCAGCCTCCCAAGGACCTGGGACCA | |
| | TAGGCGTGTACCACTGTGCCTGGCTTTTGCCTGGTTTTAAACTGA | |
| | GGCAGTATGACTTGAGCTCTTAGGCATTAATTGAAGCTGTATCTC | |
| | ATTAACTGAGGGCTTATGATGTGCTGGACACTGGGCTAATAGTGC | |
| | TGAACATATTGTCATTTTTAATCTTCACAAACAATATTTGTATAG | |
| | GACTGTTTTCTTTTCTTTTTTTTTTTTGAAACAGAGTCTCACTCT | |
| | GGTGCCCAGGCTGGAGTGCAGTGGTGTGATCTCGGCTCACTGCAA | |
| | CCTCCGCCTCCTGGTTTCCAGTGATTCTCCTGCCTCAGCCTCCTA | |
| | AGTAGCTGGGATTACAGGTGTGCGCCACCATGCCCGGCTAATTTT | |
| | TTTTTTTTTTTTTGAGAAGGAGTCTATGTGCCCAGCATTGTTCTA | |
| | GAGCACTTGCAATTAGTGGTGAACAACACGGTCTCTACTCCAAGG | |
| | GGCTCACATTCTTGTGCAGAAAACAGAAATGAACAAATAAACACA | |
| | CAAGATCATTTCCCGTGGTAGTGAGAGCTGGGATGAAAATAAAAC | |
| | AGCGTGGCAGGGAGGAGGCAAGTGTTGTGAGTCTGGAGGGTTCCT | |
| | GGAGAATGGGGCCTGAGGCGTGACCACCGCCTTCCTCTCTGGGGG | |
| | GACTGCCTGCCGCCCCCGCAGACACCCATGGTTGAGTGCCCTCCA | |
| | GGCCCCTGCCTGCCCCAGCATCCCCTGCGCG | |
| 42. | TTTTCAATAACTATGAGGTCATAATTGCATTTTTTTCTTCAGCTA | CD20 |
| | TATTCCCACGTCACAAAAGCAAAGGGAGTGATCCAGTAATCTGCA | |
| | CATGGGAGTACATCAGACATTTTTATATCTTAAAACGAATAATAA | |
| | TAAATGTTTTGCCATTTAAAGCTGTGTCACTTGAACAAATTAATC | |
| | TTTGCGGGCTTAAAATTTTTAGACTAGAATGCAATAATAGTAATA | |
| | CCTACCTAATGGGTTTACTGTGAACCTTAAATGAAACATGGAAAG | |
| | TGAGTTTATCAGTGCCTGGAACTTAAAAATCACTTAGTAAATGCT | |
| | AGTTTTTGAAATTTATAATTGAGATTTTCTTGCTCTAAACATCAC | |
| | TTATTTTACCATTTACCAGCTTTCCAACTTGGGAATGGCTTCCTC | |
| | CATTTCCTGAAATTTAGTTTCCACAATTATAAAACAGAATCAATA | |
| | TTATATGTGTGGTAAGGCTTATATGTGGACAATTCTTGACATAAA | |
| | AGGAATTATTTAACTAATATTACCTTCCTAGCTGTTCCAAATGTA | |
| | TAGTCCCTTAGAAGAGAAAGAAGACCCTATCAACTATAAAGTTCT | |
| | TAAGTATTTCAATTTACATTTTCCTTACTATGAATTATTTATAAG | |
| | TGCAACAATGGGATTTTCTGACCTGTAACCAAGTTCTATTTGCAA | |
| | TTAGATCTTTAGCCTCACCTCTGACGAGCCCTATGTCTCCCTTAC | |
| | CAATTCTTTGACACCAGAACCCTCCAACAGCAGATAATTCTGGGT | |
| | GCCAGGGCCTTGCTGAGAAGTTGAGAACGTGGCATAATGTTACTT | |
| | CCTAATATTGGAGAGCCTGGGGACAATCACCTGGGTGAAAGAAAA | |
| | AGCCAAACAGAACCATTCCTTCTCAGCACTGCCAGGGACACAGCA | |
| | ATCACAAAAACTGACCCTGCCTTCAAGGGACAAGGAGACAATGGC | |
| | TGATTAAGGCAAGGACACAAATAATTATGATGTAGCACACAGTTT | |
| | TCATATAGAAGGCAATGATTAAAAAAATTGGAGTGCTGGGACGTG | |
| | CAGAGAATAAAAAGGAAATTCACATCAGGAAAATACTAACTAAAT | |
| | AAAAGTTGGCATACTTTTATGTAGACTTTAAGATTGTCTTTTGGA | |
| | AGCAAGGTCACTAGATAATGAAAATAGGCTTAATTCAAAGAGAAA | |
| | TATTTTAAATATGTTATCAACCTAATAAAATATTCTCAAACCTTG | |
| | GGGGAAGAGTGATGGTGATACATCAAATAAGATGCTATTGACAAG | |
| | TCCAAATTGTAGTGAGAATTTCAACACACTCAAGCACTAAAATGT | |
| | TGATCATTCAAATGAGCAAAGACAAGGTAAATGTTTATGCATTCA | |
| | TACAAATTCAAATTCAAGTTTATTTGGGAAGAATCATACATAGAG | |
| | CCACCTAAGTTATCTCATTGGCCACAAAGTAGCAAACAACCTGAG | |
| | ACTGACTGGGGAAATACAGGGCTCAAACAGAGAAAAGGATGCAAA | |
| | AGACCAGGTTGTGCTGCCACAACTCAAGCCCCTTTGCTGCCATAC | |
| | ATTTGCCAAGTCCTGACCCTGTTCCCATGCCAACTAAAATGAAGG | |
| | CACATGCCTCTAACCACCCCACCATCCAGGACTCAAACCTCAGGT | |
| | TTCACAATCATCCACAACTCAGACCCTAGGTAATTGTCCTCCCAT | |
| | CACCAATCACTCAAGCATTTCAGCAGATATGCATAGACCCTCAAT | |
| | TAGAATGTTAAATGGATGATGGATCTTCTGTTCCCACTCTCCTTC | |
| | ACTCTACAGCATCTCTGAGAGGTGACTGCATGACTTTACTTGAGT | |
| | ATTCTTGAAGAAGTAAATTCTCTATTGAAATGGTAGCCAGTTCCC | |
| | CTAATGAACGGCTCCAACAATTTTTTTTTTTAGAGTGGCATCTTT | |
| | TTAAATATCACACCACAATCAGCCTCCCCAGAACTACTTCTACCC | |
| | AGTGGATCTAACCCTATGGTTCCAAACTATCTTAAATTTAAAAAG | |
| | AGAGGCAGAAAGAGAAAGAGAGAAGAGATGAAAGAGAGAGACAGG | |
| | AATGAACCTCGAATTAGGCAACAGAGCTGCCTGCCATATTTCATC | |
| | CCTTTAGATTAAATAAACAAATAATTACCTAACTATAGCTCTGTG | |
| | TCTCTAATAAAATGAAGCAATGAACAGGGAAATAAAAATGTAATT | |
| | AAAAATCCCACATATGAACATAAAAGTTGAATATTGTAGGGGGAG | |
| | AGAGAGGACATTATCTTTGGGCAAAACTTTGATTCACATCCCAAA | |
| | TATTTAGACTCTCCTGACACTACCAACTTCTTCCATTTCCCTACT | |
| | TGATCTAGTACTGTGCTTGATGACCCTGCAGCTCCAGACTAGACT | |
| | AAAAAGAGAGGGGACACAAGGGAACTTGACAGACAGAAGAAAAAG | |
| | GTGGGTTCAGAGGTAAAAGTGCTATTTCAATTTATATTTCCAAAT | |
| | ATGGAGGCTAATTCTTGCTTTGAGTGGCTGGGTACATGTACTATG | |
| | GCACTAAAGTATAATTTTAGGCCATTTCTGCTAAATCTGACACAA | |
| | GATATATACATGGTTCATAAGAAACTTAAGTGTGAGCCAATGAGG | |
| | ACAATATTTGGGGACCCCTAAGTATAGTGCTGCCAAGTGTACAGC | |
| | CTCAGTGATTCTTGAATCTCTGCATGGATGCCTCCTGCCCACCTA | |
| | CTCAGCCCTAAAAGTGAAGCCAGAAGGTAAAAGTCAGTGCTAACG | |
| | GCCCATCTTTGACCAACTTCTAGAATCTTTCTTGTCCTCAGGGAC | |
| | CTACCATCCACACCTGCTTATAGGGTGGGGGCTGCAGAAGTTCTT | |
| | CTAATTAAGGTAGCATGAGCATGCCAGGCAGTCCCCTGGGGTCTT | |
| | TTTCAAGAAGTGAAACCTGGTAAGGCAGAAACTTTTTTTGCACCT | |
| | CCTTCAGCTATGGTAAGTGTTAAACCAAAGTAATTGGAGCGAAGC | |
| | CCAAGGTAGCAGAAGCTACTGATTTCCTGTCACCTGATGTCTATC | |
| | AGCGATTTCATCTTCAGGCCTGGACTACACC | |
| 43. | TCCAGGCTCAGGTCAGCTGTCGGCCACCAGGCCAGGGTTTGCCAC | TH |
| | TGGCAAGTTGGATTTTGTGTTACGTGCCTAACCAGGAGTIGTCAC | |
| | CGCTTGAGGCTTAACCTGGTTCTGGGCGGCTCGGTGTGTGTGTCC | |
| | CAAGGCCATGCCACACAGTCCGGCTCCTCAGCCACTGGTCAGAAG | |
| | GTGCCCCCAACCCTGGTGGCCTCATCCAGGTTTGTAGGATGGGCC | |
| | CTGAGTCTCACTGGCCCCTGAGCGTCCAGGACACTGGCCAGAAGG | |
| | GTCCGCTCCACCCCCACCTCCTGTTGCCTAGCAACAAGGGCGGTG | |
| | GGTCGCAGTGGCCCTCGGGCCACAAAACTGGCTCTGGGAGCTGGG | |
| | GGGCGGGGAGTCTGGCTTTAATGTCATTTGGGAGCTTCTTCCGGG | |
| | AGCTGGGTCACCTGCCTCCCGGGGCACAGTGGGGCCGGAGTGACT | |
| | AATGCAATCAGTGGTCACCTGAGCCACTGTGCTGGGTGGGTGGGA | |
| | GAGGGCACACTGCCCTGCCCCTGCCTCTTGTGCTGCCCTGCTGGG | |
| | AGCCCCCCACGTGGGAAATGGTGGGTGAGGGGGATCCAGGGGATG | |
| | GGCAAGGCAAGAGGCACAGTGGGGCTTGGTAGCCTCAGCCCTAGG | |
| | GGTGGGCTCAGGATCCCTCAAAAACACGGGGGTGTGGCTAGATGC | |
| | CATGGGGGCTGAGCTGAGGCCTGAGACTCCCCTGCTGCATCCTGT | |
| | GCCGGCCTCGGGGGCCTGGGCAGCTGCACCTCTGCTATAGAGGGG | |
| | TCAGGAACTCAGCCCACACAGCCCCACCCACAGGTGAACTTGCCC | |
| | CAGGGACACGAAGGCCACCAGCTCACCTCAAACACCTTCACAGCT | |
| | CGGGACAGCGCCGAGGGCTTGGTGGCCCTCGGGGAGAAGAGCAGG | |
| | TTTAGCACGGCCTTCCCCTCCTTCTCCTCAAAGGCCACAGCCTCC | |
| | AGGGGGTCCCCGGGCTCCGAGGGGACTGCAGCGGCCGCTGCTGCC | |
| | ACCGCCGCCTCCCGCTCCTTGCGGGCGTCCTCGATGAGGCTCTGC | |
| | CTGCGCCCAATGAACCGCGGGGACTGTGGGGACAAGGGGCACCCA | |
| | TGCCTCCTCCACCTGCTGAGACCCGGGGACCTCCACCCACAGCTG | |
| | GTCCCACAGTCGGGCAGCGCTGATGGCACACAGAGGCAGGGGATG | |
| | AGAGCACGTTTTTGAGCGCCTACTGTGTGCCTGCTGGGGCAGATG | |
| | CTAGCCGAGGTGCCTGCGGGCATTGCACGCCCTTCCCGATGGGGG | |
| | CAGCCCAGTCAGAAGCAGTGGTGGTGGGGGAGGAGTGGGGGCTGG | |
| | GAAGGTGCTCGCCTGCTCCCCACTCTGTGGCGAGCAGACAGACAG | |
| | AGACGCTGTGTCACTCATCCCCAGGCCAGGCGCTAACTGGATTAG | |
| | TGATGGGAAGAGGGCGATGTCTTGATGGACAGTGGCATCGGCTGC | |
| | CGGGGAGGGAACGGCAGCTCAGGAAGGAGCTGCCCCCACAGGGCC | |
| | CCAGTGAACAACCCCCCTACCCTCCTGGCTGCCGCACAGGGGACC | |
| | CTTTCTCACACTCCCATCAGGACACCTGTCCTCGGGCCCTGCCCC | |
| | TCTGTGCCACCCCGCAGGCGCCCGCTCCTGGCTGCCTTGTCATCC | |
| | CTCCACCCTTTGTCATAGCTCTGGGCGCAGGTGTCTCTCCCTGGG | |
| | CTCAGCGCCTCTGACCCCTCGATCCCAGAGCCGTCCCAGGCCTGG | |
| | ACAGAGGGGGACTTGGCAGACACCTGGGGCTCATCCCTTGGAGCT | |
| | GAACTCCCAAGAAGGCTCTGGGCCCCTTGTAAGAGAAGAATCAGC | |
| | TTCATCCTGAGCTTCCAGGGTTGTGGAACATGAAGGGGAGCAGCA | |
| | GAAGCCCCTCCCAGAGTCCCCTCTTACTTACCCTTGGGGTGGGGG | |
| | TGTAGGATGCAGCTGGGGCTGCAGTTCCAGGCCACGGAGAGCCTG | |
| | TGAGGCTGGGCCCCGGGGCGCCCTGGGGAGGGGATGCCTGATGGG | |
| | GAGCCTGGTGGGGGAGGGTAGGGGAGGGCGGGGGAGGACGGGGGA | |
| | GGGCGCCCTGTGTCCCTGAGAAGGTACCTGGAAATGACACTGCTA | |
| | CAACTCACACCACATTTCAATCAAGGTCCATAAATAAAAACCCAT | |
| | TTTAAATGTGCCAGGGAGCCCAAGGTTCTGAGTGCCCAAGGAGGC | |
| | ACCGAAGACCCCTCCTGTGGGCTGAAAAGCTCCCGATTATCCAGC | |
| | CTGGCCCACACAGTCCCCTGTACACAGGGCTTCCGAGTGCAGGTC | |
| | ACAGGGAACACAGACTCCATGGTGAATGAATGAATGAATGAATGA | |
| | ATGAATGAGGGAAATAAGGGAGGAACAGGCCAATGGGAATCACCC | |
| | CAGAGCCCAGATACCCTTTGAATTTTGCCCCCTATTTGCCCAGGA | |
| | CCCCCCACCATGAGCTGCTGCTAGAGCCTGGGAAGGGCCTTGGGG | |
| | CTGCCTCCCCAAGCAGGCAGGCTGGTTGGGGTGCTGACTAGGGCA | |
| | GCTGGGGCAGAGGGAGGCAGGGGCAGGTGGGAGTAGGGTGGGGGC | |
| | TGGGTGCAGCAGCCGGGGACCTCTGGCCATCTTGGATTTTTTGGA | |
| | TGGATTTGTTTCCACATTCCGATCGTTAAGATTCAAGATGAAACA | |
| | AGACACAGAGACCCACACGACCCCCGAGAGAGGTCGGCCTAAGAG | |
| | GGGCACACACAGGGACAGGCATCACCTCACCCTCCCCCAACAGGG | |
| | ACTCAAACACCAGGCACAGGGGATGCCGCTGTGCCCAGGCCTCCA | |
| | CATCCACGCCGCGTCCCAGGGGTTTGCATGGACCCTGAGCCTGGG | |
| | GCTGCCAGCCAGGCTGGGGAGTAGCAGAGGCAGCTGGCACCAGCC | |
| | CTGGGCTCCGGTCCACTGCGGCCGCCGGGCACCTACCTGCCCTCT | |
| | TACCATGATGGCCTCTGCCTGCTTGGCGTCCAGCTCAGACACGGC | |
| | CCTGCGGAAGCCCTTGGCCTGTGGCGTGGTGGCGTCGGGGGTGGG | |
| | CATGGCTCAGTGTGGAGGTCCGGGCTCCGTC | |
| 44. | TCCAGAGGTGTCGAAATGTCCTGGGGACCTGAGCAGCAGCCACCA | TUBB3 |
| | GGGAAGAGGCAGGGAGGGAGCTGAGGACCAGGCTTGGTTGTGAGA | |
| | ATCCCTGAGCCCAGGCGGTAGATGCCAGGAGGTGTCTGGACTGGC | |
| | TGGGCCATGCCTGGGCTGACCTGTCCAGCCAGGGAGAGGGTGTGA | |
| | GGGCAGATCTGGGGGTGCCCAGATGGAAGGAGGCAGGCATGGGGG | |
| | ACACCCAAGGCCCCCTGGCAGCACCATGAACTAAGCAGGACACCT | |
| | GGAGGGGAAGAACTGTGGGGACCTGGAGGCCTCCAACGACTCCTT | |
| | CCTGCTTCCTGGACAGGACTATGGCTGTGCAGGGATCCCAGAGAA | |
| | GACTTCTGGGCTCCCTCAACTCCACCCCCACAGCCATCCCCCAGC | |
| | TGGGGCTGGCTGCCAACCAGACAGGAGCCCGGTGCCTGGAGGTGT | |
| | CCATCTCTGACGGGCTCTTCCTCAGCCTGGGGCTGGTGAGCTTGG | |
| | TGGAGAACGCGCTGGTGGTGGCCACCATCGCCAAGAACCGGAACC | |
| | TGCACTCACCCATGTACTGCTTCATCTGCTGCCTGGCCTTGTCGG | |
| | ACCTGCTGGTGAGCGGGAGCAACGTGCTGGAGACGGCCGTCATCC | |
| | TCCTGCTGGAGGCCGGTGCACTGGTGGCCCGGGCTGCGGTGCTGC | |
| | AGCAGCTGGACAATGTCATTGACGTGATCACCTGCAGCTCCATGC | |
| | TGTCCAGCCTCTGCTTCCTGGGCGCCATCGCCGTGGACCGCTACA | |
| | TCTCCATCTTCTACGCACTGCGCTACCACAGCATCGTGACCCTGC | |
| | CGCGGGCGCGGCGAGCCGTTGCGGCCATCTGGGTGGCCAGTGTCG | |
| | TCTTCAGCACGCTCTTCATCGCCTACTACGACCACGTGGCCGTCC | |
| | TGCTGTGCCTCGTGGTCTTCTTCCTGGCTATGCTGGTGCTCATGG | |
| | CCGTGCTGTACGTCCACATGCTGGCCCGGGCCTGCCAGCACGCCC | |
| | AGGGCATCGCCCGGCTCCACAAGAGGCAGCGCCCGGTCCACCAGG | |
| | GCTTTGGCCTTAAAGGCGCTGTCACCCTCACCATCCTGCTGGGCA | |
| | TTTTCTTCCTCTGCTGGGGCCCCTTCTTCCTGCATCTCACACTCA | |
| | TCGTCCTCTGCCCCGAGCACCCCACGTGCGGCTGCATCTTCAAGA | |
| | ACTTCAACCTCTTTCTCGCCCTCATCATCTGCAATGCCATCATCG | |
| | ACCCCCTCATCTACGCCTTCCACAGCCAGGAGCTCCGCAGGACGC | |
| | TCAAGGAGGTGCTGACATGCTCCTGGTGAGCGCGGTGCACGCGGC | |
| | TTTAAGTGTGCTGGGCAGAGGGAGGTGGTGATATTGTGTGGTCTG | |
| | GTTCCTGTGTGACCCTGGGCAGTTCCTTACCTCCCTGGTCCCCGT | |
| | TTGTCAAAGAGGATGGACTAAATGATCTCTGAAAGTGTTGAAGCG | |
| | CGGACCCTTCTGGGTCCAGGGAGGGGTCCCTGCAAAACTCCAGGC | |
| | AGGACTTCTCACCAGCAGTCGTGGGGAACGGAGGAGGACATGGGG | |
| | AGGTTGTGGGGCCTCAGGCTCCGGGCACCAGGGGCCAACCTCAGG | |
| | CTCCTAAAGAGACATTTTCCGCCCACTCCTGGGACACTCCGTCTG | |
| | CTCCAATGACTGAGCAGCATCCACCCCACCCCATCTTTGCTGCCA | |
| | GCTCTCAGGACCGTGCCCTCGTCAGCTGGGATGTGAAGTCTCTGG | |
| | GTGGAAGTGTGTGCCAAGAGCTACTCCCACAGCAGCCCCAGGAGA | |
| | AGGGGCTTTGTGACCAGAAAGCTTCATCCACAGCCTTGCAGCGGC | |
| | TCCTGCAAAAGGAGGTGAAATCCCTGCCTCAGGCCAAGGGACCAG | |
| | GTTTGCAGGAGCCCCCCTAGTGGTATGGGGCTGAGCCCTCCTGAG | |
| | GGCCGGTTCTAAGGCTCAGACTGGGCACTGGGGCCTCAGCCTGCT | |
| | TTCCTGCAGCAGTCGCCCAAGCAGACAGCCCTGGCAAATGCCTGA | |
| | CTCAGTGACCAGTGCCTGTGAGCATGGGGCCAGGAAAGTCTGGTA | |
| | ATAAATGTGACTCAGCATCACCCACCTTAGCCCCTTCCAGAAAGT | |
| | GCTTGAAGTTTGCGGGTGGAGGGATGGGGGAGGGGAAGGTGGGCA | |
| | GGGGTGAGAGTCGAGAGGGAAGAAAGGAGTCCCGGAAAACGTGGC | |
| | TGCCTCCCCAGGTGAGGAAGCCACAGCCCCAGAGGCCCCAAATGC | |
| | CTGGGGAGTGTGGAGGTCCCAACCAGGCTTGCGCTGACCCTGCTT | |
| | CTCGGTTTTCTCTCCGTGCTGACAAACCCCAGCCTAGAGGAAGGA | |
| | CGAGCAGGTGCAGCAGGGCCCCAGTCCCCTCCACTCTTGACGCTG | |
| | TCCTAGCTGCAGAAGAGGCGGGTTCCCAGCCTTCCCTGTGACCAC | |
| | ATGTGACCTCAGCCGGGACACATCCCTTTGCTGGCCCTGGCCCTG | |
| | AGTCCCTCCAGCCATGATGAGCCGTGAATGGGACCATCCCTGTCC | |
| | ACTCTGAGATGCCTGGAAGGGGGCTCAGTGCAGGTGGGCTGGGGG | |
| | CTGGGTCTGCTGTCTGCCCAGCACTGCCATTCTGGGAGTAGGCAG | |
| | GTGGGGAAGGGGTCGGGGGTGGAGGGTCTGTGTTCAGCCAGTCCT | |
| | GGGAAATGCTTGATGTGAGGCTTCTGAAGATGGCAGTGAGGCAGA | |
| | GGCCCAGCCGGCGGAGAAGTCCCTGGGAGTGAGTACCTGGGGATG | |
| | AACTATGCTGCCTGGTGCTGGGGAGCAGTGGCGCCCCTGGGCCAT | |
| | CCCTCTGCTGAAACCTGGGCGTCTCGCTGAAGAGACCACCTCCAT | |
| | TTCCTCTGCAGAGACTGAGCACTCAGTCAGCCCCCTTCCTGGGAC | |
| | AGGCTCAATGGAGGCTGCAGGGCCATCAGCCGACTCCTACGCAGG | |
| | CTCAGTCAGCAGCCCCCTGGCCAGCCCCACCCCTGACTGCCGGCC | |
| | TCAGAACTGGGAGCTGCTTCCTGGCAGGGCCCGCCTCTGCTGGGA | |
| | GACCGGACGGTGAGTCAGCCTTAAGCCCGGC | |
| 45. | GTTCTCGAACATGTTGCCCGAGTCAGGGTGCAGGGTCCAGAAGGA | FOXA2 |
| | GCCCTTGCCGGGCTTGTCGGGCGAGCGGGGCACCTTCAGGAAACA | |
| | GTCGTTGAAGGAGAGCGAGTGGCGGATGGAGTTCTGCCAGCGCTG | |
| | CTGGTTCTGCCGGTAGAAGGGGAAGAGGTCCATGATCCACTGGTA | |
| | GATCTCGCTCAGCGTCAGCATCTTGTTGGGGCTCTGCTGGATGGC | |
| | CATGGTGATGAGCGAGATGTACGAGTAGGGCGGCTTTGCGTGCGT | |
| | GTAGCTGCGCCTGTAGGTCTTGGGGTCGCGGGCGCGGCTCAGGCC | |
| | CGCCTGCCCGTACATGGGGCTCATGGAGTTCATGTTGGCGTAGGG | |
| | GGCCAGGCCGCCCATGGCCCCGGCCGCCTGCCCCCCGAGCGGGCT | |
| | CAGGCTGGGACTCAAGTGCGGCCCCATGCCCGCCACGCCGGCCGC | |
| | CCCGGCCGAGCCGCCCATGCCCGCCATGGCGCCCGCGCCGGGGGA | |
| | CATCCCCGCCAGGGACGGGCTCATGCCAGCGCCCACGTACGACGA | |
| | CATGTTCATGGAGCCCGCGCTCATGTTGCCCGAGCCGCTGCCCAT | |
| | GGCGGCCGCCGACATGCTCATGTACGTGTTCATGCCGTTCATCCC | |
| | CAGGCCGGCGTTCATGTTGCTCACGGAGGAGTAGCCCTGCGGACA | |
| | GAGCCCCGGGAGGGAGGCGACAGCGTTAGCACCGCGGCTGGAGGG | |
| | TGCCCAGACCTCCCACCCACCGCCCAGGCCTCCGCGTCCGGGGAG | |
| | GCCTCCGGGGAGCCCTTTCGTCCCCGATGGCCCAGTCTCCGGACT | |
| | CCGAGTCTGTTTCATTCAGGGAGAGTCATGATCGATCGCCCTTGG | |
| | GAAGGTGGCGAGCGGATTGCTGCTTGGGCATGTGGCCCGTTCCTA | |
| | GCCCTTCTCCACCTCGCCCAGATTCTAGAACAGGGGTCCCATGGA | |
| | GTACTCCCAAGCCAGCACCCCAAATACATTCCCCTACCCCAGAGT | |
| | CTCCCCAAACTCTCCCACCAGCATACTGAGCTGGCGCCGAGGCCC | |
| | GAAGTCCCCGGACGCCACCTCCGGTCGCAGCAAGCAACCTGCGGG | |
| | ACAACGCGCGGGGCGCCTCGGGCTGCCTCCAGCGAAGACTGTCCC | |
| | GGGCCCCCTCTGTCCGGTCTCCCTCCAGGGACCCCCTCCCTTGTC | |
| | CCAGGGAAACTGCAAGGCAGTGCCTGTGCGGAGTGCGGGGGCGGC | |
| | TGGAGGCACTGAACGGGGCAATAGGGAAAGAACCGAGACCTGAAC | |
| | CACACGCGGCTGAGCTTTGCAGGCAAAAACTTTCTTTCTCTTTGT | |
| | CCACTAGTCTTTGACTCTGCCTCCACTTCAGCCCCCAACTCCTAC | |
| | CCATCTTCTCCTCCTCAGCTCCCCACCCCCTCGCCGGCCGACCTC | |
| | CCACCCCTCCCCAGCCGCGCGCTGCCAAACATAACTCTGTTAGGA | |
| | TAGTGCGTGGCTCGGCCACGAAGAGGATTTGGAGGCGCCGCAAGT | |
| | CAATATTTGATCACAAAGTTAATATTATCTCAAGGCTAACAGTGT | |
| | GTCGTATAAAAAAGAGACCCATTTGATTCCAAGGAGGGCGGAAAA | |
| | GGCGGCTGCCCAGAAAGGCTGGGGTTGTGGGGCGGGGTGGGGGGG | |
| | TGCCAGCGAGGGAAGCGGTCCTGAGGTTGGGGAAGGAGCGAGCGC | |
| | CGCCGCTCCACTTCCCCCTGGAAAAGACGAGCGCTTACCTCGGGC | |
| | TCTGCATAGTAGCTGCTCCAGTCGGACGGCTCGTGCCCTTCCATC | |
| | TTCACCGCTCCCAGCATACTGGAAGCCGAGTGCATGGCAGTTTAA | |
| | AATTTAACAGCCACAACAAACGACCAGCAATCACCCCCCACCCCC | |
| | ACCCTCTTTTAAAAAAAAGTCAGCCAAAGCACCGTCCCCTCCTCC | |
| | CTCCCTCTCTCGCGCTCCCTCTCCCTGGGCTCCACTCCCTCTCTC | |
| | TCCCTGGGCAGGCCGGAGGCGGTAGTTGGAAGTGGGCGGGAGGTG | |
| | GGGGGGGGCGAGGAGCGGAGGAGGCCCAGGCCAGCGCCCCGCGGT | |
| | AGGGAGCACCCGCCGCCGCCGCGCTCACGGGCTGCCGGGTGGCGG | |
| | CTGAGGTTGGCAGTGCCGAGCTGCCCCGAGGCGGCGGGAAGCGCG | |
| | CGGCGCGGGGGCTAGTGGGGGGGTGGGGAGGAGGAGGAGGAAGGA | |
| | GGAGGAGGAGGCGGTGGTGGTGGTGGAGGAGGAGGAGGAGGAGGA | |
| | GGAGGAGGAGGAGGAGGAGGAGGTGTGGACCGCGGAGCGGACAAG | |
| | TGCCGCAGTGACGTGGGAGGCTGGTGATATAGCGCGGCGCGCTGG | |
| | CGCGGGCCTCCAATCCCCAGACCCGGGGCAGCCCATTTGAATAAT | |
| | CAGCTCACACCTAGGTGAGAGGTAGCCGCAGCCGGCGCTCCGCAC | |
| | CTGCCCCTCAGCGCCTGCCGTCCGCCCCACCGCCGCGGCGCCCCG | |
| | CACTCCTGGGCGGGCCAGGGGAGCGGGCTGGGCGGGCGATCGGGC | |
| | ACGCGGGATCCCTGGTCGAGCCCCCTTTCCTCCCGGGTCCACAGC | |
| | GAGTCCCCTGAGGAAGGAGGGACCTGGGAGGAAACCACCCTCTGG | |
| | GGCGGCTCCGGCCTCCAGCCCCCGCCCCGTCTCATCGCGCCGGGC | |
| | GCCCGGTGCGCCTGTCGGAGGGCGCGTCCAGCCTCAGTGCCCGGC | |
| | AGATCCCTATGAAAGCCGGATTTATTTATGCCGGGTTTCTTCGCT | |
| | CTCAGTGCTCATTCTCTGTCAAAACAGCAAGTCCATGACAGCCAA | |
| | TTTGCAAAGCGCTGTCCTATTTAGAAAAACTACGTACACACCTTT | |
| | AACTCGCCCGCTGCTGCTCCTGCGAGGCCCCTCCCTGTTACAGTT | |
| | CAGACCCGGAACGGCCTCGGGAGAAGCGCGGGGCGCACGGTCTGG | |
| | CCGCCTCGGCTCTCCGACTCCTCAGACACCGGCCGCCAGGGACCC | |
| | GCAGTGGGGCGGCCGGCGCCTGGCGCAAGCAGCCCCTCTAGCAGC | |
| | GGCCGACGGTTGGGAGGCTGAGATTTGTCTC | |
| 46. | TTGATTCCTTCCAGTTCTAGACCGAGAGGAAACTATATCTCCACC | GIRK2 |
| | CTCTTCAGTCCTAGGACCCTTAGAAAGAATTTGGCTGGGCCGGAT | |
| | ACGGTGGCTCACGCCTGTAATCCCAGCACTCTGGGAGGCTGAGGC | |
| | AGGCGGATCACAACGTCATGAGTTCGAGACCAGCCTGACCAACAT | |
| | GGTGAAACCCCGTCTCTACTAAAAATACAAAAATTAGCCAGGTGT | |
| | TGTGGTGTGCGCCTGTAATCCCAGCTACTCAGGAGGCTGAGGTAG | |
| | GAGAATCGCTTGAAACCGGGACGCAGAGGTTGCAGTGAGCCGAGA | |
| | TCGAGCCACTGCACTCTAGCCTGGGTGACACAGCGAGACTCCGTC | |
| | TAAAAAAGAAAGAAAGAAATAACAAACTTGGTTGGCTGGCCTCTC | |
| | GCTGACCAGGGTGCTTCCACAGCTCCCTTCTGCCATCTATGCCCA | |
| | ACAAAAAGTTAAAGAGAAGAGCGTGAGAGGGCAAGGGATGCATGC | |
| | TTTTGAAAGCCAAAGGGACTCAGCTGAAACTAGCAACCCTCGTCA | |
| | GAGGCGGATCGGGGTGTGTGTGTGTGTGTGTGTGTGTGTGTGTGT | |
| | GTGTGTGTGTCTGTGCGCGCGCGCGTCCTTTTTCTCCCCCTGAGG | |
| | ACAGAATCCTGTCTCCACTCTCAGTTCAGCTCAGGTCTTTCCCTG | |
| | TCCCAGTGTTCTTGATTCTGAATAGGTCCTCTGATGCACCGCCAT | |
| | GCATAAAATGGATGATTTGCAAGCTTCTTTGCCTCACGTAATTCC | |
| | GTGGGTAATTTGATATCCAGATTCTGCACGAGCCAAAGACTCATT | |
| | TATTTATAGAAAGTGATTGACCTCCAGCGACCAAAGGAGTGGGTA | |
| | CTGCTTGCTGGGGGCTGCAGACAGGTGAGGCTTCACCCTCGGCGC | |
| | CTGGGCTCTGGCCTTGGTGCTGAATCCGGAACCCGCTGAGGAACC | |
| | ACCACCTCGGCGGCCCCCAGGCAACTTTCGGGAGGTTTCAGGGGC | |
| | ACCCGGCCAGCTCTGGGCGCCCTCTCGAGCGTCGCCGTCAGGGAG | |
| | TTGGGCACAACCGATGCCCTGGGACCCGCCACAGGGCACGCGGCT | |
| | GGCGTATTCCTTTGCCTGGAGCCGGGAAAGTTTTGTCCAATTTTT | |
| | TTCCCCTATAACTGGAGCTGCGGTCTCCCCTCCCACCTGGGTCGC | |
| | CATGGAGACTCGAGCTCTTTTTCTTTCTCCAGGCAAAATGGGCGA | |
| | AGCTCAGTTAGCTCAGCCCTGACTGTAGCAAGCCCTTTTTTCTCC | |
| | CTAAGCAGTTCCTGGGCATCACCCTTTTCCGTTTCTTTTTTTCAG | |
| | CAAAAGAAAGGCAGGCAGGGAACAGGGCTAGCAGGATGTGGCATT | |
| | GCTCCCTGTACCGTAGGGCTCCCCGGGACCGACAGACTTTGGATA | |
| | ACAGCAGTTGGGAAACACTCTAAACATATAATACATCTCCAACTC | |
| | CCAAACACTGGAGTTGTGGGGTTTTTTTTTTTTTCTCTTCCTTTT | |
| | TTTGGGGGTTGGAGGCGGAATTCTTTGGAAAATTCTGGTATTTTG | |
| | TTTCTCCACTAACCTCACCCTCTGTGGCTGTTTCTATTAAGATAT | |
| | ATTTTCCCCCTGACAATCTCTCCATATTGCACCTAGTACAACAGA | |
| | TGTACCTGTGGATCCACTGGGCTTTTATTGGGTTTTGGGAATACT | |
| | GATGTTATTTCACTTAAAAACTAATCCAAGCTTAATGTTTTGATG | |
| | AAAATGAAAATATATCCACAGGGAAGCAAATATCCACAGCCACCC | |
| | AGTCCTCTAAATGCCACACACATCTCATTTTATCTAAAGTGTTTT | |
| | TTCTTCCCTTCCAATTACAAAATAATTCAGTTTACTCCTAGGAAG | |
| | ATCTTCCTATTCACTGTTTTTAGTCATGCTAGCACATCTTCGGTT | |
| | TTAAGGATAGAGAACAGTTAGGAGTTACAGTATTCTGTTTCCCAT | |
| | TCTCCGCATGTTTCTCCCCAACCCTCACTTTTCCTCCCTTTCGTG | |
| | TTGTCTCCTCTCCTCTCCATGCAAAGCAGAACAAGAAGACTGCCT | |
| | TCCACAAAGACAATAGGCTTTTCCCAACTGTAAATGACTACATCA | |
| | CAAGCCGCACCTGTCAGTTAATCAGTGCATTTAAGTCTTTGCCAG | |
| | CGGATGTTGAAATGATGTCTGCAATAACTGACTGGGTTTCTGCGA | |
| | GCCCAGGGATGCAGACCCCTCCTTCCCCCGCCCCCCGTGCGAGTT | |
| | TCAGTCGCTAATGAAAACCGTATGAAACAGATTCCCCTTCTGTTA | |
| | TGTAACCGGCTTCTCCCTCGCCTTTCGGCTGACTTGGGCACAATG | |
| | TTTCTCATGAGAGTGGAGATGCCTGCGGCGACGGCGGAGCTGGGG | |
| | AAGGGGACGCGGGGACCCGAGAGCACGGAACGCCTCGCCCAGGTG | |
| | GACGCCAGCTCCTCGCCTGCGAGCAGCTCAGCGCACCGCTTACCT | |
| | GGCTGCGGACGGGGTGGCTTCACTCAATCATGATCTCCTCTCTTG | |
| | AAACGGAGCAAGACTGAACAATTCACTACACGACGGATGGCGAGG | |
| | GAAGGAAATGCAAAAAAGAAAAGGAAAAGAAAAAAACTGGAATCA | |
| | GATGGTCAAATTTCACATCAGCACCGAGAGGCTGTTAGGAGACTC | |
| | CATTCTGCTCGCGGCTGCTCCGGCTCCAGGTCCGGCTTCCCGGCG | |
| | TCCGCGGGTCTCCACCCCTCCGCCCGCCCAGCCTCTCCAGCCAGG | |
| | TGCGGCCGTCTCCGGACTGGCTCCCTCTGGCCGAGCGCGGAGAGC | |
| | AGGGCTGGGCGCTGGGGCCAAGGGAGGGGCGGAGGGGCGCGCGTT | |
| | TGGGCAGGGACGGGGGAGGGAGACTGGGTGGAATGAAGTGGGGAA | |
| | AGATAAGAGTGGCAGAAGAAAAAAAAAATCCCCGGTTAGGAGAAA | |
| | AGTGGTGGACCACGCAGAGAGACGTCATGAAATCCGCAGATTCAA | |
| | TGAATGCTACTCAATTCCAGCCGACTGGCTGAGCCCCGCTGGCAG | |
| | CGCACGAAGCGACGCGGCTCCGAGATAAAAG | |
| 47. | CTTTCTGGCTCTGTGGCTGGGTTCTGGAGGGTGCAGGGGACACAG | DAT |
| | TCACTTTCCAGGGGCCTCCTCTGCCACTCCAGGCTCCAGGGCCTC | |
| | CACATGCTTTGCAAAGGCTGCCAAAGTCAGCCGCAGGCTGTTCTT | |
| | TGGACCTTTGAGAATTTTCTTTCCAAAGCGAAGATAGCCTCTGGA | |
| | AACAGGAGGCAGAGCCAAGCTGCCCCGTCTGCCGCCCCCCACCCC | |
| | CCAGCTTCTTCGCGGGCCTCCCTTTCCTAAACTCTGAAATGCAGG | |
| | CGTGGGACAAGGCAGCTCCGAGTCCTGCTCAATGGTTTTGTGACA | |
| | TCCTCTGGGAGGATCTGCACCGGCCGTGAGCTCTCACAGGGAGCT | |
| | CCGTCTTCACGCATGGGAACAGCTTCATCTCGTTTCCGTACGTGC | |
| | CTTGGCCCCGGCTGCCCCTACGACCCCCGCCCGGCCAGCATGCTC | |
| | AGGGAGGCTGAGATGGGACTTACCGCCACCATTTTTGTAGCACAG | |
| | GTAGGGGAACCGCCAGACGTTGGCCAGGTCCACAGCAAAGCCAAT | |
| | GACGGACAGGAGAAAGTCGATCTTCTTGCCCCAGGTCTCCCGATC | |
| | CTGGGCCTCCACGGGGCTCTGCCGCGGGTTGGTGAGGGTGGAGCT | |
| | GGTGAGCTGCACTCCGTTCTGCTCCTTGACAAGGATGAGCTCCAC | |
| | CTCCTTCGGGCCCACGGCATTGGGCTCCTTAGCCGGGGCCACCAC | |
| | GGAAGACATGAGTCCCACGGAGCATTTGCTCTTACTCATGGGCAC | |
| | ACTGGGAGTTGAGGAATTCTGTGCTTCTTCCCTCTTGGTCTTCAG | |
| | CCAATATGAAAAATAAACACACAACAGGAACGCAACAATTCAGCA | |
| | GCCAGGGCTAGGGACATACCTGGTGCGGAGGGCAGAGCCCCGAGG | |
| | CATTCACGGGCATTCCTCTGGGAAGGGATGGGTGCGTTCTCAGCG | |
| | CCTGAGATGGTACAGCTGGGGCACGGGAGCTGGGAAGTGCCAGCC | |
| | CTCACCACAGGGCTGGGGCACTGGCCCATGGAGGCCTCAAGACAG | |
| | ACACTCTGGTTTGCTCCTCCTTCCCCTGCACCCCTCCCCACTCCT | |
| | ATCAAAGTGCAAGGCTGTGTGAGCTCTAGCGTGGCTTTCTACTAA | |
| | CAATGCATGGATATCACTTCCACCTTCCCCCACACTTCCAGAGAG | |
| | CAATTTTACAATCTTCTAAGAGCATTCAATAATGTCTTCATGACT | |
| | GTTCATCTGTATTCTTTTTTCTTTTTTTTTTCTTTTTTAACATGA | |
| | GGTCTCGCTCTGTCACCCAGGATGGAATACAGTTGTGATTACAAC | |
| | TCACTGCCTCAACCTCCTAGGTTCAAGTGATCCTTGCACCTCAGC | |
| | CTCCTGAGCAGCTGAGACTACAGGTGTGCACCACAGCACCGGCTA | |
| | AGTTATTTTTGTGTGTGTTTTTTTGTTGTTGTTGTTGTTTTTTTG | |
| | TAGAGACAGGGTCTGACTATTGTTGCCAAGGCTGGTTTTGAACTC | |
| | CTGGTCTCAAGCGATCCTCCTGCCTCAGCCTCCCAAAGTGCTGAG | |
| | ATGACAGGCATGAGCCACCAAGCCTGGGCTTGTATTGTTTATACT | |
| | CAGTAATAAAGAGCAAAATTTATATAAGCAAAATCAGCCACTGTA | |
| | AAGAGAGTTTAGACATAACCATCACAACTGCTTCCTCAAAAAGTG | |
| | TCTGTACAAGCCACATCCACCTAGGGCAGGTGGCACCACCCCGCG | |
| | TGAGAGAGCTGGGCGGAGGATGGACAGGGCTTCATCGCGGGAACT | |
| | AGCTCCTGGGCCAGCAGGGTAACCCGGGAAATCCTGTCCAAACGG | |
| | CTACATAAACCCCCCTGCAACGCTGAGCAGGAGAGCAGGAGCGTC | |
| | TACACCGCCCCAGCCAGGGCGACTTCTGTCATTTGGATCCAGCAG | |
| | CAGCGTGTTTCAGTCGAGCGCACGCACACACAGACACACAGACAC | |
| | ACACAGACTCACAAACACACATACACACTCACACACACACAGGCG | |
| | AGCACATACACTTACACACATACACACTCACACTCACACAGGTGC | |
| | GCACACACACACAACGACTGAAGCAGGTCGCAGGTGGAGGCTCTA | |
| | ACAGGCAACTTTCCCTGCATCCAAGTTTGCACAAAACCTCACCCA | |
| | TGAGCTATCACGAGCTATGCCACGCACCCGGTGACAACCTCAACC | |
| | GGCACGACCCCTCCGGTGGGTAAAACACCCGACGAAGGGGTTCCG | |
| | CGGCGCGAGCAGGAGGCCGCGTTGGGAGAGGGCGTCGGGTGCGGA | |
| | GCTCGCGAGTCTCCGGCAAGCCGCCGCCGCCCTGGGGCCACCTGA | |
| | CCCCGCTCCTGAACGCGGGGCCTCAGAGGCGAGTTTTGGGTCTAC | |
| | ACAGCAAAATGTGGCCCGTTAGAAAGCGTCCTTCCTCACCGCGCC | |
| | CAGAACAAGAAGCGGCGCTGCCTGGCGAGTCCTCGCCCGGCCTCC | |
| | CCGCCCTGCCCAGAGCACCAGCGCGCAGCCCGCGCCCGCCACCAC | |
| | CCCCAACACAGACAAAGCCCCCGCGAGATGGAGCGGCGGTGTACA | |
| | AAACCCACTCCGATCGGATCTGGGATGCGCCGCACCCCTAGTAGG | |
| | GTTAAGTTCCACGAGAAGAAACCAGGACCCCCGAGTCACAGCCAT | |
| | AGACACCCACGCGTCCCCTAAGCCGGTGACTGCACCAGGTGCCCA | |
| | CCCGGTTCTGAGCCTGGAGCGGGGCCAGGGGCAGCCCTGCACCCT | |
| | CCTCGCGCCTCGGACGGCCTCAGGGTCCCATGTGGCGCTATCGGG | |
| | GGGCGCTCGGCACCCGCGACTGTTGGCGACTTTGGAGACGGCGCG | |
| | GTCTGCGGGACGCGGGGACCCCAGACTGTGCCCGGTCCCCGGCCC | |
| | CCGCCCCTGCGCCCCACTTCGGGGTCTGCGGAAGGAGCGGGGCGC | |
| | CCCGATGCCGCGAGCGACGCTGGCCTCACCTGGCCGCCCGGGCCT | |
| | GGGCTTTGCACGCGAGTCCTGGCGCCGAGAGCGTTCCGCGAAGCC | |
| | TCCCCTCCCGCTCCGCAGCGCTGGGCGGTCT | |
| 48. | AGCCTCCCAGCCGGCCCACCGGGCCCCGCTTTGGCCCAGCCCCGC | INS |
| | GCGAGCTCACATACCTCAGCTCCAGCAGGGCGCCCCTGTCTCTAC | |
| | CCGTGCGAGTGGGGCCGGCTGGGTGTTCCCATGGACGGCTGCTGC | |
| | CGATCCCAGCCACCGCCTCCCTCCTCTTCCGGGGCCAGTGTCCGG | |
| | CCGTCCACCCGCCTGCCCAGCCTTGGCCCCCACTCTGGAGGCGGG | |
| | GCTAGCTGGGGGCTGTGACATCTCTGGGTGGAGGGGGGCTGATGT | |
| | GGTCATGGGGAAGGGGTGAGCTCAGAGCTTCAGGAACAGGGCTGC | |
| | CCCAGTGGCCAGGTCCCCAGCTGGAGGGTGAGCTCCTGGCCTCGA | |
| | GGCCCAAGCTGGCAGTGGTCTGCAGCCACAGCCCATGCCAGCCCC | |
| | AGGCCCAGCCATCCCCAGCCCAGCCCTGGGTGGAGCTCTCAGGAA | |
| | AGGTGCAGATTGGTTTGGCCTGGGTGGCAGGCAGACTGGCCCAGC | |
| | CACAGGGTAGCTAGGGTCACCCGCTGACCTCCCAGGCCACAGTAG | |
| | GCCCAGGCAGCCCCTAGGTTCTCGTCTGCCCACTAGGCTGGGTGC | |
| | CCAGCGGAGCCACCCTGTCCTCAGGGGTGCTGCAGAAGAGGCTCT | |
| | GTGTCAGGACCCCCAAGGACCTGGAGCCACAACGACCTTGTCACC | |
| | AGGGCCCGTGGGCCCTTCTGGGTGAGCAGCAGGTCTGTAGCAAAG | |
| | GAAGCCAGCCAAGCAGCCCTGCTTACTGGGAGGGGGTGGGCTAAG | |
| | GGGCTGCAGGGGGACCCTGGCCCTGGTCTCTGCCCCTGTGGCCCT | |
| | GGCGGTTCCCTGCTTCTCCTGGGCTGCAATCCTCAGGGCCTCATG | |
| | GCCGGGGTGCCCAAGGCATGGGTTCTGAGGCCCTGCCAGGTGCTG | |
| | CCACGGTCCCTGCACCTGCCCCAGCTCATGGTGCCATCTGACCTC | |
| | TCCTGCTTCCTCCAAGGCACCAGTGGGTCCCTCAGGTGTCTGGCT | |
| | GGACCTGGGCTGGCATAAGCTGTGGCTGCAGACGGCTGCCAGCTT | |
| | GGGCCGCGAGGCCAGGGTGTGTGACTGTCCCGGGGCTGCCCAGAG | |
| | CTGGGGATAGCGGGTGGCTCTCGGCAGCCTCTCCCCACCTTCCCA | |
| | GCCCCCCGCCCTGCAGGACCCCCTCCCTCAGCCCAGCCTCCTCCC | |
| | TCCACAGGGACTCCATCAGAAATAACTCTAAAAATAGAACCTGGG | |
| | AGGGCTAGGTGGGGGGAAAATTGCTGGAATGTTCTCATTCCCTTT | |
| | CCTGAACAAGGTCTCTGGGGACTCCAAGAGTCCAGAGCTACTGAA | |
| | CAAGAAGTCACTTCTCAGTGGCCCCACCACCCCTGGCCCCTCAGA | |
| | GACCCCCGCAGCTCCCATACTGGACCCTGAGCCACAGGGTGGGGG | |
| | CAGCAGGCAGCCGACAGGCATGGCCGCTTTGGGGAGAGCCACTGC | |
| | ATGCTGGGCCTGGCCGGCGTTGGCACCTGTGGGCACCCAGAGAGC | |
| | GTGGAGAGAGCTGGGAGGGGCTCACAACAGTGCCGGGAAGTGGGG | |
| | CTTGGCCCAGGGCCCCCAAGACACACAGACGGCACAGCAGGGCTG | |
| | GTTCAAGGGCTTTATTCCATCTCTCTCGGTGCAGGAGGCGGCGGG | |
| | TGTGGGGCTGCCTGCGGGCTGCGTCTAGTTGCAGTAGTTCTCCAG | |
| | CTGGTAGAGGGAGCAGATGCTGGTACAGCATTGTTCCACAATGCC | |
| | ACGCTTCTGCAGGGACCCCTCCAGGGCCAAGGGCTGCAGGCTGCC | |
| | TGCACCAGGGCCCCCGCCCAGCTCCACCTGCCCCACTGCCAGGAC | |
| | GTGCCGCGCAGAGCAGGTTCCGGAACAGCGGCGAGGCAGAGGGAC | |
| | ACAGGAGGACACAGTCAGGGAGACACAGTGCCCGCCTGCCCGCCA | |
| | GCCCTAGGTCGCACTCCCACCCATCTCCAGCCGGGCTGGACCCAG | |
| | GTTAGAGGGAGGGTCACCCACACTGGGTGTGGACCTACAGGCCCC | |
| | AACGCCCACATGTCCCACCTCCTTCCCCCGCCCCGGGGCAGCGTC | |
| | ACAGTGGGAGCCTGAACAGGTGATCCCAGTACTTCTCCCCAGGGC | |
| | CTGTCCCCAGCATCTTCCCCATCTCCTGACTATGGAGCTGCCGTG | |
| | AGGCCTGGCGACAGGGGTCTGGCCCACTCAGGCAGGCAGCCACGC | |
| | CCTCCTCCGGGCGTGATGGGGTGTTCGCCCAGAGGCAGGCAGCGT | |
| | GGGGCACCCTGTGACCCCAGGTCACCCAGGACTTTACTTAACAAA | |
| | ACACTTGAATCTGCGGTCATCAAATGAGGGTGGAGAAATGGGCTG | |
| | CGGGGCATTTGTTTGAGGGGCGAGTGGAGGGAGGAGCGTGCCCAC | |
| | CCTCTGATGTATCTCGGGGCTGCCGAAGCCAACACCGTCCTCAGG | |
| | CTGAGATTCTGACTGGGCCACAGGGAGCTGGTCACTTTTAGGACG | |
| | TGACCAAGAGAACTTCTTTTTAAAAAAGTGCACCTGACCCCCTGC | |
| | TGGGTGGCAGCCTCCTGCCCCCTTCTGCCCATGCTGGGTGGGAGC | |
| | GCCAGGAGCAGGGGGTGGCTGGGGGCGGCCAGGGGCAGCAATGGG | |
| | CAGTTGGCTCACCCTGCAGGTCCTCTGCCTCCCGGCGGGTCTTGG | |
| | GTGTGTAGAAGAAGCCTCGTTCCCCGCACACTAGGTAGAGAGCTT | |
| | CCACCAGGTGTGAGCCGCACAGGTGTTGGTTCACAAAGGCTGCGG | |
| | CTGGGTCAGGTCCCCAGAGGGCCAGCAGCGCCAGCAGGGGCAGGA | |
| | GGCGCATCCACAGGGCCATGGCAGAAGGACAGTGATCTGGGAGAC | |
| | AGGCAGGGCTGAGGCAGGCTGAAGGCCAGGTGCCCTGCCTTGGGG | |
| | CCCCTGGGCTCACCCCCACATGCTTCACGAGCCCAGCCACGTCCT | |
| | CCCTGCTGCAGAGCTGGGGCCTGGGGTCCAGCCACCCTGGAATCC | |
| | TGAGCCCACCTGACGCAAAGGCCCTTGGAACAGACCTGCTTGATG | |
| | GCCTCTTCTGATGCAGCCTGTCCTGGAGGGC | |
| 49. | CCTGTCCCCACTGCACTTCTTCCATGTCCCCTGGCTAGTCTTCAG | Leu7 |
| | TAGGGTCCCCAAAGGTCTTTTTGCCTGGGGGCCTGGGGAGGGGGT | |
| | GCATGACTTCTCTTCTTCTCCTGAGTGCCAGGTTTCCCCAGACCC | |
| | CACATCCTTCCCCACTGCCCTGCATTCCAGGACCAGGAGGGCTGG | |
| | GGCAGGGCCAGAAGATGGAGAGGAGCCCTCAGCATTGTGAGATGC | |
| | TCAGGGGTGAAGGGTGTCCCTGAAAGTCCCTGCAAGCTCTTGATG | |
| | TGCTTGACCTCATAGCCTGGCCTCCCTCTGCCTTTGGGCTTCTGT | |
| | CTAAAAGACCCTCACATTCCTGAGAGCCACAGGAAAGCACCTTCC | |
| | TCAGTAGAATGTCCAACTGCTCTAGTGAGGGGCCTCCTGCAGGGG | |
| | TGGACAATTGCTGAGGTCGATGTAGGGGAACAGGGGGAGGCCCCC | |
| | AGCCTGCCCCAACTCACCACTCAGTCAGGGAGGCCCTGACTTCTG | |
| | CCTGTGTGCCTCCCTCTGCTGTCCGCCAGTGCGCCTCCGTGCCTT | |
| | TGTCTCTTGTCTCACTGCCTCTTTCTCTGCCTCAGTCAGGCTCTC | |
| | TCTCGGATTCCTCCTCCTCCTGAGCCCGTTCCTCTCCTCCTCTCC | |
| | ATTCTCATCAGAGAGGAACACTCATCCCTCTCTTCCAGAAACAAT | |
| | GGAGCACCATGCTCCTGTCCGCTCAGCCTTTCGGTTGGTGCATCC | |
| | TCTTCCCCATCTCACACTCCCAGCTCCTGGCCGGCCTCATCACCC | |
| | TGTGCTCCACACCTAGGAAAAGTATATCTGACTTGGTCCCCTTAT | |
| | CCCCGAGAAACCACTAAGAGAACCTTCAGGTCTCCCCAGAAGCCG | |
| | AAGCTGAGGAACTGGGCTTGCCCCAGGGCATCCTCCTGTGAAGCT | |
| | CCTTGGTCTCCGCCACGGGCTCCAATCCTGTTGCTGGAGCTTATC | |
| | TCCTCAGTGATTTACCTGCCCCCTGCTTCTGGTGGAACAGTGAGT | |
| | AACTGCCCAACCAGAAACCTACACACACATATTCAACACAGCGCC | |
| | TGCAAGCTTCCAGCTCACCTGGACCTGGAGTGATCCCCAAGCATC | |
| | CTGGGGTCAGGCACAGAAACAGGAACAGCGAGCTCCTGGCTCCAG | |
| | TGCTGAATGGGGCACAGATGCATTAACTCTCCCAGTCCCATTAAC | |
| | CTTAGCAAGTATTTCTGCTTCCATGAAAAGGGGAAGAGGGAAAAT | |
| | GTCTCTAAGCTCTGGAACCTTTGGCAGTGCTGGGCATGGACTGGG | |
| | CAGAGTGAGAAGTCTGTCTGAAGACCCTGGGAAAGGGTCTTCACC | |
| | CCATGGGAAGAAAGGGAAGGCTTGATGTTTGGTCCAGTAAGGCCA | |
| | TTAATGGTCACCAACTGAGCAGACTCTGGGAGGAAATGTCTCAGT | |
| | CTCTGCAGTGGCTACCAGCAGAGGTAGGGCGACATAAGCACAGAA | |
| | GACAAATCTTAGAAAACCCTAGAACAACAGTCCTGTGCAGCCACA | |
| | TGCCCCACAGCCCTAGTAACTTAGGGAGTGATGTGCCTCCCATTT | |
| | CCACACAAACACATCCAGGCATTGCACAGTGCTAGGCCCTACGAC | |
| | ATCCGTACCCCAACATTTTCAACAGTGCGTGCTACACAGCCCAAG | |
| | CTCTAGGCCCCATTGGGCACCCCACCTATGCAGGTGCCCATGCAC | |
| | TGGGCTAGCAACTGTCAGTAAAGGTCCCCTTCCTGTATGTCCAGA | |
| | GTCCTTCAGCTCCAGCTTCTACAAGAATCCATGCAGCTAGGCCTG | |
| | GCTGGGGGAGGAAGGGGACAGGGAAGTTGGCCAAGGCTCCCATGT | |
| | GTGTGCATGTGGGGGTGGTTCTTGACCTTAGCTGCTTGGTACGGG | |
| | GCTGTCACCTCCTTACCCTGGCACTGTCGGACCTGGTGGCTTCCT | |
| | TCTCTATTTCCTGCCTCAACTCTCAGCCTCCTTTTTCTGGAAGTT | |
| | GTGTGCTCTGACTTTGGAGGGATTGGGACCTCAGACCTGAGCCCA | |
| | GGTGCAGATGGGAGGGTGGTGAACTGGTCGCGCCTCCTTCCTCCT | |
| | TGCTACACCGAAGGTCCCAAGGGCCCTTTGCCACCCTTGCTACCC | |
| | CTGGGTCCCTCAGCGCGCCCCGCAGAGCTCGGGCCCCCTCAAGAG | |
| | AGTTAACTTGGCAGTGCCCAGGAGCCAGAGAGCGATCCAGAGAGC | |
| | GCTGTTGGGCAGCAGGGGTGGGGAGGTGGGGGACGACTGACCCCT | |
| | GACTCCTCATCCGGCCACCAGAAACCACCTGCTGAGGGGGCCGGG | |
| | AACTGACGACTGAGAGCCCGGGCCGATTGTTAGAAGCTGCTGAGA | |
| | ACCAGCTCTTCCCCTAATCCCGGTCGACGAGGGCAGGCTGTGCCG | |
| | GGTTTTGTTTCGCAGCCCCTCGGCAGCAGCGCCTCCCAGCCCGAG | |
| | CTCGGTTCTCGGCCCCCTTTCCAGCTGCCCCCAGCGCGCGCAGCG | |
| | CACACACACACACACACACACACACACACACACACCCCAGCGCGC | |
| | GCCCGGGAGGACATGGCGTGGGCACCGATGGGGACGCAGAAAGGG | |
| | GCTCTGTGCGCGGCGGGGCGCCCGCGGGGGCGCGGGGAGCACTGA | |
| | CCTGCGGCGACGAGTCCGGAGCGTCTCCGGTGCGGTCCATCGCGG | |
| | GCAACGGCCCCTACGCCCCGGCCCCGGCGCCCCTAGACCGCTGCC | |
| | CTGGGCCGCGTGCGGCTCCCTCCCCGGGGACTGTCGGGCTCCGGG | |
| | CCCCCGGGGGCCACTTCATAGCCGCGGGGTCCGCGCGCCCGCCCG | |
| | CCCCGCCCGGCCCCGCCGCCCCGGCCCGGCTCGTTCTGGGCTCAG | |
| | CGAGGCGGCGGCCGAAGGCTGGGAGCGCGCGGGAGGGAGGGCGCG | |
| | GGCAGGGAGGCGGGGGGGGGGGGGCGGGGAGGGGGAGCGGGGAGG | |
| | GGGAGCGGGGAGCGGGCGCGGGGGCGAGAGGGGCGAGGGGGGCGC | |
| | GCGGCCGGAGCCGAGCCGACCGGGCCGGCGCAGAGTCCCCGAGGT | |
| | GGCGGCGGATGCGCCGGTGCCGCCGCGGCTC | |
| 50. | GCTGATCCCAATGTAGTAAGGCAGCCAACAGGCGAAGAAAGCCA | CXCR4 |
| | GGATGAGGATGACTGTGGTCTTGAGGGCCTTGCGCTTCTGGTGGC | (CD62L) |
| | CCTTGGAGTGTGACAGCTTGGAGATGATAATGCAATAGCAGGACA | |
| | GGATGACAATACCAGGCAGGATAAGGCCAACCATGATGTGCTGAA | |
| | ACTGGAACACAACCACCCACAAGTCATTGGGGTAGAAGCGGTCAC | |
| | AGATATATCTGTCATCTGCCTCACTGACGTTGGCAAAGATGAAGT | |
| | CGGGAATAGTCAGCAGGAGGGCAGGGATCCAGACGCCAACATAGA | |
| | CCACCTTTTCAGCCAACAGCTTCCTTGGCCTCTGACTGTTGGTGG | |
| | CGTGGACGATGGCCAGGTAGCGGTCCAGACTGATGAAGGCCAGGA | |
| | TGAGGACACTGCTGTAGAGGTTGACTGTGTAGATGACATGGACTG | |
| | CCTTGCATAGGAAGTTCCCAAAGTACCAGTTTGCCACGGCATCAA | |
| | CTGCCCAGAAGGGAAGCGTGATGACAAAGAGGAGGTCGGCCACTG | |
| | ACAGGTGCAGCCTGTACTTGTCCGTCATGCTTCTCAGTTTCTTCT | |
| | GGTAACCCATGACCAGGATGACCAATCCATTGCCCACAATGCCAG | |
| | TTAAGAAGATGATGGAGTAGATGGTGGGCAGGAAGATTTTATTGA | |
| | AATTAGCATTTTCTTCACGGAAACAGGGTTCCTTCATGGAGTCAT | |
| | AGTCCCCTGAGCCCATTTCCTCGGTGTAGTTATCTGAAGTGTATA | |
| | TCTGCAAAAGAGGCAAAGGAATGGACATTCACTTCCAATTCAGCA | |
| | AGCATTAACCCAGTTAAAAAAAATTTTTAAAGCAATTTAAAAAAC | |
| | CAATTCAGGCTTGCTTTCTTCAGGAAATTCTGAAGTAGTGGGCTA | |
| | AGGGCACAAGAGAATTAATGTAGAATCCTACAACTCTCCTCCCCA | |
| | TCTTTTCCCATAGTGACTTCATTATATCCTTCTTTGGTAGAACCA | |
| | ATTACAAAATTCTTTGTTTAGAACAAAAGGGCACTGAGACGCTGA | |
| | GGGTTTCAAAGTCACATCTTGGCTAACTCCTCTGCCCCGCCCACT | |
| | AGAGGGAAGAAAAAAAACCTTCCTTAGGAGGAAAAAAAAAATACA | |
| | CACAAAGAGGCCACTCCCAGGCGGCGTGGGGGGTGGGGTGGGTGC | |
| | TGCTAGGAGGGGCAGTGATTAACTTTTTGTAAGAAGTTTTTCGCC | |
| | CAGGGAGGGAAGAGGGGAGAAGGGAGGATCACGGGGGGAGGGCGG | |
| | GGGCGTTGCAAAGACTCATTCTCCTAAAGCGCAAAAACTTAATTT | |
| | TCCCACGCCTGCCTAAATACAAACCATTCTGGGCTTCAAGCAACT | |
| | TGTAGTGGGTAAAGAGAATGCGGTCTTAAAACGAAGGCCCTTCGG | |
| | TGCTTGGGGTATATTGGGCGGGAGTGTCAGAAAATGAACAAACGG | |
| | CACCTCCTCCCCCAAGCGGGCGCTCCTCCGGTGTGTGGGTCTCTT | |
| | GCCATCCTCGTGTTTATCACTTGGCGCGTTTGGGACGTTAGGGAG | |
| | CGGGGCATTTTCCTGGGTGGAGAAGGTAACGGGGTCTGCACCCGT | |
| | GGTCCTCGCCCCAAGTTTCATTTCCTCACTCTCCCGGGTGGCTTC | |
| | CCATTACCCCGCCACTGATCCAGTTAACCCGGCCGGAGGTGGGCA | |
| | GCTGGAAGCCTCCAGGCGGTGGGCACGCGGGGGGCCGGGTCGTCC | |
| | AGCCCCGGGCCGCCGCGGCTGCCCACTACACCCACGCCAACCGCC | |
| | CGCAAGCAGCGCTGCAGGGGCTCCGCTGGGCGACACGCCAGGCTC | |
| | TGTCCCACAGGGTGCTGGGGAGCGACTGGGCGGCTCCGCCGCGAG | |
| | CGTCTTTGAATTGCGCGCCGCTGCAGGAAACCAAAAACTCCCTAG | |
| | CAAGAGGGTTTCAAAAGGTTTCTGGAAACCACCGACGGTTAAACA | |
| | TCACAACTGGACTCGGAGAGAGCCAAACGGTTTCCCCACTTGCAC | |
| | CTGCCAGTCTTCGCGGCGGCGACCTGGCAGCCCAGGTGCGGTCTT | |
| | AACCGCCCCCGCCCCTCACCCCGTACCCGCTCCTATCCCCGGAGC | |
| | GCAAATCTCAGGGCTGGCAGCTGCGCGGTGTCAAAGGGGAGGTCA | |
| | AACCACTCCGCTGACCTCTGCACGACCCCAAACTCTCGAACTGCA | |
| | GGACCCACTCGCGGCCGTGGGGAAGAGGCGCGCTTCGGACGGCGG | |
| | GAAGGTTTTCCCCCTCAAACCCAAAGCGCGCGGGCGGATCAACTC | |
| | CTAGCTGCTGCCACCACTCGATCCCCTCAGAGGATCGGCGCGGTG | |
| | GGTCCACCCGCCTCTCCCGCCCTCTGCCTACTGTGCTGGGAGACT | |
| | GGCACAGCTCCGTCGGCCGCACAGAGTTTAACAAACACGCACCCA | |
| | GTGTCAAGAACAGTCACCAGGCGCTTAACCCCGAAGTTAAAGCGG | |
| | GCGCAATCTCCTCCTGGGAACTCAGCCCAGGCACGCCGCCCTCCG | |
| | CCTCTAAATTCAGACAATGTAACTCGCTCCAAGACATCCCCGCTT | |
| | CCCCAAGGAAGAGACCGGTGGTCTGAGTCCCGAGGCAGCGCGCAC | |
| | GCCTTCTCTGCACTTGTGCACAGAATGTTCTTACGTTTGCAAACA | |
| | GCGTGCAAGCCGCCGCGCGCGGCGGGACTCAAGGGGGAGACACAT | |
| | GCAGCCACTGGAACGCTCTTTCCAGTCGTTTCTCCTCGACTCACA | |
| | GAGAAAAAGATTCCAATCCTGCTCCCCCCCCACCCACCCGCACTA | |
| | TATAGGCATGGTCAAGAAAACTCCTTTCGGTGACCCTTTTTTGGA | |
| | GTACGGGTACCTCCAATGTCCTGGCCGCTTCTGCCCGCTCGGAGA | |
| | GGGGCTGCGCTCTAAGTTCAAACGTTTGTACATTTATGACAAAGC | |
| | AGGTTGAAACTGGACTTACACTGATCCCCTCCATGGTAACCGCTG | |
| | GTTCTCCAGATGCGGTGGCTACTGGAGCACTCAGGCCCTCGGCGT | |
| | CACTTTGCTACCTGCTGCCGCAGCCAACAAAC | |
| 51. | TCTGTCTCCCTCTGCTCACCTTGGGGTTTCTCTGACTGCATCTTG | interferon- |
| | TCCCCTTCTCTGTCGATCTCTCTCTCGGGGGTCGGGGGGTGCTCT | gamma (IFNg) |
| | CTCCCAGGGCGGGAGGTCTGTCTTCCGCCGCGTGCCCCGCCCCGC | |
| | TCACTGTCTCTCTCTCTCTCTCTCTTTCTCTGCAGGTTCTCCCCA | |
| | TGACACCACCTGAACGTCTCTTCCTCCCAAGGGTGTGTGGCACCA | |
| | CCCTACACCTCCTCCTTCTGGGGCTGCTGCTGGTTCTGCTGCCTG | |
| | GGGCCCAGGTGAGGCAGCAGGAGAATGGGGGCTGCTGGGGTGGCT | |
| | CAGCCAAACCTTGAGCCCTAGAGCCCCCCTCAACTCTGTTCTCCC | |
| | CTAGGGGCTCCCTGGTGTTGGCCTCACACCTTCAGCTGCCCAGAC | |
| | TGCCCGTCAGCACCCCAAGATGCATCTTGCCCACAGCACCCTCAA | |
| | ACCTGCTGCTCACCTCATTGGTAAACATCCACCTGACCTCCCAGA | |
| | CATGTCCCCACCAGCTCTCCTCCTACCCCTGCCTCAGGAACCCAA | |
| | GCATCCACCCCTCTCCCCCAACTTCCCCCACGCTAAAAAAAACAG | |
| | AGGGAGCCCACTCCTATGCCTCCCCCTGCCATCCCCCAGGAACTC | |
| | AGTTGTTCAGTGCCCACTTCCTCAGGGATTGAGACCTCTGATCCA | |
| | GACCCCTGATCTCCCACCCCCATCCCCTATGGCTCTTCCTAGGAG | |
| | ACCCCAGCAAGCAGAACTCACTGCTCTGGAGAGCAAACACGGACC | |
| | GTGCCTTCCTCCAGGATGGTTTCTCCTTGAGCAACAATTCTCTCC | |
| | TGGTCCCCACCAGTGGCATCTACTTCGTCTACTCCCAGGTGGTCT | |
| | TCTCTGGGAAAGCCTACTCTCCCAAGGCCACCTCCTCCCCACTCT | |
| | ACCTGGCCCATGAGGTCCAGCTCTTCTCCTCCCAGTACCCCTTCC | |
| | ATGTGCCTCTCCTCAGCTCCCAGAAGATGGTGTATCCAGGGCTGC | |
| | AGGAACCCTGGCTGCACTCGATGTACCACGGGGCTGCGTTCCAGC | |
| | TCACCCAGGGAGACCAGCTATCCACCCACACAGATGGCATCCCCC | |
| | ACCTAGTCCTCAGCCCTAGTACTGTCTTCTTTGGAGCCTTCGCTC | |
| | TGTAGAACTTGGAAAAATCCAGAAAGAAAAAATAATTGATTTCAA | |
| | GACCTTCTCCCCATTCTGCCTCCATTCTGACCATTTCAGGGGTCG | |
| | TCACCACCTCTCCTTTGGCCATTCCAACAGCTCAAGTCTTCCCTG | |
| | ATCAAGTCACCGGAGCTTTCAAAGAAGGAATTCTAGGCATCCCAG | |
| | GGGACCACACCTCCCTGAACCATCCCTGATGTCTGTCTGGCTGAG | |
| | GATTTCAAGCCTGCCTAGGAATTCCCAGCCCAAAGCTGTTGGTCT | |
| | GTCCCACCAGCTAGGTGGGGCCTAGATCCACACACAGAGGAAGAG | |
| | CAGGCACATGGAGGAGCTTGGGGGATGACTAGAGGCAGGGAGGGG | |
| | ACTATTTATGAAGGCAAAAAAATTAAATTATTTATTTATGGAGGA | |
| | TGGAGAGAGGGGAATAATAGAAGAACATCCAAGGAGAAACAGAGA | |
| | CAGGCCCAAGAGATGAAGAGTGAGAGGGCATGCGCACAAGGCTGA | |
| | CCAAGAGAGAAAGAAGTAGGCATGAGGGATCACAGGGCCCCAGAA | |
| | GGCAGGGAAAGGCTCTGAAAGCCAGCTGCCGACCAGAGCCCCACA | |
| | CGGAGGCATCTGCACCCTCGATGAAGCCCAATAAACCTCTTTTCT | |
| | CTGAAATGCTGTCTGCTTGTGTGTGTGTGTCTGGGAGTGAGAACT | |
| | TCCCAGTCTATCTAAGGAATGGAGGGAGGGACAGAGGGCTCAAAG | |
| | GGAGCAAGAGCTGTGGGGAGAACAAAAGGATAAGGGCTCAGAGAG | |
| | CTTCAGGGATATGTGATGGACTCACCAGGTGAGGCCGCCAGACTG | |
| | CTGCAGGGGAAGCAAAGGAGAAGCTGAGAAGATGAAGGAAAAGTC | |
| | AGGGTCTGGAGGGGCGGGGGTCAGGGAGCTCCTGGGAGATATGGC | |
| | CACATGTAGCGGCTCTGAGGAATGGGTTACAGGAGACCTCTGGGG | |
| | AGATGTGACCACAGCAATGGGTAGGAGAATGTCCAGGGCTATGGA | |
| | AGTCGAGTATGGGGACCCCCCCTTAACGAAGACAGGGCCATGTAG | |
| | AGGGCCCCAGGGAGTGAAAGAGCCTCCAGGACCTCCAGGTATGGA | |
| | ATACAGGGGACGTTTAAGAAGATATGGCCACACACTGGGGCCCTG | |
| | AGAAGTGAGAGCTTCATGAAAAAAATCAGGGACCCCAGAGTTCCT | |
| | TGGAAGCCAAGACTGAAACCAGCATTATGAGTCTCCGGGTCAGAA | |
| | TGAAAGAAGAAGGCCTGCCCCAGTGGGGTCTGTGAATTCCCGGGG | |
| | GTGATTTCACTCCCCGGGGCTGTCCCAGGCTTGTCCCTGCTACCC | |
| | CCACCCAGCCTTTCCTGAGGCCTCAAGCCTGCCACCAAGCCCCCA | |
| | GCTCCTTCTCCCCGCAGGGACCCAAACACAGGCCTCAGGACTCAA | |
| | CACAGCTTTTCCCTCCAACCCCGTTTTCTCTCCCTCAAGGACTCA | |
| | GCTTTCTGAAGCCCCTCCCAGTTCTAGTTCTATCTTTTTCCTGCA | |
| | TCCTGTCTGGAAGTTAGAAGGAAACAGACCACAGACCTGGTCCCC | |
| | AAAAGAAATGGAGGCAATAGGTTTTGAGGGGCATGGGGACGGGGT | |
| | TCAGCCTCCAGGGTCCTACACACAAATCAGTCAGTGGCCCAGAAG | |
| | ACCCCCCTCGGAATCGGAGCAGGGAGGATGGGGAGTGTGAGGGGT | |
| | ATCCTTGATGCTTGTGTGTCCCCAACTTTCCAAATCCCCGCCCCC | |
| | GCGATGGAGAAGAAACCGAGACAGAAGGTGCAGGGCCCACTACCG | |
| | CTTCCTCCAGATGAGCTCATGGGTTTCTCCACCAAGGAAGTTTTC | |
| | CGCTGGTTGAATGATTCTTTCCCCGCCCTCCTCTCGCCCCAGGGA | |
| | CATATAAAGGCAGTTGTTGGCACACCCAGCC | |
| 52. | TAGCAGAGGCTGCTGAACCTCGGTCACCGAGAGGAAGGTGGATGA | neural cell |
| | CCCGGGCGTGGGGGTGATTACCCAGACCAAGGCGCGTCCCACGGG | adhesion |
| | GCCCTGCTACTTCGCTTTTAAACAAGCCTCAGGGAGGAAACCGGG | molecule |
| | AGCCCGGGCCAAGGGAGGAAAATGGCCACTTTCTGCTAAACAGTC | (CD56) |
| | TCAAGGCTAGATAGTATTCCAAAAAAAGCGCTGGTCCGGAGGTGC | |
| | AAAGGGTAGGGCACATCTGCGGAGGAAACAGTCACCTTAATTACC | |
| | ACGTCTAGAGAAAGTGCGATTTCATTACAGCCTCCAAAACTACCG | |
| | CCCAAAGAAGGAAACCAGAGAAGTGTTTTCAATTTGAAAGAGGCT | |
| | GGAAAGAAGCGGGTAAGACACCGACTCTCCCCACCCTCCCGGCAA | |
| | CTTTGGACCACAAGGCGCTTTCCTGCCTCAAAGACTATTACCAAA | |
| | CACAACTCGAAAATCCAACCCCGCTTTCAAACCTGTAGCCGTTAT | |
| | TTAAACGGTAAAACACCCCACTTTAGTTTCCGGGGGATGCGGCCC | |
| | AAATCAGTCCTTAAAAACACACAGAGAGCGCCTCTGGCAGCAGCC | |
| | GCCCACGCCCCCGGCCCTGCGCACCGGGCTTGCTCCGGGAGGGGC | |
| | GCGTGACGCCACCGCGGGGCAGCCGAGAGGGGACCGCGGGCTCCG | |
| | GGCAGGGCAGGCGCTCCCGCAGCTCCAGCTATAACACAAGGGAGA | |
| | GAAAGCAACGCTTTGGTTCCAGAAACTTCCATCGTCAAGGCCAGT | |
| | TCTGGCCAGACGCACAGCCAAAGAGCCCCCCTAGTCTAGCGGATG | |
| | AACCGTTCGCGAGTGGCACCCCCTAAAACCTTGGCCCCATCTCCC | |
| | AAGATGCCTCTTTACTGCGCAATCCAGAAAGCACCCGGACCGCCG | |
| | CCACCCACCAGCGGCGGCGAATAACTTGGTGAGATCTTAAATAAC | |
| | TCGGGATCCAAGGGAACTTTCTTTTCTCCCCCGAATCTCAACGCC | |
| | GCATCTCCAAGTCTCCAGAAATGTTAAGGAGGGAGGTGGTCAAGA | |
| | CAGGCTGAAGAATCTGAGTTTTAATTTGGGGGAGGGGGAGGGGAG | |
| | GAAGAGGGAAGTTAATAATGCGGCTGCGGTGTACCCGTGTGGGGT | |
| | GAAACGGAACCAGATGCGGCTGCTACATCTGGAAATTGGGGGTGG | |
| | GGGAGTGCTGGGGGAGAAAGGCACCGTGGTAATGTTTTTCAAAAG | |
| | GGTTGATAGCTTCTGGGCTCAGAACAAGAAACAGGCTTCACACCC | |
| | CACCCCCCCCCCACCACCACCAAAAAAGTGTCTGGCTGCCTCATA | |
| | GGGGGTGTGTGTTCTATATGAACCCCCATCTACAAGGTCCATAAA | |
| | ATTCCTCCGCAAACATGGGTCGTAGCCTCACTTTTCTCCCAACCC | |
| | CAAACATAACGGTAGGGGAAGCGAGGTGCAGAGGGGTCTGGGCCA | |
| | GAGAAGCATACGCATCGCTGCCGAGTGAACCACACCGGACAAAAA | |
| | CCCTCATCCCTTCCCCCACCTCTAGGAACCAGATTGGGACCGAAC | |
| | GGTCCTTCCGCCCCCATCCCACACCTCGGCTTAACGACCGTGGGT | |
| | TGTCCTCCCGTTCCCCAAACTTCCGACCCTTTCAGAGACCGTCCA | |
| | AGTCATAAGATCACCCCTTCTGGAAAGGGGAGTCGTTTTTCGCTC | |
| | TCAGCTCAAGCAGATTCAGCCATTTGGTGACGACACCTCCTTAAA | |
| | ACCTATTCCAAGAGAAGCTAACCTAGCCCTCCACGTCCTCCGCAG | |
| | TTCATCTCCCACCCCATCCGACCCCAACCCGCACCCCAGGCTTTC | |
| | CCGGGGGTGGAAAAGGACTTAGGAATGCCCCGGATTTTTAGCGTT | |
| | GAGCCCAAAGCTACCTGCTGAATTGGGGGTGAATCCCCAAAAGGG | |
| | GACCAAAAAGACATGGAGTGGAATGCAACACACACACATAACACA | |
| | CACAACGCATCCCACCTCGCCGCCCACTCCCGCCCTCCTCCCCAG | |
| | TCCAGGAAACCGCGGGCCTCGCCGCCTTCGCCCCGGGCCTGTCCC | |
| | TCCCGGGACTCACTGAGGAGCCGCCGCCGCCTCGCCAGCTCCCGA | |
| | GCGCGAGTTGGAGGAAAAGTTGGGCGGCGGGAAGAGCGGCGGGAC | |
| | GAGCGCAGGGAGGGGGCGCAGGAGACGCGGACGGAGGGAAGGGAG | |
| | GGGAGACCCAAGGGGCGCGGATCGCCCGGGAGGGAGCCAGGAGGT | |
| | GAAAGGGGGGGGCGGCGAGCGGAGGGAGGCGCCGGCCCCGGCTGG | |
| | GCTGCGGCGGGCAAAGCGCGCAGCCGGGAGGCTCCGGCGCCGGGG | |
| | GCCGCTCGGGACGCCAAGCCCGCCCCAGATCCGGGGGCGCCGCGG | |
| | CTCTTACCTCGCTCGGCGTGGAGGTTCCGCCTCGGGAGAGTCCGC | |
| | CGTGGCTTGTGCGAGCGGGCGTGTGCCCGCGTCCCCGGCTCCGGC | |
| | CCGCCGCCCCCGGGCTCTGTTCGGGTCCTGGCGGGGTCGCAAGAG | |
| | CTCCGCGGCCGGCGCTCGACTCCCGGCGGCGCTCGGTGCTCGGCG | |
| | CCTCCAGCCCGGGCGGGAACAATGGAGCCGGAGCTGGTGCCCCGG | |
| | AGGCGGGGGGGGGGAGGGCTCCCGTCCGCCACCCGGGGTCTCCAG | |
| | ACCTTGTGCCCCCCTCCTCCAGAACGCAGCGGCGGCGCCTCACTT | |
| | TCCTTGCAGACCGGGCTCCATCGCCCTGCGGAGGCCCCGGCGCCG | |
| | CGGCGTGCCCGACTGCAGCACGGGTACCGTGCGCCCTGCGGGGCG | |
| | CCCCGAGCTCGGACCGGAGAAAAGTCCTTGGGTTCCGCGGCTTTT | |
| | CAAGCAGCGGCGCGCTCGCGGCCGGGGAAGGCGAGGTCGCCGCAA | |
| | TGCGCTACGAGGGGTCTCGGTCCCGTCCGGACGTGGCCGCCCAGC | |
| | TCCCGGCACACCCGGGTTCCTCCGCGCGCTGCGGCTGCACCGGCG | |
| | TCCCGGCTCCCGCTAGCTGCCGCCCGCCGCCGAGGACGCCGCGCC | |
| | TGTGGCCGCAAGAGCGCTCCGAGCGCTATG | |
| 53. | TCTTGGCCCTACCTCCAAAATATTTCCAGATCTCCCTAGCCTGCA | DAP10 |
| | CACCCTTGCCACCTGTCATTCCCACTTGGACCAGGCCAGCAGCCT | |
| | CCCTGGTCTCTCTGACCCTCCCCCTGAGTTCGTTCACCAAAGGCA | |
| | GTAACGGAGACACCCCCTCAACACACACAGGAAGCAGATGGCCTT | |
| | GACACCAGCAGGGTGACATCCGCTATTGCTACTTCTCTGCTCCCC | |
| | CACAGTTCCTCTGGACTTCTCTGGACCACAGTCCTCTGCCAGACC | |
| | CCTGCCAGACCCCAGTCCACCATGATCCATCTGGGTCACATCCTC | |
| | TTCCTGCTTTTGCTCCCAGGTGAAGCCAGTGGTTACAGGGGATGG | |
| | TAGGCAGAGCGTTTGTGAGATGGGTGCTTGGGTGACGTCTGCAGG | |
| | GACGGGTGATGAAAGTGGGGTTCTTCTCCCTGCACCCCTTCCCTT | |
| | CTGGGAGATCCATTCTGCTTCAGGGCCTGGGTCCTTGGGGGCGGA | |
| | AGGGGGTGAGACAGGGAGTTCTGGAGGGGCTGCCTGTTAGCGTCC | |
| | CCTTCTCATGGCTGGGTCTCTGCTGCCACTTCCAATTTCTTGTCA | |
| | CTCTCCATGTCTCTGGGAGTCCCCTTCCCATGTGGTCCTGTTCCA | |
| | TCTCTCCAGCCTGGAGATTACTTCTCAGGACACTACCTTTCCTTC | |
| | TCTACACCCTATTTTTTGGTTTGTTTATTTTGAGATGGGGTCTTG | |
| | CTCTGTTGTCCAGGCTGGAGTGCAGTGGCACAATCACGGCTCACG | |
| | GCAGCCTTGACTTCCTGGGCTCAGGTGATCCTCCCAGCTCAGCCT | |
| | CCCGAGTAACTGGGATTACAGGTGTGAACCAACACTTCCAGCTAA | |
| | TTTTTGTATTTCTTGTAGAGACGAGGTCTCACTATGTTGCCCAGG | |
| | CTGGTCTCGAACTCCTGGGCTCAAGCGATCTTCCTGCCTCGGCCT | |
| | CCCAAAGTGCTGGGATGACAGGCGTGAGCCACGGTGCCAGGCTGA | |
| | GCATTCTGTTTTGTGGACCTTCTCTCCACCCTCATCCACCTTCTT | |
| | TCTCTTTCCACAGTGGCTGCAGCTCAGACGACTCCAGGAGAGAGA | |
| | TCATCACTCCCTGCCTTTTACCCTGGCACTTCAGGTATCACTTCC | |
| | ACCCCAGAAGCTTGGCCAGAGGCTCCCAGAACACCCCAGTGGTTC | |
| | TCCAGGTCACCATCCCACCTCCCGTCCCCAAATCAGAGGATCCGT | |
| | GTCCTTCTCCGAGTCCCAGAATCAGCGACCCCCAGCCTGTGTTCA | |
| | GGAGCACCCCGTGTGCCCGCCGCACAGCCCCGAGGGTCCTGGGAC | |
| | ACCCCAGCCTCTCTGCATCTGTCTCCCGTTTCATTCCCCAAGCGC | |
| | AACTCCAAGGAACCTGGGACCCGCCCCCTCGCAGGGGACTTCCTC | |
| | TCTGCCTGTGGCCAAAGCACAGCCCCAGGACGCAGAGCTTGAGTT | |
| | GTCTCCCTGTTCCGGCCCCCACTCTCCAGGCTCTTGTTCCGGATG | |
| | TGGGTCCCTCTCTCTGCCGCTCCTGGCAGGCCTCGTGGCTGCTGA | |
| | TGCGGTGGCATCGCTGCTCATCGTGGGGGCGGTGTTCCTGTGCGC | |
| | ACGCCCACGCCGCAGCCCCGCCCAAGGTGAGGGCGGAGATGGGCG | |
| | GGGCCTGGAAGGTGTATAGTGTCCCTAGGGAGGGGGTCCCAGGGA | |
| | GGGGGCCCTTGGGGAAGCCCTGGAGGAGGTGCTGGGGAAACCCTG | |
| | GGGGAGGTGCCTGGGGGAACCCCTGAGGAAACCCCTGAAGCAGGG | |
| | GGTCCCCAGGGAAGTGGAGATATGGGTGGTCAAGCTTCATGCTTT | |
| | CTCTCCCCTATCCCCAGAAGATGGCAAAGTCTACATCAACATGCC | |
| | AGGCAGGGGCTGACCCTCCTGCAGCTTGGACCTTTGACTTCTGAC | |
| | CCTCTCATCCTGGATGGTGTGTGGTGGCACAGGAACCCCCGCCCC | |
| | AACTTTTGGATTGTAATAAAACAATTGAAACACCTGTAGTCGTAT | |
| | TCTTTCTCAAAGAACCCCAGAGTTCCCAAAGCCTCCCTCCCATGA | |
| | ACTGTTTCTGGATCCAAGGCCCCCTCAGAACCCCCACATGTCCCC | |
| | ATCCC | |
| 54. | GAACAGAAACAAAAAGATATGAGACAAATTGAAAACGTATAGCAA | HLA-C |
| | AATGGTAGACCAAAACCCAACCATTATAAGTGAAGAAATGACACG | |
| | ACCTGAGTCACATTAGCAGGACTGCTGAGCACTGTGGGGAGAACA | |
| | GACATGGGCAGGAGGTGAGGGACAGTGTTAGTGCCACAATTCAGG | |
| | AGTGACAGGGTGGCGGGGACTAAAGGGGAAAGAGGGTGTGAGGGA | |
| | TGAGAGGGGCAGAGAGAAGGGCTGGAGAAGCAGGAGGTGAGGAAA | |
| | AGGAGCAGAGGAAAGAATTCTAAAGCAGTAGAAGAGCCTGGCAGG | |
| | GGGTTCTTTGCATTCGGTATTTAATACATTTTGTGTGACTGCCTT | |
| | AAAACTAATGGGCTCCTTATGATTTTTTTTTAAAAAGGGGTTACA | |
| | AAAATATCAAGTGTCCAAATAAAATATGCACACTGCTTAGATGTG | |
| | CATAGTTCACGAAAACGGGCAGTGCTGGAGCGCTGGTGAAGAGCA | |
| | TTGGGACTGCATGGAGCCCTCGCAACTTTGAGGTGATGACTACAG | |
| | GCTCCCGGTTGCAATAGACAGTAACAAACCCTGCTTCTTTGTATT | |
| | CAGGAGATGTTCTGGACTCACACAGGGAAACTCTGGCTAGAGAAT | |
| | GAGGATAACTTTAAATGCAACAACCCAGAGTCACAGAACCATAGT | |
| | CTGCGAAAGTAAAACAGGAGCTTTGAGAATTTAATTGTAATGCAG | |
| | TTTTGACACAGGTCTTTCACAGATTGGAATTCTAATCATTCAGGG | |
| | ATTACCAATATTGTGCTACCTACTGTATCAATAAACAAAAAGGAA | |
| | ACTGGTCTCTATGAGAATCTCTACCTGGTGCTTTCAGACAAAACT | |
| | TCACCAGGTTTAAAGAGAAAACTCCTGACTCTACACGTCCATTCC | |
| | CAGGGCGAGCTCACTGTCTGGCATCAAGTTCCCCATGGTGAGTTT | |
| | CCCTGTACAAGAGTCCAAGGGGAGAGGTAAGTTTCCTTTATTTTG | |
| | CTGGATGTAGTTTAATATTACCTGAGGTGAGGTAAGGTAAGGCAA | |
| | AGGGTGGGAGGCAGGGAGTCCAGTTCAGGGACGGGGATTCCAGGA | |
| | GGAGAAGTGAAGGGGAAGGGGCTGGGCGCAGCCTTGGGGTCTCTC | |
| | CCTGGTTTCCACAGACAGATCCTTGTCCAGGACTCAGGCACACAG | |
| | TGTGACAAAGATGCTTGGTGTAGGAGAAGAGGGATCAGGACGAAG | |
| | TCCCAGGTCCCGGGCGGGGCTCTCAGGGTCTCAGGCTCCAAGGGC | |
| | CGTGTCTGCATTGGGGAGGCGCCGCGTTGGGGATTCTCCACTCCC | |
| | CTGAGTTTCACTTCTCCCAACCTGCGTCGGGTCCTTCTTCCTGAA | |
| | TACTCATGACGCGTCCCCAATTCCCACTCCCATTGGGTGTCGGGT | |
| | TCTAGAGAAGCCAATCAGCGTCTCCGCAGTCCCGGTTCTAAAGTC | |
| | CCCAGTCACCCACCCGGACTC | |
| 55. | TTGGTAGAATGATTTATTTTCCTTTGGCTATATACCCAGCGATGG | NCR1 |
| | GATTGCTGGGCTGAATGGTAACTCTGTTTGTAGTTCTCTGAAATA | (NKp46) - |
| | TCTCCAAACCAAACTGCTTTCCACAGTGGCTGAACTAATTTACAC | Synthetic |
| | CCACCAACAGTGTATAAGTGTCCCCTTTGCTCCACAATCTCACCA | |
| | GCATCTGTTAATTTCTGGCTTTTCAGTAATGGCCATTCTGACTGG | |
| | TGTGAGATGGTATTGTTGAGGGATAATTTAGGAATCAGAGAGACC | |
| | GAGGGGTTGAGGAGGATTTATTATTATTATTATTATTTAGGTGCA | |
| | CCGGCCCCAGTCAGATTAACATCCAAAAAGACTGAGGCTCGAACA | |
| | GAGAGTCCGGTTACCTTTTAAGCATTTTGTGGGGTTGGGGGAGAT | |
| | CTGTGCAGGGGGAAGCATATTACAGAAGCAAGAAACAAAGGCAGT | |
| | TATTCAATTGAGACATGCATCACATTATTCCTTACTTTTCAAGAA | |
| | AAATATGTTTTACGACTTGAGGTTATCCTGTCTAGTGACCTTGCA | |
| | GCCGCACGGCAAGAGAAACAGGGTCTTCACAATGCCTGGGAAAGG | |
| | GAGAGATAAGGCTCACTAGCCACAGACAGAAAAACAGGCAGTTCA | |
| | TGTTTAAAGGACTCCACCTCTTTCTCTTCCTCGGGGGGAACTGGG | |
| | TTTTCTTAAATACAACTGAGTTTTTGTTTACACATTCTGTAATTT | |
| | CTTTTAATTCCTGTTCCAGTATCTCACTGTGAAACTCCCTATGTT | |
| | TTTATACGATTCTCAGGGGGTTTCCTCTGGGCATGATTGGGCACA | |
| | ACTTCCCACAGTCAGCTCTGGGTACGACCTCCACATTGCAGAATT | |
| | GAGAAGTTGACCCAGAAATGCATTTTGGGCTGAGCAGACAATTGT | |
| | CAGAGTTGCTGGCTAGACCACAGATGTGTCAGAGGGACCACGGCC | |
| | TTTCTGTAAGCTCATGGTCAGAGGCGGAGGGGAGTTGTGAACGTT | |
| | CTGATGAAAGCAGTCAACGTGAAAGCGCTCTGGTGATGGGCGCTG | |
| | GTGCTCACCCACCACTTCCTGTGTATCTATCTCCCTGGCCCGCCC | |
| | GGCTC | |
| 56. | TAGGACTCTGCAGCTGAGTTTATCTGCTGCAGTTTGGCACTTCTT | CD34 |
| | GCCCTAAGCTTTTTGAGACAAAGATTGCGAAGGAAGGAGATGAGG | |
| | ACAAGGGAATGAGGAGGCCCAGGAGGTGGAGCCAAAGGGATAAGA | |
| | AAAAGAGGCCCCAGGCCCCTGTGTGTTTTATCAGCCGAACAGAAA | |
| | TGCTGCTGAAGACATGCATCTGCAAAGGTGGTGCCAACTGGGTTG | |
| | GGTCTACAAGTGCGGAGCACTGGGGACCACTGAATTAGTCAGAGG | |
| | TCCCCAAGCCTGCTCCTCCTCTGTCGCCATATGGCAGAGATTCAA | |
| | AGGGTGGAGTCCTCTCCCCTCTGGAAAATAGCTGATAAAATTCTT | |
| | TTCCCTTTTATTTTTCCCCTCTTACATGTAGCCCTGGACAGGATA | |
| | TGGGTGTGGGGTCAGCAGGCACGAAGGACCCAAAGGAAGAAGAGG | |
| | AATGTGACGGGGAGGGCCTGGGGGTGTTGTTAGAGTGAGGATAGG | |
| | GACAGGCCTTGGAAAGCTTGGAGGAAATAGATACCCTGAGCCTGA | |
| | CAAAGGGGTTCCCTAATTACCTGAAAGGCTTCCTGCTCCAGCCAG | |
| | CACTCTGGCAGTGCTGCCTGGTGGCCCTTGAGCCCCTTACCCTGA | |
| | CTCTGCTCTGTGATCTCCCCCACTTCCAACAAAGACAGCCCACTT | |
| | CTGGTTAGTTGCAGTGCCTATGCACATGCCACCTTCTCAAAGGGA | |
| | CAGAAAAAAAGAACTTCCCAAAACAAGCTCTTTGTTGGGGTAAAT | |
| | GATGAGAAAACAAACCACAGAAATCCCCTCTACCCTAAATTTTAC | |
| | AATAATAATAGCATTTCTGCAATGGCCCTATGGTAGCATTTCAAG | |
| | GTACACTCAGTTATCTCATTTATCCCACAATGTATCCCAGGACTG | |
| | AAAATGACTCCTAGTATTAACAAAACAAAACAAATCCAAGCAAAA | |
| | ACCCTAGAAAGCTCTTGGGAAGGGCATAAAACTACAATTGGCTCA | |
| | AGTAGTCTGGCAAGGCTGGGAATGGGGCCTTGTGCCTCATCCCTC | |
| | CCCACCAAAAGCTGGGATATTCTCAGCATATCAGTCCAAAGTTTT | |
| | GCCAAGGTTGTGTTGCTCCCCCAACCCCAACCTCGGCAGGCCAAG | |
| | GCGAGACTGGCACTTACCAGCTGAGGAAATAAGAAAAGGGTGGAC | |
| | CTCATTCGGCTGAGGACCAGGTCATCACAAGCTGTGTAGCCAAAG | |
| | GAGAATGGAGCATCTGATTGGAATCTGCACCACTGGCCAACTCAC | |
| | CCCTGTTTTTTTGTTTTTTGTTTTTTGTTTGTTTGTTTGTTTTGA | |
| | GAAGGAGTCTCGCTCTGTCTCCCAGGCTGGAGTGCAGTGCCAGGA | |
| | TCTTGGCTCGCTGCAACCTCCGCCTCCTGGGTTCAAGGGATTCTC | |
| | CTGCCTCAGCCTCCCGAGTAGCTGGGACTATAGGGGTGTGCCACC | |
| | ATGCCCAGCTAATTTTTGTAATTTTAGTAGAGATGGGGTTTCACC | |
| | ATGTTGGCCAGGATGGTCTCGATCTCTTGACCTCATGATCTGCCT | |
| | GCCTCGGCCTCCCAAAGTGCTGGGATTGCAAGCGTGAGCCACTGC | |
| | GCCCGGCCGACTCACTCCTGTTATGAGGGCCTAGTCTAGCACCCA | |
| | CTCTAGAATTTTGAAACCTCATTGATAGCTTTTCCTTGCCCCAAA | |
| | GCACACATCCAGGACTGCCAAACAGGGCAACCCAAAACTTCTCTC | |
| | AGAGCTTCCTCCAGCCCAGGGGCAGCTGTCCCCAGCTGGGCGGAC | |
| | CCAAAGATGGACAACAGAGGAGACTGGGAACAAAGAGGTCTAGTC | |
| | CAGGTCACTCTGCAGGGCCACAGTGGGTGGCCCTTAGGATGTAGT | |
| | GAGAGTACAGAATTGGGAGCCCTAGAGTGCTGTAAGCAAAAGGGA | |
| | GAGCAATGTACAAAGTTTCAAAAGTTTCAAACCTTCCAAGTGCAA | |
| | AGAGATGAGGTGGAGAATGTCATTCCCCAAGGAAAGTAATCGAGT | |
| | ATAAATATTTACCGGGTGCCTATTTTTTTTTTAACCGAAGTGAGA | |
| | GCAAGACCTTAAGAGTAAGGATATGTGTAGGTGAGTGGGGGCAGT | |
| | GTTCTAACCCATCCACTAGCTTTAGAACTTGGAGCAGGGGCAGAG | |
| | GAAGAAAACTCCAAAAGGTGACAAGTTTTGAAATTTGTGCAGGGG | |
| | TGACGGTTTTTGGTAAGGCGAGCGAGGCTGGGCGCAGAGCAAATT | |
| | CTGTCTACTTCTACCTCCTCCGCGGTGGGGTTGCTGAAGTGGGAA | |
| | TTCAAACTCAGCTTCCCAATCTTAAATGCAAGGTTTTTGTTTGGT | |
| | TGGTTGTTTGGTTTTGTTTTTCCTTTTCACGGACTGTTCATTCCG | |
| | AACACCCCTGGCCTGGGACCTGGAGCCTCATCCCTGATTCGCTTC | |
| | CCTCGAATTCCTTAATGTCCACTTCTGGCTCTCCCAGTCCCTGTG | |
| | CAAGGTGGGCACACCGGCCCCTTCCCAGCAATCCGCTCCGTGTCT | |
| | ATACTTTCTGAATCAACCAAGAGCCACAAAACTTTTCCTGCTTCT | |
| | CTCCTCCCCAGTCTCCCGGTTCTGGCTGCAACTTCGCACTCCGCG | |
| | CCTCTGGCAACCAGAAGAGACCCTGGCATCTCCAAAGCGCTTTAT | |
| | CTCAGTCCATTGGAACCAGTCGTGCTGACCGATTCACACCTCGGC | |
| | TAACGCACACTCGCGGGGGTGATGGCCTTCCCTTCCCCTCCCACC | |
| | CCCGTCAGCTCCCAGGTGAGGCGCTGGCCCCGGGGGGAAGCAGCT | |
| | GTGGGCGGCAGTGCGTGGGTTGGGCAGGGGTCCCTTCCCTCCGTG | |
| | AGACTCTGCTCTGCTGTTCAGCCTCCCAGAAAGCCTCCACCCTCC | |
| | CCGCGGCGAAGCCAAGCGGCCGCGGCGCGCGGGCGGTACTCACGC | |
| | AGCAAACTCAGCAAGCAAAGCGCGGTCCAGCCCCGCGGCATCCTG | |
| | GGCCCTGCGCGCGCGCCCCTGCGGACCAGCATCCTTCCCGCGCGG | |
| | CTCCTAGAGAGACGCACCGAGTGGAAGACAC | |
| 57. | TATTCTTGGAACCTGCAAGGCCTGGCAAGATTTGGCCATCATCTC | CD45 |
| | CCTCTGAAACCTCCTTATTTTCCAGGCTCTGCTTCTTTCTACATC | |
| | AGCCTCACATCTCTTCCTGTTCCTTGAACACAGTCTCCTCCTTTA | |
| | AATTTATTATTTCTGCCTAGAATACTTTCCTTCTCCTCATACTCA | |
| | TTTAACTTATTTTCTTCAGGTCTAGCTCCATTGCTTTTTATTCCA | |
| | GGAAAACTTCCCAACCTCCAGAATAGACCAAATATCCTATTATAT | |
| | GCTTAAATCACAGTGTGGAAGTATTGCCTTTGTGATGAAAGTGTC | |
| | ATGATGACTGGATAATTAATTATTGTGTCTTCCACTAAAGTGTAG | |
| | ATAGGAAAGAAAGCATGCCTGTTTTTGCTCACCATTTTTTCCTCA | |
| | TGTCAGGTAAAATGACTGGATTACATTAGGAACTTGCTCTGTATT | |
| | TGTTGAATGAATGAGTGAATGAATGCATGAACAAATGAGTAACAT | |
| | CTGCCTCTGTAGTTAACTTGTAATCTGTTTCTGTCTTTATATAAC | |
| | CTCTACTGTAGGTAGCCTTAAAAATCTGGAAAATGACTTTATTGA | |
| | TTTAAGTAAATATTAAGTTTTTAAGCTTTTAAAAATCTATTGTAA | |
| | GTTGATTAACTATGATTATTTTGAAATTTTCTTCATCAAAATTTG | |
| | AGATGACTTTGGATACATACTGTTTTCAGCTACTCAACAAGTGCC | |
| | ATTGTATTTACTATGTATGAAATCTGGTGTTACTTCCTGCCCATT | |
| | TCCTAGACTCAGTTGTTCTTTGCAAAATACCCATTTATCAGCAAC | |
| | CAGCCCACCCTCCCTCTGGGAATCAGTGAGAGTGGATCACATCAG | |
| | TATGTCATAAATACTCCTCCTGTTTTCTTTGAGAGTTTTCTGCTG | |
| | ATGCAAGCGGAAGGCTTGGATGCCAGGCCTGTATGTGATACAGTA | |
| | GAAATTTCCCCAGATGCCCTGTGGAAAGACAAGCTGCTACCCGGA | |
| | GTCCTTCCTGGATGTCTGGCTTAGTGTAGTATACCATCAAGGACA | |
| | ACCTGGCTCTGTCTCCTAACAAGCTGCTAGGGCCTTCCCTCTTGC | |
| | CTACCCTCCTGACAGCCTGGACCTCAGGCTAACAAACCTCTGGGT | |
| | TGCCACTGTCTCCATTCTGAGCCATCAGAATTGTATATACATTCA | |
| | TCTGCTGATGTCTCTGTATTTCAAGCTGTATGTCTTTACACCTTT | |
| | ATAATACACAGTACAGATTTTGCTGCTTAAAATTCTAGTATTTCC | |
| | TATAGACACTAAACTCAATTTTAACACAATGAATATGTTAAAACA | |
| | TTATATATGTGTTGGATTAACACATATAATGCAACTCATTTGTTG | |
| | CTAATCTATCTTGGATTCCTTGGGTAGAATCTGTCTTCTCACCTG | |
| | TCTGAAATGGAATTGAATCTCCTAACTTCTTACTATTTTTTTAAA | |
| | GCCACAAATCCCTTTTAAAATGTCCGTTTAAGGCTGCTAAACTCA | |
| | CATTGTTATAGAGTATATTTTATATACTTTTCTTCCTTTTAAAAG | |
| | ATCATATACACTTTTCTCCTGGCCTCTAATTTTGCCAAATATTCT | |
| | GCCATAACAGCAAGCTCACAGAACACAAGTATTTGCTTAAAGATA | |
| | TTTAAAGCAAATCCCCAAAGAGTTTTCTGTTGCAAATGTATTTTA | |
| | AGACATAGGCTGAGGAGCTGAGTATTCAAATCCATGAGTTCTGAC | |
| | ACTGATGTTTTATTAGGCCTACCCACATGAAAATTAGTGCTTACT | |
| | TGCACAGTTATCTTATTTAAAAATACATTTGCAAAAAGAGAATAA | |
| | TTTCATTTTCACTGATTCAAAGGTCTATCTTTTCGAGTATTATAA | |
| | ACTTACATAGTACAAAAAGATTGGTAAAAGGGATGAGAAAAATAA | |
| | AGGGCAATAGTAAGGTGAGTAAGGAAAGCAAAATTAAACTTGGAG | |
| | ACTTGGGTTCTAAATATAGCTTGTCTACATCCTAGCTGCAAGATC | |
| | TTAGGCAACTCAACTAACCTCTCTGAACATCACAGATAAAATGGG | |
| | GAATAATAACATTATCTAGCTCAAGGGTATCGTACAAATTATACA | |
| | ATATATGTAAAGTGCAAAGCATAGTTCCTGCACATAGTAAGTGCT | |
| | CCATAAGTTAAATATAATAATAATCATAGTAAAATAATATTCTTA | |
| | ACTACAATCTTATTAAAAGGTTATTACCTAGAGTTTAAGCTGTGG | |
| | GGTTATTTCCATTTGCACAAAGTGTGATAGAAATTTGGCTCTGTA | |
| | GTTCCTAGCAGCCAATGTAAGAGTAAAACAAAAAAGTTTGTAAGT | |
| | ATCAAAAGATTTATTTACTTAAATATAAGAACCCTACAATAATGC | |
| | TTCCAAACTAGGGTTTGCAGAATTACCACAAGGGCTCTGTCTGTC | |
| | TAAAAGTCCCCCAAGCATTATCTATTGTTCAAATAAATGAGTTGC | |
| | ACATGTGTGTACTTTTATTTTTAAAGGCATATAGATATTGTATTA | |
| | ATAAAACCAATTCATCTAATTGTATTGTTGAATTTATACTTTTTG | |
| | CCAGTAAGCATTTTCCTAATAGATGGACTTAACTAAGAACATCAG | |
| | AATGGTGAAGAGCATTGTGTTAATACAATTCATTGTCTTATTTGT | |
| | TAAAAAAGTTGAAAAAGTATTGCTGAGAAAAAGGAAACAAGTTAA | |
| | CTTTTGTTAAAGGTATCTCTTGGTTTTCACTGTCCTTTCCCTCAT | |
| | CAGTAGCGCCAAGAACATCTTAAGTCACAGAAACATTAGTTTTTG | |
| | GAAGCAGGGTTTGCTGTAACTATAGTAGAAATGACATTCTGATTC | |
| | CACTCCTAGCTTCACAAGGATATCTGTGAAAGATTTGGGGCAAAA | |
| | CTGTTAAGCTGTCTGAAAGTGCTTTTGCATAAGAAATGGGTTTTA | |
| | CTGCTAAAACTGTCATATTGCTGAGTTTTGAATGCCCTAATGGTA | |
| | AATGATACTGGGTTGCCAAAAATAACCAGATTAGTAGTTTTTTCA | |
| | TTCATTTGGCCGTCTCAGTAAGTCAAATATT | |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to T2be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents.

What is claimed is:

1. A method for gene expression in a cell, said method comprising:
differentiating a cell comprising:
i. an inducible promoter configured to modulate an expression of one or more tolerogenic factor(s);
ii. an open reading frame (ORF) encoding said tolerogenic factor(s) in operable connection with said inducible promoter; and
to conditions sufficient to differentiate said cell into a differentiated cell;
wherein concurrent with or subsequent to such differentiation, said inducible promoter is activated in said differentiated cell to modulate said expression of or gene signaling via said tolerogenic factor(s).

2. The method of claim 2, wherein said inducible promoter is delivered exogenously to said cell.

3. The method of claim 1 or claim 2, wherein said inducible promoter is a cell-specific promoter.

4. The method of claim 3, wherein the cell-specific promoter is an immune cell gene promoter.

5. The method of claim 3, wherein the cell-specific promoter is a white blood cell gene promoter. In some cases, the cell-specific promoter is a neutrophil gene promoter, an eosinophil gene promoter, a basophil gene promoter, a mast cell gene promoter, a monocyte gene promoter, a macrophage gene promoter, a dendritic cell gene promoter, a natural killer (NK) cell gene promoter, a memory-like NK cell gene promoter, a lymphocyte gene promoter, a B cell gene promoter, a T-cell gene promoter, a regulatory T-cell (Treg) promoter, a hepatocyte gene promoter gene promoter, a cardiomyocyte gene promoter, a renal cell gene promoter, a dopaminergic neuron gene promoter, a pancreatic islet cell gene promoter, a macrophage gene promoter, or a retinal pigment epithelium cell gene promoter.

6. The method of claim 3 or 4, wherein the cell-specific promoter is an NK cell gene promoter or a T-cell gene reporter.

7. The method of any one of claims 4-6, wherein the inducible promoter is an L selectin (CD62L) promoter, an interferon-gamma (IFNg) promoter, neural cell adhesion molecule (CD56) promoter, a CD56 promoter, a KIRs promoter, a CD16 promoter, a NKp44 promoter, a NKp46 promoter, a NKG2D promoter, a TRAIL promoter, a CD122 promoter, a CD27 promoter, a CD244 promoter, a NK1.1 promoter, a NKG2A/C promoter, a NCR1 promoter, a Ly49 promoter, a CD49b promoter, a CD11b promoter, a KLRG1 promoter, a CD43 promoter, a CD62L promoter, a CD226 promoter, a TRAC promoter, a TRBC promoter, a CD3 promoter, a CD4 promoter, a ThPOK promoter, a CD8 promoter, a FOXP3 promoter, a Helios promoter, a CD25 promoter, a GARP promoter, a GPA33 promoter, a CD14 promoter, a CD11b promoter, a CD68 promoter, a CD138 promoter, an IgH promoter, an IgK promoter, an IgL promoter, an IgG promoter, a CD19 promoter, a CD20 promoter, a TH promoter, a TUBB3 promoter, a FOXA2 promoter, a GIRK2 promoter, a Nurr1 promoter, a DAT promoter, an INS promoter, a chromogranin A promoter, a synaptophysin promoter, a neuron-specific enolase promoter, or a Leu7 promoter.

8. The method of any one of claims 4-7, wherein the cell-specific promoter is a memory-like NK cell gene promoter.

9. The method of any one of claims 4-8, wherein the cell-specific promoter is an L selectin (CD62L) promoter, an interferon-gamma (IFNg) promoter, or neural cell adhesion molecule (CD56) promoter.

10. The method of any one of claims 4-8, wherein the cell-specific promoter is a CD16 promoter, a HLA-C promoter, a NKp46 promoter, or a DAP10 promoter.

11. The method of any one of claims 4-10, wherein the cell-specific promoter comprises a sequence having 95%, 97%, 99% or 100% sequence identity to a 60, 70, 80, 90, 100, 200, 500, 1,000, 1,500 or 2,000 base pair long fragment of any one of SEQ ID 5 to 57.

12. The method of any one of claims 4-10, wherein the cell-specific promoter comprises a sequence having at least 95% sequence identity to a sequence that comprises 500 bp of any one of SEQ ID 5 to 57.

13. The method of any one of claims 4-10, wherein the cell-specific promoter comprises a sequence having at least 99% sequence identity to a sequence that comprises 500 bp of SEQ ID 5 to 57.

14. The method of claim 1, said inducible promoter exists endogenously within said stem cell.

15. The method of any one of claims 1-14, wherein said tolerogenic factor comprises a SIRPα binding sequence.

16. The method of claim 15, wherein said SIRPα binding sequence is a CD47 sequence or functional variant thereof.

17. The method of any one of claims 15-16, wherein said SIRPα binding sequence comprises a sequence having at least about 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identity to any one of SEQ ID NOs: 1-4.

18. The method of any one of claims 1-17, wherein said tolerogenic factor comprises CD47, cluster of differentiation 24 (CD24), complement receptor 1 (CR1), complement decay-accelerating factor (CD55), cluster of differentiation 46 (CD46), cluster of differentiation 59 (CD59), HLA-A, HLA-B, HLA-C, HLA-E, HLA-F, HLA-G, cluster of differentiation 39 (CD39), cluster of differentiation 73 (CD73), programmed death-ligand 1 (PD-L1), group 2 cluster of differentiation 1 (CD1D), adenosine A_{2A} receptor (A2AR), B7 Homolog 3 (B7-H3), B7 Homolog 4 (B7-H4), B- and T-lymphocyte attenuator (BTLA), cytotoxic T-lymphocyte associated protein 4 (CTLA-4), indoleamine-pyrrole 2,3-dixoygenase (IDO), killer-cell immunoglobulin-like receptor (KIR), lymphocyte-activation gene 3 (LAG3), NADPH oxidase 2 (NOX2), programmed death 1 receptor (PD-1), T-cell immunoglobulin and mucin-domain containing-3 (TIM-3), V-domain Ig suppressor of T cell activation (VISTA), Sialic acid-binding Ig-like lectin (SIGLEC7), or any variant, functional fragment, or combination thereof.

19. The method of any one of claims 1-18, wherein said tolerogenic factor comprises CD47 or a variant thereof, and one or more tolerogenic factors selected from the group consisting of CD24, CR1, CD55, CD46, CD59, HLA-A, HLA-B, HLA-C, HLA-E, HLA-F, HLA-G, CD39, CD73, PD-L1, CD1D, A2AR, B7-H3, B7-H4, BTLA, CTLA-4, IDO, KIR, LAG3, NOX2, PD-1, TIM-3, VISTA, SIGLEC7, or any variant, functional fragment, or combination thereof.

20. The method of any one of claims 1-8, wherein at least a subset of said one or more tolerogenic factors is recombinant.

21. The method of any one of claims 1-20, wherein said ORF is in operable connection with said inducible promoter.

22. The method of any one of claims 1-21, wherein said stem cell further comprises an additional ORF encoding an additional tolerogenic factor.

23. The method of claim 22, wherein said additional ORF encoding said additional tolerogenic factor is in operable connection with said inducible promoter.

24. The method of claim 22, wherein said additional ORF comprising said additional tolerogenic factor is in operable connection with another inducible promoter.

25. The method of claim 23, wherein said additional ORF comprising said additional tolerogenic factor is in operable connection with a constitutive promoter optionally selected from a beta-2 microglobulin (B2M) promoter, a class II major histocompatibility complex transactivator (CIITA) promoter, and a CAG promoter.

26. The method of any one of claims 1-25, further comprising providing in said cell a cell-specific gene configured to modulate the expression of said tolerogenic factor.

27. The method of any one of claims 1-26, further comprising tagging an activator or repressor molecule to said inducible promoter.

28. The method of any one of claims 1-27, wherein said ORF encoding said tolerogenic factor is comprised in sequence having a transcription activator in operable connection with said inducible promoter.

29. The method of any one of claims 1-28, wherein said differentiated cell is a natural killer (NK) cell, a T-cell, a B-cell, a hepatocyte, a cardiomyocyte, a renal cell, a dopaminergic neuron, a pancreatic islet cell, a macrophage, a nephrocyte, or a retinal pigment epithelium cell.

30. The method of any one of claims 1-29, wherein said cell is an embryonic stem cell (ESC), a mesenchymal stem cell (MSC), an induced pluripotent stem cell (iPSC), or a hematopoietic stem cell (HSC).

31. The method of any one of claims 1-30, wherein said stem cell further comprises a sequence encoding a translation control sequence.

32. The method of claim 31, wherein said sequence encoding said translation control sequence is positioned 5′ or 3′ of said open reading frame encoding said tolerogenic factor.

33. The method of any one of claims 31-32, wherein said sequence encoding said translation control sequence is located between said ORF encoding said tolerogenic factor and another ORF encoding a native gene of said differentiated cell.

34. The method of claim 33, wherein said native gene is B2M or CIITA.

35. The method of any one of claims 1-34, wherein said stem cell further comprises another ORF encoding a chimeric antigen receptor (CAR) or T-cell receptor (TCR), or functional fragment thereof.

36. The method of any one of claims 1-35, wherein said another ORF encoding said CAR or TCR, or said functional fragment thereof, is in operable connection with said inducible promoter.

37. The method of any one of claims 1-36, wherein said cell further comprises another ORF encoding a costimulatory protein, optionally selected from a cluster of differentiation 28 (CD28) protein, an inducible T-cell costimulator (ICOS) protein, or any functional fragment or combination thereof.

38. The method of any one of claims 1-37, wherein said inducible promoter and said ORF encoding said tolerogenic factor are comprised in a nucleic acid molecule.

39. The method of claim 38, wherein said nucleic acid is comprised in a vector.

40. The method of claim 39, wherein said vector is a plasmid, a minicircle, a CELiD, an adeno-associated virus (AAV) derived virion, a lentivirus, an adenovirus, or a herpes simplex virus (HSV).

41. The method of claim 38, wherein said nucleic acid is comprised in a genome of said cell.

42. A construct for gene expression in a cell, said construct comprising:
(a) an inducible promoter; and
(b) an open reading frame encoding a tolerogenic factor,
wherein said inducible promoter is configured to modulate an expression of said tolerogenic factor, wherein said inducible promoter is configured to activate upon or subsequent to differentiation of said cell into a differentiated cell.

43. The construct of claim 42, wherein said ORF encoding said tolerogenic factor comprises said inducible promoter.

44. The construct of claim 42, wherein said ORF encoding said tolerogenic factor does not comprise said cell-specific inducible promoter.

45. The construct of any one of claims 42-44, wherein said tolerogenic factor comprises a SIRPα binding sequence.

46. The construct of claims 42-45, wherein said SIRPα binding sequence is a CD47 sequence or functional variant thereof.

47. The construct of any one of claims 42-46, wherein said SIRPα binding sequence comprises a sequence having at least about 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identity to any one of SEQ ID NOs: 1-4.

48. The construct of any one of claims 42-44, wherein said tolerogenic factor comprises CD47, cluster of differentiation 24 (CD24), complement receptor 1 (CR1), complement decay-accelerating factor (CD55), cluster of differentiation 46 (CD46), cluster of differentiation 59 (CD59), HLA-A, HLA-B, HLA-C, HLA-E, HLA-F, HLA-G, cluster of differentiation 39 (CD39), cluster of differentiation 73 (CD73), programmed death-ligand 1 (PD-L1), group 2 cluster of differentiation 1 (CD1D), adenosine A_{2A} receptor (A2AR), B7 Homolog 3 (B7-H3), B7 Homolog 4 (B7-H4), B- and T-lymphocyte attenuator (BTLA), cytotoxic T-lymphocyte associated protein 4 (CTLA-4), indoleamine-pyrrole 2,3-dixoygenase (IDO), killer-cell immunoglobulin-like receptor (KIR), lymphocyte-activation gene 3 (LAG3), NADPH oxidase 2 (NOX2), programmed death 1 receptor (PD-1), T-cell immunoglobulin and mucin-domain containing-3 (TIM-3), V-domain Ig suppressor of T cell activation (VISTA), Sialic acid-binding Ig-like lectin (SIGLEC7), or any variant, functional fragment, or combination thereof.

49. The construct of any one of claims 42-48, wherein at least a subset of said one or more tolerogenic factors is recombinant.

50. The construct of any one of claims 42-49, wherein said ORF is in operable connection with said inducible promoter.

51. The construct of any one of claims 42-50, wherein said stem cell further comprises an additional ORF encoding an additional tolerogenic factor.

52. The construct of claim 42-51, wherein said additional ORF encoding said additional tolerogenic factor is in operable connection with said inducible promoter.

53. The construct of claim 52, wherein said additional ORF comprising said additional tolerogenic factor is in operable connection with another inducible promoter.

54. The construct of claim 52, wherein said additional ORF comprising said additional tolerogenic factor is in operable connection with a constitutive promoter.

55. The construct of claim 54, wherein said constitutive promoter is a beta-2 microglobulin (B2M) promoter, a class II major histocompatibility complex transactivator (CIITA) promoter, or a CAG promoter.

56. The construct of any one of claims 42-55, further comprising a cell-specific gene configured to modulate the expression of said tolerogenic factor.

57. The construct of any one of claims 42-56, further comprising an activator or repressor molecule tagged to said inducible promoter.

58. The construct of any one of claims 42-57, wherein said open reading frame encoding said tolerogenic factor is comprised in a transcription activator in operable connection with said inducible promoter.

59. The construct of any one of claims 42-58, wherein said inducible promoter is a cell-specific promoter.

60. The construct of claim 59, wherein the cell-specific promoter is an immune cell gene promoter.

61. The construct of claim 59, wherein the cell-specific promoter is a white blood cell gene promoter. In some cases, the cell-specific promoter is a neutrophil gene promoter, an eosinophil gene promoter, a basophil gene promoter, a mast cell gene promoter, a monocyte gene promoter, a macrophage gene promoter, a dendritic cell gene promoter, a natural killer (NK) cell gene promoter, a memory-like NK cell gene promoter, a lymphocyte gene promoter, a B cell gene promoter, a T-cell gene promoter, a regulatory T-cell (Treg) promoter, a hepatocyte gene promoter gene promoter, a cardiomyocyte gene promoter, a renal cell gene promoter, a dopaminergic neuron gene promoter, a pancreatic islet cell gene promoter, a macrophage gene promoter, or a retinal pigment epithelium cell gene promoter.

62. The construct of claim 61, wherein the cell-specific promoter is an NK cell gene promoter or a T-cell gene reporter.

63. The construct of any one of claims 59-62, wherein the inducible promoter is an L selectin (CD62L) promoter, an interferon-gamma (IFNg) promoter, neural cell adhesion molecule (CD56) promoter, a CD56 promoter, a KIRs promoter, a CD16 promoter, a NKp44 promoter, a NKp46 promoter, a NKG2D promoter, a TRAIL promoter, a CD122 promoter, a CD27 promoter, a CD244 promoter, a NK1.1 promoter, a NKG2A/C promoter, a NCR1 promoter, a Ly49 promoter, a CD49b promoter, a CD11b promoter, a KLRG1 promoter, a CD43 promoter, a CD62L promoter, a CD226 promoter, a TRAC promoter, a TRBC promoter, a CD3 promoter, a CD4 promoter, a ThPOK promoter, a CD8 promoter, a FOXP3 promoter, a Helios promoter, a CD25 promoter, a GARP promoter, a GPA33 promoter, a CD14 promoter, a CD11b promoter, a CD68 promoter, a CD138 promoter, an IgH promoter, an IgK promoter, an IgL promoter, an IgG promoter, a CD19 promoter, a CD20 promoter, a TH promoter, a TUBB3 promoter, a FOXA2 promoter, a GIRK2 promoter, a Nurr1 promoter, a DAT promoter, an INS promoter, a chromogranin A promoter, a synaptophysin promoter, a neuron-specific enolase promoter, or a Leu7 promoter.

64. The construct of any one of claims 59-63, wherein the cell-specific promoter is a memory-like NK cell gene promoter.

65. The construct of any one of claims 59-64, wherein the cell-specific promoter is an L selectin (CD62L) promoter, an interferon-gamma (IFNg) promoter, or neural cell adhesion molecule (CD56) promoter.

66. The construct of any one of claims 59-64, wherein the cell-specific promoter is a CD16 promoter, a HLA-C promoter, a NKp46 promoter, or a DAP10 promoter.

67. The construct of any one of claims 59-66, wherein the cell-specific promoter comprises a sequence having 95%, 97%, 99% or 100% sequence identity to a 60, 70, 80, 90, 100, 200, 500, 1,000, 1,500 or 2,000 base pair long fragment of any one of SEQ ID 5 to 57.

68. The construct of any one of claims 59-67, wherein the cell-specific promoter comprises a sequence having at least 95% sequence identity to a sequence that comprises 500 bp of any one of SEQ ID 5 to 57.

69. The construct of any one of claims 59-68, wherein the cell-specific promoter comprises a sequence having at least 99% sequence identity to a sequence that comprises 500 bp of SEQ ID 5 to 57

70. The construct of any one of claims 59-69, wherein said differentiated cell is a natural killer (NK) cell, a T-cell, a B-cell, a hepatocyte, a cardiomyocyte, a renal cell, a dopaminergic neuron, a pancreatic islet cell, a macrophage, a nephrocyte, or a retinal pigment epithelium cell.

71. The construct of any one of claims 59-70, wherein said cell is a stem cell.

72. The construct of claim 71, wherein said stem cell is an embryonic stem cell (ESC), a mesenchymal stem cell (MSC), an induced pluripotent stem cell (iPSC), or a hematopoietic stem cell (HSC).

73. The construct of any one of claims 59-72, further comprising a sequence encoding a translation control sequence.

74. The construct of claim 73, wherein said translation control sequence is an internal ribosome entry site (IRES) or 2A peptide.

75. The construct of claim 73 or 74, wherein said sequence encoding said translation control sequence is positioned 5′ or 3′ of said open reading frame encoding said tolerogenic factor.

76. The construct of any one of claims 73-75, wherein is said sequence encoding said translation control sequence is located between said ORF encoding tolerogenic factor and another ORF encoding a native gene of said differentiated cell.

77. The construct of claim 76, wherein said native gene is B2M or CIITA.

78. The construct of any one of claims 59-77, wherein said construct further comprises another ORF encoding a chimeric antigen receptor (CAR) or T-cell receptor (TCR), or functional fragment thereof.

79. The construct of claim 78, wherein said CAR or TCR, or said functional fragment thereof, targets a tumor-specific antigen.

80. The construct of claim 78 or claim 79, wherein said CAR or TCR, or said functional fragment thereof, comprises a TCR-α chain, a TCR-β chain, a TCR-γ chain, a TCR-δ chain, a TCR-ζ chain, a CD3 molecule, or any functional fragment or combination thereof.

81. The construct of claim 79 or 80, wherein said another ORF encoding said CAR or TCR, or said functional fragment thereof, is in operable connection with said inducible promoter.

82. The construct of any one of claims 59-81, further comprising another ORF encoding a costimulatory protein; wherein is costimulatory protein optionally comprises a cluster of differentiation 28 (CD28) protein, an inducible T-cell costimulator (ICOS) protein, or any functional fragment or combination thereof.

83. The construct of any one of claims 59-82, wherein said construct is a nucleic acid molecule.

84. The construct of claim 83, wherein said construct is a vector.

85. The construct of claim 84, wherein said vector is a plasmid, a minicircle, a CELiD, an adeno-associated virus (AAV) derived virion, a lentivirus, an adenovirus, or a herpes simplex virus (HSV).

86. The construct of claim 85, wherein said construct is integrated into a genome of said cell.

87. A method comprising: subjecting a cell comprising the construct of any one of claims 59-86 to conditions sufficient to differentiate said cell into a differentiated cell.

88. A method, comprising: subjecting a cell that does not express a tolerogenic factor to conditions sufficient to differentiate said cell into an immune cell, wherein upon or subsequent to differentiation, said immune cell expresses said tolerogenic factor.

89. The method of claim 88, wherein said tolerogenic factor is a CD47 sequence or functional variant thereof.

90. The method of any one of claims 88-89, wherein said tolerogenic factor comprises CD47, cluster of differentiation 24 (CD24), complement receptor 1 (CR1), complement decay-accelerating factor (CD55), cluster of differentiation 46 (CD46), cluster of differentiation 59 (CD59), HLA-A, HLA-B, HLA-C, HLA-E, HLA-F, HLA-G, cluster of differentiation 39 (CD39), cluster of differentiation 73 (CD73), programmed death-ligand 1 (PD-L1), adenosine A_{2A} receptor (A2AR), B7 Homolog 3 (B7-H3), B7 Homolog 4 (B7-H4), B- and T-lymphocyte attenuator (BTLA), cytotoxic T-lymphocyte associated protein 4 (CTLA-4), indoleamine-pyrrole 2,3-dixoygenase (IDO), killer-cell immunoglobulin-like receptor (KIR), lymphocyte-activation gene 3 (LAG3), NADPH oxidase 2 (NOX2), programmed death 1 receptor (PD-1), T-cell immunoglobulin and mucin-domain containing-3 (TIM-3), V-domain Ig suppressor of T cell activation (VISTA), Sialic acid-binding Ig-like lectin (SIGLEC7), or any variant, functional fragment, or combination thereof.

91. The method of any one of claims 88-90, wherein at least a subset of said one or more tolerogenic factors is recombinant.

92. The method of any one of claims 88-91, wherein said cell comprises an inducible promoter and an open reading frame (ORF) encoding said tolerogenic factor.

93. The method of claim 92, wherein said inducible promoter is delivered exogenously to said cell.

94. The method of claim 93, wherein said inducible promoter exists endogenously within said cell.

95. The method of any one of claims 92-94, wherein said ORF is in operable connection with said inducible promoter.

96. The method of any one of claims 92-95, wherein said ORF is provided on a vector.

97. The method of claim 96, wherein said vector is a plasmid, a minicircle, a CELiD, an adeno-associated virus (AAV) derived virion, a lentivirus, an adenovirus, or a herpes simplex virus (HSV).

98. The method of any one of claims 92-97, wherein said ORF is integrated into a genome of said stem cell.

99. The method of any one of claims 92-98, wherein said stem cell further comprises an additional ORF encoding an additional tolerogenic factor.

100. The method of claim 99, wherein said additional ORF encoding said additional tolerogenic factor is in operable connection with said inducible promoter.

101. The method of claim 100, wherein said additional ORF comprising said additional tolerogenic factor is in operable connection with another inducible promoter.

102. The method of claim 100, wherein said additional ORF comprising said additional tolerogenic factor is in operable connection with a constitutive promoter.

103. The method of claim 102, wherein said constitutive promoter is a beta-2 microglobulin (B2M) promoter, a class II major histocompatibility complex transactivator (CIITA) promoter, or a CAG promoter.

104. The method of any one of claims 92-103, further comprising tagging an activator or repressor molecule to said inducible promoter.

105. The method of claim 92, wherein said inducible promoter is a cell-specific promoter.

106. The method of claim 105, wherein the cell-specific promoter is an immune cell gene promoter.

107. The method of claim 106, wherein the cell-specific promoter is a white blood cell gene promoter. In some cases, the cell-specific promoter is a neutrophil gene promoter, an eosinophil gene promoter, a basophil gene promoter, a mast cell gene promoter, a monocyte gene promoter, a macrophage gene promoter, a dendritic cell gene promoter, a natural killer (NK) cell gene promoter, a memory-like NK cell gene promoter, a lymphocyte gene promoter, a B cell gene promoter, a T-cell gene promoter, a regulatory T-cell (Treg) promoter, a hepatocyte gene promoter gene promoter, a cardiomyocyte gene promoter, a renal cell gene promoter, a dopaminergic neuron gene promoter, a pancreatic islet cell gene promoter, a macrophage gene promoter, or a retinal pigment epithelium cell gene promoter.

108. The method of any one of claim 92, and 105-107, wherein the inducible promoter is an L selectin (CD62L) promoter, an interferon-gamma (IFNg) promoter, neural cell adhesion molecule (CD56) promoter, a CD56 promoter, a KIRs promoter, a CD16 promoter, a NKp44 promoter, a NKp46 promoter, a NKG2D promoter, a TRAIL promoter, a CD122 promoter, a CD27 promoter, a CD244 promoter, a NK1.1 promoter, a NKG2A/C promoter, a NCR1 promoter, a Ly49 promoter, a CD49b promoter, a CD11b promoter, a KLRG1 promoter, a CD43 promoter, a CD62L promoter, a CD226 promoter, a TRAC promoter, a TRBC promoter, a CD3 promoter, a CD4 promoter, a ThPOK promoter, a CD8 promoter, a FOXP3 promoter, a Helios promoter, a CD25 promoter, a GARP promoter, a GPA33 promoter, a CD14 promoter, a CD11b promoter, a CD68 promoter, a CD138 promoter, an IgH promoter, an IgK promoter, an IgL promoter, an IgG promoter, a CD19 promoter, a CD20 promoter, a TH promoter, a TUBB3 promoter, a FOXA2 promoter, a GIRK2 promoter, a Nurr1 promoter, a DAT promoter, an INS promoter, a chromogranin A promoter, a synaptophysin promoter, a neuron-specific enolase promoter, or a Leu7 promoter.

109. The method of claim 108, wherein the cell-specific promoter is a memory-like NK cell gene promoter.

110. The method of any one of claims 101-109, wherein the cell-specific promoter is an L selectin (CD62L) promoter, an interferon-gamma (IFNg) promoter, or neural cell adhesion molecule (CD56) promoter.

111. The method of any one of claims 101-109, wherein the cell-specific promoter is a CD16 promoter, a HLA-C promoter, a NKp46 promoter, or a DAP10 promoter.

112. The method of any one of claims 101-109, wherein the cell-specific promoter comprises a sequence having 95%, 97%, 99% or 100% sequence identity to a 60, 70, 80, 90, 100, 200, 500, 1,000, 1,500 or 2,000 base pair long fragment of any one of SEQ ID 5 to 57.

113. The method of any one of claims 101-109, wherein the cell-specific promoter comprises a sequence having at least 95% sequence identity to a sequence that comprises 500 bp of any one of SEQ ID 5 to 57.

114. The method of any one of claims 101-109, wherein the cell-specific promoter comprises a sequence having at least 99% sequence identity to a sequence that comprises 500 bp of SEQ ID 5 to 57.

115. The method of any one of claims 88-114, further comprising a sequence encoding a translation control sequence.

116. The method of claim 115, wherein said translation control sequence is an internal ribosome entry site (IRES) or 2A peptide.

117. The method of claim 115 or 116, wherein said sequence encoding said translation control sequence is positioned 5′ or 3′ of said open reading frame encoding said tolerogenic factor.

118. The method of any one of claims 115-117, wherein said sequence encoding said translation control sequence is located between said ORF encoding said tolerogenic factor and another ORF encoding a native gene of said differentiated cell.

119. The method of claim 118, wherein said native gene is B2M or CIITA.

120. The method of any one of claims 88-119, wherein said cell further comprises another ORF encoding a chimeric antigen receptor (CAR) or T-cell receptor (TCR), or functional fragment thereof.

121. The method of any one of claims 88-121, wherein said cell further comprises a cell-specific gene configured to modulate the expression of said tolerogenic factor.

122. The method of any one of claims 88-121, wherein said immune cell is a natural killer (NK) cell, a T-cell or a B-Cell.

123. The method of any one of claims 88-121, wherein said cell is an embryonic stem cell (ESC), a mesenchymal stem cell (MSC), an induced pluripotent stem cell (iPSC), or a hematopoietic stem cell (HSC).

124. A method for reducing immune response in vivo, administering a cell comprising an inducible promoter in operable connection with a tolerogenic factor to a subject, subjecting the cell to conditions that induce the inducible promoter to modulate signaling from the tolerogenic factor.

125. A method for enriching a cell population in vivo, comprising administering a heterologous cell population to a subject, wherein said cell population comprises at least one cell comprising an inducible promoter in operable connection with a tolerogenic factor, subjecting the cell populations to conditions that induce the inducible promoter to modulate signaling from the tolerogenic factor.

126. The method of claim 125, wherein modulated signaling from the tolerogenic factor reduces the immunological rejection of the cells comprising the inducible promoter.


SEQUENCE LISTING (57 sequences)

SEQ ID NO: 1 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 292 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 3 - protein, 305 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 4 - protein, 311 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

SEQ ID NO: 5 - DNA, 3001 bp, synthetic construct
  source 1..3001: mol_type = other DNA
  misc_feature 1..3001: note = TP53 regulatory T-cell (Treg)
cctatttcaacaagtggagacagaataactggatttccatacaggaaagataccagagactgactcctacacctcacaccataaacaattaattttaagaattaattaatggctcaaggaccttactgtaaaacttacaaccataaaggtcctaaaagaaaatgtaagataatatcttcatgaccctggggttaaaaaaaaaaaaaaagatgtcctaaacaggacaaggcaaatactgaacataaaaaagataaatccactcctcttaagatactgtaaactctgtaaagcaaacaaataggcaagcaacagatcagaagaaaacattcacgacacatggatctgataaaggacttgtatccagaatgtataaagcagtcccacaactgaacaataaaaacaaacaaaaaaccaaaataacaggtaaaagactcgaagagctactttacaaacaaaatacgaatggccaataggcacatgaaaaaatgctgaacatccttagtcaatagagaactgtaaattacaaccacaaggatataccacattagaaagactgacaatacctaatgtccggaaggctgtggcacaaccataataactcccataccttgctagttggagtgtaaaatggtacaaccgctctggaaaactcagagcttctgaaaaagttcaaaatacagctactttttacttccaaactcgcaattcccctcctaagtatttctccaagaaacacgaaaacatatgatcacaaaaagaattgtacaagaatgtttatagcagctttatttcataaccgcaaatgggaaacaactcaaaaggccatcaaaaggacggatatacaatcgatggactatactaaatgaaaaggagcaaaatactgatatatacaacatgaacgaatgtcagacagtacattgaaggacagaagcccgacaaaaatgagcacataatgtatgattccccccttttttttgagacggagtttcgttcttgtcgcccaggctggagtgtagtggcacgatcttggctcactgcaacctctgcctcccgggttcaagcgattctcctgcctcaccctcccgaatagctgggattacaggcacccaccacgcccagctaatttttgtattttttagtagagacggggattcaccacgttggccacgctggtctggaactcctatcctcaagtaatccgcccgcctcggcctcccaaagtgcaggcgtgagccacagcgcccagcctgattccattctatatgaagttctccaacaggcaaaatggttatggagatcaaaataaaggtggggtcgggaatcgactgggaagagacgtgatgaaacgtttctgggacgatgaaaagggtctgtgacttggtaggcatcacggagcggttaggggccaaaactcatcttcctgtgcacttgctgtgtgcactggcgctgtgtgtaaatgccacctcgatttaggaaaaagatgacgtaagtacggcacaaagtggccggtacgcggcaggtgcatgggaagaaactgcggaatgaaacaaccgcgagctaagagatggggcagcgggagaaatgaattcgagttccgcctcctaccaggaagaaccggctcgggccggagggctgcacggaggaccacacggacgcctgcgggcccgccccttccgcttcacgacgttcagcctgcgtctggaactggaatggcctagcccaaagctagataacaggtagattgtttttccgacaaattatcaaacgacccatcattgcactctttcaaaatttgattctcagacgtacccattctttttttttttcctccgggaagatgagatatactcattcttgaaaatacctccgggcttgccttctgcacacttctttccctccctgtctcacgccatggtagcgtccgcctaggttgcaggcgacccgcggggtggggcacaccattcaaagaaggggagggattgaggtttgcatcaaaacaaatacccctgcctttgcaaaggccataactaagtaatccagaaaaagaaatgcaggcggagaatagcagcctccctctgccaagtaagaggaaccggcctaaaggacattttctctctctctcctcccctctcatcgggtgaatagtgagctgctccggcaaaaagaaaccggaaatgctgctgcaagaggcagaaatgtaaatgtggagccaaacaataacagggctgccgggcctctcagattgcgacggtcctcctcggcctggcgggcaaacccctggtttagcacttctcacttccacgactgacagccttcaattggattttctccatctagcggagccgggggctgcctggaaagatcgctccaggaaggacaaaggtccggaagttgtgggaccttagcagcttgggctccccggatcacccccaaatgatcatttcggaatggagccccagttttcactaggatgccatgggctctaaaatatacagctatgagttctcaatgtttcgagatccaaaagtctcagacctcaatgctttgtgcatcttttatttcagggattccctacgcccagcaccgggtggatgtgcaaagaagtacgctttaggccggctcaaggttccccaaagctccactcctctgcctaggcgttcaactttgagttcggatggtcctaacatccccatcatctacacccaggtctcccaacaatgcaactcctatgatgatccctctagccaagcttccatcccactcacccccaaactcgctaagtccccactgccccacccccagccccagcgattttcccgagctgaaaatacacggagccgagagcccgtgactcagagaggactcatcaagttcagtcaggagcttacccaatccagggaagcgtgtcaccgtcgtggaaagcacgctcccagcccgaacgcaaagtgtccccggagcccagcagctacctgctccctggacggtggctctagacttttga

SEQ ID NO: 6 - DNA, 3001 bp, synthetic construct
  source 1..3001: mol_type = other DNA
  misc_feature 1..3001: note = TNF IFNg
tctgtctccctctgctcaccttggggtttctctgactgcatcttgtccccttctctgtcgatctctctctcgggggtcggggggtgctctctcccagggcgggaggtctgtcttccgccgcgtgccccgccccgctcactgtctctctctctctctctctttctctgcaggttctccccatgacaccacctgaacgtctcttcctcccaagggtgtgtggcaccaccctacacctcctccttctggggctgctgctggttctgctgcctggggcccaggtgaggcagcaggagaatgggggctgctggggtggctcagccaaaccttgagccctagagcccccctcaactctgttctcccctaggggctccctggtgttggcctcacaccttcagctgcccagactgcccgtcagcaccccaagatgcatcttgcccacagcaccctcaaacctgctgctcacctcattggtaaacatccacctgacctcccagacatgtccccaccagctctcctcctacccctgcctcaggaacccaagcatccacccctctcccccaacttcccccacgctaaaaaaaacagagggagcccactcctatgcctccccctgccatcccccaggaactcagttgttcagtgcccacttcctcagggattgagacctctgatccagacccctgatctcccacccccatcccctatggctcttcctaggagaccccagcaagcagaactcactgctctggagagcaaacacggaccgtgccttcctccaggatggtttctccttgagcaacaattctctcctggtccccaccagtggcatctacttcgtctactcccaggtggtcttctctgggaaagcctactctcccaaggccacctcctccccactctacctggcccatgaggtccagctcttctcctcccagtaccccttccatgtgcctctcctcagctcccagaagatggtgtatccagggctgcaggaaccctggctgcactcgatgtaccacggggctgcgttccagctcacccagggagaccagctatccacccacacagatggcatcccccacctagtcctcagccctagtactgtcttctttggagccttcgctctgtagaacttggaaaaatccagaaagaaaaaataattgatttcaagaccttctccccattctgcctccattctgaccatttcaggggtcgtcaccacctctcctttggccattccaacagctcaagtcttccctgatcaagtcaccggagctttcaaagaaggaattctaggcatcccaggggaccacacctccctgaaccatccctgatgtctgtctggctgaggatttcaagcctgcctaggaattcccagcccaaagctgttggtctgtcccaccagctaggtggggcctagatccacacacagaggaagagcaggcacatggaggagcttgggggatgactagaggcagggaggggactatttatgaaggcaaaaaaattaaattatttatttatggaggatggagagaggggaataatagaagaacatccaaggagaaacagagacaggcccaagagatgaagagtgagagggcatgcgcacaaggctgaccaagagagaaagaagtaggcatgagggatcacagggccccagaaggcagggaaaggctctgaaagccagctgccgaccagagccccacacggaggcatctgcaccctcgatgaagcccaataaacctcttttctctgaaatgctgtctgcttgtgtgtgtgtgtctgggagtgagaacttcccagtctatctaaggaatggagggagggacagagggctcaaagggagcaagagctgtggggagaacaaaaggataagggctcagagagcttcagggatatgtgatggactcaccaggtgaggccgccagactgctgcaggggaagcaaaggagaagctgagaagatgaaggaaaagtcagggtctggaggggcgggggtcagggagctcctgggagatatggccacatgtagcggctctgaggaatgggttacaggagacctctggggagatgtgaccacagcaatgggtaggagaatgtccagggctatggaagtcgagtatggggacccccccttaacgaagacagggccatgtagagggccccagggagtgaaagagcctccaggacctccaggtatggaatacaggggacgtttaagaagatatggccacacactggggccctgagaagtgagagcttcatgaaaaaaatcagggaccccagagttccttggaagccaagactgaaaccagcattatgagtctccgggtcagaatgaaagaagaaggcctgccccagtggggtctgtgaattcccgggggtgatttcactccccggggctgtcccaggcttgtccctgctacccccacccagcctttcctgaggcctcaagcctgccaccaagcccccagctccttctccccgcagggacccaaacacaggcctcaggactcaacacagcttttccctccaaccccgttttctctccctcaaggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcggagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagcc

SEQ ID NO: 7 - DNA, 3001 bp, synthetic construct
  source 1..3001: mol_type = other DNA
  misc_feature 1..3001: note = IL6 CD56
aaaatacaaaaagtagctgggcgtggtcgcatgcatctgtagtcccagctactcaggaggctgaggcaagagaattgcttgaacctgggaggcggcggttgaagtgagccaagatcatgccattgccctccagcctgggcaacagagcaagactccttctcaagagaaaaaacaaaacaaaacaagaaaaaacaaagaatgagctctccacgcgaaaaatccattgagatgcaaaggaaggaagctatcattgtggaattgcacatgtcagttacattaacgtttttggagcaaggtagagctcatctctcccacaagcaaattccagcccaaagcattgatactaataaagtgccatgctgcgatgtgcagggggcagacagtgtctccaagctccctacacacatgccttcccacagtttgccctttcttgaccccagaagcatcaggccccttcaccctcgagggccactatcaggagtttgaattaatggcaatcaccatgcacagggaaggctgtggaattctgacataaaaacacttagtggagggcttggaaaaagtctagtaggagcaagacgcaagctggactaattatctaaaacaagagacctggtttggggatcttaatgttctcaaaaaagaaaattattattatttttcattttgcactttgtgccataaaacattttcaacaaaacatagaatctcatttcttttgagggaaaatgattgggagaccagctcattgctggcacagaggcctggttcattcataattccttcataggcaagacaccaggtgaaccgatatagccgagctggaagagctctccaaggcagagactctgagccaaggaatgttcaaagagctagcatgtattgtgggattactatgcgccaggaattttttacactgcatcacgttccatcttcacaacagccctagaaaggaagaactattattacccccgttttataggtgaataaacaagggcacaggtccttgatgtaacagccaggatcaaacagctgggaagacgagaaaacctttcccaggctaggataacagaggatttggttgaaaatacaggcaattaggtgctacctctgggaaaaggggccaggagaggaaggagacacttttccctgcatgccctgatgtcctatttgaacattttatcatgaacacgaacttcctatttaaaaaacactttttattgaaaagataaatctgtgtgttgtattgtgtcactcagttcaagtacttgaaatttattgaattgtattttctaaaaaatagatagttgagtaaaagcaagctcacattacatagacggatcacagtgcacggctgcggagctgggagcagtggcttcgtttcatgcaggaaagagaacttggttcaggagtgtctacgttgcttaagacaggagagcactaaaaatgaaaccatccagccatcctcccccattttcattttcacaccaaagaatcccaccgcggcagaggaccaccgtctctgtttagacaatcggtgaagaatggatgacctcactttccccaacaggcgggtcctgaaatgttatgcacgaaacaaaacttgagtaaatgcccaacagaggtcactgttttatcgatcttgaagagatctcttcttagcaaagcaaagaaaccgattgtgaaggtaacaccatgtttggtaaataagtgttttggtgttgtgcaagggtctggtttcagcctgaagccatctcagagctgtctgggtctctggagactggagggacaacctagtctagagcccatttgcatgagaccaaggatcctcctgcaagagacaccatcctgagggaagagggcttctgaaccagcttgacccaataagaaattcttgggtgccgacgcggaagcagattcagagcctagagccgtgcctgcgtccgtagtttccttctagcttcttttgatttcaaatcaagacttacagggagagggagcgataaacacaaactctgcaagatgccacaaggtcctcctttgacatccccaacaaagaggtgagtagtattctccccctttctgccctgaaccaagtgggcttcagtaatttcagggctccaggagacctggggcccatgcaggtgccccagtgaaacagtggtgaagagactcagtggcaatggggagagcactggcagcacaaggcaaacctctggcacagagagcaaagtcctcactgggaggattcccaaggggtcacttgggagagggcagggcagcagccaacctcctctaagtgggctgaagcaggtgaagaaagtggcagaagccacgcggtggcaaaaaggagtcacacactccacctggagacgccttgaagtaactgcacgaaatttgaggatggccaggcagttctacaacagccgctcacagggagagccagaacacagaagaactcagatgactggtagtattaccttcttcataatcccaggcttggggggctgcgatggagtcagaggaaactcagttcagaacatctttggtttttacaaatacaaattaactggaacgctaaattctagcctgttaatctggtcactgaaaaaaaattttttttttttcaaaaaacatagctttagcttattttttttctctttgtaaaacttcgtgcatgacttcagctttactctttgtcaagacatgccaaagtgctgagtcactaataaaagaaaaaaagaaagtaaaggaagagtggttctgcttcttagcgctagcctcaatgacgacctaagctgcacttttccccctagttgtgtcttgccatgctaaaggacgtcacattgcacaatcttaataaggtttccaatcagccccacccgctctggccccaccctcaccctccaacaaagatttatcaaatgtgggattttcccatgagtctcaatattagagtctcaacccccaataaatataggactggagatgtctgaggctc

SEQ ID NO: 8 - DNA, 3001 bp, synthetic construct
  source 1..3001: mol_type = other DNA
  misc_feature 1..3001: note = NCAM1 CD56
tacccaggtccacctgttcttatcttatcttggaattgcaaatctgggctcctttttgcctcctttcccactgtcgctttcttaattagtccctgtcaccctatccagagctatcatggcctcctagcttgtctccttgccatggctgtcttcttggaccattctctgcttcctctctaaagaactttctctctaaagcacagtctggagtctccttcctctgcctcaatcccatggatgttcactttgtagatgctcattaatgtgtgcctgacagcctgggcctctctctggggagagaggtccagtgagggccacagttttgtccattttaaaatgaggatcaggaagggattgaaacgcaacagagctcacagatgtaggcaccgactagcggagatatacagaattggagacaagtgacatatcctgttaaaagaatttgggagtgatactgggagtgtggactggaatgactggggcagcattctagaatgaatcattacaacagagatcaacaaccacacggggtagggaggggctggagagagagagggaaaaaaaagaaattcagtcatactcaagtgagattggatttccacacttgcctacattgaaaagctaaaaatagggacatttggggagaaaaatagtgaggagtcttttaattctactgacaggactataaggagggaggtttgcagggaagaatgtgtctctcactcttaggggcaaaattggaaaaatttttattttgggggccccaaccttcactctccccactctaccccctgcctgttctctctatctttcctcaccatcaaaacctcttttttaaggaaagagtctgataacattcatttacataaatctgtaatatttaatgtggtgattttagatgttagaagcagaagtctgaaatcaaagacacttaaatacagcaaccattaaaaaaagacgtatataattcactgcatctagcacagggctaaacccagtggttgctccgcaaatatttgttgaaagatgttcaaaaaagccttatttatattatcaaactccacaaacaatataaatatgcaaaaatggggaaaccattgcaaaatcatccatatgaagaaatattacatagctataaaaatatctagaaaagctttttaatgatccagaggaataaatgcttatactgatgttaggcaaaatcagcaggattcaaattgctactataatatgtcaattataaaaatcatatgcacatacagaagacaaaatctatgagtatctctggaatctgtgggatttggggttatttttagttctttctacttttctgtacttttgaattgtttatacttggaatgagagaaatagcccaaagtaaacgttgtggaagatttacttgggggaagaaatacttttcaggggtccaagctaattacactgtactggtgtggcccacttggtctattatagaggaggaaagtggggaagagatggggcctgtccaataaggatggatgtcctgacagtagaggatggacttttaaaaactgctacagcggctgggtgcggtagctcacggctgtaatcctagcactttgggaggctgaggtgggcagatcacgaggtcaggagttcgagaccggcctgaccaacatggtgaaacaccgtctctactaaaaatacaaaaattagctgggcgtggtggcgcgcgcctgtaatcccagctactcaggaggctgaggcagcagaatcgcttgaaccgggagcagaggttgcagtgagcagagatggcgccactgcactgcagcctgggcgacagagcgaggctccatctcaaaaaaacaaaacagaaacaaaacaaaacaaaacaaaacaaaaaacctgctacaggagtggggaggccgacctttgaagaaaaacggagtacccggtaacattagtgctttaatgcctttgaacttatgcagacttcctctgttagagggtttcagtgttctaggctaatgggttaacctgacatctagaacacctttctcacattagttccttacatacccaagccttcaggtgctgagacatgattcttttcaccccgctttctccaccccctacttttgaaaacacgggtggaattttaattaaagcctattgtgttggtacctcagtaatattatacattaatatctttaagaattaaggtcacgtccccatgtaagaaaatattatttaatgacgcttctatatcataatacctatataaaagcctggctattttaataaagagaccacagatttcagaatttataaacaggaaaacattttcttcgggttatttctggaaatctcttccaaacatcggagttttcttctaacttaagtctcttcccacctccttcccaggggatctgctgaagggtgttgtatgtctctctgtgggagagcaaacttcacagttaggataaaacaaacaaacaaacaatatccaaacaacacccagcaacggcaacccccatccctctccaaagttctaatttcccgcacttaaagtcctgggctatccttgtgttgcaaggatcttagaatcgaaatggagggatttgacaactttacctaaccaaatctaaaattttgcttttattatttactagttatcaaaatatgcaaactgctgattaaggaaggctgggtagcaggagcgcctgcgaaggcgtagggtagaagtgtgaaaagaaatcccagctctcccagggagactgcgtgtgaaagagcccggctcccccaaaagctccaggccgcgttttgcaggcttccgcatctgcctcccctgtctctcttacctccttgatgttcggcactatttgtggccggcgtggtggaaggacacagtgaggttctcacccccgccccccgctcctcgctcccatcccagttccatcaaaacgaacccgggccagcgcaaggatctccgagttgcgagtgtgctgaggctgggactgtc

SEQ ID NO: 9 - DNA, 3001 bp, synthetic construct
  source 1..3001: mol_type = other DNA
  misc_feature 1..3001: note = FCGR3A CD16
ggagattgaggtcctaacaccaaattcattaatttattcaataaatattattggccaggtgtggtggctcacgcctgtagtcccagcactttgggaagccaaggctggtgaatcacctgaggtcaggagtttgagaccagactgaccaacatggtgaaaccctgtctctactaaaaatacaaaaattagctgggcgtggtggcgggtgcctgtaatctcagctactcgggaggctgaggcaggagaatcactacaacccaagggggcagaagttgcagtgagctgagattgccgcattgcactccagcctgggtgacagagtgatactctgtctcaaaaaataaaattaaaaaaaatgaaaataaatatataaataactattattgagcatctatgccatggcctaaccttcacatgacacattcacattgtatgcactccatatggggattcttggaattgtgcaatgcagcagtcctatatctactgtctgacaggctttgacattttgcccaagacctacttagagctaggactacaactctgataccattcagtgggaccacacatcatctcatcttagcttttatctctaagaatatcatctctggctcttaccaagtatttcaggaccctttgtttcaccctttattggtgattttcctcttccccttcatcaactcaccgatatggacttctagctgcaccgggtcactgagggtggagaggtttgtctggcacctgtactctccactgtcgtcgactgtggcagcgtcaatgaagtagctcgaggcctggcttgagatgaggctctcattgtgaaaccactgtgtggaattgtcctcaggggagtaggctccctggcacttcagagtcacactgtccttctcgagcaccctgtaccattgaggctccaggaacaccacagcctttgggagatcttctgaggagccaagataatgtggggtgaggacagggagaggagcaggctctacactgccattcccagggagcctcaaagccagaatgagctcattgcaaacccatgcttggtggctcagtcttagagcatcttggccccatttttggcctgttcagtatcttaaggaaagctggccagagaagcacagggccaagttctgctgtgttggaggaactatccctgctaaccccacatcagcattttcccattcaacaagcatttcccaatatcttatggcctttgtccccatatgtgccccactgggtcaatccaagaccatgaagctgactcaccagtccgcatgccagctgaaactgcaagaaaaaagataaatcaaatattgagtaggggcagagatcagagtgattagaacatagagtgagtttaaaactcccctgccctcctctgccccaggagcccaattttcccaagaatcaggatgtttctggtggaaaccttgctacctgctctctggtctccactgttcatgcctcttgcgccactgtcaacacaaatccctattttcaacactgctcccttaccccttgctccctgtgcaaactcacaaattaagggtacaggttgaatttccctgaaccaagctacagaaagagcctcaacccatatccccacaagaaagggtagaaattgaaaatcatagaggagaaccctggaatgtcaaactgaaagagacagaccctagggaccatctagtcgaagctctttggttccacagagtgattctgggacccagaggggtgaagtgactggcctcactcatgagtatgccccaattggaaccagcattctcctcatttctagccccatcttggcttgtcctaggagctcaatccacagctatagatgtggtgaggggtcccatcccttcgtgggagtctcattcgtagcctgaaaaggggtctctgctgaacccaaggcatctcaaacttctccctcaaccagggagaccctgacttacctagaagtagcagagcagttgggaggagcagctgccacatgatgccacactggagtggacaagtcaccaaagatatccggagccctaaagggaccaaaccgactagacaggaggaagtaaacagcctttccccagcccctccacccatctctgtcacccaccaatttccttttcttgaaacttcatctgacttctcagtctgaagtctggcaagggagccccaccatagaacaggaccaggaaggaaagagcctggaggcaaggtgggtgggtctgccccctttactccctcaaaggtctgtggctgagcatctgaggacacacacagaatctgccagagtgtgccctcagccatcccaggatgcttgccccatctcctggatttggatccacccagcaccaagaatgggacagtgagaccctggggatgagattcaaggtgggaggagcattctctgagggcttccttcatttcacctcagaacttgtcactctcctgcctcgcccagacccatctatctccagctgaggccctgcctgcacacagaaagtggtcctttcaaatcttcagaccactagcagtgtctctgcctcaatattatctccacgcagaatttctttcaaaattcaaaattcaaaatctatatgctcctgggatatgtaatccacaggagctactgactttttagtgtgatttgatcattagatttccaggttagaagaaacccaggtagggtagagaaatgggccctggaagaacaagtcaccagcgaaaggctgaaaagatcacagaaaaggtgggggtggggaggggcgaggacaggaactctttaccttcctcgtgttacccaggtcctgcggatttagctcaggcccctccgggccactggatctgggctggtctgtcagcctaggagccggggctccagggctccttaccagaaaaggtggaggggggtggccaggaagtgggagggttgttaacctcttctcttctctccagattctcccccgtaaccccacgttggttactctcacaa

SEQ ID NO: 10 - DNA, 3001 bp, synthetic construct
  source 1..3001: mol_type = other DNA
  misc_feature 1..3001: note = NKp44
cctgagcccaggcctcccgcagggtgcttagggtggaagcctggttctgtgcctgacccaccctgaacttcctgtgagggagctgtgcgcccagactgcagccttgtgtgggcagtagctcaggtccctctgacctctctggcagggaaaaagctgctctgagaccagatgctttgaacctggcatcctgggaccaggaagggccattgttatgggaccattgtcatgggagcaggaataaccctgtcagggcaattgatgactgaaggatcctgctggagctggggcagagagccattggggtcagaggtataagcagacttgttggggttcagggagaagactccttggatctgcaaaaggcaagcctggatgaagagtaaggaagtgcccatggagaatctctgagcccaggaggagcaccaggtcaggagagaggaggcctgggtgctagtcctggtttggccctgaaatagctgagtgactgtggaaaggctacttgccttctctggacctcatctccctcagttgtgaaatcagcactgagagaaattcccttttgtccctgctacttctggttctatcatgggggaaatttacagctaccccttcacccctgcccagcctagcttagccctgtgctgcgttttggggatcacctccagacctctacggctccctgctttccccatttctgcatcagaggccagccctcccttcccatggacctacccaggctcggagccactgttgcaggctagactgggccttgctagaaaagcaagtacatgaagtctccagctctcccagctccttctctgggttcctgtgagtgtgtgtgacaatgagtttgggagaggaggttgctgaggttcccaagttctctgagggaccttctcagagggtgggcagggaacccagaatcccccagaaccccaggctctttgacaggactttgctgtgccctgctctgatcgtgccaagggggaggcactatccctagctcttcaagagacttctgaaagggccccagatcccaccacagctacctgccccagtccctcctgcagaaattcaacttgtgacaagggctgttgtgggtccccagggagcagggccagtcccagcctggatgaggggctgcctaagcccaggcttccctgcccccaggtcccagatgccagaggaagctgagcgtgcagatggttccagcaggggcctccccctagggtgctcaaggccccaagtctatgtggagggtgcccaccccactcacacaggtggggaagccacaacacagagagaggggcagattccccgctttgagagctcctgtcccctggggagagacgacacactctgacacagcagagaacgatacagagctggctgcaggctgaagagacacacagctagtggcacatagcaaggggtgctcttgtaagctacaagaaggagctgtcacttagagtcagcaggggtcagagaaggggaaagtcagcatgggaacacagttgctgacacacacacacacacacacacacacacacacacacacacggtgacgtgcctcggtgccatctgaggacagggccgccttcctccccctcgggaggatgcccccaccctgatttggtatgtaattatgttcagggagaagctgggccccactgtcggcgaggaaaccccctaaaagctcacctccgcctgtgtgtgcaattcagtgggagcctctttctcacccctggtttcctgtcatgtctgtgggtcgcatttcaaccagaccagccggaaggctccagaccgaagtcaggaggcctgttctggtcctgtgtgactctgagcaggtggctgaccatatctgggcctcagttctcacatctgtgaaaaagagaagtaatgtctccctcgcgaacctgctgagggtcttgagaaagtgtgcatgagaatgctttgtaaataatgcttgtgtgagtgagtggcgggcatcgctccctgagctgtcttctagctctggagctccagcctgctttcccaccttgggcccttgggcgccaacatctgatagccctactgtgtcctccttttgcatacccctctgcctttcccattctcagtctctgcatactcctactgtaaggagaagggcagtgtactgagatggagtggagagcaggagtccaggggcaggcaaagcagtggacttggaatcgagtccacttgacaagttccaattcccgtctcagttattacctctgttcacagagctcccggcagcagatcgccagtccagcagagcccccagtggtgcaactgcaattacacacagtgtctctaccagtcctaagatatattagctcgttctctttgtaaattgcatcgtattttaaaactgaatcttcaaagtgctgacattaactaaagaaaaatatgctagagtctttcatctgtttatgttttaattaaacaatatttctccagggtccttaagtggcagaggtccaggatgcagagatgaggccaggtcctggttcctgggctccctctaccccaccccactgatgcatcccatgctccgtgtgctcttcccaccctcccctccccgacctttgccagggaccctccctagcctcccttgctggttttgaatctccacctcctctcccctctcctttcccgcacccacgaaactcccagagtggaagcacatcactgacccaccatggaggcctgggcatgacgtccctggccatcctgtgaccagtgaggcttggggcttgtgatcagggttccaagtgcccctgagtttgtcatccaccctcagcctgcctttccctggaaagggcggcaaagctagggccctgcaaagaagagaggcagcacacagagggagcaagggggaagcagtccataactcgcccacagtttcctcctggccttttcagctcctccctcaggggtaggtcatggtggggaggggcagctgggcacaggggagagc

SEQ ID NO: 11 - DNA, 3001 bp, synthetic construct
  source 1..3001: mol_type = other DNA
  misc_feature 1..3001: note = NKp46
gtgtaaacaagagcagggcatgtgtgagtagttgagaatggtgaataggagtatgactagacagaagatagtagggatgacaagtttttgggggcacattccaagttggtctggtgtctggaatgagactggggcttaataaaaaggagcgtctatacaggagctcaaatgggctgtaccctttagcattctgaggacaggcctgaattctgagaaaagaaagtggtaaaagtattgtccagtcttttttaagttggtggctgagcttggtgaggtgtgtttttaaaagactattagtctgttctacttttcctgaagactgaggactgtaagggatataaaggtttcactgaataccaagagcctgaaaaactgcttggctgatttgactaataaaggccggtctgctatcagactgtatagaggtgggaaggccaaactgtggaattatgtctgacagaagggaagaaatgacctcggtggccttctcagaccctgtgggaaaggcctctacccatccagtgaaagtgtctacccagaccaagaggtattttagtttcctgactcagggcatgtgagtaaagtcaatttgccagtcctaggcgggggcaaatccccgagcctgatgtgtagggaagggaggggacctgagcaatccctgaggggtagtagaatagcagatggaacactgagaagtggtttccttgaggatagatttccaggatggaaaggaaatgagaggttctaagagatgggctagcagcttgtaacctacatggaagaggctatgaaatatcgaccgaatagaatgggcctgtgaggctggaaggaggtattttccttggtctaagaaccatttgccttgtgtgggaagagattgatgggtggaagtttcagtgggggagtaggtgggagtgactgatgagaaggagaaaaactggctgtgggggacagaaattggcatgctagctgcttgtctagctaccttatcagcataagcatagatgtgagagacagaagttggaaagctagctgcttgtctagccaccttgtcagcataggcattgtctagagcaatgggatctgatgacttttgatggcctttgcagtgaatgactccagcttcctctgggagtaaagcggccttgagcagagtttttattaaggaggcattaaagatggaggacccttgtgtagtgaggaaacctctttcagcccatatgaccgcatggtggggcagaatatgaaaggcatatttagagtcagtataaatattgatgcatagtccttttgcatcagtgagggcttgagttaaggcaactaattcggcttgctgagaggtagtggagggggcagagcggtagcctcaatgatagatgtggaagatactatagcatagcctgcctttgctggtgagtggcgattaggcctggtggaactgccatcaataaactaaatgtgattagggtgaggaataggaaagaaggaaatgtggggaaatggggtgaatgtcaggtggatcagagagatacagtcatgggggtcaggtgtggtatccggaataatgtgggaggccggattgaagtatgggccagtaacaatggtaattgtgggagactcaacaaagagtgagtacagctgaaggagccggggagcagaaagtatatgcgtcaggtgtgaggaagaaaatagatttttggaagttatgagaactgtagagagtgagttgagcatagtttgtgattttgagggcctctaaaactattaaagcagcggcagccgctgctcacagacgtgagggctaggctaaaacagtaagatcaagttgtttggacagaaaggctacagggtgcggtcctggctcttgtgtaagagttctgaccacgctaaccatgcctaggaaggaaaggagttgttgttttgtagaaggtgctggggtttgagagatcactaggacacgattggcagggagagcacgtgtgtttttatgagaattatgccgagataggtaacagaggaggaagaaatttgggcttgactgaagtaatgggggctgtctgtgaagccttgcagcagtacagcctaggtaatttgctgagcttgatcggtgtcagggtcagtccaagtgaaagcgaagagaggctgggatgaagggtgcaaaggaacagtaaagaaagcatgtttgagatccagaacagaataatgggttgtagaggcaggtattgaggataggagagtatatgggtttggcactacggggtggataggcaaaacaatttggttgataaggtgcagatcctgaactaatgtgtaagccttgtctggttttaggacaggtaaaatgggagaattgtaaggggagtttataggctttaaaaggccatgctgtagcaggctttaatccttttaaagcatgctgtgggatgggatattggcattgagcggggtaaggttgattaggttttaatgagatggtaaggggtgcatgattggtcaccaaggagggagtagaggtatcctatacttgtgggttaaggtggggggatacaagaggaggacacaaaggaggctttggattgggaagaagggcagcaatgagatatagctgtagtccaggaatagtcagggaagcagataatttagttaaagtgtctcagcctaataagggaactgggcaggtggggataactaaaaaggagtgcttgaaagagtattgtctaagttggcaccagagttggggagttttaagaggtttagaagcctagctgtcaatacctacaacagttatggaggcaagagaaacaggcccttgaaaagaaggtaatgtggagtgggtagcctccatattgattaagaaggggacgggcttaccttccactgtgagagttacctagactgtctgtgatggtcctgtaggcttccgaggcgatcgggatcgggcagtgtcagtcttcagctgctaagccgagaagatctgggaaggagtcagagagccttgggc

SEQ ID NO: 12 - DNA, 3001 bp, synthetic construct
  source 1..3001: mol_type = other DNA
  misc_feature 1..3001: note = NKG2D
gtcatggcctttctgccatttggggcataaattttaaaaattcagcttgccatttccatctatgtacttcccctgactatcctttttaaagtacaacatgaacttacttttgttttatagtactacattattatgtttacactttactatataaataaaactatatttatgccatggaaagtacccttagcaaaatcctatatactattaaaattagttaattcgtaatgtataatgaaacctaacattaccagaaagggacttggttagtgaaaaggcagatgtagggagaaagacatgtatttaaatcacagatccacatatacaaacttgataacttcacaaataacctagcttttgcatcccagttttcctaatttgcaaaagacgaaagaaataaaaaagaaaaggatactgtcacaatccagtatgggtattttaaaaataaaataaaaatataatgaacttagcatcaataacatcaatcatttagtaagcttttagtaaatagcagtgtgtctaattttcttcttttctttttttttttttgagatggagtgcaatggcgtgatattggctcactgcaaccgccgcctcctgggttcaagcggttctcctgcctcagcctcttgagtagctgggattacaggcttgtgccaccatacctggctaatttttgtatttttagtacagacggggtttcaccatattggccaggctggtctcgaactcctgaccacaggtgatctacccacctcagcctcccaaatttctgggattataggcatgagccactgtgcccagccatctaattttctttataatgaaggacatacataggctaaaatagtggggtctaatgatttagaacaaagacagataatcttgcctctgaacaaaatacctaaagaatcattttgctctcttgaaatattaattatttactgatgattaatatttacttaaaagccttttttaaacttttatttttggttcaggggtgcatgcgaaggtttgttacatatgtaaattcatgtcacgggggtttgttgtacaaaagccttttataaattaaatgataatatgaattatattttataggtaaaattagacattgattgcgcatgtaatacctaactcagttattttacatatggagtaaagagagtaggtatcactttatggatgaagaaattgaatgctagagatggccaaaatcataaagcttatttaatataatgttaccaactgtgaataagttataaatttataaacacctttaggataaaaagtgctttaacatacatgtcagttgatttgaatattacacaaatctaagataaatatggaatgtttcatgcctgtatttgatagaaaaagaaactaaactaaactacacagaggttgcaaattgacccttcaaatctagagagcggtaaaagtttgagtaaaattcaagtctgactccacatctatttcactatttcaagctgtcccaagttgtgagcgactttaccataatatagttaataaaataaagggaaaaaagtttttaagttttggaagacttgatttaaaatacaatatgaacataacatgaaatcaacataaatagaacatgaaaagaatcagagtaaattgcttgcctatgccttatatgcttgtatgaaattcttggtatcttaaaattgtataaaaacaaaactacacacttatgtggtaaaaacataccccagctgtgtcgagacctccgaccacgaatccaccccatcaaatacttataagtgcacgtctaccgcagagaggaatctaaagtcttcaatgcacaaaggattcctgaataaaataaaactgggcatttgttttttgttctctttcccgtggtgagataaaatattttgaatagaaaagtaaaatgcagaaacttttcccctgtgcatgcctttaagaaaagaatagaaatgacaattcacataaagccaggcaaaattaattttatacatttctcttggttgtaaaataatcgccaattacttcaaaatgtcataacaatataatgaacctattaattcacctacattgagatatttaataccaatattttactttactgatattagataacaacacacccagttttctgtttatagtcgtttattgcttcttggccttttagctaagatcaagtgtttccagttgtttattttatctggcaataatttagttttctttctgcaagctaaactcaggtaagttatatgcacttttgttgaatgtccttttctagtgcattttaatccaacgtgcccatatcatcatcatcaacaaattatcatcaccatcatcattaacaaaactaatctcacatgtttatatagtaacatcttagcaatgcacagcagagcttccagaaaaaaccatttaataaataagcatagtgcttagatttcaaaaataaaagctatttcatatttgattatgatagttggaaagcacagatataacttgtttcaccttgcatgtaaagatgtgtatcaaaggtaagtgccccctacagcatcagaaaactcaaaaatttgacaatttaaattgaatctcaattcttctcatcctaaatgtaaagattcatttttctagttttgcttagagacaaggcttcttcttatagtaatattacaacttctgattcttcaataagtatatcctaattacctatcatcagatacagtgatagaatatacatcagaaatgataagcattccgtatcctcaggagttctattcaagcatctaagaaacaattagaattaccttatagtgtaaatatttttatttttttgaggttgaaatataatttctcatatcttgaataataaacacacacacatacacacatacacacacctgctcagagagggaagatcttgattcttgtggataaaagccttctataaaactagaaaatttagatatgtcctcagttcctgatctagaattgaaag

SEQ ID NO: 13 - DNA, 3001 bp, synthetic construct
  source 1..3001: mol_type = other DNA
  misc_feature 1..3001: note = TRAIL
acttattgtcaacacaggaatgccacataactaaaagcaaaacatttcaaagatgtgaaagttcaccaggactagctacatctggaatacaaaacgggagtgttcagtgacttagggcctggctttgggttgagcctgactctacccttgtcgcctgcatattcgtggtgcctaccacatctgtgaaggctgttcattcactcaataagtatgtagtgaaccacaagtattagtattagtggctacttgggaaccacatgctagacccagaggtatactaatgagcagaagcagacacagctctgatacagctaccctcaccctcaagaattcatgagtcaagaactgtaactccaacctgcaacaacaaagcagggaagatggctacagttaccaaaacagcatgttactggtatggaaacagatacatagaccaatggaacagaacagaggcctcagaaataacactacacatctacaaccacctgatctttgacaaacctgacaaaaacaagcaatggggaaaggattccctatttaataaatggtgctgggaaaactggctagccatatgcagaaaacagaaactgggccccttccttacagcttctacaaaaattaactcaagatggattaaagacttaaacgtaaaacctaaaagcataaaaaccccagaagaaaacctaggcataccattcaagatggcatgggcaaagacttcatgactaaaacaccaaaagcaattgcaacaaaagtcaaaattgacaaatgggatctaattaaactaaagagcttctgcacagcagaagaaactatcatcagagtgaacagggaacctacagaatgggagaaaatttttgcaatctacccatctgacaaaggtctaatatgcagaatctacaaggaatttgtttcttgtaaaacaaattgacaagaaaaaaaccacatcaaaaagtgggcaaaagatatgaacagatacttctcaaaagaagacatttatgtggccaacaaacatgaaaaaaagctcatcatcactggtcattagagaaatgcaaatcaaaaccataatgagataccatctcactccagttagaatggtggtcattaaaaagtcaggaaacaacagatgctggaaggatgtggagaaataggaatgcttttacactgttggtgggagtataaattagttcaaccagtgtggaagacagtgtggcaattcctcaaggaaccagaaataccatttgactcagcaatcccattactgggtatatacccaaaggattataaatcattctactataaagacacatgcacacatatgtttattccagcaccatttacaatagcaaagacttggaaccaacccaaatgctcatcagtgatagactggataaagaaaatgtggcacatatacaccatggaatactatgcagccattaaaaagaatgaattcatgtcctttgcagagacatggatgaagctggaagccatcattctcagcaaactaacacaggaacagaaaaccaaacactgcatgttctcactcataagtgggagttgaacagtgagagcacatggacacagggagggcaacacacacactggggcctgtcgggggtgggaggctaggggagggagagcattagaacaaatcctaatgcctgtggggcttacaacctagatgatgggttgatgggtgcagcaaaccaccatggcacatgtatacctatgtaacaaacctgcacgttctgtacacgtatcccagaacttaaagtaaaataaaaaaatttaaaaataaagaaatctgtttgccagaattcacaaagagcaatagacctcatgtgagttatgaatctgaacttcatcctgctgtgtgctattggataaagactctaagagctacattacagattttttcgacaggaagtatcacagtaacaaggccctatttaaaggctgttgctactgctgtggaacagatttacccatttgtgtttgaaagcaggaaagaaattttatcattcaccacttaattggttagaatctcttactgtgcaccttttaaaacctgctgcacattggactcaaaaggaaaactgaaccaacggtaattgaggaaatagactcttttatttattcatggctacagtgtaagctccagtccctttggattttattccaaaccttgttataatataaaaaaggaagtttacaagacatgtcattgctgcttttacaaaaggacattctatttattttcgcaggaattctcatgtccccataagcagagctgtcacagtgtgcactaccttaggttgttttattgttgtcattgttatttttttccattttgagctaatgtgttttatctgtgaatagtcttttacatttttgtatgctgaaaatgggcaccaaagaacctgtaaaaattatctttttcaattgaatgtgcacaaataaaagtttggaaaaggaaaacaaaagaactacaaagcatttagcagaaagctttcatgaagagttgcaatggctgatattataaaatacacatacttttaaaaacaaaaaatccctcaggagttttgttgcccacatctgctagtaactagtgaagccagttgtgtaactgcacttagtttacacaatttcttgaacttacagttttgcaaagacctcagttgttatgtgatttagcagttagagtgtacccacagagaaaggaagcaggaaagtcttcagagaggggcaagctgacatgcaaagaagcaggtaaccagacatttgctaagcgcctcgaagactgagtgcactgcactgcactgacctgcttcagctcgttggtaaagtacacgtaagttacagccacacagagagactgcaggagcactgtgaagatcacgatcagcacgcaggtctgtcccaggctgggtcccccctggacctccatcatagccatgatcctgtcagagtctgactgctgtaagtcagccaggcagccggt

SEQ ID NO: 14 - DNA, 3001 bp, synthetic construct
  source 1..3001: mol_type = other DNA
  misc_feature 1..3001: note = CD122
ctctcaagtaggtggtttgcatttctgggacctttggcacctcagttgaggctctctggttgtgagcaagaaaactcaactattaactaaaggtaaagtaaaaaaaaaaaaaaaaaaagtgatgttggggatggaggcaaggggctagagccccaaccaaacctgcaggaaggaaaagccagggtagccccagctccttggcgcccgctgtgcttgggatccctggctgcacctcctaatgcactcggctccatcagctcctggctgcggtcctgcttcccaaatctgcagagcaggactcaagctggccctgctagggttggggcctcccttggagccgtcaaccatccaatagggcccagaatgaggtagagagatgagcccaagggtggggtaaggggctgaggttttgggggtgggcagtggcgaggggtccctggagaagaggggaacttgtgagcctggggccacacccaaagtgcctccttcactgggcccagcccccacccaagccgcatttctaatgaaggctttgttttgtaagaatttttaaatgctttaactagaaattggcaaactgcaacatctgccaatttctagcagtatatatcctgccaccatgacccatttcaagctagcatcgtgatgtctctgaacctgcgatcgggaagacatgcatgcagtcggctctggtgcaagctggctccagccaccattggctgtggggctgagggcacaagcctcctcatagactcctgtaaggagccctgccctgtcttgagaaccaccgaaagagctagagagtgtttcccatcctccagccatcctctgagaagtcatagctcaggtcctggatgagcctcccctctttcccaggaaccatcccttccttctggtgtggcccactggacaagatcgaatatcccaggtcctgcaaaacctggctccttccggcctctgggtctttctttcagttcctgggaggggctgagcagcctcctacctcctggccttgacccatattggcccctccaccagctgtgctatgtctctcatctttgggtctcagctgaaccacctcttcctcagagaggccttccctgaccacttctccctcccaaatttgcttctcccttgccagcacttaccccacgctgaagccctgctgtgactgtgtaatgtctctctcccctattagactggaagccgcatgagggcagggaccatgtggggttttggctcaccacactgtcccctgtgcttagcacagggccaagcctatagaataggaatcaatccataattattgattgggtaaacaaatgacacatgaaggaaggaaattaatcagtttttctgtacttcacagtttcttcttctgtaaaataaatataagaaagtgtctaccttaaaataatttttaacatttatagagcacctcctacgtaccagtattttacatatacttactcctttaatcctcacaacagcccagatagtgttaatatcatccccattttacaggtgacaaaactgaggcacagagagtttcagtgacctcccctcatttactcagatttgaacccaggcagtgcctccagcagcctcagcattaaccacacactaaactgccagggggggttggggaggggtcctggaggtggtagctttggcgctgctgtagccctgctgccccaagagcttgaagcctcgtgagcagagccaactattgctaaacactataaccaggggacgctcagggatccagctgcctctcacatctgggaagatttcctggaaccaagacaccctggctgagtcaggcacccgcatctgacaatatggccagagagtttttacgaaatgtgaaccaactggactctcaacagcttctccccaccccaccccaacctgtgagtgggactctgatcttgcctggggctccagcccagcccctcagggttcaggggtaaatctcacttccccgaggttgggttaggtatggttatgtgaccagctgtggccaatgagaagcgagaagaggcttttggacagaagaatctctgattctctttcacccacccttgccacatccatgaaacggaagggttcgtcgctgggttttgagacttttttccatgaagctgaaggccaagggagctgaaagccttgccactgaatgctgaaacaaccttcactggctactttagagataacattcataggtcaccatggtaatggtggcttcggttgtttttcaggaattgggccagctgaaaccagttgagacccttcagctggggaaccgtatatgtgcccaagagatggccatttgatattggaggaccaaaagcaccacgctcagatcaggccaatgcatctgttttctgtatgtgtcctatgaaatgccatgaaccccaactatgcttgtacagaacaaaccggttacgtcattcttccccactgccaatcacctgtccccacaccttagatcaccccacttctctaacccattcatatccctaaaccttgtctttggggaggcagatttgaacgctgttctcccacctccttgcttggcaggcttgtgaatcaatcttttctcttttgcaaatcccaagtcacagcgattggctgcacactggcagaacagattgggacctggcgggtgatacccagatatgacacctggacccagaacagaaatctcatggccaccatcttgagactgccatcctgtggccctgagggaagctggctgtattagttcgttctcacactgctatgaagaactgcctgaggctggataatttagaaaggaaagaggtttaattgactcacggttccacatggctggggaggcctcaggaaacttacaatcgtggtggaaggcgaaggggaagcaaggcaccttcacatgctggcaggagagcgaagtgcagagtgaagggggaagggtcccttatacaaccatcaaaactcgtgagaactcactcactat

SEQ ID NO: 15 - DNA, 3001 bp, synthetic construct
  source 1..3001: mol_type = other DNA
  misc_feature 1..3001: note = CD27
ccacaggcacgtgccaccacacccagttaatttatttttattatgtgtagagacggggtgtcactctgttgcccaggctggtcttaaactcctaggctcaagcaatccttccgcctcggcctcccaacgtgactgggattccaggtgtgagccgcctcacctggccaattttacatttgtctttgagattatatgatcgatgtctgactctcacactggtgtgtcaactccgtcagggcagaaaccacatatttgtctgtttgtttttcatctttgtgccccattacttggctggttcctaacacaaaatgaatgtgcaaaaaacgcttgtcaaatgaatgaggaggtctaaggggccttgagccctctgacgtttccattttataattgtggacagagccgctacagcacaaagctgagctggaccatccaatgtttagccagtgccttacgtgctaggagacactgggagctgtgctgggcttctgaggagtgggcagaggctggaaatacccatagttggtcagtctcctgtgtcacccagactggggaggtgcagaaatgaaaattacagagctgcgaggacccagaacctcattaatgagcttaactcactgtcctgaccaggtggcctctcaaagccttgctgggaaggaggctttaaggtggtgggggcagcagtgctggtggtggtcatcgaagaaagtgggagtcggggaggaagcaatggggagcaggggacaaagagggccagacaaactcagagcttcaggtgatgagactcaggaagcctaagtcaggaacagtgattgcggacatctttgtcccataacagaacttcctcctaaggaaaggtcttgcagagactggtcctgctgagcgctttaacaggttattgattcataaaaattggagaaaaaagacaaagtctgccacacctaacagccttctgtttagagctggtttagaggaaactttaagcaagtcttgtgtccaacaatttgggcctgaggcagggtacaggtaagggaacagacaggagacacggaggcaagaacaagactttagctgaaagagaaatcagagccagaggcagccacaagaacacccaacctactggaactcatgcaggtggacagcctgcccaggacttgccctgagtgggaagcaaagagcatcttatctagattttaaggagcagggtgcccctggctgacatctaggctacagtcactgggccaggtggagggttggtcaggccctctcaaaagtcatgagaggccaggcacagtggctcacgcttgtaatcccaacactttgggaggccgaggagggcagatcacttgaggccaggagtttgagaccagcctggccaacatagtgaaaccccgtctctactaaaaatacaaaaaattagccgggtatggtggccggtgcctataatcccagctacttgggaggctgatgtataagaatcgcttgaacctgggaggcaaaggttgcagtgagctgagatcatgccactgcactccagcctgggcaacagtgagagaccctttctcaaaaaaaaaaaaaaaaaaaagaaccaaaaaaagtcatgagagtcctcagagttctcaagaagggctgctgtgggcctgtttctggaaaggaaagaaaagcagggagctgttgagaaaataccatatgtttttatgccccagccaggcatgggggagaggctgaccccaaaagagaaagtggttgtgtgagatttcaaggaagatgtcagcgggctgcatcccagggcctctagacaaaaggaagggcccagccaggcatggtggctcgtgcctgtaatcccagcactttgggaggccgaggcaggcagatcacctgaggctaggtgttctggtccagcctggccaacatgacaaaaccccatctctactaaaaatacaaaaaataagctgggcgcggtggctctcccctgtaatcccagccaaggtgggcggatcaacttgaggccagcctgaccaacgtggcaaaactccatctctactaaaaatacaaaaattagctgagcgtggtggcacgcacctgtcatcccagttactcaggaggctgaggcgtgagaatggcttgagcctgggagacagaggttgcagtgagctgagattgcatcattgcactctagcctgggtgacagagtgaaatggagggaggaaaaaaaaaaaaaaggaaggtcccgtccagggagccagcctaggatggggaaggctcaccagaagtggatgcaaagagtgctggagatgagctcattctatttgcctaggaaagaaaaacgtccagaaacctggccttcctcctcttggccgaggccctccaggaaagccaggcagaccctgctcctgctctgaccccctggagaagtgatatgtagaaaagaggagatgggctttggcccagaggacagcagcgttaggctagtgagcagttcccagtcggttcaaggttgctaggctcagcgaactgcaagtccctgttcgacaagtttcttcctaaaggtccccagttcctcatgattcttctgagggtctcatccaccctcccagggcctgcagtcagctagccaccccactgccccatgcctgcagtgaacacatttaccacaacctggaaatggctggtgacagaaaagtcctcacggccacaacactctctgcggtgggaattcaggtgccttgcctgatggtgacaaagctggttttgcataaacagctctctgaatcacacccaaatgtggcttctgacttgagtggacccccatgaagagctgcagagggggctgggaaaagacaagagaatggagaaaatgaggagaaaggaaactacagagtgagatagagaacagatctgacaggtgtgaccacagcagtgtgacatttatgaacagtgtggaaacaacggcaaagcccctaaattaaaaacttcctggggaaataagccactccacatatcg

SEQ ID NO: 16 - DNA, 3001 bp, synthetic construct
  source 1..3001: mol_type = other DNA
  misc_feature 1..3001: note = CD244
ggtgaaagaaccagatcctctcttgaaaaataataataggccgggcacagtggctcacacctggtatcccagcacttttgagaggcggaggtgggtggatcacttgaggtcaggagtttaagaccagcctggccaacatggtgaaaccctgtctccaccaaaaatacaaaaattagccaggcttggtggcatgcatctgtagtcccagctactcgggaggctgaggcaggacaatcacttgaacccgggggagcagaagttgcagtgagccgagattggaccactgtactccagcccaggccacagagcgagactccatctctaaaaaataataataatgatgatataaaataaataaaaatgcatatgataacctgaatcatatgagggaacatcagacaaacccagattaaggaaccctctacaaaacaactggcttgtcttcttcaagaatgcaatatcatgaaagatgaagaaaggttaaagtatggtcatagactgaacatgagaaacataacaactgaatgtaatacatgatcttgattgcatcttgggtcagcaaaaaaaaaaaataaagcatattcttgggacaattggtaatatgtgaatatggaaagcatatttgatggtattaatgttaaatttactgaatttgacaattgtgctgtagtaacatgagaatgtctttgttcataggatataagtgctaaaatatttaggagtaaggggacataatgtcacaacttgttctcaagtggttcagcaaaaattattgtacatttatacacacatatacatacagctagagaaagagaaataaagtaaatgtgataaaatgttaattggtgaatctagatgaaagaaattcaagaatcgtttgaactattcttgcagcttccctggaacttcacaattatttcaaactaaaaagcttaaaaattaaagaaatagagcagcttcagtcaacttatgccaggcctgccctatagacacaggatggaggaggaagcagctcaggaagacccaagtccatccctccctgtgcctaacaagtcactcaccagcttcaccgtgcaagggccggcatagccaagcccagctatggtgctgaagccctgggaatgggggtggagggagcattgccctctgtcacacaggctcatgacctcagggtcataacctcagcttctctgttactctcatccccacagccaggcaccaatcccatcagtcctccttcggctgtctgagctgcattcaaggatccttagaccaggttccaacctagctctcctgcctagaatcctgggccactaggccccccaacccacccacaagagctgcaggctccactcctccctccctgctccctgttcctcatcatgctctgccctaggctgcctgcatccttacaaatgttgagtccacctgagccttgactgtgtcttcctgaaggacacaagaatgctcagggactcggtaactgaattgagtacctgggcatgggactttattgcatatggggcttctacctggccctgaacccacacaacactatgcctcagtttcccctctttgtgcccagagtgtaccctcctctaattccacgctctccacctccgtgaagcccctgcagagtcactgatctaaaatgcagaaccctggctgggcatggtggctcacacctgtaatcccagcactttggatgggggagaccaaggtgggcagatcacctgaggtcaggagttcaagaccagcctggccaacatggtgaaacctggtctctactaaaaatacaaaaattagccaagcatggtggcgcacgtctgtaatttcacctactcgggaggctgaggcatgagaatcacttgaacccgggaggcagagtttgtagtgagccaagatcacgccactgcactccagcctgggtgacagaaggagactctgtctcaaaaaataaataaataaataaaaattaaaaatgaaataaataaattaattcaattaaattaaatgcgggatccaggtttcagagcatcctggaccccagaactggagaaagaggggagcagccctcagctgggatccccaggagtaaatgttagatggctccatgcctggctgacagctttaggtttgctgtggagcttcctcatattgtctccttcttctgccccaaccctgcatgtccctaataatttccaggctcctgtggcatctggatggctgaagaagccggggagatgagggaccatgccttccagcttcttcctggctgctggcctcgccctcccctccctgggctcagctttcctgcctcaggggtccatccatctgtctctgagctcgagaaccacccacttccttgggactgcttctcttctctcatgccacaattggaggatgcaattttcatcatctttacaggcggctggaaggaggcttctctctccactacacggaagggtaaattgaagccagtgggaaggaatctcctcacccaagacttcctggattgctggtggcagggccagatacccctctcacagacctcccagtgcactgccagcctgtcctgtctgatttagactgtcagagctgtcaggagctgctcggagtcaagttccagatgactgtgtatgtggcacaagcagaggctctggctctgatctccctgaggggcacagctcctagtccctccctcgccccacgccaggtaggacctccttacctttgccctgatacaccttgaggagcaggaggagtatgagggtgaccacttgccccagcatttccacaggacagaggggccaggccagcccctccaccccaccagactctctgccgtgcacgggctcagcagtccccagtcagcaagaggacgatggggagcagaactgccttgcaacctgtccagccacagtttcctcaattagaggctgttaacgcctgctgtgagctgacaaggccactgagaaagccccagcgcctggagctggtc

SEQ ID NO: 17 - DNA, 3001 bp, synthetic construct; identical to SEQ ID NO: 11
  source 1..3001: mol_type = other DNA
  misc_feature 1..3001: note = NCR1

SEQ ID NO: 18 - DNA, 3001 bp, synthetic construct
  source 1..3001: mol_type = other DNA
  misc_feature 1..3001: note = Ly49
tgcacaatcagtggttactgctgctactcggacgagaaagctgtattttattactcactatttgtcaccaacactgtgactatcatcagaagaagtaaacagaggatcccaagagtcactgcaatgaggtgccagggcactgaaaattctttaaatacaaaaataaggaaaataatcaccaggggaagagcttggctcactactaatcttgacctgagaaaccatttgaaacaaagagacaatatcctctaaaactctgacaaaaatatatcacacaagtatatttaaatttgtaatctggaaattacatgcatgcattgtagaaatcccgcacacaggaatagttctataggatttaaaaagtccatcccacataaaagcaccaatagcagtagacacaaaacatgtagcaaaatctaagctaacgtgtgtgttccttcctgtgatcacaactaatcatgaagagaaaggaaaaagcactcaataatgctaagaaattagtagtcaatagtggatattctgaagctgcaaatgtggatgggtccatctttgagggagtttctagaaatcaacttttctttctttttcatcaattctatttttctctaaacaatgctatatttatcctttagcttttgagatcctcagtgtctgaaagttgtatctctgattatctctatattaaagcagcaaaagttattctcaatgcatagtttaacaatgaccacatttgaagggagagagagagagcatagaaaaattagaatgccccttttgattcaaacattgttactgcatcattgccattaagatttggtaaatacttaaggcacataaaatatctgctatctgcataaaaatgttgtttttaacattgatttttcatttcttacacttcctctcatacttttatttctctaggaaatgtcatgttttcttcagatctagtctgtcctttgttatcaaggcctcacaaattcttcttgaccacatttaaataaaaacattctactagtttctcaaatttaaaagaattcactcatttctccattctcacaacttaaaacaaaaatacttctactactctgccccattatagtttcttatcaccagccaagaacaagaagtacagataggaatcttttttcaaatgtcaaaagtttgacaatccataagaaacaggtgaatgtaaatgcttaaaacgcatacctttgtcatcagttttcccaggcctttgagtatcatcaggccttaatctattctgtgactctgaaggagactgcaaaaatctcagggttgaataaatctctccctgatcattcatctttagagtaagaaggtaagttttgcattcaaatcctgaaataggatggtttctgttgaaagaaaaattaaaatgtcagccaataagaacacaccaggatttgatgctgagagatgtatgtgtgcctgtctgtgtgtctattggcgtgtgtttattaactaagtgcctaaagcatttagagtgagcctcatttatagaagaaatgaaaaatctttcaatacttttgttaaagtcatttatacttctagtgatcacagccatgataatgagaaaatacagaaatgatgagaaaaaaatctaccaatagattattgttctattcacccagccaattttgctcaaaatattcaacatacatatacaaatgttaggtaaaattgataccactgcaaatcactgaaacactttatgagttcaacaaacaaaaaatctaataaattctggaaaaataaaaatatcttggcttttaaaaacttacatgtattctaatatacatgtaaatataacaaagttcttgcattctttccaactatacttgactctatcagagtgctttccaaaatgtatcgcatataccttctacaaaagaattatttgagggttaatattgcaaattggttaacatgccagactcagtgccttttgcaagatgtactaaataaaaatcacttcggttggagcccagataaatgtattttaaatctcaaatgattccatgtacgttaaaattttagaaataatcttccggtatattgccagagaagcctatgtgaaaataacagttgtcaacaggcagtcattaatgtccataaaagattctattgattaatgaatgtcaatatagaaaagaataattaaatagtaaatacctaaaatttacttcttatgttagtcagtataaaaagatgatatccaagaaatgagaaatatgaaaaaacaaattgcacatgtaaccatattcttttctaatttttttaatttacaaaattttctaacctagtcacattttccctaatttcctaagaaaaagaagtattttctattatagagtcattcaagatggataaaatatgtaaatcattagacttaagtaaaaagtaattaacccctcaaatctctattctttattttgataacacaaagcaaaaatcacagcatattgagaaagagaaaataactttttgttaatactggactacaatttccttcaaagtagtgcagcattaatacaatcagtagcgaatgctttcctatcatttctgttataatgctaagtcatttgtagttaaatgccgtatttctagaattagtaagactaaggataatctggtagaaagatctgagaaaataaaagtggatattacctgtatctgtgctatgcagtggtgcaggtggagaaaactgatttcaaaatgagggagaagctggcctttcgggtcctggatcccttaggggctataacatgaaaggcagaaataaattctcttcgaatgatactgtaactcaaaattttctgccccacctcttgaccaaagtgataagaggaaattgtatcatgaaagctggtactgacgggaaaggaaagatagtcaaaagctgcatattcctctatctcaactaaccaaccacttagtactctttgcaaaataatgttcaaaatcaatatttgagggctga

SEQ ID NO: 19 - DNA, 3001 bp, synthetic construct
  source 1..3001: mol_type = other DNA
  misc_feature 1..3001: note = CD49b
tttggataactgtgacaacaggtataaaagatgaactggaacaggagagaaggaagcacagagagattatatgggagtttgtcaccatggtgaaagatcaaaagggtttgaaccaaagagagattagcagtggaacagaaaataggagatggcacagaaaaccctagtagggtgtaaagacagaattggaatgggatgttagcagggggctggtggtgcacctggggaagaaagcagaggtttctggtttgggtgattgtctatatgaaaacgggataaacaaaggagggcagggcatgacgatacaacagatcagaaggaaacactggggaaacttatctgagccgtgttccccaacaaaactctgtcttgagctaaggaaaagaaaaagaggggaaggcctcacttcacccctgagtgctttttggagtagggtctctgggtctctcaagatcctttcaagtggccagtgaagtcaaaactattttcataacaatactgagacgttactgcgtttttcactgtgttgacatttgccctcatggtgcaagaacaaggatggggactggtggtcattgtattcttcatcactgggcactcacaggaagagaaagggagagggacacggagaggccagcagatcaatgaattttaatgccacggaataggaaaaaccattgatatggtttcagattccacattgcaactttaagaaactaccacttgttgagttttggtgtagtatcaggggattatccagaattatctcctgtttttctaagtccatatctgtgtaacaccagattaatcaatataatttcaccaaaactacatgccgtaacagattggatgcagaagcagatctgagaatccaaatgtctgtttttaagccagatatttaaaatattagcaaaaatgtaaagcaatgccactcttctcccggattctttcatttggagaaatatagttatttttcataaaatgtacattatttatgttcacacataataggatttttgttaaaggacttactgagtaaagggctcttgagaccaaagagtttgagaagccccagcttaaccttccatatttcctgctctgaatgtggtgtccctcacaactgcaactaggcaggagagcaccctggaaggggttctcaaactactttgtctcaggaacactttatcctcttaaaattaatgtgaggcaggagtttgttctttgtttctaaaagttttgcctggctctggaaaatcgtagctcctgacttattctgcacttcatgacagtgtaatgttaatttgtccagttctgagaacaaaaaccaagaaggatgcatgttatttccccccacccccagagataaaacacaaattcctgtctcacattaaaggaagtgtcctgcctcccaaacacaggtctgttccaaacacttactcacatttcagtgagtcagaaggaaacagacagatattcatctccctgaaacctggctgacttccttcaaaaagtctgtaaggagcgtcaaaatcgtgtcaccggttagtgagcagccacctaagagctctcaggtgatagccggagttttagtaagcaggtgagtaccccagacaattcagaaaaaaatgccttcaggtgtttgtcaaggtgagtaagtgcagccagtaagtgactgataagaaaacaggaaagagcctgggactatataatttgggttcaaagggtattagtcataagccatcagcagcaacatctgatgcataccaatttaatactttaacaatgtgacacatcagccctatctatagtaaagagaagagcttgatttttatgaattattaagtccttcccaccttgagccactttgcttcctagaggcagtatctcaggcatcccattcagctcatagtagcattccatgaagatctggaatgatgacaataatgatgattattagactcataactccttaagcttgatttatgaaaacagttcgttccttcacaaaagtagtcactggaagaattctggtttcagcaatgtgctggaaatttgtggccaaacttttggataaaacataagattcagacaagaaaaattaatatcctatttcttaggtttttaactaaaaatctcggatatagaatgttctctttccagatgtgagactgaaataatggaagaaaatggtcacagtctatgtcatggtgtaatgtagacagggagagtcacaagtgcatgtatgggctctcagccttttctctggccttgaagttaatctcctaaactcagtcattaaatcagctcttaattatctcggaggagaggaagtaatgatgatttctctctctctgtctctctgtcacacacacacacacacacacagctcttgcagcaggtattgcttaaatatcaccttggataatcataacttgtgagcagatcttctttcctgatgagttagtttctaacctggtcattctgcgcttatttttgtccctttctccacccacttaggaaaaacagagaaagggacgcaccgcgcagcccctaggcactgtggtttagggctagtgccctcggcacccgctgccaggagccgggcgctgccaagggctgcggaggggccacgttctcccggggactggggcatctcctgcgtgctggcgacaggctgcgggggcggagtggtgccagggcgggcgctcgcccgtcggatatgcccacccgtcccgtccagggcaggaaagcctgccagggcgccatccccatccccaccgcctccaggctgccggggctgggccgctgtacgggagccaaggtgcggtgccccgcgtgtggacgagccgaggtgcagcccgcggggccgcagggccggggtggggcggggcgcggccggagcagatccggtgtttgcggaatcaggaggggcgggctggggcgggccctcggcgctgcaggagctgcccagaaacttttccctgctctcaccgggcggggg

SEQ ID NO: 20 - DNA, 3001 bp, synthetic construct
  source 1..3001: mol_type = other DNA
  misc_feature 1..3001: note = CD11b
tggttttcctgcatatatatatatatatatatatatatatatatatatatatatatatgatttatttttgtatgtatgtacttatttatttaattttaattttttattttttgaaactgagtcaagctctattgcccaggctggagtgcaatggtgcaatctcagctcactgcaacctccacctcccaggttcaagtgattctcctgcctcagcctcccaagtaactaggattacaggtgcctgccaccacgcctggctaattgttgtatttttagtagagatggggtttcaccatgttggccaggctggtcttgaactcctaatctcaggtgatcctcccacctcagcctcccgaagtgctgggattacaggcgtgcctgtatatattacatgccctgcctatatatattttagagacagggtctggctctgttttccaggctggagtgcagtggtgcaatcatatctcactgcagcctcaacctccttggctcaagcgatcctcccacctcagcctgttgagtagctgggaccacaggtgtgcatcactatgcccagctaatttttaaacatttttttgtagagtcagggtcttgctgtgttgcccaggctggtcttgagttcctgagctcaaatgatccctctgccttggcctcccaatgtgctgagattacaagcaggagtcaccacacctggctatatataaaaattataaacagataatttttaaccaaatgtacataattttaattgcttttttttttttcagccagcaagcatttttttccatctagttatcgggaaagtagaactagaggcaaaagaaaatgtgttgaatggtgaaaccctgtctctactgaaaatacaaaaaattagccaggcgtggtggcaagcacctgtattctcagctactcgggaggctgaggcaggagaatcacttgaactcgggaggcagaggttgcagtgagctgagatcacgccactgcactccagcctgggcgacagagggagactccgtttcacaaaaaaaaaaaaaaaaaaaaaaagaaaatgtgttgagaaagagtgaaaaaaatcattggcttgagaatcacattggcggtcacataaaggtgaggtttgtgttccatgtatgtgtggaaggatacctattttcctgtgcctttgaaaacagaattaaaagtgacagacacaggggagggtaaggatatatcacagagtgaagtatcattgaaccattcctctgttgaatattcaggtttttttagcttttcatattaacaataatgctgcaatgaacaagcttgcaaatatatatatatatatatatttttttttttttagagataagagtcttgctctgtcgcctaggctggagtgcagtggcacaatctctgctcactgcaacctccgcctccagggttcaagtgattctgctgcctcagcctcccaggtgggattacaggtgcctgccaccacgcctggctaatttttttgtctttttagtaaagatgaggtttcaccatgttgggcaggctggtttcaattgctgacctcaagtgagccaccccgcctcagcctcccaaaatgctaggattacaggcatgagccaccgcacccagccaagtttgtacatatatttttgactacacttcttaactattcttaggataaattactagaagtgaaaattcttgggtgaagagcttgaggcctttacacacacacacacacacacacacaaaaataggctggatgcagtggctcacacctgtaatctcagcagtttgggaggctgaggaaggaggatcacttgagtccaggaggttgagaatagcctgaacaacatagcaagatcttgtctctacaaaaaatttaaaaaaaattagctggccatggcagcatgtgcctgtagtaccagctactcggaaggctgaggtaggaggatcgcttgagcccaggaggttgattgaagctgcagtgagctgtgattacaccactgcactccagcctgggcaacagagctagactctgtctctaaaaaaagcacaaaataatatttaaaaagcaccaggtatgcctgtacttgagttgtctttgttgatggctacaaatgaggacagctctggctgaagggcgcttccatttccatgggctgaaggagggacattttgcaaagtgtgttttcaggaagacacagagttttacctcctacacttgtttgatctgtattaatgtttgcttatttatttatttaattttttttttgagacagagtctcactctgtcacctgggctggagtgcagtggcattattgaggctcattgcagtctcagactcctgagctcaaacaatcctcctgcctcagcctctggagtagctaggactacaggcatgtgccaccatgcctggctaattttttaaatgtatttttttgtagagtcggggtctccctatgttgcccaggctggagtgcagtggtgtgatcctagctcactgcagcctggacctcgggctcaagtaattctcacacctcagcctgtccagtagcaggggctacaggcgcgcaccaccatgcccagctaattaaaaatatttttttgtagagacagggtctctctatgttgcccaggctggtttcaaactcccaggctcaagcaatcctcctgccttggcctcccaaagtgctggcattacaggcgtgagccactgcgcctggcccgtattaatgtttagaacacgaattccaggaggcaggctaagtctgttcagcttgttcatatgcttgggccaacccaagaaacaagtgggtgacaaatggcaccttttggatagtggtattgactttgaaagtttgggtcaggaagctggggaggaagggtgggcaggctgtgggcagtcctgggcggaagaccaggcagggctatgtgctcactgagcctccgccctcttcctttgaatctctgatagacttctgcctcctacttctccttttctgc

SEQ ID NO: 21 - DNA, 3001 bp, synthetic construct
  source 1..3001: mol_type = other DNA
  misc_feature 1..3001: note = KLRG1
agactgacttacccttgcaaaacactttactctggtatctgttaatgatccctcaggccattgctacttgtttattccaagagtatcacggacaatttcctgatttaactcttctaaacttttctcacttacaaaaaacgctgcaagtctggactttggtttgaatattctttaaagttctctagttctttttcatgcgggtccacttactccacctcatttttaaaatataaaacttgaaacaggcagaattttcatgggtactaatgagtttgaagatgggaaaatggaagctccaaccttggccgtgaatgcactttccccattcccctaaacaacctaacatctgaccttccacttcccacaccccactaaagcaaaaaatcccgggaaattgtctacctgacttcctgtaccccaaacatcaattctctactgtaatattatggtagtttccatagaataatcatagctaaccaagaggtccaaggccctcataacacatttaatgcactgaaaccacacaacagcaggaaacaacagactactgccttgggccaggtgctgttcaaagcacttaacatatattaattaactaattaatcctcacaaccttatgagtaaatacaattattgttgctattttaccagtaagaaaactgagacacagctagtggagtgagccagagagtctagctccagagctcatggtctgtgttgaacaactacatgtatatttccactatgtgcataacaattttttggagatgagagtagacagccaaaactgtgggtttttttttttttctactaagaagtgaaggggaagtgattcctcacacagttgactgaataggaggtgaaataaagaactcagacctccaactcccaccttccctaagttaacccactgctcaacacacaacttgaccagctgactcactaaaaagaaataggagactccttcttctagactgacttagcctgccattttcctttccagtctctcaggacctaattaaaaactgacagttgatttcttttcctaaattagaacaaggaatctcacaaaattaggaagggatgaaaggttaaacacctcagccattagctgagttgaactatctttcatttgcttattattcaaaaagttaggataataaatttatactgtattcttctttcctgcttaaggcttgtctttaaatttaagctgcttctctttaattcagtggcatatgcgctaaatgttttccctagattatgttcattcatcttcacaacccagaaaataggtataatttttcatacttcacagatgagaaaactgatgctttacagaattacttgttaaattacttgctgtacattacacagatattaaatgtgtgtgaagttactggggcaatttagacgattcgtggcataaagataactgctggaaaacttataaacataggggtgcaacaatcaggaagagaatgaaatggaagggcccagtagcccccttttaatatgctacaattagattctcaaggtacaagagtttgtctatatactttggaaataaggagcaggattttatgatcttgaaaaatctttcagttttcttattctctaacgacagatctattttatttggcaatgctaaaaattatcctttattagggtggccaatgactaagccagcaactgggaagcagcttacaccactgggcctctgtagcactcacttccccatctgttaagacagccatggccatttaccagtggaatttctactgtcttcccgctggctcctttctgatgtagcagtccttatccctttttctccagcagcctacatgtttagaaaagcagcctcaattggctaaaaggcctaatgctgaaagaactcaagagagaaggagcctcagacaagtagcggctatgtcccaagagcggaagctccccacaacgaggaacacaagataatccgtccatcagccattcttatccacaccacagtcgcatccccctcaaagctctcccagggtgccagaaattctcgctctgtctagtgtcccgactttccaagcgcttgtagccataccctctccaaatgatgccacttgacctcccaccgtgatgcaggccaaaattcctcctacaaataaatccaccaacacctccaacggactccatacttggggtattttcctttccctgctttctaaatatcgtaaatcccctctctggtcttggaagtcctttctccctcacacctttcggaactccgtgactctccgctctcatgcctccgcctccattctacccttagggctcctcagccagatctttggctagaaccatggtcaagggagaatgcgattccggctcctgtcacagacgcttaagtgtgagcgcaacccagcccgcctcctcctccacgcccctcacactcacgtgtcgtgggaaactggagtagccaaagctcagaatccccagtgaatacccttctggggatgggagacggggccagctaggggccggccgcagaccccggtcccagagctagtttcagaggccagcgttccccctagcgcctcgctgtgccccactctgggaaccggaaacaggaagcgccaagaggccgctgggcaaccgctcctcccaccagaccccgcggcatgtgaccatagaaccatttcgcccctttccccttcccgggctccctgataatcttaaactctgattggccagtattctgggactttagctccgccctcgttccatcagctccaatcactatcgaggttgtcgtttcgccttttctggttgcctttctttcaatggggtcccagagtcccgcttccagggcctctcggctcgcgcaccccggtcacgcggggtcacgcgcctgaccgctcccgggtagggttcgagtgaagagggtgagaggggcaggtggcggaagcggaagttacgcaggcagctcgccctcgggctccgcgggagttgt

SEQ ID NO: 22 - DNA, 3001 bp, synthetic construct
  source 1..3001: mol_type = other DNA
  misc_feature 1..3001: note = CD43
cgggcgcggtggctcacgcctgtaatcccagcactttgggaggccgaggcaggtggatcatgaggtcaggagatcgagaccacggtgaaaccctgtctctactaaaaatacaaaaaagttagccgggcttcgtggcgggcgcctgtagtcccagctactcgggaggctgaggcaggagaatggcttgaacccgggaggcggagcttgcagtgagcagaaatcgcgccactgcactccagcctgggcgacactgtgagactccatctcaaaaaaataaattaattaaaaataaataaataaataaaatatagtgtatttctggccgggcgcggtggctcactcatgtaatcccggcactttgggatgctgaggctggcagatcacaagatcaggagatcaagaccatcctggctaacgcggtgaaaccctgtctctattaaaaatacagaaattagctgggcatagtggcatgtgcctgtagtcccagctacttgtgaggctgagacaggagaatcgcttgaacctgggaggtggaggttgcagtgagctgagatcgagacactacgctccagcctgggtgacagagcaagactccgtctcaaaaaaaaaaaaaattatagtgtatgccatggaatattatgcaactgttatatatgtgtgtgtgttcccaaagcattgttcagtaacaaaggaaaagtgcaaaacactaaaatggatgaatacatgcttgtgtaaacacagacactgtcaggaatgtttgtctggaggtgatgggtatggagcctttttgtctgatatattgggtgtatcaagaatgccaggtcctgagcagttttttttttttttttttgagttggagtctcactctgttgcccaggctggagtgcagtggcacaatctctgctcactgcaacctccacctcctgggttgaagcgattctcgtgcctcagcctcccgagtagctgggattgtaggcgcccgccaccatgcccagctaattttttgtgtttttagtagagacggggtttcaccatgttggccaagctggtctcgaactcctgacctcaggtgatctgcctgcctcggccttccaaagtgctgggcttacaggcatgggccaccgtgcccagccgtttatatgtattaactcttaatcatcaaaacaaactatgtggtattaccataattatcgccatttatacaaagcaacagagggataaggagattaaggcacttgcccaagtgcaagaggtaggacaggaacccagagtcccgtgctggtagctgcttttctgtgcacttaacattatctcatttaattcttgcagtcacttcatgaagaaggtggcggtcctagggcattggagaagttatgtgcttgttcaaggtcatgcagctggaagatggtggtggtaggacaggaagtctgtagggcctgattctgcagccctctagtctatactcctttgctcctgtgtccctcatcccccctgcagaatgggcaccccgttacctttctgagccactgtgcgcagaaaagagagcatgttggccaggctggtctcgaactcctgacctcaagtgatcagcctgccttagcctcccaaagtcctgggattacaggcgtgaaccaccacgctcagcctctgaatactttgtactcaagccatttttcagtgctgtgtttgcagtgagcacacccgagggatgaagacacgtctccctgtgggaacctgggcttaccagggcccctagaggaggggaatctctcaagctcagagctctatggctgcggtgcaggcccactgtgtgcatggtgtcagtctgggcccttccatgttgcccccgtgggacttggggtaaggggaactgatgcaaacatcacgctgctgttgcttggtgtgagcaattaattcctgtggctctcacccaggagtctcatgtctttgggtcagacaaactcatcagcttgtagaaatggcacagtcccacgggcctgttagaatcttctattgtgcacatgttgctcttaaaatatacaaatcagttttgattttaaaaaattatttatttttttagtgataggagttttgctacgttgcccaggctggtttcaaactcttgggctcaggaggtcctcccactttggcctggactgccagcataatgtatcaccacacccgggactgattttcgtttttcaagaacaaaaaccaaaaacatacacaaaccgagagtcaaagcttgctaattagaggaaagtcaggaaatgggaaccattcaaagaagaaaatacccccacctcctactctcacctatccaaagacaattaggtgaatcccttagtagatatctttccagacggttttccatatagattcccatatctggccaggcgcggtggctcacacctgtaatcctagcgcttggggaggctgaggcggatggaccacctgaggtcaggagttcgagaccagcctgaccaacatggagaaacctcgtctctacgaaaaatacaaaattagccgggcacagtggtgcaagcctgtaatcccagctactcaggaggccgaggcaggagaattgcttgaacctaggaggcagacattgtgctgagccgagccaagatcatgccattgcactaaactccgccttaaaaaaaaaaaaaaagattcccacatctttactagtttgcagaaataagatcctagcatatgcagtgtgtaggaaccaccttggtttagccacgtctctgtgactgggggccactgtggtgacccccagctccccggacagagtcaagagctcaccagcctgcaaaggttttcacggcccccagccagactcgggggcttcctcttgccctgctacttcctgggagctctgagggcaggaaatggcgccactcagctcctggcctaacagcttggggaccacaaatgcaaaggaaaccaccctcccctcccacctcctcctctgcacccttgagttctcaggctc

SEQ ID NO: 23 - DNA, 3001 bp, synthetic construct
  source 1..3001: mol_type = other DNA
  misc_feature 1..3001: note = CD62L
tcctatccagtagtaagaacgactgaaaggcagagtcttctccagatactcaatttccgccttgttttgtatggcaactaaatctgtgtaattgtctcggcagaatcttctagccctttgccagttcatgggtttttcagaataatggtaagtccagcagtcggttccatgatgtgccaggaaatctgcaagacatcagtgtgacctatgcagacttacataatgttacagctaaaaagaacctagcactactccaggctgagctagacacttagagatgaggaaacagagcctaagagtgtatgtgaccatctcaggatcacagaatagttgtttgcagatttgaagtagaacctagaccttctggcttgaatataagatgcttttatctaaggttctatttgaaacaaatttagtggttttctaggtttattttcttattaatttttttctcaaaattatttcaggtgaaatttaaccaacatattttagacattcatatttctttttctttgtagctgttaatgatttacaactaattaccgtgtaatatcatataactatacaatttacgtatactttttaatcctggaatcatttcttgaaggccaacacatatgtacctatgggagaagcataataaggacaggaagaacagtgacatacttttaagtaacctcttttacataaaaaacattttattttaccataggaagaactgcttctggaaaagcccaatataccactcaactcttatatatctaactgtataatttttaaaaagaacaatttacaaagccaaatggtataggattatgaaattcattagatcatgttctatacacaaagagactcaactgatgatgtttaataaacatatggacccatcaaatatgagggctttgaagatatctaattaaacacataattacacaatgacttcataataatatatggcattctaagcatggtatgatctacatgaatcactatttaatacagtaaagaaacagatataattgatggtaaagagcatcataaaataaacattttgaacagagttttgaatgagcattccactagaatgcaagttctaagagggaaaaaactgttgtgtccactgctgtatccttagtgcctagcataaatttcacacattgtagggactcagaaaatacctgttgtatgaaaagagcactaagtttctatgtgacacagtgcagacatggcataaggaatgtgtgaacgggagagttagcatgtttgcttggctagagctgaaaatccaggctagggagaaagaagacattagtttacttaggaaatgaaaaaccaagttcaaagctattgctggagagtcttcaagaatcagatataaaatttgtcacaacaatgggagaaggaccaaaaaatgataaacccccgtcccttaataagctcgtattgtaattgtagaaatgacattaatgtacactgaactatgaataaaaaatagaaaatgaggtgctaaatatttggtacagattgtaagtaccttaacagagatttcttaattaacattattcctttataattgagggattttgtggggttattgggatttgaactctacagcatgggctattataggttaaaaatagtgttcaggagtttctggggaagaactaaaggtaagaagaaaagagatgtttacagaagggatagaattaacagctctgtgaaataattttcccttagactatgtataactagtggatatttaagaaaaatgaatataagtaaaatagacttagcgatatataaatatcataacataccacaacagagcattgtccacccccacaacttgaagatgttccataagtccctctgggtgctctgacatttccatggaaatatctgcaaatgaaatacaaaattatatttagatgtatactcttaaaccacacatttatagcctttgaggtggtgcttacaactttcttaataatcagaataaaacacatatgtctactaaccctgtctgaggtaacaggtttctcagacatagatgaaaaattacttcaaatttacatcagaactgatgcacagttttgttttgttctattttatttttacgctttagtctcaagttgctaatcggtactgccctgaattttttctatggtttggtaatttttatacctgcttttctgctgagctattagataaaactatttaatatttactatgtatattttttaaagtattgttgctgcttaattaactattgatgcttatatttaatgttatagcctcactcttgatcataatgggtcaatgcctcaaatacctaaaaaaaaaaaaaattagatagccagacaccaggaaagaaaagtatttctttttttaataaaaagaaatacctttttgagcaactgaaatgacaaagtcacaaatttcctgcacaccttaaaatatacttaatgtaaatgacgagttaatgggtgcagcacaccaacatggcacatgtatacatgtgtgacaaacctgtatgttgtgcacatgtaccctagaacttaaagtataattttaaaaaaattctatcttccaaagcatatcacttctcaggtagacacagtgtttattgcaaaagatctgatttcaatagtatttcttcaagagtctccccagagacaaagtcaagaagaggaaatcagcatatctgagaagaaagatttcaggatcactttttttgagggtctgagaaaatgtttagtttctatattatttaaaaccagaattgaaatggggtgattcctatccttgccacctgcctctacaaccccaagagtttctatctgagcatctaaacgtcttttaggctgaaaggctcaccatggctttgcttggtccttctctagttcttctgcagcccattgagcctcttgacttagcacaagggtctcaggtccttgcccaaagggagtgtgctgtgctgcaggtagactgcactgaatgt

SEQ ID NO: 24 - DNA, 3001 bp, synthetic construct
  source 1..3001: mol_type = other DNA
  misc_feature 1..3001: note = CD226
tcagtgttcacaggcaaacacacacacacacacacacacacacacacacacacacacatccttctccttatcacccataataaagctattgctattttgttttttatttttattatttagctattgctatttttaagggaggcacaatggagataccttttgttgtttctctgtagaggtttatcctctctggtttatttagcttcctcttggaggctctcgaaatatggtaataaatacaaagaacccaagacttagctttgaatcatttccaaagaaacacaggcatcccagtatcttggccccaaggatgcaacattgttcccccaacatggtaactacagaattttactcagcaaacaggggaaatgttgaaatatcacatgatcgataataaattacaaaacaaacaataaaaataacctgagacaaattaaattgccctatccctccagagaacagatgtcctaaatgttctaaccagcagaacaaatggacgtaaagagggaaagcagtgagatagaatgaagattaccactcagagatcaacaagagccctctctccaacattttttcatgacccttgcaaaaggctttttgaacaacttaaagcaaagacactcttcaaccttaccagttgctgacagaagcaggaaacagcaaaagtcatcacatccctagttgcagcatcgcatcagctacctccttcctctttttttctaaggtcaccaaggcactaacttgctgttctaacaaagggtaaggaaagaaagaatcaagcatttgctctgcctttcctgtacaaattataattcaaggtgacccgagtttctctttataggaaaattcagctactaagtggaaaaaacaatcatagttattttttaagtcaccattttgcaacccctattaaaataatagatggagacaattgttaatagatgctaaaaccatgaggtggaaggttaatggggaaccctatgatgaaacgtgctgctgactgacgatacctgagccctcagatcagccgtaatcttgctaaatatggcacagccaggcatgagagggactcccaacacgatgcagcaggaagaaagcagcgccacctaggaagtcctcttccccgagtagctgagcctgaatgcagccaagcctctagaagtaaccagcagtgcatagaaaatgcaggcaatggctgggcacggtggttcacacctgtaatcccagcactttgggaggccaaggcaggcggatcacatgaagttcgagaccagcctagccaatatggtgaaaccccgtctctactaaaaatacaaaaattagccagttgtggtggtgcacgtctgtaatcccagctacttgggaggctgaggcacgagaaccacttgaacctgggaggcagtggttgtagtgagccaagattgtgccactgcactccaacctgggtgacagagcgagactccatcttaaaaaaaaaaaaaagaaaagaaaagaaaaagaaaataaaaagaatacaggcaataaaagactgagttaaaggacactacgagaccacaaggaagcaacgaactgaacatagaaagcaggagctactgcagaacaaacatctcagtttcctccagcaacaaaaggcaattatgatgaacacaaccagtaaatgccaaaaagatacttgacaattttcgttgttgttgttgagatggaggcttgctctattgcccaggctggagcacagtgacacgatcttggctcactgcaacctccactacctgggttcaagtgattctcctgcctcagcctcccaagtagctgggattacaggcatgcaccaacacgcatggctaatttttgtaattttagtagaaacagggttttgccttcttggccaggctcgtcttgaattcctgacctcaagtgatccgcctgcctcggcctcccaaagtgctggaattacaggcatgagccactgtgcctggccagatacttgataattttaaatatcctttcctgggaaagacttacagcaattttacaatttcatcagcatgataaagaaaatttatgagaaactaacagcaaccttgatagtgagacactagaaaaatctcaattcaattttgaaataagacaaggatggccacaatcactgtcaatatttaacatattctggaggttttaactaaatgtaatggattttaaaaaggaataggagctgtaaaccttgaaaattatttaaaatgtaggatttgtaggtgatccacctagagaacatggtcaaataaaacagaaaagaatttaataagctgcctagcaaaataattttataagactcagtagctttattgtatatcctatccaaaagaacagcaaaatttacacgtgtatgtatatgtaggtataattgtgtgcctgtgtgaatatatatgaatgtacctcaagaatttgtaggatttaaacaaagaaatttgctaatttatcaaaggaaataaaagaagtcttgaataaatggaggtatgtcctatgctcctctatggcaagacccaattttgagaatgcatcaaaaatatttttttagttacctggaattttatactcttttacattattaagcaggtcaggctatgaatggctattcatgaggcacttattgacggcactctgcaaaaaggtcttcacctagtatattgtggaaatgttatatgtttgcacaatcaatcattgaagaccttttacaacacacatatccccatgaaagtaagcaatggatgaactgctgtgaaaggtgctccctggagcagaaatccacaccctggttcttacctggttttagcattgccaagggcccagcaaacttcacccacgactcccttggccactgttcttttcttcctccactgtttttagtccccctctcttgagggtctcaaggtttcaaggaaataagtacttacacacacaccttaaggactccagacctcaagcatcaggcccgggtaagtgc

SEQ ID NO: 25 - DNA, 3001 bp, synthetic construct
  source 1..3001: mol_type = other DNA
  misc_feature 1..3001: note = TRAC
gcctgtctcctctgccttaaaatctcccttcccacagacaggaccccaggagaaccctccggctgggttctgcgtaggtaccacccccgcctcccatctccgagtgaagcaccaacttctccacccacactctcagatgggggttgggaacacagaactatgaatgcgccccctctcagccagggggaaacggggccaggcagcctccacatccagcatgaatggcagacgctgggggccaggactcctggggctagcttccaactccctcggtgggtgtctgcgctgccagcgccccctgccccccacccccaggccccgagaacaatcaagccgtccccagcccagggcttccatgcagtaaattcgtgtttgcagagctctgggaaatctttaggaggtggcatgagtattattaattggggaaaaagaacagattatgccaaataaatccttcccacataaccaaaccctctccgagagggggagggggagagcgcagccccaggaggggtctccatccctcaccagggcctgacagccacattaccatttacatggatttatcaatagtattgatccccagcagcgtaaatagctcagtgggaagaggccagggctggggcctgctgcgctcccgcccccatgtctgggcaccggcaccacagactcccccggcacccgctggcggtggcagcagtgaatttccaagcagggacacggcaggcggctcagggaagccagacagagcccgggatgccctggcagccgcgctgccgactcgccagtcaccgctggctccacccccccttcattgctctgctgggcctgactcagtctttgcatctgttcaatgggcgggggcacctgcccttctccatcctctccggacaagcagaggggagtgggaaaaggccccctccctcccccagatccccccactgtccacccccagccaaaggtaaggtggccccttttcacagggcttccagaccacgttggattaaaggcccaatcttagaacccaggaataataataccgacacagtagtcatagtaacacatacaagtaccaagggattcttatggccggacggtcagctctcacaaccactctgtgaactcagtaccattttgaccctcattttgcagatgggaaacctgacaggcacagagaagttaagcatctgtccagggtcacactgccaggaaagtggcaaaggtcagagtcaaaccccaggagctgaggtgcagagtgggccatcccacctcactgctgcttgccatttctcggcccctcctcagcacccccaaagaggccacccacgaggtacacagcctgccccgggtcgccctcccggcgcagtctggcccggagagccccttatatcaaagctgccgcaaggacccagatctggctgaaaaggggcaaagtcaaggccaccggtaacacatcccaacgcgcccagaactcccccaccccgcccacagggtcctccccttatcccctcctcccctcttccctccctcacacatccttcctccaaatctgcaaaaagggaaaaataataataatcacaataaaaccagttctcaccaacgggggagaggaggagggggaggaagggaggaggctgagccgacagaacagatccgcccccgctgcctgcaccagaccctcggagagccggagcgcgcgacaggcggcgggaggacactcgcttccaaaaaaataaacctacaatgtaaaaataacgccgggcgccccacgctaattgggcccgggcgacagcagctcccaggtacagcctgtctctccctcccgcccgccgtgccctcccgccctcggcttgctcctcctgcaagtgtctggggcggggagggcgtaccccacgggtgccctcactccccgcaggcctctgaggaagctgccctctagacaagggtctgggtcttcccagtcgtgcccaagtgggatcagctctgaattccaacttcgcaaagtagtggcatcccaagtatccgcacagcccgggggagggactaggaggcggcccaatgaggcgtcaccagccacgggaggcaggcccagacaccaggaacaccggcccgcgggggagggggaccaggggcgactctcccgagggaccccgcccagcgccccgtggccccacgcctaggagggacaaggctggctctccccctcggctggtgagagaccctcatgcgcccctccacaacagccaggccagggagctaggggtaccatctccaagggctttcaaacccgcgctgcccgatggaaaataaggcgcagtgtcgccccgccaggtccggggctacaccgggtacaagggacgcctggcggccaagtccccagagccggcgcacactgtggggggccgctctgtgcatttccagcgcgccccccgcccaccccaacgccacttccctccttcccgccccctcgctagggagctggagttcccctaagttccccggcgcccccgagcacgcgggccgcagtgtcggcggggcgccttgctccccagcaggccaccccccgagagcgccaactcgggagtgcaggggccccgccaactccgccgcgtgcgccgcccgatcctccccggatcccccggaagcgcgctccgcgcctccccccgcccttcgcggggaccctcggccccaactcacctcgcggaggtggcagcggtggcgcccgggcttggggcgcgctcgggccgccggggcgctcaggcaagtggcggtgccgatcgggctgggggcgcgcagcaggcccggcctcgggtcgggacgcgatcctcccgccgccgcctcctagggcgccggccctgggtccccggcccgcaagttggcgcgcatcgcggagcgcagggcgcgggcgcagggctcgggcggggcgccggcggacgcggggctccggtgcggcccggcctgcccgcggctccgcgctccccggcggccgcggcggcggtgg

SEQ ID NO: 26 - DNA, 3001 bp, synthetic construct
  source 1..3001: mol_type = other DNA
  misc_feature 1..3001: note = CD4
gactctggggaggaagtcctgtgcaggagaatggtagagcaacaaaatataaacagccccaatcttggccctggaggacggcgtgctgctccctctcttctgcaccacccacctccacacttcctacatgtgaaaaataacttcaatcttgttgaagcctctattattttgggttttcttttgcttgtagcagaacccagtgcttaccgatagacaattctcccagttgaaagaaggttcttttgtgataagatctcccagcaccttgtgctttttcctttgtagcgcttgtcgaattgtaatgaagttgttcgttgtttagtgtctatgtcataagctggagtggtgaccatatctgtcttcttcaacatcacatccccagctacctagcagagtgccttgggctggtacacgcttaactacgtacatgaatgaatggagcagggctcaaaacatcaatgcctaccagagcctgccaggtaatgtaaatgagaaaagcagactggctgtgaacaatagggagggtgtttgctttctagtctgaaggaagaaggcctgaggaggatacattttccccactttgaggagcaactctgaggttgcacctaatactttcacttctatctattaaccaaaattgagtcacttggacacatccagctgtgagggaggctgggaagtatattttaaatctgggtagctaagagctcagctaaaaattgagggttctattaccaagaaggagaaaatagactttggggacagctaaagcgatagctgagatctgaagaatgaataaaaattacctagatcagtggttctcagagtgaagtgtggcctgcagacccttgggagtccctgagaccctttcaggggatcttcaaggtcaagctattttcataaaaaaatacaggatgttattttcctttgtcactatgttgacattgcaataatgttgcaaaaacaatctggataaaactgctgccttagcataaatgaaggcagtggtgccaaaccatatgatagtcattgcattcttcactgccaggtacttacagtaaaaaaagaaaaaaaattaatgccacttttacttaagaatgcccttaatgaggccgggcgcagtggctcgtgcctgtaatcccagcactttgggaggccgaagcgggcagatcaggatcacctgaggtcaggagttcaagaccagcctggccaacgagtgaaaccctgtctctacaaaaatacaaaaattagccaggcatgttggcgggtgcctgtaatcccagctactcgggaggctgaggcaggagaatggcttgaatccaggaggcggaggttgtagtaagccgaaatcatgctgctgcacttcagcctggatgacaaagcaagactctgtctcaaaaaaaaaaaaaaaaaaaagagagatcaagaccatcctgggcaacatggtgaaaccccatctctactaaagatacaaaaattagctgggcattgtggcgcatgcctgtagccccagctacttgggaggctgaggcaggagaattgcttgaacccgggaggcagaggttgcagtgagccaagatcacgccactgcactctagcctgggcaacagagtgagactccctctcaaaaaaaaaaaaaaaaaaaaatatatatatatatatatatagaatgcctttaatgaagcagtaaatactaattttattaaatctcaacccttgagtacggtgtgtcatgaaatgagaagtagcacacagtactatatgctacagatgaagtacaatgctgtcaaataggggtacttgtgttaattgttggagtcgcaagctgaactagagttttcttttcttttcttttcttttcttcttttcaagacaggttctcactctgtcactcaggctagagtgcagtggtgcaatcacggttcactgcagcctcaacttcctgggctcaagcgatccccccacctcggcctcctaaaatgctgggattataggcatgagccaccactcccagccccacttttttcagactggaaaacgcacactcacatgtgcatctttaaatgatcacttgggctgtggtatggagaatggcgaccagtgaggaggcaggagctgttgtccgagcaagggatgatattggcatcttggattggcatggtggcagtagtggtagtgcagagtgacttgggtagattttggagccatttagaaggtaacatccacaggaactggtaaataaatacgtgggagaagttgggtgaagggggtgtcaaagattacacccaatttattttgcttgggcaagttggtggatggtgagcccctcactgagtgagaagcctggagaagcaggtttggagggtggtagtatgcaggtggtatgcatagttggggatgtgtgttgagtttgctatgtccggtgagcttcccagtggagatgtccaatgggcagacggatactcacatagagagttcatggtagattcgggctagaggaaagcacctgaggcctggccagagacgcctagaggaacagagcctggttaacagtcactcctggtgtctcagatattctctgctcagcccacgccctctcttccacactgggccacctataaagcctccacagatacccctggggcacccactggacacatgccctcagggccccagagcaaggagctgtttgtgggcttaccactgctgttcccatatgcccccaactgcctcccacttctttccccacagcctggtcagacatggcgctaccactaatggaatctttcttgccatctttttcttgccgcttaacagtggcagtgacagtttgactcctgatttaagcctgattctgcttaactttttcccttgactttggcattttcactttgacatgttccctgagagcctggggggtggggaacccagctccagctggtgacgtttggggccggcccaggcctagggtgtggaggagccttgccatcgggcttcctgt

SEQ ID NO: 27 - DNA, 3001 bp, synthetic construct
  source 1..3001: mol_type = other DNA
  misc_feature 1..3001: note = ThPOK
taaagacggggtttggctcttgttgcccaggctggagtgcaatggcgcgatctcggctcaccgcaacctccgcctcccaggttcaggcaattctgcttcagcctcctgagtagctgggactacaggcgtgtgccaccacgactggctaatttttgtatttttactagagacagggtttcaccatgttggttaggctggtctcgaactcctgacctcaggtgttccacccgcctctgcctcccaaagtgttgagattacaggcgagagccaccgcgcccggcatgggcataggctcttatttgcatcatatttacatctgtcttgcatgtgagttatttccctccacccaactttctcagttactctcagactctagagtgttcgccagccttggagcgtacccgttcctccagccattcttcggcctcagttcctttccatcattcgccctcaggccttcgcctaccccagcttcaaagaagcgaccgtaacctcctagctggctgggttcgagaagccggggacctgcgcctcctggtggctgatcggaaggaggctgagtgcagaggggttgtcttgccttctctgggacgtggagttttggaaactgttcccctgagcttcctgaagctaaatttgcctcccccggccttggggggcgcagagatcccggcggcgattagcgctgcgcggcagccggctccaacccagaggcccggaataggcgcggagttataaatagtgccacccgcaggtgttggggggagtcggcgggaggggggtacccctggcggccacggccccttcaggtgggttgggcggtcgcggtgggagcgctggggggcggggggcgtggggggtctgcggtctgagcgcccccagcggtttcctgggcggcgggtttttcgagggaagcggaggcagcggaggatgggggggagcagcggagaagaggtgggcgcctggcgcatgctgacgctccgcttgcagctcagccccccacattccgaaacaggcccccagacccactcacgcaggaccccgcccaggcaaagcctcgcctttcttaacacctgaaggcctcattcccccaggttctgcaagcatctaacttctatgcttctacccttcggcgcacgtggcccggccagtccgcgcacctggccgtcattccgagtcgctgtccccagccccaggtattcagtcctcccactcggggctttcattcctctcttagggtaaacacttccttctctgggtatttgtccaactcgtgctactctccccaaatttaaggtctagctggtcacctaaaaatttaagtcttaacatgcggatctgggacgctctcctatccccaggcggcccctgcaggcccctcacttgccctccagctcccagttccagtctgggcctagtggcggggcgggggcggccggcccctggggcccagcaggggtttcatgggagggggcggggtccccagtcagcgtttctctgccgccgctgtttcagtcgtttggcgtggttaggggttggggtaagagggaatttcaagtaggggttccccgctccgatttctgagtaaggaggggagctatttgcaccaggagaaaaagagagacaagggagggtgggcgtcactgaggaactaactgcgggttccaggaccattgggaatgaggtgaggagtggcaatctagccgcagaaagatcagtcatgttcagccagacgcagaacgcgaccctcaaacatgggggaagttgggcgaggcgagcctgaggcactgggcacctttcgagtttctgtacccaaaggctggtgccagggagcccgcccttctattgcctctggaagccctggctctgccctccccgggccacgttcccaccccccccgccccccgccccctgccccccgccccctttccttggctatcctccttcctccccaaagcagccccctctccatttcacttgtggcactgccgcctagcccctagcacagcccttcaccaggcctgcacgacccagacggctggagtaggaaggggaaaaccccggcctggacggctccgggcccctccggaccaggatctgtccggcggggctgagaagaaaacccggagcagtgatcagcctcgcgacccaagggggtcagggacgataggaaaacccggagcggattactcgagccagagcgccccatgcctgccgctacctcctccttcctatagcccccctaattctccagtgagataatgtctggggtggggggctgtttggtttggcctgagccctcaggggcgcctcacactttccgcccctggctggggcgcagggcagcggccccgaggctgagtcacggcccggcgcggagagggagagagggagaaagagggagggagggacctggactccgcccctcgtgcgcggggatcccgggtcagcccgggtgagggccagggcccccctggcgccgcccattgtggtccaacaggttgagctggtgtcccgagaagccacctgctggaaagggagggaggccccctgcggaggggggttgggaggggaaagcgagtccgacccggttcggcctggcttgtcgcaggggagccaaacctggccctaggtagccggatttgattccatttgacactagtgggaggggccctttggggtacgcagctccgcaggctcagtcagaaacatccttcacccgctacctttctcagttgggggactcctaagtatttatcccggtcctccccaaaacctacacataccacacatcccaaaacatccaggcacccctcgccacctgggtctctcagcgaattcatgtcaagctatccctcctcggtggagactcagtttctccacttagggaacccgcgggatggaggtggggagcaaaagcgtcggcgcgccccctcccccttcgcagactgggcccctcccctccgacaatgccaaggccgcagcgggcactggcgacagc

SEQ ID NO: 28 - DNA, 3001 bp, synthetic construct
  source 1..3001: mol_type = other DNA
  misc_feature 1..3001: note = CD8
gcttttgatacatttttctccgaagtcatggttccagtccacactcacaccagctgtggaggagttttcatttacccacgagctttgttatttctggttttgtcagtcttttcaattctgccaatctcatagggtgtgatggtatctcgttgtttaaacctgacttctcttgttaaggttgagaccgagcatctggccccatgtttgctggctattcagctttcctcttctgcagactgcccgtttgtccactgctctgtttcagtaacttccatataccaagaactctctgccttccataactcacatcacttaaaatacgaatttttttgatgcttagagaacctgatttccttccaaccaattgtgctctcctaattccaacaaccaaatgaagcttcaactcttctttttgttcagagccctcctgagtcctgtcctactcacagtaaaggctgtggcaaaataaataaataagtaaataaaggggaaaaatacactgcatccaaaaaaggctcgtttctttttctttctttctctctctctctttctttttctttttctcttgttgtgacaattgtcacaacaaaatgtgtatgtgacaatttgttatcttaaaacagtttacccaaaatagaaaccttacaaagccagtggaaacttttctttttgcataagttggttaattcttttgagtgctggcctgggatccatgaaataacctcaactcgaccataaaacttttcactcccacgttatgcaaacggtgatgaccttgttggtggcaagccccccgtgtgactttccgaggggatctgagtggtgcaaggtggaggacgcagtgatggaaacattgcgagagggaaggcgtctgtttccacccacttaccctcagagcatgagggctgggcgaaggctctgactcctgtagggggtgacccatttctaggactatagaaggagaggtgtggacctggaaaaaggaaggaaagaccacggcaaggaaagtcaagaagtgggagaagtcaagggctctctcctcccttcctccttgtccttggcctgcagcaactcctctttctctttttgtggggacagactgaagaggatttcttagtatggtttgtaccttcccagtagagcaggaaaaagaggaacgagctgctccctccacacctcagacgctgaggtcaactgccctttgggcggggcacgttggctcacgcatctaatcccagcactttgggaggccgtgaggggagcatcacttgagcccaggagttggagaccggcctgggccacatagtgaggccccatcctccggcttgcaccactcaaattccctcgtaaagtcacacggggagcttgcccccagcaaggtcgtcagtttgcaaaacatgggagtgaaaagctttatggtggagtgtaggttatttcatggatcccaggccagcacccaaaagaattaaccatcttgtgcaaaaagaaaattttccactacttttttaaagtttctattttgggtaaatcgtttcaagataacaaattggagagagacctagagagaaatgagcctttttaagatgcagtttattttgccccatgatgtttttgtttttgtttttgccagagcctttactgtgagtaggacaggactcaggaggcctctgaacaaaaagaagcgttgaagcttcatttggttgttggaattgggagagcacaattggttggaaggaaatcaggttctctcaaaaaataaaataaaataaaataaaaaaaaaagccagcgtgctggcgcgggtccatggtccaagctacttgggaggctgaggtggggggatcgcttgagcccggaagtcgaggctgtagtgagccgtgattgcaccactgcactccagcctgggcgatagaacaagaccctgtctcttaaaacaaacaagaaacaaaacaaaacaaacaacaaaaaaattcatttgggaatgtttctgcgcgtgcccataagcagagccatgctataggatctcccctgtgccccaacaatcagcttcttacttggagggtagagaacggtgcttccccacgctgctgtgcaatggataggagctatgtgaagctggcgtatgggtggggtttgcaggcttcgtccggcttcatcgccggctgctgacccggcaccaattcctgttctgcaggtctcaccgcagaggggcacgccagccatgaggacagatgagggaacacgtgatgccacgatgggggtgccaggatgaggtgggtgcggtcgcggacaggcgcacgaggagcccagcggagcgccacccggagcaggcgcggaggagggctggggagggccaccaaggcgacgagagccggtgtgcctgaatcagcctaaaggagacggaggaggagtgtggtgggcgcaggggcagggagctgggggaagggcggggggctagcccaggctgaaggcaggcaggagcagggccgcgatgtcagacaagaaacggcagcagtgtgtttgggaacctagcaccaaccgcaccggtggagacagggtctgctgtagaggaggtggggccgggtcccagctaagtaaggcggtggatcttgcagcccttccatcctcagccgctcattctgcgcaaatctcggggccagccttggtggagccgtaaagcgtccaccagaacctggatccctccgccacctttcccatgaattcacctttctgtacacagcaagcgcctgagcggagacggccgacacgtttcccactgttaccccaggaaaccgcggctcctgagggggtcagggcctgggcagggggcagagctcagcgcgccgtactgaggcagaaacggggtccagagagggtggggtgggggtacagggaagggtcgcccgaaggtccctggcgcagggaggacagaggagggatctagaattcgtaggggagaagagaactcagaaaagatccggcccagcgcatttattttacaga

SEQ ID NO: 29 - DNA, 3001 bp, synthetic construct
  source 1..3001: mol_type = other DNA
  misc_feature 1..3001: note = FOXP3
tgagaaggcagaagggggaatggtagcccaggttccccttcccccttctgggtgctgaggggtaaactgaggcctgcagttggggagagagccagaaccagggtcccacctagagtcctgagatctaggcttggatttcaactctgccgctgcatttcggtgaggccctgagatctctggtcttcaatttgcccttctacactgagcacggagaggcgtggagtagacaagggccagggcccttctacgctgtctggttaagtcattaggtgtctgcagggcttcaagttgacaattgcccctctatccaggggactggctgagagatagggatacatagagacaaagagacacacacaaagagcgagcaagagagaacaagagatagtgagagacattgagagaaatggacacatgcatggagagccagagtgcatgtgtgcgagaggaggattgcctcaaataagaacatttgctggtctctggctggttcaactgatgctgcctgaaataatcaagaataaagaagggcaaggtgccagggacacccatggctgggtattgaattgtattgcaaagcaacaatcagcaaaacagtgtggccctggtataagaacagatactggggaagagaacagaatagaaagcttggacatggacccacatatggagagaactgaatttgtgatgaatgtggcatttcaaactggaggaccatggagtatggtttaacaaatgtgtctgagataattagggagaagataaagttattgagtgaatagtcagtccattatcccaacaacccctccctgcccagtttgaaatgtcaccatcatcatataccctaaaatgcccagatccattcaagatataagttttaacacctaatgctgatcttgggtttattgtgtgtcaggccttgtgctaagtatttactgtggttaaaaattttaatctaaacaaagactcctgaggaaggtactattataaccattgcagtacatatgaggaaatggaggtatggagaggttaagtgcctggctaaaaatcacacatagggcttggggtgacgctgggtttgtcccagacagtctggctccagtacccacactcttaacctctatagtaaatggaaaaaatgaagccataaaagagactagaagccaacataggtgaacatttatctcattttcaagtaggaaaggactttctaagcacaaaaccagagacagaagccacagaaaaaaagactaacctatttgactgtataaaaccatcataagcatcacaaaaaacaccataaacaaatagaaaaaaagcaaatgatgaattggggaaaatatttgctatatatgtaatggctgatgaaaggttaataaccattccaaataaagagctgtggcaaatcaataagggaaaaataagatgaacaccctattaggagtaaggacatgaccagacaaccaaaaaaaaaaaaaaaaaagcatgaatggccaatgaatagtaaaagtaatcacaagatgcaaatttcaacaatgtgataatgtgtttttcttacctgtcatgttgggaaataattaaaacataataatactcacctagggttagcttaagtagagggagcataaaataggacaaccttttggaaggagaattagcagagagggtcataaactttaaaaatgcacgccccctttgccccagcaactcccttttcaggaatccaaggaagcagtcagggatgtttatagaaaaaaacgagaaacaaccggaatgtccaacaatcggcacttggtcaaatcaatcaaagttcatgctgatgtgaggacagtcttgtccatcatgaaagatcatgtgttcaaacaatttttagtaagcttcaaaaacactactgttaattgaataaagcaggaacaaaaccaatatatagcatgattctaatttggttacagaaatactaatagctaacacttcgtgagcacttactttgtgccaaacgctgtgctaagcctgcagaatcgagctcaccccagccctgaacaacctgtttgcttcctgaatatggactctggtcacacacatgcagtcctggggtaggtccacacagctaaactacggttgacaatggtgtgaagtgctccctgcccccccgccccaagggtctcctctaaagcgatacaagcaaagttcagttaagtgctcagcttgccccggcaccttgcaatcctcctgctactagggtgaacagaactgatgctcactctcataaaatgtaaaggtcctcggcgacattactattattaaacgccagctgtgtacaaagctctaggctggatgctggctgggaaggcaggtgggggaaggcaagaaaagagagcgggagagatggaggaaaggagatcgatggagtgtggtcaagatggaggagacagagataggggagatggtcagaggccaggagagatgcggggaaagagagtctgagtgtagcgacagacagatggcgggagaaagagaggcagagaaacatgtaaaagagcaagacagggtgagcagagagacagagaaggatgagaggcatcaagagctaagagacaaagagatgagagagatgcagttgaaattttcagttgcacctggacagcatttcaagttgttcaaagctctgaaatccataaagactggcagctgacatattttaaaaatcctatccatctacgtatcaattgatgaattcatttatttttgcccctgcccatgcattaagtacttcacctttaagtcttctgccatttattctattatttttttaaagaccttacctggctggaatcacggtagctgggtacatcccactgtaccagagggcccctgacccccccgccgtgcctacctccctgccatctcctccaatggggcccacatctggtaggggagagcagggacactcaccttggtgaagtggactgacagaaaaggatcagcctggcttgtgggaaac

SEQ ID NO: 30 - DNA, 3001 bp, synthetic construct
  source 1..3001: mol_type = other DNA
  misc_feature 1..3001: note = Helios
ctttgattcacataatgaaaagatcgataatctaactgtattctgtggggaaaaaagtttaagaattgacaagtcctgagaagatttttacttttttttccccctttggccttggtcctttttgcctttagtttcaaaactctcactgcaccagcagctaaattattcacaatgagccatttctgtattgatcgccaagtatatttagccctggctcctggaatttctccattacttactggaaaacaggcagcttcacttcttgtctccaaaaccacttcccttctccctcccctccccttcttcaccaccacactcccttaccccccccccaaaaaaaaacttttatttctttctttatggaataatcagatcaaattcccaactcagtcactacatagcacttaaatttcaacctgctgtactgttatcacattcttttaaattatgattacataaggctttaaagaaagagatccgtaaagtttaaaagaggagcagtttcttcggatattttacaaatgagcttatctctttaaaaatgtacacatttaacacacacatattaaaaagaaagaaacgtcagggagaagcgaaagaaggctgccccatcaatgaaatggttattaaccctcagagaaggaaagaaggaaaaagaaaaaggagaaagagaaacgaaatgtacatacaaaagaaattgtcctttgattaaaaaagattcatcaccatttccagctctgtcgggagatctcagcttcttctaacccctcaaagaggaggtgacaatgtcgggctgaagataaacggagggagaaagaaagaagttttttgtgtttcccccttctctctttccctcccttgccccctccaagccaagccccctgcagagttcaaggggaggaggaaggaaaagcactttacaggtgggtcattccaagcccaaatccagcaaacagatcggctgataaacaaaaccaagaaatgagagagaagaacacccccctcctcctcctcctcctcctcgtcctcctcctcctcctccttccacccctccccctcgtcccttagggatggtctagtaggaaaagtcactcaggaatggcaccacgtactttcaggaaagaggaaaaaaaagagagagagagaggtcagtcagtgctatagcaatgcgattaacaagagaaaaaacttgatccaccggttccttaagaatcgaccaaaagctaagacaagaaaactgaaagaaaagggggggggggcgggtagaggggagggacagtcaggcagaacccagctaggaacccaggccaacttcacaggactgtttttggcccttggcaaaaaagaaaaaaaataaataaatcttaattccatctctttcgcccatactagaacctgtcaaagtgatttaactgctgcctgtttacatgtgtcataccagggtttttggtttttttttttttttaattccaacaggatctggatattaccttctatctggacagcttcaatttattccagcctacccaatctttcttcttggaattcaagttaaaacaaagcaaaacaagtccaaattcatgacagctatagagatgagaactgattaatcgattaacatcccagaaacagattacaaggaggggacgataaattaaggcagaggacatcatcttcaacccgattccctgagcgtcctttacaaaaatcattaccctaatcagaaccacaatccatccctcccagagaaaaatacctatattattattattattactggttaacagcaacaagtcatttgatcacatcacagtgcaaaacgagatgctatataattacacttaactttgaaaacactcccccctttgcaaatcatacacatatttatatataatctatgaatcagaagacaaaacaagagctgttcttcaccacgcaaaagccaagcggagatttacctcagcagtgcatgcagtaaaataatcccatcacccttttgtttgtgtttactgttaatgtgtcctctgtctttctttctttcctgtgcctaacgtgtgtttgtgcactgcagttggtggtggaaactaaggcagtggatctgtagctaagggtaatcctgtttttacttcctccatcttcagcgaggagcagggttagcccgggacagctggtcaaaccccgagaaaccgatccggtggagcccgagcacatctcccccgccggccgggctcgcgcagacgcccgcgggcggagggcgggctggcggcggctgcggcggcggcggcggcgggcggctggcggcgggcagcggagccccgggcgcgggcgagcggacgtgcgtgcgcgcgcgtgcgtgtgtgcggggcccgagcgcctgtgcgtgtgtgtatgagagcgcgtgtgcgcgcgcgcgcgggctggcgggcgcgtgtgcgcgtgtctgcgcgctaagccgctcggcgcgctcggcaatcgattacaggacaagtgctgccccggaggctccgcgacgcgcgcactccctcgcgcccacccgcgcgcccggccgcgtcgcccccagcccgttggtccccggactgcggaccccgcccccgacgggggccaggtaacccgcttccgagtgtgccacggcgacccccatctcccctccccgtgtcataataaatctcacgttttctgccggctggaggcttggagcggtgaggtgacgttcatttcggccgcgtgaagttttctttcaaactgggattggtggggaggccgggggaagggagcggggcggcgatcttggcgccctgcgcggagtccgggagccgcgtcccggccgagtcggggagtgtgcgtggagcctcccacctactggtctccgccggcaactctgcggccccatttcggaaagcgcaccgcattgaccgggacctgaagcgggataagaggggtcctggggaatagactgagaaagacattcatgtctgagatgaagctcaactattactatttt

SEQ ID NO: 31 - DNA, 3001 bp, synthetic construct
  source 1..3001: mol_type = other DNA
  misc_feature 1..3001: note = CD25
ggttcttgcttcatctaggggttcatgatatccatactgcaaagtggctatgaagattacaataggacacatatctggcacatagaagttactttataaatggtagttttaaattttcttgtagttctttattgcctactaggtgaagttaagaagttatcactaggagaattctctttctgaccacatcccagggcccagcaatatacagactaaaaatgcctaatgccttagatgattcattttacctcatcctaactttccagtttgaattccccatatccccaccctccctgtccagggcaggagcacattggaccacctgctgctccctaaggcactcctgtgttttggctcattggggtctctctccctggaatctcattgatggaaatttactggtcccttggggctccctttacatgtcatcctcatgaagccttctctgatagcccaacgctccgtagatcttaccacactctgactggtattactatatttatatctatgtttttctcctcctcaccaggttataagttgcttaagtgtcaggactatatctccctcatttttgtatttcctatagtgttgaggggcagatggggtatggatagatgttgagaatggcgtgaaggagtccttatgggactctagttcccctgctccctccaagaccactcagacccttggataagtcaccaatagctagaaataacacgagtttacttgaaatgtgtcttccagaggcagagcatgattaattgtcaagattttcctgttcttggcttagactgagttagcaaagaccctgtggatgtagaaatcacattgaaaaaccttcctaaaagtatgccctctttataagaacggcacgatggactctgaggactcaggggaaagggtgcgaggtgggtgagggaaaagagactacgcactgggtacagcatacatgctcaggtgatgggtgcaccaaaatcttacaacgcattgggtacagcatacatgctcaggtgatgggtgcaccaaaatcttacaaatcaccactaaagaacttattcatgtaaccaaacaccacctgtcctccaaaacctattaaaataaaacacataggtcaggcgcgatggctcacacctgtaatcccagcactttgggaggccatggctggtggaccacaaggtcaggagtttgagaccagcctgaccaatatggtgaaaccccatctctactaaaaatacaaaaattagctggtcgtggtggtgcacgcacctgtaaacctagctactcaggagtctgaggcacgagaattacttgagcccatgaggtggaggttgcagtggactgagatcacgccactgcactccagcctgggcaacagagtgagactctgtctcaaaaagtaaaaaaaaaataaataaaaatgtaataagtaaataaaatgccctctttaaagacactcctacatctgggccagtacacagcccagcatgactgggcacttcccctaaacaagcaagcagactcctgaaatgcatcattatgtttcatttcttaataaattctttctacagccagttagcattctacttgttgccattatagactttcaaaaaacactataaactttcttatctgtcttcctgagtaagcaaagggaagacattattagaaaattcacactcaattaggaagagtggtttgaggtagaagttggtggttttttatttctttaaaaaaaaatattggccaggcacggtggttcatgcctgtagtaccagcagtatgggaggccagggtgagtggatcacttgagcccaggagttcaagaccagcttgggcaacatggcaaaaaccccatctctacaaaaaaataaattaaccaggtgtggtggcgtatgcctgtagtcccagctactcaggaggctgaggtgggaggatcgctaagcctgggaggcgaaggttgcagtaagccaatatcgcgccactgcactccagtctggctgacagagacagtgtcaaaaaaaaaaaatcttcgagaaaatccatttgaaacaaggaatgaaatttaaaatttgcatatataaacacgtatacatatacatatcatgtatatacataggattatgagaagtagtaatgttctaaaagtgagtatgcatatttttaaaaatttctgtaaagttgcacttgtaaggaaattcctaggtgttatttcctaagacgtaaaagcagtgtatacatccttgaagctaaacttttctgagatcttaaattctcccagtgtttgatatatgagtactcatgtctccagcaaattaacctgaacttaaagagcttcctgatagcttcttagttggggggtgggagtgcagagaataaaaggaaattcattgaagaataaaagataatttcttgagaaacctgctcatcgggaccctgttaacatccaaatatcccaaatatcatgccacactattcttcccagaattaattgtcaaggactgagaaggacaggagaacctaatgctatttcaggctctcttgacagaccaaggaacagtgaggcataaactggggtccccgtgggtcacaggcaagaggtggaacccaagattcaactcccttcttggaaccatctacctggggactccctctggttctgtggctgggaagagggatgcccatcagccttcccggaattccgggggcacccacaggcccttacctgcctggcagccaggcaccatgatgaacgtgagcagtccccacatcagcaggtatgaatccatcttcctgacccttgggaccagccggggcagtgaagcggaggtctttctctgcagaaggcccagttgccgtcagcctctttttggcatcgcgccggaggatgtgggatgggaagatcggtccgcctgggctgtcacccttgtgggtccatccagtctctatcggagtcaggagttgctctctttaagtattgggctggcgtgttcagccaggaaac

SEQ ID NO: 32 - DNA, 3001 bp, synthetic construct
  source 1..3001: mol_type = other DNA
  misc_feature 1..3001: note = GARP
tgccctgcctggccagctctgctctccagcctgcttccccggtgtcccctctcacattctgggttctggacacctcatatttccttcagttccctgagcagattttcactgccccaaaccttcactttctcccctctcctcttctgctaaagacctcctcatccttggggtcccagcttagatgggccaagaggccttctttggttccccaagtctggactatgtcctccagtcctcccagagcccctgagcttccagtgagcacagcaccaagccctccgtggctactgggccccaccccctcccatgggaagtgcccctagagaggggctcaccatcctttaagatgcatctcacagcacacaacaggtcagcagcggacagaaaaccgacactaggagtctcggtgacatggggtcgttactgagagcagttctccttggctctcccttcacctctctgcaacgttcccttcacccatccatgccgtcagtgggctctgactgctctccctgaaagagaccccggatcttcctacttccctcttatcactgccacagaccgagacgccaccccaatcctggccttgaccacagcaccctgacctcttataatctgtcctgtacctagcagccagagtgataatataaaatcatctgtcagatcctaaacctcttctgtttgaaaatctccaagaaaatccaggctatccacagtctcctctctgactcttgcccattttcccttccctcaccacattccagctttcctgctgttccttcaacacagcaaattcattcctgccacagggcctttgctcctgcagtttcctctccctgaatgctcttcctcagctcctcacacatgtcattgttttcccacatttatcatttcctcatcatttatttcttatcatgtgggaacaggggcctactaagtaggtactcactaaagactagtgaacaagtggcaaaatcagggcagaagctcagctgcagtggactgagtggtgagtgccaggtgaggacaaggaagggtcctaggcccttatttccccttcctctgccaaaccccactctatccccaaggtggccctgtgcacatccagaagggatcgtccctgcagcacatgggcactctgtcctggcatctgacgggaagggcccctcagctgtgggccctcctcagtggtgcccaagtacagggtgtagcatttaaagactactctctctcaaaaaggaggacggtgtcacttagacccaggcttgcatccaaagcccaccactcaccagctctgctccctaaggtaagtcccttcctttcttggtgccccagtgtcctgtctgcagggtaggcagctgggttccagttctgccactggccaaccccaactattgtgatgatcaggcaaagaatggtggcgaaggtgccctgcatcctcactgtccttcccagctgacacagtgaccctcgcagggctgagcatcccaggtgaggtgtgctcagtggggaggccccaagcctgcagacaagtggcctgggcaagaggaggcaggagagtcggactctcaggagtccaactcagaaaagtgccaaagaatgtgtgtgtgtgtgtgtgtgtgtgtgtgtgtgtgtgtgtgtgagagagagagagagagtgagagagaaagagcaggggtacggggggcaatgactactaccacaataagcccacccctacatttaaggatgttagtcttctaagcacttgacctatatcatctcatttaattctcacaatcacctattgaggaaattactgtaattcccattttacggatgagaaaacagtttccggaaagtgaagtgacttacccccagagttacccagctcataagggctttggagctgggatttatccagttctacatgacctgaatactgtgttttctccagcacatgctgagccgactgcccccaatagaactgtcttttttttgcccatgaagaaaaggaggctcagagaagtgacttacccaaagtcacacaaggagaaaatagcagggtcacgatcagaatccagttcatctgtctccaaagcaaatgttcccactataccaaatcaaggtctccttccctaagccctgccaacgacccctcacacgcaggagcagggcagactagctcacagtctggcctctgaggtcctctctccctcctctcggcctcagctgacttactggcagagtggggtcctgggagagctgctgcggacgttcctgtcaagcagtgaatcactaagggcaagcctcgttctggagacatgtgcatcccacgggggaattagaccttaacccaaaggaatgtggctgtgatggcggaatttcaggcagctcggcgtgggggtgggggcccctcacatcttaccacccatgaagaaggctgggttcagagccccttggagagcggtgggtgccccctaccccactacgagaaatgggcaggccatctagtgctgtgggatgggcagccccacgcctggctgggatcggagcgttgcaggaaacctgaattcaaattccggtttgcccttgcctagctgtgtgttcctgggcaagtcacggtcttcaggccattgttttcccagctgtcaaatgggaaggccggcctcacccctttgcgggaaagatccgccaagacaggagcgctggggaactcccagggaccgagggagggtcagccccggctccacgcgcgcagtggggatggcgggaggggagtgtttggggggcgtcccttgggaccagcccccgccttgcgctcgctagaggcgcgcgacagtgcgccccccgagggccggccagcctcgcctgcccggcggctcctacctggctcagcgcggccgcagctcctctcccgggcagtctcggcacattttgcgcaaacccgcccccgggtcccaccccgcgccagaccccggggtcacggcccgaggaggagcaggccgctcagctgccccac

SEQ ID NO: 33 - DNA, 3001 bp, synthetic construct
  source 1..3001: mol_type = other DNA
  misc_feature 1..3001: note = GPA33
cctataatagccctgttggggcacagtgtgaattttacacccctccatttcagactagaagactgtgcaagaagactttagcaaagtgaagccatttgcccacaccatagtcagttaagaggcactggggtcagccccctttggtcccctgggatggcagtggagtgttaggagggacgggtggaacatttcccaggcatggaggtctgttttgtcactgactctgtttagaattgtcagcacatgataatcataaaaagcagaccaacggtggctcacacctgtaatcccagcactttgggaggctgaggcgggcttattgcgaggtcaagagttcaagaccagtctggccagcatggtgaaaccctgtctctactaaaaatacaaaaaattagctgggcatggtggtgcatgcctgtaatcccagctactagggaggctgaggcaggagaatttcttgaacctgggaggcggaggttgcagtgagctgagatcacaccactgcactccagcctgagcaagccagactctgtctcacacacacacacacacacacaaagcagaccaacttttagtcggttttatgattgcttttaaactccctactttaaaagcacggccttgcgtttgtgccccctgcatggaaccttatgaatagcttctgcttcctccaacaccacttgagtcttgaggcccattcatcagtgcagtgcttccctgcccaggtcttagcagaatcaattcctcggaaaggtcagatgggaagagaaagaagagaaaagcccagtaagtcctctggaacccaagtcaagctccacctcctccaggaagcctttcctaatgtccctgctcctaactgggtctcccttacctgcgcaaaggaagaggtggtatcctctcatgaggcatggtggggagggccgcctggttgcttttggtcgacctttgaaggtcttccttgctcacctgcagtggcaatggctatttcactatttggtaaactctgtttatatacctcaagtcgaccaccaagcctggcttctcctgccccactgctctggccccttcttcctcttaatgtaagatatggaggaagaaaagttggtgggcacgtgattctgtgctgggcaggctgtggtgggagggatggggcggaggtagaagacacttcatcagtgtgtctgaaaacatgcccaagactcgttctgctgggtgagttccccaggggtcacttgccttctcataggaagaagcaaaatgtcctgctccaggcaattcagagttacggccacttttccctccaagctgggtgagcatcttgttttaagatcgtttttctaagccaaaatttcttatttttattcatgcatgggtcgcagccagtctacaagtgtggggatatgtgttgggggttggggtggtgcaggcatggaggagcttctggcttaccaggagaagatgggagaatcactggcccagccagggaaggatgggtatttccttcagcctccctctctcaggccctggagcatcctccctgcctgaatctgatggctgagataggcccacttatgactaatcattccagactcctcacttctatggcacctgcacaaactatgtgctcagtaaagagttgtttggataaaggaatgagccaaccagtccatcttggtggatgatgaggggctttctgccttcagtcagatgaacttgagccagcctgagagaatgatcttacccagatcttcctccaaaccgtgtgatccagcccttccccagcagcaggctgcccttccctcgagagccccaaggccaggtgttgagagaaaagcatgtctagggcctgggagaaccagaatgagctgtaggccccgtggcccggggctttcctgtgtatgctaaaaagtgagctctaaaccagtctcctggtttccacttccccaggcttggtgctcctgagtttcctggtatagctcttctcctccccgttgcagctggatcctggaagccctcaacagtgtctgaattgcaaggtgatggccaaatacactaaaccaaagatattcctaaactttgccaaaccctgcgtctctcaacttccttctctccattctatttccttccaatgcccagcaaaatggttggtaccttaatgccaagactttcattctaacccttagacactccataggtccccacagaacactgtgctattaagcacttgatgtggattcaaccatatactcatgaatgtctgattttgttctgggaggtttccctcccagagggaaatgctcagagtcccagaattttcaatgaccagggtcccagaagtggcaaggagctttggcctttggaaaagacccatctagtttgttgtttgtttttagggtttgaatcttctcttgctctgtgaatgatacgggataagttgcattgtctcttctgcctcagtttcctgtctctttctctggttttaacacaaacgttaatatactagataatgaacatatctagatccacacatcctggattttctaggacaactccgatttcaaatactttgtccacattgtcaaatcctgtgtctgatcctataatctaatttgggataagaaaatatggtcttgagagtactttgaattcttcatttcagcagtgggctagaatgttatttactaatatgtaaataaaaaccattctgattctgggcactggtccggaatgtgtaattctggcccagcaggaacgcagctgtgttgctggaggcccttcctgggaaggctctgacagagcctctccttcagccctaggtccccatgtctcacccacccctgggcactggagagaaaaggggcctactcacctgcacagagtgtccacaacacaggccacatcttccccaccatggtcttgcttcttctctgccctaaacctaacctgtcactggcagcctccagacaggtctgagctgtctggttaaagctacagcagcttc

SEQ ID NO: 34 - DNA, 3001 bp, synthetic construct
  source 1..3001: mol_type = other DNA
  misc_feature 1..3001: note = CD14
ccagtgagtgtccagctaatttaactgttttctgacatattttgagtctggttctggtaatgtctgtaactctcttatcaatgttaatttccattccctattcctatctgtgtggctctggacattggccagaggcaggtgctgggcctatatgcctcctcccacctttctcccatgctccctgtgaggcaggcatctagctctctgatggatgtggctgcctcctcattgaaatgaagaaggggctcagaagaaacagcacctcttgatggctgatgctgtaggtctctgagctaatgtggactagaggacaaatcctgtttattctgaagttttgaagagtctgagatgctttcctatttgccaatcagtcagcctgaaaaggttatctgcatatgtgttctaacagaacacaggatctctaattcagagaccgactgtatcttgcgtcagttccctaggcccaaatttcccattatgtcggggagtgacacagtgaaagcctccgggtgaagagaggttttacaagagaggaaccctaatattatgaaactcagagcttgtgcaggataggaggctgtccctcttccttgagtggtaagggagacagacagagagggagagggagggtgtgtgagtgtgtgtgtgtgtgagagagagaaagaataaggaaataagcaaatggctgtgggttttattacctttgaaagggagcagctggctcttgggaagtgcataggagaggaaatattttgtatcttccaaatatcatgcaaataccttttaatccagatgccaataatctgctcagaaagccctgactgcacactcattgtttctcccatgcagccctagccaggagtcctttcctggctccttctatccccgcagttcttttcttgaggaggacagatagggtttcttagggagttaggatgaagaaagcctaagtatgatgaattccccatccagcactgtgcccagcacatagcagacatccaataaaggtctgttaaatgaatgacacggacccgttgtttaagattttaataaaggtggggcaaagggttgaattggtcgaaaagtcctcaacgtcctgacgggactcccctgaagccaaggcagtttgagtccattcattattctgtcttggatcttaggcaaagccccgggccccttggagcagcaccagggttcccgacacccccaccgacagggtcgaacgtgcacaggctgggaccacgccggagttcattgagccctcgtgggggagggcagttccagggaccaggaagggattcccgtccagtgtcaggttatccacctcgggcagctcgtcaggctgcggcgccctgttcagtctgttgcagctgagatcgagcactctgagcttggctggcagtcctttaggcacctgttccagcccagcgaacgacagattgagggagttcagggcgctggaccacatgcatctcggagcgctagggtttacggtggcgcgcagcgagttgtggctgaggtctaggctgtggggctgcacacctgccgccgccagtgcggcgcacacgcctgtgggcgtctccattcctgtgttgcgcagcgctagattctggatggccgggaacttgtggggacagagagccgccatcagtccgcgttcgcccagtccaggattgtcagacaggtctaggctggtaagggccgggaaggcgcgaacctgttcgcaggaaaaggcaggcgagtgtgcttgggcaatgctcagtaccttgaggcctggcttgagccactgctgcagctcggcgagccaagaacgccctgtcgcccacgacacgttgcgtaggcgcaagctggaaagtgcaagtcctgtggcttccagaggcagcggaggcatggtgccggttatctttaggtcctcgagcgtcagttccttgaggcgggagtacgctagcacacgcagggcgcctaccagtagctgagcaggaacctgtgcggctcccactgtgagccgccgcacgcggagagccttgaccgtgtcagcatactgccgcgggtcggcgtccgcatcgacgcgctttagaaacggctctaggttgagaccgccggcatggatctccacctctactgcagacacacactggaaggcttcggaccagtcgggctgaggttcggagaagttgcagacgcagcggaaatcttcatcgtccagctcacaaggttctggcgtggtcgcagagacgtgcaccagcggcagcagcagcagcaacaagcaggacgcgcgctcctggggagagagcagaggtctaggaggccccatccaacccctgtggctcccgagtggcacgcgttcgaccccaagaccctacactcaccatggtcgataagtcttccgaacctctgagctccggacaggctctggaagtgctttagcttctttcctacacagcggcacccgccggcttccaggcttcacacttgtgaactcttcggctgcctctgacagtttatgtaatcctgggatgtcattcagttccctcctctgtgaaccctgatcacctccccacctctcttcctccgagccagcccccttcctttcctggaaatattgcaatgaaggatgtttcagggagggggaccgtaacaggaaggattctgcagggcatctagggttctgtgtctcctggcagtgtcctgatgactcaggcgccccaggcggtgaatgccctgttgactcgggagcctaagccttctctggtgggtgtgggaaaaggatgatcctcagtgccttaggccagtaccatactctgcactatccaaccccccaatccccctaccttatatcccagagaatctacttgattcatttctttgacttcttccttgtcttggtttatgttgatctcctgccaccaaatccaagtccctgaatatcctcagatatttaactgcatgttttgtggaagagattgtgaacctcatctgttggcaccaaggggggtagaat

SEQ ID NO: 35 - DNA, 3001 bp, synthetic construct; identical to SEQ ID NO: 20
  source 1..3001: mol_type = other DNA
  misc_feature 1..3001: note = CD11b

SEQ ID NO: 36 - DNA, 3001 bp, synthetic construct
  source 1..3001: mol_type = other DNA
  misc_feature 1..3001: note = CD68
catcatcgtgggtacccctggccgtgtgtttgatatgcttaaccggagatacctgtgtgagtaattcggttctccaatcccctgggtcactttgctcttgtgcacgctttccagtctttcagcgtaagccagagtcattcccaaggatgctggtttctctctgggggaagagctgctctgtgatggagcccatgcgtgtcatctgagcctctggcttccctgccagtgcagccctggcagtgtcctacttcccagggctgttgtctgcctggcgggaaggtcctgggcaaaggatcagtctttgtactctgagagcagactacttggctcctctctgttttttatcagcgaagttggatatatctctcccacatttccctaatcatatgctatatattggctttttttttcttctctagcccccaaatacatcaagatgtttgtactggatgaagctgacgaaatgttaagccgtggattcaaggaccagatctatgacatattccaaaagctcaacagcaacacccaggtgagggcagtcttgcttgaatagctaatgattcttgaaaaatagtaagtgccaggggaaccatatactggattcttgagcctttttatgcatctgcttcagttttaggtgtggctagggaagggagcaggcctcaggaaggaaccagcactctaagactggcctttttttccactaggtagttttgctgtcagccacaatgccttctgatgtgcttgaggtgaccaagaagttcatgagggaccccattcggattcttgtcaagaaggaagagttgaccctggagggtatccgccagttctacatcaacgtggaacgagaggtggggcccagtgcaggaggcgggcctggtagtgagttgttgggtatagcccctgactgatttttgtcccccaacctccaggagtggaagctggacacactatgtgacttgtatgaaaccctgaccatcacccaggcagtcatcttcatcaacacccggaggaaggtggactggctcaccgagaagatgcatgctcgagatttcactgtatccgccatggtgtgtttgcccgctgccagcctgttgtgggtctgcccgtcagaagtgtcctacttgaagccagggttcctggaacccaggtgcctacctggtctgctgcatatttgttttctcttccagcatggagatatggaccaaaaggaacgagacgtgattatgagggagtttcgttctggctctagcagagttttgattaccactgacctgctggtgagtagagggaactgatagcaaaggcagaagggaggatccaaggtgattccctctccaaggggacatcagtgcctctcaggaaagtagcagcttggaatagaatctggcatgcctaaggcctttggggaactgggatgcttatttcctctgccttccttggctgcccacatggatgcctaagtgtcttccctccgggatagagtgtcctccgtgcacatgctgaagagttgtctttcttgacgtaggccagaggcattgatgtgcagcaggtttctttagtcatcaactatgaccttcccaccaacagggaaaactatatccacaggtaagcgtagatctggaacactcccctaccccttcacacctggccctccctgggcttaaagctcctgatattcctcatccccttccttgttttccagaatcggtcgaggtggacggtttggccgtaaaggtgtggctattaacatggtgacagaagaagacaagaggactcttcgagacattgagaccttctacaacacctccattgaggaaatgcccctcaatgttgctgacctcatctgaggggctgtcctgccacccagccccagccagggctcaatctctgggggctgaggagcagcaggaggggggagggaagggagccaagggatggacatcttgtcattttttttctttgaataaatgtcactttttgaggcaaaagaaggaaccgtgaacattttagacacccttttctttggggtaggctcttgccccaggcgccggctcttctcccaaaaaaaaaaaaaaaacactaatccatttccctaacctagtaacctccagatcccagaggctctcctcacctcagctgagctcctttgaaagtgattcaagggactatgtcactcagcctcatttgctggaccaaatctggagggagaacccctaaaacccctaagtgaggttgcccagggggttgtccccaggtggggggaagcaggggagagaaaatggtagccatttttacattgttttgtatagtatttattgattcaggaaacaaacacaaaattctgaataaaatgacttggaaactgcctgtttgggcttctcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagctacttgccacaactgccaagcaagtttcgctgagtttgacacatggatccctgtggatcaactgccctaggactccgtttgcacccatgtgacactgttgactttgccctgatgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggt

SEQ ID NO: 37 - DNA, 3001 bp, synthetic construct
  source 1..3001: mol_type = other DNA
  misc_feature 1..3001: note = CD138
ctttgtaagatcagaaaaagcaaaaagaacacctaagcttcaagccagagtctgaccccaaagctcaggtggggctttcaggctcagctcaagcacctcctccaggaagccttccctccctgcagctccagggagtctccctcctttccccttttgagactctcctgcctcctgggccctatgtccaagtttctgggcaagaaataatgcctcacactgcagcatctgccccagcccacagaaaactccaggaaaggcagttcacggggaagcccaaactccacctgcctccccagcattaacgggtgcggtgggatcccagggctctggaatgtctgctttaccccaggcaggcaagggaaccagcagccctcctaaatccagccggaaataggtggaggacccaacttcagccgcccctgctgaggccccatcatgtgcgagaatggggcagtgaggtgatgcccagcggtccagggaggtaaggcacccagctccattctcttctccaaactcggagcgaagactccaagcacagtctcagttccacagaaaccaccaccctagagagcagcttcttggacctgaaagccaggagtgcatgagacccagctcggaaatcatggccctccccgtgctcccagatacccctcacagtccagcccattcacagttcaataggcacagggacctctcctggaccggaaaatgtctggaagcacgagtcacctgggaagcagggagggcgctttacacatcggtctcagaaacgactgtccctacctcatctcccatgagacagactctgtaaagaaaaaaaaaaccaaagcgctcaataactggcagctactacacgaggcaacgcttacggagggtcagcgctgctgagacttgtccagatgccacattagcagtggagaacccgagtgcccaccccgcccccgccccttccctgcccacgtgcctccctttgccagagctggggaagtcgaactgtccagatgtagtggcgagacaccgccacttccggaaggggaagcgcccctggctccgcacctcccctctgcatccctggggatggccgagccggcccgcaggagggaccccgggacgccagcaccgggcgcgggggctggggaggcgaactcgggcggcagactcgggcaccgggcggcccgcgctgacacctggctgcggcccaggtcccagcccccgtcaaggccggcgttacggggctgttcccaggaaagggttacaggggccgagaacaaagcgcccccattggggaaggcgggctagcggccggtggccggggcgggagagccactttctggggaccctcaggctgctctttgtcggccacgtccctctttagtggagcgtcagccccgctgaaggtggatgctgcatcccacccaaaggcctcttacagccccgagctccacatctgcccctcttttctcccagctgcactgggcgggccaagggacccgccttccctccgcgcggcttcagcacaaagccgagccggggagcgggaggccattcccgggtcccctcgcgtggaaggcgcctgcgcctcggccgtgcccggcacgggaacgcgccctccggggccagctcaacttcagcagcccagaagttgggctcctccgggtctccagcgttccgaggccaacttcccggaacctcccgctgccgggccggctcagctcacctcgagccgcccacggcaagcccgagggccctgcagacgctcgcccgcgccccccacgcagccctggcccggctccccgggccaacgcggccgcctcccgctcccgcgccggccgcgctgcgcccaaacttgccctggcgcaaggggcggggcgcccgggctcggcggcgctggggcgcaagcccgcgggtctggtttgaattagggtctgcagggggggcgttgtggccgcggtcactgcatccccccgggtcaccgacgggggcccggccgccgcggtggccggggcgaggagggtgggaacgggcgaccccgggcgccgctgtgggctggcgggctccgacagatgtggagacgttttacataattgagcgcggggccccggccccccaccctccccagcgcgggaccggtgcggcacccaccgacagcggaggaaattggggctggagccctggtctcggggctcaccgtcccgggacccgctgggctagcgcgggaagaagggaagtcttcgctccccctccccctccacgtgcacccgccggcatccgcgggtgaccagtcccggcttcccgccgcctccccgcctggccgccggccgcactcaccggcagggccggctgcaggctcagcgccagcgcgcacagccagagccagagcgccgcgcgcctcatgctgcccggaccggcggcgggagagcggcaggctgcgcgggtcgcggctgcgggccggcttcgcgggttccgctgctcgatgctctcttgggcgcctgcccagcgcgccgctgtcccaggcgagggctgcagggtccgccggctggagtccgctctctactgccggattcctctccgctcggctcggattcggcccgcacctctcccgccgagctccgccttataataaacccacaggcccttccttagccgttgcaaaaactggccccccacccccagctccgccaggtctcccggccagccccagtccacaccccccaggaccccgccccccagccacccctcccggccccttcggaacgccccacccccggcccgctcctaaggttcttgcctgcttggctcccgctcctccggtggccgaacgtcgctttcgggagctgtggtccccgcaagccgcggttcccggcctttcagctcggctgctccctgggtggtggggccgaggcgcacccccttctgcccgggcccggagtgcggaggcgcagcccgggagaggggctgaaggggggcaggcagtcctcgggcgcgagtgtgcagcgccctc

SEQ ID NO: 38 - DNA, 3001 bp, synthetic construct
  source 1..3001: mol_type = other DNA
  misc_feature 1..3001: note = IgH
tagaaagataaactcaagtcgcaaaacatgtttttccttgaaaaggaagaaatgatgtaatacatgtttcaattgaataactgtctttgtttctcacttctgtagtatgcttcaccctgcacagatgtcccctctcccaccccacaaaatgcttaaaaggtaactgaactctgttctgggctcagtcctttggatgttaattcgacttggccggtgcacctaaataataaatatcctcctgaacctaaataataaatatcctcctgaacagagtcggtctctctgattccttaaaaaatcccagaacagtatggcacttaaatgtgcaatggtatttattttaaatatcagtctgttgttaataaattgaataactaataagacaaacatcacttttaaaactgtattaccttatttgttgaatttagatggtaattttcaaactaggcaagataaaattttttacttgaaaagttttgaaaaaaacttctttggcatgaatattcaatacaaactgtagtctttatttgccaacatgcctttataatagagaacatccagcaatatgaagctaaacccagcccatgctttttagggctcactcacaatggcagcttcctagagggtggtctgagagagtgtaagcacatggggatttgggcttcatcatcaaagtgaagaagtaatttatgaattttacaatatgtaaagccagaaaattaactttcccctcagggcttactgtagtgtgtagcccctcccccgtagtctaagttagagaatactaactgcctgtttttccttctgtgctcagtgagccttatctgttctcattggtttcacattccttgaggctcagagggttcttgcttacctccccagcacagctgcaaggtcataagattgttaagtatatgttacagaaccatgtattcccaaggatgtaagacatgaagtaacaaataactgcctttgttctctcttctgtaattacgcttcctgcatcatgtagctcccagccactgactgtgtaaaaggtggctgctttctttgtccagggctcagactttcctggatgctagtccaactgagccaggtgatcaccttttaataaaggactctcctgaactctgtttggtctctcccatctttgatcgtcctgcaacaaaagcactagggagccccagggctgagcacacagaaggcagcaggagctgcagagcccactctgtcgtacttagggaagggagggaatggaatgagggtgatgtctgcaggaccttagaaaagggtgaggagggcagagagtctgcaggtagatgagcatattctaaggagaactgttatcctcctaaacttggttggcttcagttattatgaagagaaggaaactgttcaccagacttggaggacagaaagtaaagggaatttttatttcctgcatggtgactgaggaagataaaagacttttaaagtaaaagggaagatggagaaatagtctgaaaaacaggacaccaggagccaaccaaagtggagaacaagagcctttaaagtggtgtttaatttccattccactttcagctgatgaaagaaaaagaaacaaaacaccatgcatatggtgagttaatgttagaaaacatatataattgtttgagtatcacagcacaaaaacactaaactctattcatggaaacaatgtgtatcgaggatacacattttttaagacagggtctcactctgtcaaccaggctggagtgcagtggtacaatcacatctcactacagcctcaacctcccaggattaagggatccccccacctcagccttccaagtatctgggaccacaggcagcatcacacccagctattttttttgtatagaaaggatttcattgtgttgccaggctgttctaaaactcctgagctcaagtgatctgcctgcctcaacctcccaaagtgctgagattgcaggtgcaatttttacaattttactcttttaataatagtattttaataaaatatataaataacctattttaaaaattgttctgacagatcattgctagcgttactcagaaaacacgcagcattaaaacttcaaataggctgggcatggtggctcacgcctgtaatcccagcactttgggaagcctagcggggcagatcacctgaggtcatgaattcgagactagcctggtcaacatggtgaaaccccgtctctactaaaagtacaaacattagccaggcatggtggcaggcacgtggtggcagctactcagaagtatgaggcaggagaattgcttgaacccgggagttggaggttgccatgagccaagatcatgccactgcactccagcctgtgcgacaagagggagactttgtcaaaaaaaaaaaaattcccggttctaagttaatgttttggaagtaacatttgtaaagaaatcatggatttacaaggaagtaatggttctgagcgtcccctggtgtcctgagtgccccctggtggctcggagcaccccctggtgtcctgaacaccccctggttgtcctgagcgtcccctggtgtcctgagcgccccctagtggttctgagtacctcctggtagttctgagcgacccatggtagttccaatcagcatctaccatgcgattccctcctgtctccctgcagaggattttgtgtctgggctcacgcagatgttcctgctcctatgtcactcacagtaatacaaggtcttgtccttggctttcacattggtcattttaaggtagactgcacttgaaagggtgttgcttgggactgttaatttacttgtactcatggagagtaaccctgataactcactgttgccacccacacaaatccctgtcatgaagcctgctggaccaagctcatgctgtagccagtacaagtgaaatcagaggctttgcaggagagtctgtgagaaccgctgggctgtacaatgtttccccctctgactccatcagtaagcttcaacaggact

SEQ ID NO: 39 - DNA, 3001 bp, synthetic construct
  source 1..3001: mol_type = other DNA
  misc_feature 1..3001: note = IgK
cctgcacccagtttctcctgttggttacttcttacgtaattacagtacgatattaaaaccaagaatttgacattgataaaataagtgtgtacagttctatgtcattttatcacatgtctagatgcctgcaactaccactgcaatcaagatacagatttatcccatgatcacaaagatctccctttatagtttcgcctactcttctttccccaccaccctaacccctggcaaccagaatctgttctctatccctataattttgtcacttcaacaatgttacataaaaggaatcacatggtatgtgaccctttcagattggcttttttcactcagtagaaagtccttcagaatcactccagttctctgtatcaacacttcgttcctttttattgcctcatagcattctatggcatggatatattacaatttgttaggctagttgcctattgagggatgggttggctctttctagctttggctacaacaaataaagctgctgtgaacaatcatgtatgggttctgtgtggacataagtttttatttcctgggggtaaattcccaaggagtacaattcctgggtcatatggtaggtgtcttttgtttagtttttcaagaaactgacaactattttctagagtagctatacccttttatattcccaccagcaatatatgaatgatccattttcaccgcatcctcacaaacatttcagtatgacctaattttgacttgattacctctgcaaagaccctatttccaaataaagtcacatttatgtgacccatttatgagtaccaagagcaaggagttgagcatacctttttgaaggacacaattccacctgcagcacctgtcttcacttgcttaggttatgggtgggtttcccttcagagtgttatcttctgagaagaaggtttgaattcatagctggattatgcctgcctttgattgttcaatttggcccccagggatgagtgagcaagttcatgtcattggaagggacattactgagtctcacccctactcaaacctccaaagtttgaaagatttgatactcatcaacattctcaagaggcagaagggcaaggtgcttccttatgtagtttctccatgactgtccttccccactttcccagtactgactggaaagaaaaacaaagcaggacaaagggattgtcagtcatttgagggaagggatgctgtgttagttactcctgaaccccttcagagtgtaggcagagagaggtacttagaaaggtgtgttggatggatgagtagctgagaggggcagggccagagtgccccatagaatgaggaagagtcctcatttacatgtgcccttcccccttcccaaacatgtgagtgttcatacatgggtttccagtgtctgggcaaggttaattctgtcctctgtccccttgctgaaggtctgtttcatttaatttctatgtatgtgtgctcctagctagccaaacagggcacagatgccaggaaaatagcaggcctgttgttttgttgagcaccttgagccctggaaggcagactcattcagggctcaagagggcaggattaggcaggtagctatttgatcccaggcccccccacttaccagctgcgtggtcttgacctattacttattcctcttctatctattaactgggtataagatgttctttatgtgaagttattttgaagcataatgtgtatacagaacagtgcacagatcataactgcacaaccccacacgaggaacagaacattaccaagaccccagaagccccttcttgtcttctcccaggcactaactcgcccacccccagctaagagtaatttccatccaaatttctaactttatttttacctggttttgaactttatatggatgaaaacatacagaacatatcttttgcattgagtttctcttatgtaacattatttttatgagattcatctgtgtgagtaggtatagttgtagcttgttaattctcctcattgggtattctaatgtataaacataccacagtttaataactcattctattgttggacaatcgggctatttccaatttttggcttttgtaaataaagctgctttgaacattcttgcatagttttttggtgggtggggacactcagtcatctagaagtgggattgctgagttatagaatatatttgtctctcagctttatagaacctttgcaacattttaatgaatgtttttaatctcacagtatagtgtggattaaattaggttgtggaaagtactcaatacggcccttggcagacaggagtccctcagtacctgtttaaaaatgtaaataaggcatgacattccattaagtctaagtaccatgatgaacttaaacatttccctattgtttgtttatttaattgcttttattgtgttgttattttatatcatgttgcaatgaatgattctctttatagcaggtttcttcttttgaaaaatttgctttggttgattctcagggaattgaatcaaaggatatatgactgtaagacctgtcacccttaaaaaggactatgagggcttgctgaggaggggaaaacaaggaagcaagtctctcctaccatggcccaggggactgtgaggacagaaggcttgtgggtttgagggaggactgtcttgcagaggatgatagggtaaaatagaatgaaggatgatttttataaatggttatgcgccttaggatgactacatatttagtcccttataagagaaattgagtagttggtaaaacaacagataataattattaaatgaggaaagagagaaaccacaggtgcaaagattcactttatttattcattctcctccaacattagcataattaaagccaaggaggaggaggggggtgaggtgaaagatgagctggaggaccgcaataggggtaggtcccctgtggaaaaagggtcagaggccaaaggatgggagggggtcaggctggaactgaggagcaggtgggggcacttctccct

SEQ ID NO: 40 - DNA, 3001 bp, synthetic construct
  source 1..3001: mol_type = other DNA
  misc_feature 1..3001: note = IgL
gaacacaagatccatatggggaaggatttctatgagggtgtggcagtgagagcgggtggcaagttgccaaagaggcctaagccacagaagtgggtttcatgtgggactttcggaatgagcaggagggcgatgggtggagggtgagggtcagaagattctaatcagaggtgagaacccaaagatgagttattggtcattcaagtcaaacagtgaaattaccttggcctgtgcctccagtcagtggagcagggaaggggccaggatgtgcttcctgcaggtgttactggaaaggggtcctgattcagatcccaagagagggttcttggatctcatgcaagaaataatttgggaggagtctataaagtggaaacaagtttattaagacagtaaatgaataaaggaatggctactccatagacagggcagcaccaaacactgctggtcggctatttgtatagatagtccttgatcatatgctaaataaggagtggattatttattagcttttcaggaattctttttaaaaatatagaccacataaggtaacttgtggatgttgccatagcatttctaactgtgaggacaaccagaggtcactttcctcaccgtcttggtttcagtaggttttggccggcttctttagcgtatcctgtcttatcagcagggtctttatgacctgtatcttgtgatcccagtctggttgacttcctagctcatcctgtgactaagaatgcctaacctcctggaaatgcaggccagcatgcctaagcctcatttaatcaagcccctattcaacatggagtcactctggttctaacacctctgacttagggtggctgcagagctggaggtgcagattggggcagatcagcctcctgagaaaagcacacagagccactgcggggctgtgaggtgggagagatgaactcacattggcatttgatcagtgactctccagctgtaatgagaatagaatggagaggcctgggacaggaagacaggatgtggttgctcatgccatcctggtgggaagatctaggtgagttactgtggggtaaagaggagagagcagattccttgtcactttatgctgtagaatacattggacatggagagggcttggagcttgtgtgtgagaaaagcagatctatctcggctgactcccaacaaactggctgagaatttagatggaggattattattaaataaaagaggaagactgaaatgaatattgtcatcgtatttttttacccagggttgggataggtggaaatgtgtgtgtgtgtgtgtgtgtgtgtgtgtgtgtttaaatattcactaatttttcaattcaaaaatatttactgcgtgactactaagtaataatattatacaggttttagcgttaccatggagaatcaaattgtcaaggtcacaggcatctttttgttttgtgtggatcactgtgaaagatactatgctaaattttttctgaatcatgcagatgaaagcattaaacacatacacaaacatttacgtttgtgtgtgtgttgtgtgtgtgtgtgtgtgtatgtgtaatctatctcagcatacaatgccactgaaactacacttgcggattaggtcctcggaaaaataatcgtgttaacattcacgatgcaaatagaatggtacctgacacaaagtaagtgctataaaagaatacttgaggctgggcgcggtggcttatgcctgtaatcccagcactttgggaggccgaggccggtggatcacctgaggtcaggagtttgagaccagcttgggcaacacggtgaaaccccgtctctactaaaaatacaaaaattagtcaggtgtggtgatgggtgcctgtaatgccagctactcgggaggcttaggcaggagaatcgcttgaacctgggaggcggaggttcaacaagagcaaagcccattctcaaaaacaaaaaatttgaatggttaaatcacattggactgtgagtaaagggcttagacaaggtgtctcggaacttttgggtcatcttcttgatctttccataaaactctaacccttctattcttaacctcccccattcccaatttcaccctgatgtgcacatcctacctggtgcctagtcatcccggtggcccctctctgggctgtatctagggagagtggtgaccttggtcatcttcctcaggtccaagtatccataagagaagcagacttttctctctttcataatgatggtctcttcttgaataatttccctgtgcattttcattaagggtctcaccccatcaagggtgtgatgaggacagtgatgagggtcacggagcccggcctcccacggggggatgtggggtatgatgagagagagaagcaggtgccctcaggcccatatggaggctggggagggttgttaaccctcctgccctcctggccactgggtgcaggacatgttctctctgaggctcactcacctatggccccaaccacacaaaaatctaaatggggaagaagcagaggatgacttcatgctgaggaggcccatcttgcagcgagaaatgtgctgtggtgagcacctgcagcacaaagagcctggggtgggggtggaggggcgtgagcgctgcctctgtgtggggatggtgtgcgcaaggcagtgctgaggaccttcaagccagtcaggaatgaacctgggctctgagacagtggagggaagtgtggcctgtccctgagggtctgtgggggtttcaactgggaaaccaggccctggacccctaccctgggccctctgctctgtgctttctggggcccagcagctgggacctcccaggagcagaagaggctcctctagccccaccccacactcaactttccccaagttcaggcccagggatagcagggggaggggtgtgatttgcatgaagggtgctctgtgccctcttaggaggagaggataagagagactctgtgcagttctgctcagctgtgggcttaggaagcagagcctggggcatctccacc

SEQ ID NO: 41 - DNA, 3001 bp, synthetic construct
  source 1..3001: mol_type = other DNA
  misc_feature 1..3001: note = CD19
ctcagtctctcaaagtgctgggattacatgagctaccttgcctggcccctaacttcttttctttcctttatccagcaaatacctattgagtgtctactagatgcctgtgcacatctagggatggtaaagatttctgttctcaaagaacattctggtgcaggaggcagatgagaagactagcaaatacacatgcataatttgagattggggcaagtgctgggaatgaaataggggcatgcgatcaaaaaagcatgtgagtaaagggaatttgagaaggttttctttttcttttttcttttcttttctttcttttttttttttttttttttttgagacagagactctgtcatccaggctggagtgcaaaggtgcaatctcggctcactgcaacctctgcctcccaggttcaagcaattctcctgcctcagcctcccgagtagctgggattataggcatgagttaatttttttgtatttttagtagatactgggtttcaccatgttggccaggctggtctcattctcctgacctcaagtaatccacccgcctcagcctcccaaagtgctgagattacaggcatgaaccactgtgcccagtctttttttcatattttttgtagagatggtgtttcaccatgttgcccaggctagacttgaactcctgggctcaagtgatcctcccacctcagcctcctgagtagctgagactacaggtatgcaccatcatgcccagatacattttttttggtatttttagtaaagacggatttctccattttgcccaggctggtttcgaactcctgggctcaaatgatctgcccacctgggcttcccaaagtgctgggattacaggcgtgagccaccgcgcccagccgagatatatatatatatatatatatatatatatatatatatatatatatatagagagagagagagagagagagagagagagagagagagagaaagaaagagagcaggctcttgttgcccaggctggagtgcagtggtgccatcttggctcactgcaacctccatccccctgatcctcccgcctcagcctcccaagtagctgggaccacaggcatgtgccaccacacccagctaattttttgtatttttggtagagatgggttttgccatattgcccaggctggtctccaactcctgagcacaagcaatctgcctgccttggccccacaaagtgctgggaggtgtgagtcacctcgcctggcctgagatatatttttttaaataataatatatagatttttgaagcactggggggcacagggaaggcctctttgaagaagtaccatttgaactgagacctaagtgatgaaaagaaccagtccagcaaagaaccagagcccccagcagagatccaggaaaaagggctgcaagtgcaagggccctgaggcagggaagcacttggcaaggagagtggtggaggcacgaggtggaaaatgtaggtaggtcagcaacactcggcctactaggccttgggttggagtttttattttagctagatgaaaagcaactgacattttttgttttttaaaaaatttctatagagatgggttctcgctgtgttgcacaggctggtctcaaattcctgtcctcaaaggatcctctcgcctcggcctcctaaagtattgggattacaggcatgagcctctgtgcctggctgtaactgacatgttttaagcaggggaatgacatgctctagtgaaagccagtctgggcagctgggtagctaatgaggggattagagagattttgttgaatgaaaggcagattgagtcctgctactcgcccccttcattccccttcattcatgcctcattcttccgcctcccagccgcctcaactggccaaagggaagtggaggccctgccacctgtagggagggtcccctggggcttgcccacagcaaacaggaagtcacagcctggtgagatgggcctgggaatcagccactgagaaagtgggtctcttgggtccctgaattctttttctgagtccctgcagcagtgaaaaagacacagaggcacatagagagtgacagagaaagagagagacagagaggagaggcatggggcagaataagaacagatttaggagttagaactcctgggttcttttaaaacaatttttcttttagagacagggtcttgttgtgttgcccggactggagcacagtggctattcccaggcataatcatggtgcactgcagccttgaactcctgggctcaagcgatccttctacctcagcctcccaaggacctgggaccataggcgtgtaccactgtgcctggcttttgcctggttttaaactgaggcagtatgacttgagctcttaggcattaattgaagctgtatctcattaactgagggcttatgatgtgctggacactgggctaatagtgctgaacatattgtcatttttaatcttcacaaacaatatttgtataggactgttttcttttcttttttttttttgaaacagagtctcactctggtgcccaggctggagtgcagtggtgtgatctcggctcactgcaacctccgcctcctggtttccagtgattctcctgcctcagcctcctaagtagctgggattacaggtgtgcgccaccatgcccggctaatttttttttttttttttgagaaggagtctatgtgcccagcattgttctagagcacttgcaattagtggtgaacaacacggtctctactccaaggggctcacattcttgtgcagaaaacagaaatgaacaaataaacacacaagatcatttcccgtggtagtgagagctgggatgaaaataaaacagcgtggcagggaggaggcaagtgttgtgagtctggagggttcctggagaatggggcctgaggcgtgaccaccgccttcctctctggggggactgcctgccgcccccgcagacacccatggttgagtgccctccaggcccctgcctgccccagcatcccctgcgcg

SEQ ID NO: 42 - DNA, 3001 bp, synthetic construct
  source 1..3001: mol_type = other DNA
  misc_feature 1..3001: note = CD20
ttttcaataactatgaggtcataattgcatttttttcttcagctatattcccacgtcacaaaagcaaagggagtgatccagtaatctgcacatgggagtacatcagacatttttatatcttaaaacgaataataataaatgttttgccatttaaagctgtgtcacttgaacaaattaatctttgcgggcttaaaatttttagactagaatgcaataatagtaatacctacctaatgggtttactgtgaaccttaaatgaaacatggaaagtgagtttatcagtgcctggaacttaaaaatcacttagtaaatgctagtttttgaaatttataattgagattttcttgctctaaacatcacttattttaccatttaccagctttccaacttgggaatggcttcctccatttcctgaaatttagtttccacaattataaaacagaatcaatattatatgtgtggtaaggcttatatgtggacaattcttgacataaaaggaattatttaactaatattaccttcctagctgttccaaatgtatagtcccttagaagagaaagaagaccctatcaactataaagttcttaagtatttcaatttacattttccttactatgaattatttataagtgcaacaatgggattttctgacctgtaaccaagttctatttgcaattagatctttagcctcacctctgacgagccctatgtctcccttaccaattctttgacaccagaaccctccaacagcagataattctgggtgccagggccttgctgagaagttgagaacgtggcataatgttacttcctaatattggagagcctggggacaatcacctgggtgaaagaaaaagccaaacagaaccattccttctcagcactgccagggacacagcaatcacaaaaactgaccctgccttcaagggacaaggagacaatggctgattaaggcaaggacacaaataattatgatgtagcacacagttttcatatagaaggcaatgattaaaaaaattggagtgctgggacgtgcagagaataaaaaggaaattcacatcaggaaaatactaactaaataaaagttggcatacttttatgtagactttaagattgtcttttggaagcaaggtcactagataatgaaaataggcttaattcaaagagaaatattttaaatatgttatcaacctaataaaatattctcaaaccttgggggaagagtgatggtgatacatcaaataagatgctattgacaagtccaaattgtagtgagaatttcaacacactcaagcactaaaatgttgatcattcaaatgagcaaagacaaggtaaatgtttatgcattcatacaaattcaaattcaagtttatttgggaagaatcatacatagagccacctaagttatctcattggccacaaagtagcaaacaacctgagactgactggggaaatacagggctcaaacagagaaaaggatgcaaaagaccaggttgtgctgccacaactcaagcccctttgctgccatacatttgccaagtcctgaccctgttcccatgccaactaaaatgaaggcacatgcctctaaccaccccaccatccaggactcaaacctcaggtttcacaatcatccacaactcagaccctaggtaattgtcctcccatcaccaatcactcaagcatttcagcagatatgcatagaccctcaattagaatgttaaatggatgatggatcttctgttcccactctccttcactctacagcatctctgagaggtgactgcatgactttacttgagtattcttgaagaagtaaattctctattgaaatggtagccagttcccctaatgaacggctccaacaatttttttttttagagtggcatctttttaaatatcacaccacaatcagcctccccagaactacttctacccagtggatctaaccctatggttccaaactatcttaaatttaaaaagagaggcagaaagagaaagagagaagagatgaaagagagagacaggaatgaacctcgaattaggcaacagagctgcctgccatatttcatccctttagattaaataaacaaataattacctaactatagctctgtgtctctaataaaatgaagcaatgaacagggaaataaaaatgtaattaaaaatcccacatatgaacataaaagttgaatattgtagggggagagagaggacattatctttgggcaaaactttgattcacatcccaaatatttagactctcctgacactaccaacttcttccatttccctacttgatctagtactgtgcttgatgaccctgcagctccagactagactaaaaagagaggggacacaagggaacttgacagacagaagaaaaaggtgggttcagaggtaaaagtgctatttcaatttatatttccaaatatggaggctaattcttgctttgagtggctgggtacatgtactatggcactaaagtataattttaggccatttctgctaaatctgacacaagatatatacatggttcataagaaacttaagtgtgagccaatgaggacaatatttggggacccctaagtatagtgctgccaagtgtacagcctcagtgattcttgaatctctgcatggatgcctcctgcccacctactcagccctaaaagtgaagccagaaggtaaaagtcagtgctaacggcccatctttgaccaacttctagaatctttcttgtcctcagggacctaccatccacacctgcttatagggtgggggctgcagaagttcttctaattaaggtagcatgagcatgccaggcagtcccctggggtctttttcaagaagtgaaacctggtaaggcagaaactttttttgcacctccttcagctatggtaagtgttaaaccaaagtaattggagcgaagcccaaggtagcagaagctactgatttcctgtcacctgatgtctatcagcgatttcatcttcaggcctggactacacc

SEQ ID NO: 43 - DNA, 3001 bp, synthetic construct
  source 1..3001: mol_type = other DNA
  misc_feature 1..3001: note = TH
tccaggctcaggtcagctgtcggccaccaggccagggtttgccactggcaagttggattttgtgttacgtgcctaaccaggagttgtcaccgcttgaggcttaacctggttctgggcggctcggtgtgtgtgtcccaaggccatgccacacagtccggctcctcagccactggtcagaaggtgcccccaaccctggtggcctcatccaggtttgtaggatgggccctgagtctcactggcccctgagcgtccaggacactggccagaagggtccgctccacccccacctcctgttgcctagcaacaagggcggtgggtcgcagtggccctcgggccacaaaactggctctgggagctggggggcggggagtctggctttaatgtcatttgggagcttcttccgggagctgggtcacctgcctcccggggcacagtggggccggagtgactaatgcaatcagtggtcacctgagccactgtgctgggtgggtgggagagggcacactgccctgcccctgcctcttgtgctgccctgctgggagccccccacgtgggaaatggtgggtgagggggatccaggggatgggcaaggcaagaggcacagtggggcttggtagcctcagccctaggggtgggctcaggatccctcaaaaacacgggggtgtggctagatgccatgggggctgagctgaggcctgagactcccctgctgcatcctgtgccggcctcgggggcctgggcagctgcacctctgctatagaggggtcaggaactcagcccacacagccccacccacaggtgaacttgccccagggacacgaaggccaccagctcacctcaaacaccttcacagctcgggacagcgccgagggcttggtggccctcggggagaagagcaggtttagcacggccttcccctccttctcctcaaaggccacagcctccagggggtccccgggctccgaggggactgcagcggccgctgctgccaccgccgcctcccgctccttgcgggcgtcctcgatgaggctctgcctgcgcccaatgaaccgcggggactgtggggacaaggggcacccatgcctcctccacctgctgagacccggggacctccacccacagctggtcccacagtcgggcagcgctgatggcacacagaggcaggggatgagagcacgtttttgagcgcctactgtgtgcctgctggggcagatgctagccgaggtgcctgcgggcattgcacgcccttcccgatgggggcagcccagtcagaagcagtggtggtgggggaggagtgggggctgggaaggtgctcgcctgctccccactctgtggcgagcagacagacagagacgctgtgtcactcatccccaggccaggcgctaactggattagtgatgggaagagggcgatgtcttgatggacagtggcatcggctgccggggagggaacggcagctcaggaaggagctgcccccacagggccccagtgaacaacccccctaccctcctggctgccgcacaggggaccctttctcacactcccatcaggacacctgtcctcgggccctgcccctctgtgccaccccgcaggcgcccgctcctggctgccttgtcatccctccaccctttgtcatagctctgggcgcaggtgtctctccctgggctcagcgcctctgacccctcgatcccagagccgtcccaggcctggacagagggggacttggcagacacctggggctcatcccttggagctgaactcccaagaaggctctgggccccttgtaagagaagaatcagcttcatcctgagcttccagggttgtggaacatgaaggggagcagcagaagcccctcccagagtcccctcttacttacccttggggtgggggtgtaggatgcagctggggctgcagttccaggccacggagagcctgtgaggctgggccccggggcgccctggggaggggatgcctgatggggagcctggtgggggagggtaggggagggcgggggaggacgggggagggcgccctgtgtccctgagaaggtacctggaaatgacactgctacaactcacaccacatttcaatcaaggtccataaataaaaacccattttaaatgtgccagggagcccaaggttctgagtgcccaaggaggcaccgaagacccctcctgtgggctgaaaagctcccgattatccagcctggcccacacagtcccctgtacacagggcttccgagtgcaggtcacagggaacacagactccatggtgaatgaatgaatgaatgaatgaatgaatgagggaaataagggaggaacaggccaatgggaatcaccccagagcccagataccctttgaattttgccccctatttgcccaggaccccccaccatgagctgctgctagagcctgggaagggccttggggctgcctccccaagcaggcaggctggttggggtgctgactagggcagctggggcagagggaggcaggggcaggtgggagtagggtgggggctgggtgcagcagccggggacctctggccatcttggattttttggatggatttgtttccacattccgatcgttaagattcaagatgaaacaagacacagagacccacacgacccccgagagaggtcggcctaagaggggcacacacagggacaggcatcacctcaccctcccccaacagggactcaaacaccaggcacaggggatgccgctgtgcccaggcctccacatccacgccgcgtcccaggggtttgcatggaccctgagcctggggctgccagccaggctggggagtagcagaggcagctggcaccagccctgggctccggtccactgcggccgccgggcacctacctgccctcttaccatgatggcctctgcctgcttggcgtccagctcagacacggccctgcggaagcccttggcctgtggcgtggtggcgtcgggggtgggcatggctcagtgtggaggtccgggctccgtc

SEQ ID NO: 44 - DNA, 3001 bp, synthetic construct
  source 1..3001: mol_type = other DNA
  misc_feature 1..3001: note = TUBB3
tccagaggtgtcgaaatgtcctggggacctgagcagcagccaccagggaagaggcagggagggagctgaggaccaggcttggttgtgagaatccctgagcccaggcggtagatgccaggaggtgtctggactggctgggccatgcctgggctgacctgtccagccagggagagggtgtgagggcagatctgggggtgcccagatggaaggaggcaggcatgggggacacccaaggccccctggcagcaccatgaactaagcaggacacctggaggggaagaactgtggggacctggaggcctccaacgactccttcctgcttcctggacaggactatggctgtgcagggatcccagagaagacttctgggctccctcaactccacccccacagccatcccccagctggggctggctgccaaccagacaggagcccggtgcctggaggtgtccatctctgacgggctcttcctcagcctggggctggtgagcttggtggagaacgcgctggtggtggccaccatcgccaagaaccggaacctgcactcacccatgtactgcttcatctgctgcctggccttgtcggacctgctggtgagcgggagcaacgtgctggagacggccgtcatcctcctgctggaggccggtgcactggtggcccgggctgcggtgctgcagcagctggacaatgtcattgacgtgatcacctgcagctccatgctgtccagcctctgcttcctgggcgccatcgccgtggaccgctacatctccatcttctacgcactgcgctaccacagcatcgtgaccctgccgcgggcgcggcgagccgttgcggccatctgggtggccagtgtcgtcttcagcacgctcttcatcgcctactacgaccacgtggccgtcctgctgtgcctcgtggtcttcttcctggctatgctggtgctcatggccgtgctgtacgtccacatgctggcccgggcctgccagcacgcccagggcatcgcccggctccacaagaggcagcgcccggtccaccagggctttggccttaaaggcgctgtcaccctcaccatcctgctgggcattttcttcctctgctggggccccttcttcctgcatctcacactcatcgtcctctgccccgagcaccccacgtgcggctgcatcttcaagaacttcaacctctttctcgccctcatcatctgcaatgccatcatcgaccccctcatctacgccttccacagccaggagctccgcaggacgctcaaggaggtgctgacatgctcctggtgagcgcggtgcacgcggctttaagtgtgctgggcagagggaggtggtgatattgtgtggtctggttcctgtgtgaccctgggcagttccttacctccctggtccccgtttgtcaaagaggatggactaaatgatctctgaaagtgttgaagcgcggacccttctgggtccagggaggggtccctgcaaaactccaggcaggacttctcaccagcagtcgtggggaacggaggaggacatggggaggttgtggggcctcaggctccgggcaccaggggccaacctcaggctcctaaagagacattttccgcccactcctgggacactccgtctgctccaatgactgagcagcatccaccccaccccatctttgctgccagctctcaggaccgtgccctcgtcagctgggatgtgaagtctctgggtggaagtgtgtgccaagagctactcccacagcagccccaggagaaggggctttgtgaccagaaagcttcatccacagccttgcagcggctcctgcaaaaggaggtgaaatccctgcctcaggccaagggaccaggtttgcaggagcccccctagtggtatggggctgagccctcctgagggccggttctaaggctcagactgggcactggggcctcagcctgctttcctgcagcagtcgcccaagcagacagccctggcaaatgcctgactcagtgaccagtgcctgtgagcatggggccaggaaagtctggtaataaatgtgactcagcatcacccaccttagccccttccagaaagtgcttgaagtttgcgggtggagggatgggggaggggaaggtgggcaggggtgagagtcgagagggaagaaaggagtcccggaaaacgtggctgcctccccaggtgaggaagccacagccccagaggccccaaatgcctggggagtgtggaggtcccaaccaggcttgcgctgaccctgcttctcggttttctctccgtgctgacaaaccccagcctagaggaaggacgagcaggtgcagcagggccccagtcccctccactcttgacgctgtcctagctgcagaagaggcgggttcccagccttccctgtgaccacatgtgacctcagccgggacacatccctttgctggccctggccctgagtccctccagccatgatgagccgtgaatgggaccatccctgtccactctgagatgcctggaagggggctcagtgcaggtgggctgggggctgggtctgctgtctgcccagcactgccattctgggagtaggcaggtggggaaggggtcgggggtggagggtctgtgttcagccagtcctgggaaatgcttgatgtgaggcttctgaagatggcagtgaggcagaggcccagccggcggagaagtccctgggagtgagtacctggggatgaactatgctgcctggtgctggggagcagtggcgcccctgggccatccctctgctgaaacctgggcgtctcgctgaagagaccacctccatttcctctgcagagactgagcactcagtcagcccccttcctgggacaggctcaatggaggctgcagggccatcagccgactcctacgcaggctcagtcagcagccccctggccagccccacccctgactgccggcctcagaactgggagctgcttcctggcagggcccgcctctgctgggagaccggacggtgagtcagccttaagcccggc

SEQ ID NO: 45 - DNA, 3001 bp, synthetic construct
  source 1..3001: mol_type = other DNA
  misc_feature 1..3001: note = FOXA2
gttctcgaacatgttgcccgagtcagggtgcagggtccagaaggagcccttgccgggcttgtcgggcgagcggggcaccttcaggaaacagtcgttgaaggagagcgagtggcggatggagttctgccagcgctgctggttctgccggtagaaggggaagaggtccatgatccactggtagatctcgctcagcgtcagcatcttgttggggctctgctggatggccatggtgatgagcgagatgtacgagtagggcggctttgcgtgcgtgtagctgcgcctgtaggtcttggggtcgcgggcgcggctcaggcccgcctgcccgtacatggggctcatggagttcatgttggcgtagggggccaggccgcccatggccccggccgcctgccccccgagcgggctcaggctgggactcaagtgcggccccatgcccgccacgccggccgccccggccgagccgcccatgcccgccatggcgcccgcgccgggggacatccccgccagggacgggctcatgccagcgcccacgtacgacgacatgttcatggagcccgcgctcatgttgcccgagccgctgcccatggcggccgccgacatgctcatgtacgtgttcatgccgttcatccccaggccggcgttcatgttgctcacggaggagtagccctgcggacagagccccgggagggaggcgacagcgttagcaccgcggctggagggtgcccagacctcccacccaccgcccaggcctccgcgtccggggaggcctccggggagccctttcgtccccgatggcccagtctccggactccgagtctgtttcattcagggagagtcatgatcgatcgcccttgggaaggtggcgagcggattgctgcttgggcatgtggcccgttcctagcccttctccacctcgcccagattctagaacaggggtcccatggagtactcccaagccagcaccccaaatacattcccctaccccagagtctccccaaactctcccaccagcatactgagctggcgccgaggcccgaagtccccggacgccacctccggtcgcagcaagcaacctgcgggacaacgcgcggggcgcctcgggctgcctccagcgaagactgtcccgggccccctctgtccggtctccctccagggaccccctcccttgtcccagggaaactgcaaggcagtgcctgtgcggagtgcgggggcggctggaggcactgaacggggcaatagggaaagaaccgagacctgaaccacacgcggctgagctttgcaggcaaaaactttctttctctttgtccactagtctttgactctgcctccacttcagcccccaactcctacccatcttctcctcctcagctccccaccccctcgccggccgacctcccacccctccccagccgcgcgctgccaaacataactctgttaggatagtgcgtggctcggccacgaagaggatttggaggcgccgcaagtcaatatttgatcacaaagttaatattatctcaaggctaacagtgtgtcgtataaaaaagagacccatttgattccaaggagggcggaaaaggcggctgcccagaaaggctggggttgtggggcggggtgggggggtgccagcgagggaagcggtcctgaggttggggaaggagcgagcgccgccgctccacttccccctggaaaagacgagcgcttacctcgggctctgcatagtagctgctccagtcggacggctcgtgcccttccatcttcaccgctcccagcatactggaagccgagtgcatggcagtttaaaatttaacagccacaacaaacgaccagcaatcaccccccacccccaccctcttttaaaaaaaagtcagccaaagcaccgtcccctcctccctccctctctcgcgctccctctccctgggctccactccctctctctccctgggcaggccggaggcggtagttggaagtgggcgggaggtgggggggggcgaggagcggaggaggcccaggccagcgccccgcggtagggagcacccgccgccgccgcgctcacgggctgccgggtggcggctgaggttggcagtgccgagctgccccgaggcggcgggaagcgcgcggcgcgggggctagtgggggggtggggaggaggaggaggaaggaggaggaggaggcggtggtggtggtggaggaggaggaggaggaggaggaggaggaggaggaggaggaggtgtggaccgcggagcggacaagtgccgcagtgacgtgggaggctggtgatatagcgcggcgcgctggcgcgggcctccaatccccagacccggggcagcccatttgaataatcagctcacacctaggtgagaggtagccgcagccggcgctccgcacctgcccctcagcgcctgccgtccgccccaccgccgcggcgccccgcactcctgggcgggccaggggagcgggctgggcgggcgatcgggcacgcgggatccctggtcgagccccctttcctcccgggtccacagcgagtcccctgaggaaggagggacctgggaggaaaccaccctctggggcggctccggcctccagcccccgccccgtctcatcgcgccgggcgcccggtgcgcctgtcggagggcgcgtccagcctcagtgcccggcagatccctatgaaagccggatttatttatgccgggtttcttcgctctcagtgctcattctctgtcaaaacagcaagtccatgacagccaatttgcaaagcgctgtcctatttagaaaaactacgtacacacctttaactcgcccgctgctgctcctgcgaggcccctccctgttacagttcagacccggaacggcctcgggagaagcgcggggcgcacggtctggccgcctcggctctccgactcctcagacaccggccgccagggacccgcagtggggcggccggcgcctggcgcaagcagcccctctagcagcggccgacggttgggaggctgagatttgtctc

SEQ ID NO: 46 - DNA, 3001 bp, synthetic construct
  source 1..3001: mol_type = other DNA
  misc_feature 1..3001: note = GIRK2
ttgattccttccagttctagaccgagaggaaactatatctccaccctcttcagtcctaggacccttagaaagaatttggctgggccggatacggtggctcacgcctgtaatcccagcactctgggaggctgaggcaggcggatcacaacgtcatgagttcgagaccagcctgaccaacatggtgaaaccccgtctctactaaaaatacaaaaattagccaggtgttgtggtgtgcgcctgtaatcccagctactcaggaggctgaggtaggagaatcgcttgaaaccgggacgcagaggttgcagtgagccgagatcgagccactgcactctagcctgggtgacacagcgagactccgtctaaaaaagaaagaaagaaataacaaacttggttggctggcctctcgctgaccagggtgcttccacagctcccttctgccatctatgcccaacaaaaagttaaagagaagagcgtgagagggcaagggatgcatgcttttgaaagccaaagggactcagctgaaactagcaaccctcgtcagaggcggatcggggtgtgtgtgtgtgtgtgtgtgtgtgtgtgtgtgtgtgtgtgtctgtgcgcgcgcgcgtcctttttctccccctgaggacagaatcctgtctccactctcagttcagctcaggtctttccctgtcccagtgttcttgattctgaataggtcctctgatgcaccgccatgcataaaatggatgatttgcaagcttctttgcctcacgtaattccgtgggtaatttgatatccagattctgcacgagccaaagactcatttatttatagaaagtgattgacctccagcgaccaaaggagtgggtactgcttgctgggggctgcagacaggtgaggcttcaccctcggcgcctgggctctggccttggtgctgaatccggaacccgctgaggaaccaccacctcggcggcccccaggcaactttcgggaggtttcaggggcacccggccagctctgggcgccctctcgagcgtcgccgtcagggagttgggcacaaccgatgccctgggacccgccacagggcacgcggctggcgtattcctttgcctggagccgggaaagttttgtccaatttttttcccctataactggagctgcggtctcccctcccacctgggtcgccatggagactcgagctctttttctttctccaggcaaaatgggcgaagctcagttagctcagccctgactgtagcaagcccttttttctccctaagcagttcctgggcatcacccttttccgtttctttttttcagcaaaagaaaggcaggcagggaacagggctagcaggatgtggcattgctccctgtaccgtagggctccccgggaccgacagactttggataacagcagttgggaaacactctaaacatataatacatctccaactcccaaacactggagttgtggggtttttttttttttctcttcctttttttgggggttggaggcggaattctttggaaaattctggtattttgtttctccactaacctcaccctctgtggctgtttctattaagatatattttccccctgacaatctctccatattgcacctagtacaacagatgtacctgtggatccactgggcttttattgggttttgggaatactgatgttatttcacttaaaaactaatccaagcttaatgttttgatgaaaatgaaaatatatccacagggaagcaaatatccacagccacccagtcctctaaatgccacacacatctcattttatctaaagtgttttttcttcccttccaattacaaaataattcagtttactcctaggaagatcttcctattcactgtttttagtcatgctagcacatcttcggttttaaggatagagaacagttaggagttacagtattctgtttcccattctccgcatgtttctccccaaccctcacttttcctccctttcgtgttgtctcctctcctctccatgcaaagcagaacaagaagactgccttccacaaagacaataggcttttcccaactgtaaatgactacatcacaagccgcacctgtcagttaatcagtgcatttaagtctttgccagcggatgttgaaatgatgtctgcaataactgactgggtttctgcgagcccagggatgcagacccctccttcccccgccccccgtgcgagtttcagtcgctaatgaaaaccgtatgaaacagattccccttctgttatgtaaccggcttctccctcgcctttcggctgacttgggcacaatgtttctcatgagagtggagatgcctgcggcgacggcggagctggggaaggggacgcggggacccgagagcacggaacgcctcgcccaggtggacgccagctcctcgcctgcgagcagctcagcgcaccgcttacctggctgcggacggggtggcttcactcaatcatgatctcctctcttgaaacggagcaagactgaacaattcactacacgacggatggcgagggaaggaaatgcaaaaaagaaaaggaaaagaaaaaaactggaatcagatggtcaaatttcacatcagcaccgagaggctgttaggagactccattctgctcgcggctgctccggctccaggtccggcttcccggcgtccgcgggtctccacccctccgcccgcccagcctctccagccaggtgcggccgtctccggactggctccctctggccgagcgcggagagcagggctgggcgctggggccaagggaggggcggaggggcgcgcgtttgggcagggacgggggagggagactgggtggaatgaagtggggaaagataagagtggcagaagaaaaaaaaaatccccggttaggagaaaagtggtggaccacgcagagagacgtcatgaaatccgcagattcaatgaatgctactcaattccagccgactggctgagccccgctggcagcgcacgaagcgacgcggctccgagataaaag

SEQ ID NO: 47 - DNA, 3001 bp, synthetic construct
  source 1..3001: mol_type = other DNA
  misc_feature 1..3001: note = DAT
ctttctggctctgtggctgggttctggagggtgcaggggacacagtcactttccaggggcctcctctgccactccaggctccagggcctccacatgctttgcaaaggctgccaaagtcagccgcaggctgttctttggacctttgagaattttctttccaaagcgaagatagcctctggaaacaggaggcagagccaagctgccccgtctgccgccccccaccccccagcttcttcgcgggcctccctttcctaaactctgaaatgcaggcgtgggacaaggcagctccgagtcctgctcaatggttttgtgacatcctctgggaggatctgcaccggccgtgagctctcacagggagctccgtcttcacgcatgggaacagcttcatctcgtttccgtacgtgccttggccccggctgcccctacgacccccgcccggccagcatgctcagggaggctgagatgggacttaccgccaccatttttgtagcacaggtaggggaaccgccagacgttggccaggtccacagcaaagccaatgacggacaggagaaagtcgatcttcttgccccaggtctcccgatcctgggcctccacggggctctgccgcgggttggtgagggtggagctggtgagctgcactccgttctgctccttgacaaggatgagctccacctccttcgggcccacggcattgggctccttagccggggccaccacggaagacatgagtcccacggagcatttgctcttactcatgggcacactgggagttgaggaattctgtgcttcttccctcttggtcttcagccaatatgaaaaataaacacacaacaggaacgcaacaattcagcagccagggctagggacatacctggtgcggagggcagagccccgaggcattcacgggcattcctctgggaagggatgggtgcgttctcagcgcctgagatggtacagctggggcacgggagctgggaagtgccagccctcaccacagggctggggcactggcccatggaggcctcaagacagacactctggtttgctcctccttcccctgcacccctccccactcctatcaaagtgcaaggctgtgtgagctctagcgtggctttctactaacaatgcatggatatcacttccaccttcccccacacttccagagagcaattttacaatcttctaagagcattcaataatgtcttcatgactgttcatctgtattcttttttcttttttttttcttttttaacatgaggtctcgctctgtcacccaggatggaatacagttgtgattacaactcactgcctcaacctcctaggttcaagtgatccttgcacctcagcctcctgagcagctgagactacaggtgtgcaccacagcaccggctaagttatttttgtgtgtgtttttttgttgttgttgttgtttttttgtagagacagggtctgactattgttgccaaggctggttttgaactcctggtctcaagcgatcctcctgcctcagcctcccaaagtgctgagatgacaggcatgagccaccaagcctgggcttgtattgtttatactcagtaataaagagcaaaatttatataagcaaaatcagccactgtaaagagagtttagacataaccatcacaactgcttcctcaaaaagtgtctgtacaagccacatccacctagggcaggtggcaccaccccgcgtgagagagctgggcggaggatggacagggcttcatcgcgggaactagctcctgggccagcagggtaacccgggaaatcctgtccaaacggctacataaacccccctgcaacgctgagcaggagagcaggagcgtctacaccgccccagccagggcgacttctgtcatttggatccagcagcagcgtgtttcagtcgagcgcacgcacacacagacacacagacacacacagactcacaaacacacatacacactcacacacacacaggcgagcacatacacttacacacatacacactcacactcacacaggtgcgcacacacacacaacgactgaagcaggtcgcaggtggaggctctaacaggcaactttccctgcatccaagtttgcacaaaacctcacccatgagctatcacgagctatgccacgcacccggtgacaacctcaaccggcacgacccctccggtgggtaaaacacccgacgaaggggttccgcggcgcgagcaggaggccgcgttgggagagggcgtcgggtgcggagctcgcgagtctccggcaagccgccgccgccctggggccacctgaccccgctcctgaacgcggggcctcagaggcgagttttgggtctacacagcaaaatgtggcccgttagaaagcgtccttcctcaccgcgcccagaacaagaagcggcgctgcctggcgagtcctcgcccggcctccccgccctgcccagagcaccagcgcgcagcccgcgcccgccaccacccccaacacagacaaagcccccgcgagatggagcggcggtgtacaaaacccactccgatcggatctgggatgcgccgcacccctagtagggttaagttccacgagaagaaaccaggacccccgagtcacagccatagacacccacgcgtcccctaagccggtgactgcaccaggtgcccacccggttctgagcctggagcggggccaggggcagccctgcaccctcctcgcgcctcggacggcctcagggtcccatgtggcgctatcggggggcgctcggcacccgcgactgttggcgactttggagacggcgcggtctgcgggacgcggggaccccagactgtgcccggtccccggcccccgcccctgcgccccacttcggggtctgcggaaggagcggggcgccccgatgccgcgagcgacgctggcctcacctggccgcccgggcctgggctttgcacgcgagtcctggcgccgagagcgttccgcgaagcctcccctcccgctccgcagcgctgggcggtct

SEQ ID NO: 48 - DNA, 3001 bp, synthetic construct
  source 1..3001: mol_type = other DNA
  misc_feature 1..3001: note = INS
agcctcccagccggcccaccgggccccgctttggcccagccccgcgcgagctcacatacctcagctccagcagggcgcccctgtctctacccgtgcgagtggggccggctgggtgttcccatggacggctgctgccgatcccagccaccgcctccctcctcttccggggccagtgtccggccgtccacccgcctgcccagccttggcccccactctggaggcggggctagctgggggctgtgacatctctgggtggaggggggctgatgtggtcatggggaaggggtgagctcagagcttcaggaacagggctgccccagtggccaggtccccagctggagggtgagctcctggcctcgaggcccaagctggcagtggtctgcagccacagcccatgccagccccaggcccagccatccccagcccagccctgggtggagctctcaggaaaggtgcagattggtttggcctgggtggcaggcagactggcccagccacagggtagctagggtcacccgctgacctcccaggccacagtaggcccaggcagcccctaggttctcgtctgcccactaggctgggtgcccagcggagccaccctgtcctcaggggtgctgcagaagaggctctgtgtcaggacccccaaggacctggagccacaacgaccttgtcaccagggcccgtgggcccttctgggtgagcagcaggtctgtagcaaaggaagccagccaagcagccctgcttactgggagggggtgggctaaggggctgcagggggaccctggccctggtctctgcccctgtggccctggcggttccctgcttctcctgggctgcaatcctcagggcctcatggccggggtgcccaaggcatgggttctgaggccctgccaggtgctgccacggtccctgcacctgccccagctcatggtgccatctgacctctcctgcttcctccaaggcaccagtgggtccctcaggtgtctggctggacctgggctggcataagctgtggctgcagacggctgccagcttgggccgcgaggccagggtgtgtgactgtcccggggctgcccagagctggggatagcgggtggctctcggcagcctctccccaccttcccagccccccgccctgcaggaccccctccctcagcccagcctcctccctccacagggactccatcagaaataactctaaaaatagaacctgggagggctaggtggggggaaaattgctggaatgttctcattccctttcctgaacaaggtctctggggactccaagagtccagagctactgaacaagaagtcacttctcagtggccccaccacccctggcccctcagagacccccgcagctcccatactggaccctgagccacagggtgggggcagcaggcagccgacaggcatggccgctttggggagagccactgcatgctgggcctggccggcgttggcacctgtgggcacccagagagcgtggagagagctgggaggggctcacaacagtgccgggaagtggggcttggcccagggcccccaagacacacagacggcacagcagggctggttcaagggctttattccatctctctcggtgcaggaggcggcgggtgtggggctgcctgcgggctgcgtctagttgcagtagttctccagctggtagagggagcagatgctggtacagcattgttccacaatgccacgcttctgcagggacccctccagggccaagggctgcaggctgcctgcaccagggcccccgcccagctccacctgccccactgccaggacgtgccgcgcagagcaggttccggaacagcggcgaggcagagggacacaggaggacacagtcagggagacacagtgcccgcctgcccgccagccctaggtcgcactcccacccatctccagccgggctggacccaggttagagggagggtcacccacactgggtgtggacctacaggccccaacgcccacatgtcccacctccttcccccgccccggggcagcgtcacagtgggagcctgaacaggtgatcccagtacttctccccagggcctgtccccagcatcttccccatctcctgactatggagctgccgtgaggcctggcgacaggggtctggcccactcaggcaggcagccacgccctcctccgggcgtgatggggtgttcgcccagaggcaggcagcgtggggcaccctgtgaccccaggtcacccaggactttacttaacaaaacacttgaatctgcggtcatcaaatgagggtggagaaatgggctgcggggcatttgtttgaggggcgagtggagggaggagcgtgcccaccctctgatgtatctcggggctgccgaagccaacaccgtcctcaggctgagattctgactgggccacagggagctggtcacttttaggacgtgaccaagagaacttctttttaaaaaagtgcacctgaccccctgctgggtggcagcctcctgcccccttctgcccatgctgggtgggagcgccaggagcagggggtggctgggggcggccaggggcagcaatgggcagttggctcaccctgcaggtcctctgcctcccggcgggtcttgggtgtgtagaagaagcctcgttccccgcacactaggtagagagcttccaccaggtgtgagccgcacaggtgttggttcacaaaggctgcggctgggtcaggtccccagagggccagcagcgccagcaggggcaggaggcgcatccacagggccatggcagaaggacagtgatctgggagacaggcagggctgaggcaggctgaaggccaggtgccctgccttggggcccctgggctcacccccacatgcttcacgagcccagccacgtcctccctgctgcagagctggggcctggggtccagccaccctggaatcctgagcccacctgacgcaaaggcccttggaacagacctgcttgatggcctcttctgatgcagcctgtcctggagggc

SEQ ID NO: 49 - DNA, 3001 bp, synthetic construct
  source 1..3001: mol_type = other DNA
  misc_feature 1..3001: note = Leu7
cctgtccccactgcacttcttccatgtcccctggctagtcttcagtagggtccccaaaggtctttttgcctgggggcctggggagggggtgcatgacttctcttcttctcctgagtgccaggtttccccagaccccacatccttccccactgccctgcattccaggaccaggagggctggggcagggccagaagatggagaggagccctcagcattgtgagatgctcaggggtgaagggtgtccctgaaagtccctgcaagctcttgatgtgcttgacctcatagcctggcctccctctgcctttgggcttctgtctaaaagaccctcacattcctgagagccacaggaaagcaccttcctcagtagaatgtccaactgctctagtgaggggcctcctgcaggggtggacaattgctgaggtcgatgtaggggaacagggggaggcccccagcctgccccaactcaccactcagtcagggaggccctgacttctgcctgtgtgcctccctctgctgtccgccagtgcgcctccgtgcctttgtctcttgtctcactgcctctttctctgcctcagtcaggctctctctcggattcctcctcctcctgagcccgttcctctcctcctctccattctcatcagagaggaacactcatccctctcttccagaaacaatggagcaccatgctcctgtccgctcagcctttcggttggtgcatcctcttccccatctcacactcccagctcctggccggcctcatcaccctgtgctccacacctaggaaaagtatatctgacttggtccccttatccccgagaaaccactaagagaaccttcaggtctccccagaagccgaagctgaggaactgggcttgccccagggcatcctcctgtgaagctccttggtctccgccacgggctccaatcctgttgctggagcttatctcctcagtgatttacctgccccctgcttctggtggaacagtgagtaactgcccaaccagaaacctacacacacatattcaacacagcgcctgcaagcttccagctcacctggacctggagtgatccccaagcatcctggggtcaggcacagaaacaggaacagcgagctcctggctccagtgctgaatggggcacagatgcattaactctcccagtcccattaaccttagcaagtatttctgcttccatgaaaaggggaagagggaaaatgtctctaagctctggaacctttggcagtgctgggcatggactgggcagagtgagaagtctgtctgaagaccctgggaaagggtcttcaccccatgggaagaaagggaaggcttgatgtttggtccagtaaggccattaatggtcaccaactgagcagactctgggaggaaatgtctcagtctctgcagtggctaccagcagaggtagggcgacataagcacagaagacaaatcttagaaaaccctagaacaacagtcctgtgcagccacatgccccacagccctagtaacttagggagtgatgtgcctcccatttccacacaaacacatccaggcattgcacagtgctaggccctacgacatccgtaccccaacattttcaacagtgcgtgctacacagcccaagctctaggccccattgggcaccccacctatgcaggtgcccatgcactgggctagcaactgtcagtaaaggtccccttcctgtatgtccagagtccttcagctccagcttctacaagaatccatgcagctaggcctggctgggggaggaaggggacagggaagttggccaaggctcccatgtgtgtgcatgtgggggtggttcttgaccttagctgcttggtacggggctgtcacctccttaccctggcactgtcggacctggtggcttccttctctatttcctgcctcaactctcagcctcctttttctggaagttgtgtgctctgactttggagggattgggacctcagacctgagcccaggtgcagatgggagggtggtgaactggtcgcgcctccttcctccttgctacaccgaaggtcccaagggccctttgccacccttgctacccctgggtccctcagcgcgccccgcagagctcgggccccctcaagagagttaacttggcagtgcccaggagccagagagcgatccagagagcgctgttgggcagcaggggtggggaggtgggggacgactgacccctgactcctcatccggccaccagaaaccacctgctgagggggccgggaactgacgactgagagcccgggccgattgttagaagctgctgagaaccagctcttcccctaatcccggtcgacgagggcaggctgtgccgggttttgtttcgcagcccctcggcagcagcgcctcccagcccgagctcggttctcggccccctttccagctgcccccagcgcgcgcagcgcacacacacacacacacacacacacacacacacaccccagcgcgcgcccgggaggacatggcgtgggcaccgatggggacgcagaaaggggctctgtgcgcggcggggcgcccgcgggggcgcggggagcactgacctgcggcgacgagtccggagcgtctccggtgcggtccatcgcgggcaacggcccctacgccccggccccggcgcccctagaccgctgccctgggccgcgtgcggctccctccccggggactgtcgggctccgggcccccgggggccacttcatagccgcggggtccgcgcgcccgcccgccccgcccggccccgccgccccggcccggctcgttctgggctcagcgaggcggcggccgaaggctgggagcgcgcgggagggagggcgcgggcagggaggcggggggcggggggcggggagggggagcggggagggggagcggggagcgggcgcgggggcgagaggggcgaggggggcgcgcggccggagccgagccgaccgggccggcgcagagtccccgaggtggcggcggatgcgccggtgccgccgcggctc

SEQ ID NO: 50 - DNA, 3001 bp, synthetic construct
  source 1..3001: mol_type = other DNA
  misc_feature 1..3001: note = CXCR4 (CD62L)
gctgatcccaatgtagtaaggcagccaacaggcgaagaaagccaggatgaggatgactgtggtcttgagggccttgcgcttctggtggcccttggagtgtgacagcttggagatgataatgcaatagcaggacaggatgacaataccaggcaggataaggccaaccatgatgtgctgaaactggaacacaaccacccacaagtcattggggtagaagcggtcacagatatatctgtcatctgcctcactgacgttggcaaagatgaagtcgggaatagtcagcaggagggcagggatccagacgccaacatagaccaccttttcagccaacagcttccttggcctctgactgttggtggcgtggacgatggccaggtagcggtccagactgatgaaggccaggatgaggacactgctgtagaggttgactgtgtagatgacatggactgccttgcataggaagttcccaaagtaccagtttgccacggcatcaactgcccagaagggaagcgtgatgacaaagaggaggtcggccactgacaggtgcagcctgtacttgtccgtcatgcttctcagtttcttctggtaacccatgaccaggatgaccaatccattgcccacaatgccagttaagaagatgatggagtagatggtgggcaggaagattttattgaaattagcattttcttcacggaaacagggttccttcatggagtcatagtcccctgagcccatttcctcggtgtagttatctgaagtgtatatctgcaaaagaggcaaaggaatggacattcacttccaattcagcaagcattaacccagttaaaaaaaatttttaaagcaatttaaaaaaccaattcaggcttgctttcttcaggaaattctgaagtagtgggctaagggcacaagagaattaatgtagaatcctacaactctcctccccatcttttcccatagtgacttcattatatccttctttggtagaaccaattacaaaattctttgtttagaacaaaagggcactgagacgctgagggtttcaaagtcacatcttggctaactcctctgccccgcccactagagggaagaaaaaaaaccttccttaggaggaaaaaaaaaatacacacaaagaggccactcccaggcggcgtggggggtggggtgggtgctgctaggaggggcagtgattaactttttgtaagaagtttttcgcccagggagggaagaggggagaagggaggatcacggggggagggcgggggcgttgcaaagactcattctcctaaagcgcaaaaacttaattttcccacgcctgcctaaatacaaaccattctgggcttcaagcaacttgtagtgggtaaagagaatgcggtcttaaaacgaaggcccttcggtgcttggggtatattgggcgggagtgtcagaaaatgaacaaacggcacctcctcccccaagcgggcgctcctccggtgtgtgggtctcttgccatcctcgtgtttatcacttggcgcgtttgggacgttagggagcggggcattttcctgggtggagaaggtaacggggtctgcacccgtggtcctcgccccaagtttcatttcctcactctcccgggtggcttcccattaccccgccactgatccagttaacccggccggaggtgggcagctggaagcctccaggcggtgggcacgcggggggccgggtcgtccagccccgggccgccgcggctgcccactacacccacgccaaccgcccgcaagcagcgctgcaggggctccgctgggcgacacgccaggctctgtcccacagggtgctggggagcgactgggcggctccgccgcgagcgtctttgaattgcgcgccgctgcaggaaaccaaaaactccctagcaagagggtttcaaaaggtttctggaaaccaccgacggttaaacatcacaactggactcggagagagccaaacggtttccccacttgcacctgccagtcttcgcggcggcgacctggcagcccaggtgcggtcttaaccgcccccgcccctcaccccgtacccgctcctatccccggagcgcaaatctcagggctggcagctgcgcggtgtcaaaggggaggtcaaaccactccgctgacctctgcacgaccccaaactctcgaactgcaggacccactcgcggccgtggggaagaggcgcgcttcggacggcgggaaggttttccccctcaaacccaaagcgcgcgggcggatcaactcctagctgctgccaccactcgatcccctcagaggatcggcgcggtgggtccacccgcctctcccgccctctgcctactgtgctgggagactggcacagctccgtcggccgcacagagtttaacaaacacgcacccagtgtcaagaacagtcaccaggcgcttaaccccgaagttaaagcgggcgcaatctcctcctgggaactcagcccaggcacgccgccctccgcctctaaattcagacaatgtaactcgctccaagacatccccgcttccccaaggaagagaccggtggtctgagtcccgaggcagcgcgcacgccttctctgcacttgtgcacagaatgttcttacgtttgcaaacagcgtgcaagccgccgcgcgcggcgggactcaagggggagacacatgcagccactggaacgctctttccagtcgtttctcctcgactcacagagaaaaagattccaatcctgctccccccccacccacccgcactatataggcatggtcaagaaaactcctttcggtgacccttttttggagtacgggtacctccaatgtcctggccgcttctgcccgctcggagaggggctgcgctctaagttcaaacgtttgtacatttatgacaaagcaggttgaaactggacttacactgatcccctccatggtaaccgctggttctccagatgcggtggctactggagcactcaggccctcggcgtcactttgctacctgctgccgcagccaacaaac

SEQ ID NO: 51 - DNA, 3001 bp, synthetic construct; identical to SEQ ID NO: 6
  source 1..3001: mol_type = other DNA
  misc_feature 1..3001: note = interferon-gamma (IFNg)

SEQ ID NO: 52 - DNA, 3001 bp, synthetic construct
  source 1..3001: mol_type = other DNA
  misc_feature 1..3001: note = neural cell adhesion molecule (CD56)
tagcagaggctgctgaacctcggtcaccgagaggaaggtggatgacccgggcgtgggggtgattacccagaccaaggcgcgtcccacggggccctgctacttcgcttttaaacaagcctcagggaggaaaccgggagcccgggccaagggaggaaaatggccactttctgctaaacagtctcaaggctagatagtattccaaaaaaagcgctggtccggaggtgcaaagggtagggcacatctgcggaggaaacagtcaccttaattaccacgtctagagaaagtgcgatttcattacagcctccaaaactaccgcccaaagaaggaaaccagagaagtgttttcaatttgaaagaggctggaaagaagcgggtaagacaccgactctccccaccctcccggcaactttggaccacaaggcgctttcctgcctcaaagactattaccaaacacaactcgaaaatccaaccccgctttcaaacctgtagccgttatttaaacggtaaaacaccccactttagtttccgggggatgcggcccaaatcagtccttaaaaacacacagagagcgcctctggcagcagccgcccacgcccccggccctgcgcaccgggcttgctccgggaggggcgcgtgacgccaccgcggggcagccgagaggggaccgcgggctccgggcagggcaggcgctcccgcagctccagctataacacaagggagagaaagcaacgctttggttccagaaacttccatcgtcaaggccagttctggccagacgcacagccaaagagcccccctagtctagcggatgaaccgttcgcgagtggcaccccctaaaaccttggccccatctcccaagatgcctctttactgcgcaatccagaaagcacccggaccgccgccacccaccagcggcggcgaataacttggtgagatcttaaataactcgggatccaagggaactttcttttctcccccgaatctcaacgccgcatctccaagtctccagaaatgttaaggagggaggtggtcaagacaggctgaagaatctgagttttaatttgggggagggggaggggaggaagagggaagttaataatgcggctgcggtgtacccgtgtggggtgaaacggaaccagatgcggctgctacatctggaaattgggggtgggggagtgctgggggagaaaggcaccgtggtaatgtttttcaaaagggttgatagcttctgggctcagaacaagaaacaggcttcacaccccaccccccccccaccaccaccaaaaaagtgtctggctgcctcatagggggtgtgtgttctatatgaacccccatctacaaggtccataaaattcctccgcaaacatgggtcgtagcctcacttttctcccaaccccaaacataacggtaggggaagcgaggtgcagaggggtctgggccagagaagcatacgcatcgctgccgagtgaaccacaccggacaaaaaccctcatcccttcccccacctctaggaaccagattgggaccgaacggtccttccgcccccatcccacacctcggcttaacgaccgtgggttgtcctcccgttccccaaacttccgaccctttcagagaccgtccaagtcataagatcaccccttctggaaaggggagtcgtttttcgctctcagctcaagcagattcagccatttggtgacgacacctccttaaaacctattccaagagaagctaacctagccctccacgtcctccgcagttcatctcccaccccatccgaccccaacccgcaccccaggctttcccgggggtggaaaaggacttaggaatgccccggatttttagcgttgagcccaaagctacctgctgaattgggggtgaatccccaaaaggggaccaaaaagacatggagtggaatgcaacacacacacataacacacacaacgcatcccacctcgccgcccactcccgccctcctccccagtccaggaaaccgcgggcctcgccgccttcgccccgggcctgtccctcccgggactcactgaggagccgccgccgcctcgccagctcccgagcgcgagttggaggaaaagttgggcggcgggaagagcggcgggacgagcgcagggagggggcgcaggagacgcggacggagggaagggaggggagacccaaggggcgcggatcgcccgggagggagccaggaggtgaaaggggcgggcggcgagcggagggaggcgccggccccggctgggctgcggcgggcaaagcgcgcagccgggaggctccggcgccgggggccgctcgggacgccaagcccgccccagatccgggggcgccgcggctcttacctcgctcggcgtggaggttccgcctcgggagagtccgccgtggcttgtgcgagcgggcgtgtgcccgcgtccccggctccggcccgccgcccccgggctctgttcgggtcctggcggggtcgcaagagctccgcggccggcgctcgactcccggcggcgctcggtgctcggcgcctccagcccgggcgggaacaatggagccggagctggtgccccggaggcggggcggggggagggctcccgtccgccacccggggtctccagaccttgtgcccccctcctccagaacgcagcggcggcgcctcactttccttgcagaccgggctccatcgccctgcggaggccccggcgccgcggcgtgcccgactgcagcacgggtaccgtgcgccctgcggggcgccccgagctcggaccggagaaaagtccttgggttccgcggcttttcaagcagcggcgcgctcgcggccggggaaggcgaggtcgccgcaatgcgctacgaggggtctcggtcccgtccggacgtggccgcccagctcccggcacacccgggttcctccgcgcgctgcggctgcaccggcgtcccggctcccgctagctgccgcccgccgccgaggacgccgcgcctgtggccgcaagagcgctccgagcgctatg

SEQ ID NO: 53 - DNA, 2075 bp, synthetic construct
  source 1..2075: mol_type = other DNA
  misc_feature 1..2075: note = DAP10
tcttggccctacctccaaaatatttccagatctccctagcctgcacacccttgccacctgtcattcccacttggaccaggccagcagcctccctggtctctctgaccctccccctgagttcgttcaccaaaggcagtaacggagacaccccctcaacacacacaggaagcagatggccttgacaccagcagggtgacatccgctattgctacttctctgctcccccacagttcctctggacttctctggaccacagtcctctgccagacccctgccagaccccagtccaccatgatccatctgggtcacatcctcttcctgcttttgctcccaggtgaagccagtggttacaggggatggtaggcagagcgtttgtgagatgggtgcttgggtgacgtctgcagggacgggtgatgaaagtggggttcttctccctgcaccccttcccttctgggagatccattctgcttcagggcctgggtccttgggggcggaagggggtgagacagggagttctggaggggctgcctgttagcgtccccttctcatggctgggtctctgctgccacttccaatttcttgtcactctccatgtctctgggagtccccttcccatgtggtcctgttccatctctccagcctggagattacttctcaggacactacctttccttctctacaccctattttttggtttgtttattttgagatggggtcttgctctgttgtccaggctggagtgcagtggcacaatcacggctcacggcagccttgacttcctgggctcaggtgatcctcccagctcagcctcccgagtaactgggattacaggtgtgaaccaacacttccagctaatttttgtatttcttgtagagacgaggtctcactatgttgcccaggctggtctcgaactcctgggctcaagcgatcttcctgcctcggcctcccaaagtgctgggatgacaggcgtgagccacggtgccaggctgagcattctgttttgtggaccttctctccaccctcatccaccttctttctctttccacagtggctgcagctcagacgactccaggagagagatcatcactccctgccttttaccctggcacttcaggtatcacttccaccccagaagcttggccagaggctcccagaacaccccagtggttctccaggtcaccatcccacctcccgtccccaaatcagaggatccgtgtccttctccgagtcccagaatcagcgacccccagcctgtgttcaggagcaccccgtgtgcccgccgcacagccccgagggtcctgggacaccccagcctctctgcatctgtctcccgtttcattccccaagcgcaactccaaggaacctgggacccgccccctcgcaggggacttcctctctgcctgtggccaaagcacagccccaggacgcagagcttgagttgtctccctgttccggcccccactctccaggctcttgttccggatgtgggtccctctctctgccgctcctggcaggcctcgtggctgctgatgcggtggcatcgctgctcatcgtgggggcggtgttcctgtgcgcacgcccacgccgcagccccgcccaaggtgagggcggagatgggcggggcctggaaggtgtatagtgtccctagggagggggtcccagggagggggcccttggggaagccctggaggaggtgctggggaaaccctgggggaggtgcctgggggaacccctgaggaaacccctgaagcagggggtccccagggaagtggagatatgggtggtcaagcttcatgctttctctcccctatccccagaagatggcaaagtctacatcaacatgccaggcaggggctgaccctcctgcagcttggacctttgacttctgaccctctcatcctggatggtgtgtggtggcacaggaacccccgccccaacttttggattgtaataaaacaattgaaacacctgtagtcgtattctttctcaaagaaccccagagttcccaaagcctccctcccatgaactgtttctggatccaaggccccctcagaacccccacatgtccccatccc

SEQ ID NO: 54 - DNA, 1461 bp, synthetic construct
  source 1..1461: mol_type = other DNA
  misc_feature 1..1461: note = HLA-C
gaacagaaacaaaaagatatgagacaaattgaaaacgtatagcaaaatggtagaccaaaacccaaccattataagtgaagaaatgacacgacctgagtcacattagcaggactgctgagcactgtggggagaacagacatgggcaggaggtgagggacagtgttagtgccacaattcaggagtgacagggtggcggggactaaaggggaaagagggtgtgagggatgagaggggcagagagaagggctggagaagcaggaggtgaggaaaaggagcagaggaaagaattctaaagcagtagaagagcctggcagggggttctttgcattcggtatttaatacattttgtgtgactgccttaaaactaatgggctccttatgatttttttttaaaaaggggttacaaaaatatcaagtgtccaaataaaatatgcacactgcttagatgtgcatagttcacgaaaacgggcagtgctggagcgctggtgaagagcattgggactgcatggagccctcgcaactttgaggtgatgactacaggctcccggttgcaatagacagtaacaaaccctgcttctttgtattcaggagatgttctggactcacacagggaaactctggctagagaatgaggataactttaaatgcaacaacccagagtcacagaaccatagtctgcgaaagtaaaacaggagctttgagaatttaattgtaatgcagttttgacacaggtctttcacagattggaattctaatcattcagggattaccaatattgtgctacctactgtatcaataaacaaaaaggaaactggtctctatgagaatctctacctggtgctttcagacaaaacttcaccaggtttaaagagaaaactcctgactctacacgtccattcccagggcgagctcactgtctggcatcaagttccccatggtgagtttccctgtacaagagtccaaggggagaggtaagtttcctttattttgctggatgtagtttaatattacctgaggtgaggtaaggtaaggcaaagggtgggaggcagggagtccagttcagggacggggattccaggaggagaagtgaaggggaaggggctgggcgcagccttggggtctctccctggtttccacagacagatccttgtccaggactcaggcacacagtgtgacaaagatgcttggtgtaggagaagagggatcaggacgaagtcccaggtcccgggcggggctctcagggtctcaggctccaagggccgtgtctgcattggggaggcgccgcgttggggattctccactcccctgagtttcacttctcccaacctgcgtcgggtccttcttcctgaatactcatgacgcgtccccaattcccactcccattgggtgtcgggttctagagaagccaatcagcgtctccgcagtcccggttctaaagtccccagtcacccacccggactc

SEQ ID NO: 55 - DNA, 1085 bp, synthetic construct
  source 1..1085: mol_type = other DNA
  misc_feature 1..1085: note = NCR1 (NKp46) - Synthetic
ttggtagaatgatttattttcctttggctatatacccagcgatgggattgctgggctgaatggtaactctgtttgtagttctctgaaatatctccaaaccaaactgctttccacagtggctgaactaatttacacccaccaacagtgtataagtgtcccctttgctccacaatctcaccagcatctgttaatttctggcttttcagtaatggccattctgactggtgtgagatggtattgttgagggataatttaggaatcagagagaccgaggggttgaggaggatttattattattattattatttaggtgcaccggccccagtcagattaacatccaaaaagactgaggctcgaacagagagtccggttaccttttaagcattttgtggggttgggggagatctgtgcagggggaagcatattacagaagcaagaaacaaaggcagttattcaattgagacatgcatcacattattccttacttttcaagaaaaatatgttttacgacttgaggttatcctgtctagtgaccttgcagccgcacggcaagagaaacagggtcttcacaatgcctgggaaagggagagataaggctcactagccacagacagaaaaacaggcagttcatgtttaaaggactccacctctttctcttcctcggggggaactgggttttcttaaatacaactgagtttttgtttacacattctgtaatttcttttaattcctgttccagtatctcactgtgaaactccctatgtttttatacgattctcagggggtttcctctgggcatgattgggcacaacttcccacagtcagctctgggtacgacctccacattgcagaattgagaagttgacccagaaatgcattttgggctgagcagacaattgtcagagttgctggctagaccacagatgtgtcagagggaccacggcctttctgtaagctcatggtcagaggcggaggggagttgtgaacgttctgatgaaagcagtcaacgtgaaagcgctctggtgatgggcgctggtgctcacccaccacttcctgtgtatctatctccctggcccgcccggctc

SEQ ID NO: 56 - DNA, 3001 bp, synthetic construct
  source 1..3001: mol_type = other DNA
  misc_feature 1..3001: note = CD34
taggactctgcagctgagtttatctgctgcagtttggcacttcttgccctaagctttttgagacaaagattgcgaaggaaggagatgaggacaagggaatgaggaggcccaggaggtggagccaaagggataagaaaaagaggccccaggcccctgtgtgttttatcagccgaacagaaatgctgctgaagacatgcatctgcaaaggtggtgccaactgggttgggtctacaagtgcggagcactggggaccactgaattagtcagaggtccccaagcctgctcctcctctgtcgccatatggcagagattcaaagggtggagtcctctcccctctggaaaatagctgataaaattcttttcccttttatttttcccctcttacatgtagccctggacaggatatgggtgtggggtcagcaggcacgaaggacccaaaggaagaagaggaatgtgacggggagggcctgggggtgttgttagagtgaggatagggacaggccttggaaagcttggaggaaatagataccctgagcctgacaaaggggttccctaattacctgaaaggcttcctgctccagccagcactctggcagtgctgcctggtggcccttgagccccttaccctgactctgctctgtgatctcccccacttccaacaaagacagcccacttctggttagttgcagtgcctatgcacatgccaccttctcaaagggacagaaaaaaagaacttcccaaaacaagctctttgttggggtaaatgatgagaaaacaaaccacagaaatcccctctaccctaaattttacaataataatagcatttctgcaatggccctatggtagcatttcaaggtacactcagttatctcatttatcccacaatgtatcccaggactgaaaatgactcctagtattaacaaaacaaaacaaatccaagcaaaaaccctagaaagctcttgggaagggcataaaactacaattggctcaagtagtctggcaaggctgggaatggggccttgtgcctcatccctccccaccaaaagctgggatattctcagcatatcagtccaaagttttgccaaggttgtgttgctcccccaaccccaacctcggcaggccaaggcgagactggcacttaccagctgaggaaataagaaaagggtggacctcattcggctgaggaccaggtcatcacaagctgtgtagccaaaggagaatggagcatctgattggaatctgcaccactggccaactcacccctgtttttttgttttttgttttttgtttgtttgtttgttttgagaaggagtctcgctctgtctcccaggctggagtgcagtgccaggatcttggctcgctgcaacctccgcctcctgggttcaagggattctcctgcctcagcctcccgagtagctgggactataggggtgtgccaccatgcccagctaatttttgtaattttagtagagatggggtttcaccatgttggccaggatggtctcgatctcttgacctcatgatctgcctgcctcggcctcccaaagtgctgggattgcaagcgtgagccactgcgcccggccgactcactcctgttatgagggcctagtctagcacccactctagaattttgaaacctcattgatagcttttccttgccccaaagcacacatccaggactgccaaacagggcaacccaaaacttctctcagagcttcctccagcccaggggcagctgtccccagctgggcggacccaaagatggacaacagaggagactgggaacaaagaggtctagtccaggtcactctgcagggccacagtgggtggcccttaggatgtagtgagagtacagaattgggagccctagagtgctgtaagcaaaagggagagcaatgtacaaagtttcaaaagtttcaaaccttccaagtgcaaagagatgaggtggagaatgtcattccccaaggaaagtaatcgagtataaatatttaccgggtgcctattttttttttaaccgaagtgagagcaagaccttaagagtaaggatatgtgtaggtgagtgggggcagtgttctaacccatccactagctttagaacttggagcaggggcagaggaagaaaactccaaaaggtgacaagttttgaaatttgtgcaggggtgacggtttttggtaaggcgagcgaggctgggcgcagagcaaattctgtctacttctacctcctccgcggtggggttgctgaagtgggaattcaaactcagcttcccaatcttaaatgcaaggtttttgtttggttggttgtttggttttgtttttccttttcacggactgttcattccgaacacccctggcctgggacctggagcctcatccctgattcgcttccctcgaattccttaatgtccacttctggctctcccagtccctgtgcaaggtgggcacaccggccccttcccagcaatccgctccgtgtctatactttctgaatcaaccaagagccacaaaacttttcctgcttctctcctccccagtctcccggttctggctgcaacttcgcactccgcgcctctggcaaccagaagagaccctggcatctccaaagcgctttatctcagtccattggaaccagtcgtgctgaccgattcacacctcggctaacgcacactcgcgggggtgatggccttcccttcccctcccacccccgtcagctcccaggtgaggcgctggccccggggggaagcagctgtgggcggcagtgcgtgggttgggcaggggtcccttccctccgtgagactctgctctgctgttcagcctcccagaaagcctccaccctccccgcggcgaagccaagcggccgcggcgcgcgggcggtactcacgcagcaaactcagcaagcaaagcgcggtccagccccgcggcatcctgggccctgcgcgcgcgcccctgcggaccagcatccttcccgcgcggctcctagagagacgcaccgagtggaagacac

SEQ ID NO: 57 - DNA, 3001 bp, synthetic construct
  source 1..3001: mol_type = other DNA
  misc_feature 1..3001: note = CD45
tattcttggaacctgcaaggcctggcaagatttggccatcatctccctctgaaacctccttattttccaggctctgcttctttctacatcagcctcacatctcttcctgttccttgaacacagtctcctcctttaaatttattatttctgcctagaatactttccttctcctcatactcatttaacttattttcttcaggtctagctccattgctttttattccaggaaaacttcccaacctccagaatagaccaaatatcctattatatgcttaaatcacagtgtggaagtattgcctttgtgatgaaagtgtcatgatgactggataattaattattgtgtcttccactaaagtgtagataggaaagaaagcatgcctgtttttgctcaccattttttcctcatgtcaggtaaaatgactggattacattaggaacttgctctgtatttgttgaatgaatgagtgaatgaatgcatgaacaaatgagtaacatctgcctctgtagttaacttgtaatctgtttctgtctttatataacctctactgtaggtagccttaaaaatctggaaaatgactttattgatttaagtaaatattaagtttttaagcttttaaaaatctattgtaagttgattaactatgattattttgaaattttcttcatcaaaatttgagatgactttggatacatactgttttcagctactcaacaagtgccattgtatttactatgtatgaaatctggtgttacttcctgcccatttcctagactcagttgttctttgcaaaatacccatttatcagcaaccagcccaccctccctctgggaatcagtgagagtggatcacatcagtatgtcataaatactcctcctgttttctttgagagttttctgctgatgcaagcggaaggcttggatgccaggcctgtatgtgatacagtagaaatttccccagatgccctgtggaaagacaagctgctacccggagtccttcctggatgtctggcttagtgtagtataccatcaaggacaacctggctctgtctcctaacaagctgctagggccttccctcttgcctaccctcctgacagcctggacctcaggctaacaaacctctgggttgccactgtctccattctgagccatcagaattgtatatacattcatctgctgatgtctctgtatttcaagctgtatgtctttacacctttataatacacagtacagattttgctgcttaaaattctagtatttcctatagacactaaactcaattttaacacaatgaatatgttaaaacattatatatgtgttggattaacacatataatgcaactcatttgttgctaatctatcttggattccttgggtagaatctgtcttctcacctgtctgaaatggaattgaatctcctaacttcttactatttttttaaagccacaaatcccttttaaaatgtccgtttaaggctgctaaactcacattgttatagagtatattttatatacttttcttccttttaaaagatcatatacacttttctcctggcctctaattttgccaaatattctgccataacagcaagctcacagaacacaagtatttgcttaaagatatttaaagcaaatccccaaagagttttctgttgcaaatgtattttaagacataggctgaggagctgagtattcaaatccatgagttctgacactgatgttttattaggcctacccacatgaaaattagtgcttacttgcacagttatcttatttaaaaatacatttgcaaaaagagaataatttcattttcactgattcaaaggtctatcttttcgagtattataaacttacatagtacaaaaagattggtaaaagggatgagaaaaataaagggcaatagtaaggtgagtaaggaaagcaaaattaaacttggagacttgggttctaaatatagcttgtctacatcctagctgcaagatcttaggcaactcaactaacctctctgaacatcacagataaaatggggaataataacattatctagctcaagggtatcgtacaaattatacaatatatgtaaagtgcaaagcatagttcctgcacatagtaagtgctccataagttaaatataataataatcatagtaaaataatattcttaactacaatcttattaaaaggttattacctagagtttaagctgtggggttatttccatttgcacaaagtgtgatagaaatttggctctgtagttcctagcagccaatgtaagagtaaaacaaaaaagtttgtaagtatcaaaagatttatttacttaaatataagaaccctacaataatgcttccaaactagggtttgcagaattaccacaagggctctgtctgtctaaaagtcccccaagcattatctattgttcaaataaatgagttgcacatgtgtgtacttttatttttaaaggcatatagatattgtattaataaaaccaattcatctaattgtattgttgaatttatactttttgccagtaagcattttcctaatagatggacttaactaagaacatcagaatggtgaagagcattgtgttaatacaattcattgtcttatttgttaaaaaagttgaaaaagtattgctgagaaaaaggaaacaagttaacttttgttaaaggtatctcttggttttcactgtcctttccctcatcagtagcgccaagaacatcttaagtcacagaaacattagtttttggaagcagggtttgctgtaactatagtagaaatgacattctgattccactcctagcttcacaaggatatctgtgaaagatttggggcaaaactgttaagctgtctgaaagtgcttttgcataagaaatgggttttactgctaaaactgtcatattgctgagttttgaatgccctaatggtaaatgatactgggttgccaaaaataaccagattagtagttttttcattcatttggccgtctcagtaagtcaaatatt